(19) United States
(12) Patent Application Publication
(10) Pub. No.: US 2024/0287625 A1
(43) Pub. Date: Aug. 29, 2024

(54) PLANT RESISTANCE GENE AND MEANS FOR ITS IDENTIFICATION

(71) Applicant: KWS SAAT SE & Co. KGaA, Einbeck (DE)

(72) Inventors: Ottó TÖRJÉK, Einbeck (DE); Dietrich BORCHARDT, Einbeck (DE); Wolfgang MECHELKE, Einbeck (DE); Britta SCHULZ, Einbeck (DE); Jens Christoph LEIN, Gottingen (DE)

(73) Assignee: KWS SAAT SE & Co. KGaA, Einbeck (DE)

(21) Appl. No.: 18/021,756

(22) PCT Filed: Aug. 5, 2021
(86) PCT No.: PCT/EP2021/071944

§ 371 Date: Feb. 16, 2023

(30) Foreign Application Priority Data
Aug. 17, 2020 (EP) 20191416.5

(51) Int. Cl. C12Q 1/6895; A01H 1/00; A01H 5/06; A01H 5/12; A01H 6/02

(52) U.S. Cl. CPC C12Q 1/6895; A01H 1/1255; A01H 5/06; A01H 5/12; A01H 6/024; A01H 6/028; C12Q 2600/13

(57) ABSTRACT

A more efficient breeding against Cercospora leaf spot disease, or the development of new resistant lines, is enabled via the provision of the Cercospora resistance-mediating gene according to the invention; in particular, a dominant resistance effect in the target plant is evoked by the property of the identified gene alone. The Cercospora resistance-mediating gene, and embodiments of the present invention that are described in the preceding, offer additional applications, e.g., the use of the resistant gene allele in cis-genetic or trans-genetic approaches, with the goal of developing new resistant cultivars.

### CROSS-REFERENCE TO RELATED APPLICATIONS

This application is a U.S. National Phase of International Patent Application No. PCT/EP2021/071944, filed on Aug. 5, 2021, which claims priority to European Application No. 20191416.5, filed Aug. 17, 2020. The entire contents of these applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 15, 2023, is named 245761_000207_SL.txt and is 324,588 bytes in size.

### FIELD OF THE INVENTION

The present invention relates to a nucleic acid molecule which encodes a polypeptide, which nucleic acid molecule is able to confer a resistance to Cercospora—in particular, to the fungus Cercospora beticola in a plant, and, in particular, in a plant of the species Beta vulgaris in which the polypeptide is expressed—as well as to the polypeptide encoded by the nucleic acid molecule according to the invention. In particular, the nucleic acid molecule according to the invention is characterized in that the resistance effect to Cercospora that is conferred by the polypeptide is dominant. Furthermore, the invention relates to a Cercospora-resistant plant, plant cell, plant organ, plant tissue, plant part, or a seed or descendant of a plant, which comprises the nucleic acid molecule or portions thereof as an endogenous gene, as an edited gene, or as a transgene. Furthermore, the present invention also encompasses methods for increasing the resistance to Cercospora in a plant of the species Beta vulgaris, as well as methods for producing or identifying and possibly selecting a Cercospora-resistant plant. The present invention also encompasses methods for monitoring an infestation by the pathogen Cercospora beticola, as well as oligonucleotide probes and primers for hybridization with the nucleic acid molecule according to the invention.

### BACKGROUND OF THE INVENTION

Cercospora leaf spot is one of the most important, globally prevalent leaf diseases of different plants including the species Beta vulgaris and Spinacia oleracea. It is caused by the fungus Cercospora beticola. Plants infested by this disease typically form small, relatively round leaf spots (2-3 mm) that are light gray in the middle and are surrounded by a red-brown border. In a severe infestation, the leaf spots overlap, so that entire portions of the leaf blade dry out. Small black dots (pseudostromata) are visible within the fully formed spots, and a gray, felt-like covering (conidia bearers with conidia) forms under damp conditions—predominantly, on the leaf underside. Severely infested leaves first turn yellow, then turn brown and die. New leaf growth occurs in parallel, wherein the leaves become diseased again and die, however. At first, damage symptoms only on individual plants are visible; however, with spread of the disease, formation of persistent infestation nests often occurs. The further propagation over the entire field takes place via rain and wind. In case of chard plants the damaged leaves are not accepted by the consumers and even a low disease pressure can lead to severe crop shortfall.

The pathogen Cercospora beticola was first described in the second half of the 19th century, in Italy. Up to 40% crop losses may occur due to a severe infestation, which may be triggered by humid weather, early row closure, a high infection potential from previous years, or strong irrigation. These losses result from a reduced beet crop and reduced sugar content; see Holtschulte ((2000) “Cercospora beticola—worldwide distribution and incidence,” pp. 5-16, in “Cercospora beticola Sacc. Biology, Agronomic Influence and Control Measures in Sugar Beet,” vol. 2 (M. J. C. Asher, B. Holtschulte, M. R. Molard, F. Rosso, G. Steinrücken, R. Beckers, eds.). International Institute for Beet Research, Brussels, Belgium, 215 pp.). In order to fight back against the disease, intercropping or fungicides are often used. A chemical control of Cercospora beticola via fungicides incurs costs to the farmer and pollutes the environment. Moreover, the treatment of the edible chard leaves with chemicals reduces consumer acceptance. Repeated applications of fungicides additionally increase the selection pressure on fungicide-tolerant Cercospora beticola strains. This is contrary to a sustainable agricultural practice. It is worth mentioning that during the last few years stems of Cercospora beticola occurred which showed resistance against one or more fungicides; see for Trkulja, Nenad R., et al. “Molecular and experimental evidence of multi-resistance of Cercospora beticola field populations to MBC, DMI and QoI fungicides.” European Journal of Plant Pathology 149.4 (2017): 895-910. The problem became such severe that the German Federal Office of Consumer Protection and Food Safety (BVL) approved the exemptional admission of copper-based fungicides for combating Cercospora. However, copper-based fungicides are generally regarded (depending on the dosage) as harmful for humans and environment. Copper is a heavy metal which may accumulate in the soil.

Indirect combat is done via the selection of cultivars with healthy leaves and cultivation of the beets with at least a 3-year crop rotation. Markedly better control of the infestation may be achieved with a combination of tolerant or resistant cultivars. Less susceptible Cercospora-tolerant beet cultivars have been offered on the market since 2000 (Steinrücken 1997, “Die Züchtung von Cercospora-resistenten Zuckerrüben.” [“The breeding of Cercospora-resistant sugar beets.” ], Vorträge für Pflanzenzüchtung [Lectures on Plant Breeding], Volume 37, Lecture symposium, Mar. 4-5, 1997, Kiel). These cultivars are fumished with a quantitative resistance to Cercospora beticola. The resistance of these cultivars is based upon several genes and is quantitatively passed down, wherein the exact number of the genes that are responsible for the resistance is not known; see Weiland and Koch (2004), Sugarbeet leaf spot disease (Cercospora beticola Sacc.), The Plant Journal, 5(3), 157-166. The complex quantitative heredity was confirmed via several Quantitative Trait Loci (QTL) analyses. This method allows the mapping of polygenic inherited resistances and is a reliable technique for identifying the number and position of genetic resistance factors on the genetic linkage map of a host plant. In this way, multiple causative QTL's could be determined on each chromosome of the sugar beet.

The mappings were performed with different Cercospora-resistance donors, wherein the observed QTL effects were, for the most part, small. The maximum declared phenotypical values were at 5%.

In continuative studies, lists of differentially expressed genes have been described. In a study by Weltmeier et al., ((2011) Transcript profiles in sugar beet genotypes uncover timing and strength of defense reactions to Cercospora beticola infection, Molecular plant-microbe interactions, 24(7), 758-772), a genome-side expression profile for various genotypes of sugar beet (i.e., Cercospora-resistant, -tolerant, -susceptible, etc.) was created with the aid of a microarray-based technology during the pathogen infection in order to analyze transcriptional changes in the expression profile in connection with leaf spot. Via these analyses, the authors were in a position to create a pathogen-induced transcription profile in various tested genotypes of sugar beet and to determine potential candidate genes. However, these genes have not yet been characterized in detail. The genetic and functional background of Cercospora resistance and the identity of the resistance genes have until now been entirely unclear.

However, with the quantitative heredity of QTL, not only is the desired resistance to Cercospora beticola introduced into the plant, but, rather, often unwanted features as well, such as, for example, reduced yield, due to the inheritance of additional genes that are linked with the positive feature of Cercospora resistance. This phenomenon is also known by the term, “linkage drag.” Furthermore, the enormous breeding cost that is required in order to select for multiple resistance loci without thereby reducing the yield may have negative effects on the vitality of the plants; see Weiland and Koch, 2004.

Breeding companies have offered Cercospora-tolerant cultivars on the market for more than a decade. The resistance of these cultivars is inherited via multiple resistance genes with small effect. However, a disadvantage of these cultivars consists in the cultivar development being very laborious and complicated due to the complicated heredity, and in such cultivars having a markedly poorer yield performance relative to normal cultivars, in the absence of an infestation. Among other things, this may be linked to the epigenetic interaction of some resistance genes with genes that are responsible for sugar production, which leads to reduced fitness of the plants, in the absence of the pathogen. Furthermore, Cercospora shows the tendency to overcome the tolerance of long-established cultivars. Moreover, the so far available resistance scores of non-adapted, wild genetic resources is usually not reliable and not comparable among each other as the underlying studies took place at different environment conditions, under different infestation pressure and with different pathogenic stems of Cercospora. In this regard it should me mentioned that environmental parameters like moisture, temperature, wind etc. (which tend to be unstable) have significant influence on the progress of the Cercospora disease after infection. It is common that a specific genetic resource shows high level of tolerance/resistance in one study and tends to be completely susceptible in another study. Due to the above given factors it was so far not possible to identify a dominant resistance gene having a major effect towards Cercospora although there is a strong demand for such a gene which could be easily transferred into already existing cultivars and varieties to establish resistance towards Cercospora.

The use of new breeding techniques based upon gene editing, e.g., by means of TALE nucleases or CRISPR systems, and of transgenic approaches, is not applicable on the so far available genetic material due to the complicated heredity and the multitude of the genes which are involved in the resistance development, the majority of which have not yet been identified and characterized.

For sustainable breeding against Cercospora leaf spot that is to counteract the danger of Cercospora variants that overcome resistance, it is necessary to continuously identify new resistance genes and integrate these into the gene pools of cultivated plants such as sugar beets. In particular, the aim consisted in the provision of suitable resistance genes that, after expression in the plant, on their own already produce a very large, dominant resistance effect against Cercospora beticola.

According to the invention, this aim is achieved via the embodiments characterized in the claims and in the specification.

### ABSTRACT OF THE INVENTION

The present invention relates to a nucleic acid molecule that is able to confer a resistance to Cercospora—in particular, to the fungus Cercospora beticola—in a plant, and, in particular, in Beta vulgaris subsp. vulgaris. The polypeptide which is encoded by the nucleic acid molecule is thereby produced in the plant. The nucleic acid molecule, after whose expression the polypeptide is produced, on its own, already produces in the plant a very large, dominant resistance effect against Cercospora beticola.

Furthermore, the invention relates to a Cercospora-resistant plant, plant cell, plant organ, plant tissue, plant part, a seed, seed stock, or descendant of a plant, which endogenously or transgenically comprises the nucleic acid molecule or portions thereof. According to a specific optional embodiment, those plants and their components that have been obtained via an essentially biological process are excluded.

Methods for increasing the resistance to Cercospora in a plant of the species Beta vulgaris, as well as methods for producing or identifying and possibly selecting a Cercospora-resistant plant, are likewise encompassed by the present invention. The present invention also encompasses methods for monitoring an infestation of the pathogen Cercospora beticola, as well as oligonucleotides as probes and primers for hybridization with the nucleic acid molecule according to the invention.

The present invention therefore relates to the embodiments that are listed in the following points and illustrated in the examples and figures.
[1] Nucleic acid molecule encoding a polypeptide that is able to confer resistance to Cercospora in a plant in which the polypeptide is expressed, characterized in that the nucleic acid molecule comprises a nucleotide sequence which is selected from
   (a) a nucleotide sequence encoding a polypeptide having an amino acid sequence according to SEQ ID No. 3;
   (b) a nucleotide sequence that comprises the DNA sequence according to SEQ ID No. 2;
   (c) a nucleotide sequence that comprises a DNA sequence selected from the group consisting of SEQ ID No. 1 or SEQ ID No. 53;
   (d) a nucleotide sequence that hybridizes to a nucleotide sequence which is complementary to the nucleotide sequence according to (a), (b), or (c), under stringent conditions;
   (e) a nucleotide sequence encoding a polypeptide which, via substitution, deletion, and/or addition of one or more amino acids of the amino acid sequence, differs from a polypeptide encoded by the nucleotide sequence according to (a), (b), or (c);
   (f) a nucleotide sequence encoding a polypeptide which has an amino acid sequence that is at least 70% identical to an amino acid sequence according to SEQ ID No. 3;
   (g) a nucleotide sequence that is at least 70% identical to a DNA sequence according to SEQ ID No. 1 or SEQ ID No. 2;
   wherein the resistance to Cercospora is preferably a resistance to Cercospora beticola, or wherein the plant is preferably a plant of the subspecies Beta vulgaris subsp. vulgaris, and is, particularly preferably, sugar beet.
[2] Nucleic acid molecule according to [1], characterized in that the resistance effect to Cercospora that is conferred by the polypeptide is dominant in the plant—preferably, wherein the polypeptide confers a resistance effect of at least one rating score, and, preferably, of more than one rating score, particularly preferably, of at least two rating scores, particularly preferably, of at least three rating scores, and, especially preferably, of at least four rating scores.
[3] Nucleic acid molecule according to [1] or [2], characterized in that the nucleic acid molecule originates from Beta vulgaris subsp. maritima.
[4] Polypeptide encoded by the nucleic acid molecule according to one of [1] through [3].
[5] Vector or expression cassette comprising the nucleic acid molecule according to one of [1] through [3], wherein the nucleic acid molecule is preferably heterologous to the vector or to the expression cassette.
[6] Cell which comprises the nucleic acid molecule according to one of [1] through [3], or the vector or the expression cassette according to [5], wherein the nucleic acid molecule or the expression cassette are preferably present as an endogene or transgene.
[7] Cercospora-resistant plant or a portion thereof, characterized in that the plant or its portion contains the nucleic acid molecule or the nucleotide sequence according to one of [1] through [3], or the vector or the expression cassette according to [5], wherein the plant which endogenously contains the nucleic acid molecule is a plant of the species Beta vulgaris—but not Beta vulgaris subsp. maritima—or of Beta vulgaris subsp. vulgaris. The nucleic acid molecule or the nucleotide sequence may be contained endogenously or transgenically. Seeds of a plant according to this paragraph may be obtained from a deposition with the NCIMB, Aberdeen, UK under access number NCIMB 43646. The plant according to this paragraph is obtainable from the deposited seeds.
[8] Plant according to [7], characterized in that the plant is a hybrid plant.
[9] Plant according to [7] or [8], characterized in that the nucleic acid molecule is present heterozygously or homozygously in the genome of the plant.
[10] A seed or descendant of the plant according to one of [7] through [9], wherein the seed or the descendant comprises the nucleic acid molecule or the nucleotide sequence according to one of [1] through [3], or the vector or the expression cassette according to [5]. The nucleic acid molecule may be present transgenically or non-transgenically or endogenously. Seeds according to this paragraph are obtainable from a deposition with the NCIMB, Aberdeen, UK under access number NCIMB 43646.
[11] Method for increasing the resistance to Cercospora in a plant, including the following steps:
   (i) integration of the nucleic acid molecule according to one of [1] through [3], or of the vector or of the expression cassette according to [5], by means of homology-directed repair or homologous recombination—preferably, supported by a site-directed nuclease—into the genome of at least one cell of a plant, and optional regeneration of a plant from the at least one plant cell; or
   (ii) increase in the expression of the nucleic acid molecule according to one of [1] through [3] in at least one cell of the plant—preferably, via modification of the native promoter, e.g., comprising a DNA sequence according to SEQ ID No. 7, or via linking of the nucleic acid molecule according to one of [1] through [3] with a heterologous promoter that has a higher level of activity in comparison to the native promoter, e.g., comprising a DNA sequence according to SEQ ID No. 7—in particular, after Cercospora infection—and optional regeneration of a plant from the at least one plant cell; or
   (iii) increase in the activity and/or stability of the polypeptide according to [4] via modification of the nucleotide sequence of the nucleic acid molecule according to one of [1] through [3] in at least one cell of the plant, and optional regeneration of a plant from the at least one plant cell; or
   (iv) transformation of a plant cell with the nucleic acid molecule according to one of [1] through [3], or the vector or the expression cassette according to [5], and optional regeneration of a (transgenic) plant from the transformed plant cell;
   wherein the resistance to Cercospora is preferably a resistance to Cencopora beticola, or the plant is preferably a plant of the species Beta vulgaris—preferably, Beta vulgaris subsp. vulgaris—and, in particular, is sugar beet.
[12] Method for producing a Cercospora-resistant plant according to one of [7] through [9], including the following steps:
   (a) transformation of a plant cell with the nucleic acid molecule according to one of [1] through [3], or the vector or the expression cassette according to [5]; and
   (b) regeneration of the transgenic plant from the transformed plant cell; or
   (i) introduction of a site-directed nuclease and a repair matrix into a cell of a plant of the species Beta vulgaris, wherein the site-directed nuclease is able to generate at least one double-strand break of the DNA in the genome of the cell—preferably, upstream and/or downstream of a target region—and the repair matrix comprises the nucleic acid molecule according to one of [1] through [3];
   (ii) cultivation of the cell from (i) under conditions that allow a homology-directed repair or a homologous recombination, wherein the nucleic acid molecule is incorporated from the repair matrix into the genome of the plant; and
   (iii) regeneration of a plant from the cell modified in (ii).
[13] Method according to [12], characterized in that the target region comprises an allelic variant of the nucleic acid molecule according to one of [1] through [3], wherein the allelic variant encodes a polypeptide not conferring resistance or a slight resistance to Cercospora.
[14] Method according to [12] or [13], characterized in that the at least one double-strand break occurs at a position that is at most 10,000 base pairs upstream and/or downstream of the target region, or that is at most 10,000 base pairs distant from the allelic variant as defined in [13].
[15] Method according to [12] or [13], characterized in that the allelic variant of the nucleic acid molecule comprises a nucleotide sequence which is selected from
   (a) a nucleotide sequence that encodes a polypeptide having an amino acid sequence according to SEQ ID No. 6;
   (b) a nucleotide sequence that comprises the DNA sequence according to SEQ ID No. 5;
   (c) a nucleotide sequence that comprises a DNA sequence according to SEQ ID No. 4;
   (d) a nucleotide sequence that hybridizes to a nucleotide sequence which is complementary to the nucleotide sequence according to (a), (b), or (c), under stringent conditions;
   (e) a nucleotide sequence that encodes a polypeptide which, via substitution, deletion, and/or addition of one or more amino acids of the amino acid sequence, differs from a polypeptide that is encoded by the nucleotide sequence according to (a), (b), or (c); or
   (f) a nucleotide sequence that encodes a polypeptide which has an amino acid sequence that is at least 80% identical to an amino acid sequence according to SEQ ID No. 6.
[16] Plant, or a portion thereof, obtained or obtainable according to a method according to one of [12] through [15].
[17] Method for identifying, and optionally providing, a plant of the species Beta vulgaris that is resistant to Cercospora, characterized in that the method includes at least step (i) or (ii):
   (i) detection of the presence and/or expression of the nucleic acid molecule according to one of [1] through [3], or the presence of the polypeptide according to [4], in the plant or a portion of the plant; and/or
   (ii) detection of at least one marker locus in the nucleotide sequence of the nucleic acid molecule according to one of [1] through [3] or in a cosegregating region; and
   (iii) possible selection of the Cercospora beticola-resistant plant,
   wherein the detection in step (i) or step (ii) is based on the use of at least one molecular marker.
[18] Method for identification of a nucleic acid molecule which encodes a polypeptide that is able to confer a resistance to Cercospora in a plant of the species Beta vulgaris in which the polypeptide is expressed, characterized in that the method includes the following steps:
   (i) comparison of the amino acid sequence of the polypeptide according to [4] with amino acid sequences from a sequence database, or identification of allelic variants which encode the polypeptide according to [4] in genotypes of the species Beta vulgaris;
   (ii) identification of the amino acid sequence, or an allelic variant, encoding an amino acid sequence, wherein the amino acid sequence is at least 80% identical to the amino acid sequence of the polypeptide according to [4];
   (iii) introduction of a nucleic acid molecule, or the allelic variant, encoding the identified amino acid sequence into a plant of the species Beta vulgaris, and expression of the nucleic acid molecule in the plant; and
   (iv) detection of the resistance to Cercospora.
[19] Method for farming of plants of the species Beta vulgaris, including
   (i) the provision of plants according to one of [7] through [9], the planting of a pelleted seed of a sugar beet plant or of a plant of the genus Beta according to one of [26]-[39], the production of plants of the species Beta vulgaris with the aid of a method according to one of [12] through [15], or the identification and selection of plants of the genus Beta with the aid of a method according to [17], and
   (ii) cultivation of the plants from (i) or descendants thereof,
   wherein the method counteracts an infestation of the cultivated plants with Cercospora.
[20] Oligonucleotide of at least 15, 16, 17, 18, 19, or 20—preferably, at least 21, 22, 23, 24, or 25, particularly preferably, at least 30, 35, 40, 45, or 50, and, especially preferably, at least 100, 200, 300, or 500—nucleotides in length, which oligonucleotide hybridizes with a nucleotide sequence as defined in one of [1] through [3].
[21] A pair of oligonucleotides—preferably, oligonucleotides according to [20] or a kit containing these oligonucleotides—wherein the oligonucleotides are suitable for hybridization as forward primer and reverse primer to a region in the Beta vulgaris genome that, in Beta vulgaris, has a cosegregation with the Cercospora resistance conferred by the polypeptide according to [4], or with the nucleic acid molecule according to one of [1] through [3].
[22] Use of the nucleic acid molecule according to one of [1] through [3] in the production of Cercospora-resistant plants of the subspecies Beta vulgaris subsp. vulgaris.
[23] Method for the production of an organism which comprises a mutated version according to [1] and/or a mutated version of a promoter comprising a nucleic acid sequence selected from
   (a) SEQ ID NO: 7
   (b) a nucleotide sequence, which hybridizes under stringent conditions with a sequence which is complementary to the sequence according to (a)
   (c) a nucleotide sequence which is at least 70% identical to a sequence according to SEQ ID NO: 7
wherein the method includes the following steps:
(I) Provision of an organism or a cell comprising the nucleic acid molecule and/or the promoter
(II) Increase of the mutation rate of the organism or the cell or mutagenesis of the organism or the cell
(III) Phenotypic selection of an organism, which as a result of a mutation exhibits an altered resistance or altered resistance level towards Cercospora beticola or Genotypic selection of an organism or a cell which comprises a mutation in the nucleic acid molecule and/or the promoter wherein the mutation has been created via step (II) and optionally
(IV) Regeneration of the organism from the cell obtained via step (III).
[24] Method according to [23], wherein the organism is a plant.
[25] Method according to [24] wherein the plant is a Beta vulgaris, preferably a Beta vulgaris subsp. vulgaris, more preferably a sugar beet or red beet.
[26] A pelleted seed of a sugar beet plant or a plant of the genus Beta including red beet comprising a nucleic acid molecule according to [1].
[27] The pelleted seed according to [26], wherein the beet body is suitable as raw material for industrial sugar production or for consumption as food.
[28] The pelleted seed according to [26] or [27], wherein the pelleted seed is a monogerm seed.
[29] The pelleted seed according to [26] to [28], wherein the sugar beet plant is harvestable before bolting.
[30] The pelleted seed according to [26] to [29], wherein the resistance to Cercospora is a resistance to Cercospora beticola.
[31] The pelleted seed according to [26] to [30], wherein the sugar beet plant is biannual.
[32] The pelleted seed according to [26] to [31] and [124], which has been technically treated, wherein the technical treatment is selected from the group consisting of:
   (a) polishing;
   (b) dressing;
   (c) incrustation; and
   (d) coloring.
[33] The pelleted seed according to [26] to [32], wherein the pellet comprises at least one chemical selected from the group selected of:
   (a) insecticide;
   (b) fungicide; and
   (c) fertilizer.
[34] The pelleted seed according to [26] to [33], wherein the seed has been subjected to priming or pre-germination before or during pelleting.
[35] The pelleted seed according to [26] to [34], wherein the sugar beet plant is a hybrid sugar beet plant.
[36] The pelleted seed according to [26] to [35] wherein the nucleotide sequence includes at least one mutation.
[37] The pelleted seed according to [36] wherein the at least one mutation is a mutation relative to SEQ ID No. 1 or SEQ ID No 2.
[38] The pelleted seed according to [36] or [37] wherein the nucleotide sequence including the at least one mutation encodes a polypeptide which has an amino acid sequence that is at least 99% identical to an amino acid sequence according to SEQ ID No. 3.
[39] The pelleted seed according to [38] wherein the nucleotide sequence including the at least one mutation encodes a polypeptide having an amino acid sequence according to SEQ ID No. 3.
[40] A packing containing the pelleted seed according to [26] to [39] or containing seed stock comprising the nucleic acid molecule according to [1] wherein the seed stock preferable is seed stock of a plant of the genus Beta.
[41] A mixture of a pelleting mass and a sugar beet plant seed wherein the sugar beet plant seed comprises a nucleic acid sequence which encodes a polypeptide that is able to confer resistance to Cercospora, wherein the nucleotide sequence is selected from the group consisting of
   (a) a nucleotide sequence encoding a polypeptide having an amino acid sequence according to SEQ ID No. 3;
   (b) a nucleotide sequence that comprises the DNA sequence according to SEQ ID No. 2;
   (c) a nucleotide sequence that comprises a DNA sequence selected from the group consisting of SEQ ID No. 1 or SEQ ID No. 53;
   (d) a nucleotide sequence that hybridizes to a nucleotide sequence which is complementary to the nucleotide sequence according to (a), (b), or (c), under stringent conditions;
   (e) a nucleotide sequence encoding a polypeptide which, via substitution, deletion, and/or addition of one or more amino acids of the amino acid sequence, differs from a polypeptide encoded by the nucleotide sequence according to (a), (b), or (c);
   (f) a nucleotide sequence encoding a polypeptide which has an amino acid sequence that is at least 70% identical to an amino acid sequence according to SEQ ID No. 3;
   (g) a nucleotide sequence that is at least 70% identical to a DNA sequence according to SEQ ID No. 1 or SEQ ID No. 2.
[42] A method for producing the pelleted sugar beet plant seed according to [26] to [39] comprising the following steps:
   a) providing a sugar beet plant seed comprising a nucleic acid sequence which encodes a polypeptide that is able to confer resistance to Cercospora,
   wherein the nucleotide sequence which is selected from the group consisting of
   (i) a nucleotide sequence that encodes a polypeptide which has an amino acid sequence that is at least 95% identical to an amino acid sequence according to SEQ ID No. 3; and
   (ii) a nucleotide sequence that is at least 95% identical to a DNA sequence according to SEQ ID No. 1 or SEQ ID No. 2
   b) embedding the sugar beet plant seed in a pelleting mass
   c) allow the pelleting mass to dry or dry the pelleting mass.
[43] The pelleted seed according to [36] to [39] wherein the nucleotide sequence including the at least one mutation is an artificial nucleotide sequence which does not occur naturally.
[44] The method according to [23] wherein during step (11) a mutagenizing chemical like EMS or mutagenizing radiation is applied.
[45] A variety or cultivar of the genus Beta comprising the nucleic acid molecule according to [1] or a pelleted seed the variety or cultivar.
[46] A leaf of a swiss chard plant comprising the Nucleic acid molecule according to [1].
[47] A bag—preferably a plastic bag—comprising one or more chard plant leaves according to [46].
[48] A method for identifying a plant, preferably a plant of the species Beta vulgaris or a plant of the subspecies Beta vulgaris subrp. vulgaris, that is resistant or tolerant to Cercospora, characterized in that the method includes the following steps:
   (i) detection of the presence and/or expression of the nucleic acid molecule according to [1] or the nucleotide sequence as defined in [1], or the presence of the polypeptide which is encoded by the nucleic acid molecule according to [1] or of the nucleotide sequence as defined in [1], in the plant or a portion of the plant; and/or
   (ii) detection of at least one marker locus in the nucleotide sequence of the nucleic acid molecule according [1] or in a cosegregating region,
   wherein the cosegregating region is a genomic region which cosegregates with the Cercospora resistance conferred by the polypeptide, or with the nucleic acid molecule or the nucleotide sequence and wherein the cosegregating region comprises and is flanked preferably by marker s4p1395s01 and s4p0421s01. More preferably the cosegregating region comprises and is flanked by a marker pair selected from the list consisting of: sxi0123s02 and s4p0238s01, s4p1396s01 and sxh1195s02, s4p1398s01 and s4p0234s01, s4p1462s01 and s4p0232s01, s4p1464s01 and s4p0323s01, s4p0044s01 and s4p0322s01, s4p0056s01 and s4p0320s01, s4p0058s01 and sxh0942s04, s4p0059s01 and sxh1834s05, s4p1564s01 and s4p0317s01, s4p1998s01 and s4p4308s01, s4p1343s01 and s4p4305d01, s4p1408s01 and sxh0678s01, s4p1565s01 and s4p4301s01, s4p1409s01 and s4p8772s01, s4p1411s01 and s4p4295s01. Most preferably the cosegregating region comprises and is flanked by a marker pair selected from the list consisting of: s4p1343s01 and s4p0421s01, s4p1408s01 and s4p0238s01, s4p1565s01 and sxh1195902 s4p1409s01 and s4p0234s01, s4p1411s01 and s4p0232s01, s4p1414901 and s4p0323s0l, s4p1485s01 and s4p0322s01, s4p0257s01 and s4p0320s01, s4p0258s01 and sxh0942s04, s4p0260s01 and sxh1834s05, sxh0876s05 and s4p0317s01, s4p0262s01 and s4p4308s01, s4p0263s01 and s4p4305d01. For mapping purposes the following marker pairs are recommended. s4p4293s01 and s4p8772s01, preferably 94p4295s01 and s4p8772s01. The structural features of the markers are given in Table 1B. The method may involve one or more of the following steps:
      Provision of at least one plant, its tissue, a seed or at least one cell thereof
      Extraction of DNA, preferably genomic DNA from the at least one plant, its tissue, the seed or from the at least one cell thereof and
      Performing the detection on the extracted DNA
      Selection of the Cercospora-resistant plant.
[49] The method according to anyone of [48] or [104]-[113] characterized in that the method includes a further step:
   creating electronically transmittable and/or electronically storable data representing the detection of the presence of the nucleic acid molecule or the nucleotide sequence.
[50] The method according to [49] characterized in that the method includes a further step:
   storing the data on a computer readable medium.
[51] The method according to one of [48] to [50] wherein the markers are molecular markers and/or diagnostic markers.
[52] A process of identifying a plant that displays resistance or tolerance to Cercospora, the process comprising detecting in the plant at least one polymorphism—preferably a single nucleotide polymorphism—by at least one marker, wherein the at least one marker is within a chromosomal interval flanked by the two markers s4p1395s01 and 94p0421s01 or within a chromosomal interval flanked by a pair of markers disclosed in [48] and wherein the plant comprises the nucleic acid molecule or the nucleotide sequence according to [1]. The markers may be diagnostic and/or molecular markers. The process may involve one or more of the following steps:
   Provision of at least one plant, its tissue, a seed or at least one cell thereof
   Extraction of DNA, preferably genomic DNA from the at least one plant, its tissue, the seed or from the at least one cell thereof
   Performing the detection on the extracted DNA and
   Selection of the Cercospora-resistant plant.
[53] The process according to [52] wherein
   (i) said at least one single nucleotide polymorphism is genetically linked to the nucleic acid molecule or nucleotide sequence according to [1] or has a recombination frequency of about 10%, preferably 5%, more preferably 1% or less to the nucleic acid molecule or nucleotide sequence according to [1], or
   (ii) said at least one single nucleotide polymorphism is located within 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less of the nucleic acid molecule or the nucleotide sequence according to [1].
[54] The process or method according to anyone of [48], [52], [104]-[113] wherein the plant displays increased resistance or tolerance to Cercospora, compared to a plant lacking the nucleic acid molecule or nucleotide sequence of [1]
[55] The process according to anyone of [52]-[54], [57]-[70], [106]-[113] wherein the single nucleotide polymorphism is a nucleotide the presence of which is genetically and/or statistically linked to the presence of the nucleic acid molecule or the nucleotide sequence according to [1].
[56] The process according to [52]-[55] wherein the at least one marker is a marker given in Table 1b or the at least one marker is a marker pair as disclosed in [48].
[57] A process of identifying a plant that is resistant or tolerant to Cercospora, the process comprising detecting in the plant or in the DNA of the plant at least two single nucleotide polymorphisms by at least two markers, wherein at least one of said markers is on or within a first chromosomal interval flanked by a marker selected from the group consisting of: s4p1395s01, sxi0123s02, s4p1396s01, s4p1398s01, s4p1462s01, s4p1464s01, s4p0044s01, s4p0056s01, s4p0058s01, s4p0059s01, s4p1564s01, s4p1998s01, s4p1343s01, s4p1408s01, s4p1565s01, s4p1409s01, s4p1411s01, s4p1414s01, s4p1485s01, s4p0257s01, s4p0258s01, s4p0260s01, sxh0876s05, s4p0262s01, s4p0263s01, s4p0264s01, s4p2271s01, s4p4288s01, s4p4290d01, s4p4293s01
   and the nucleotide sequence according to [1], and at least one of said markers is on or within a second chromosomal interval flanked by the polynucleotide of claim 1 and a marker selected from the group consisting of:
   s4p0421s01, s4p0238s01, sxh1195s02, s4p0234s01, s4p0232s01, s4p0323s01, s4p0322s01, s4p0320s01, sxh0942s04, sxh1834s05, s4p0317s01, s4p4308s01, s4p4305d01.
[58] The process according to [57] wherein the at least two markers and the nucleotide sequence according to [1] flank the first and second chromosomal interval without being part of the first or second chromosomal interval.
[59] The process according to [57] or [58] wherein the at least two markers are each located within 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less of the nucleic acid molecule or the nucleotide sequence according to [1].
[60] The process according to [57]-[59] wherein at least one marker is located on a genomic region having an identity of at least 95%, at least 96%, at least 97%, at least 98%, at least 99% or 100% to SEQ ID No. 74 or SEQ ID No. 75, wherein the identity is preferably over the whole length of SEQ ID No. 74 or SEQ ID No. 75.
[61] The process according to [57]-[60] wherein one of the at least two markers is located on the genomic region having an identity of at least 95%, at least 96%, at least 97%, at least 98%, at least 99% or 100% to SEQ ID No. 74 and one of the at least two markers is located on the genomic region having an identity of at least 95%, at least 96%, at least 97%, at least 98%, at least 99% or 100% to SEQ ID No. 75, wherein the identity is preferably over the whole length of SEQ ID No. 74 or SEQ ID No. 75.
[62] The process according to [57]-[61] wherein at least one of the at least two markers is located within 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less of the nucleic acid molecule or the nucleotide sequence according to [1].
[63] The process according to [57]-[62] wherein each of the at least two markers is located within 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less of the nucleic acid molecule or the nucleotide sequence according to [1].
[64] The process according to [57]-[63] involving the detection of at least two single nucleotide polymorphisms in the first chromosomal interval and at least two single nucleotide polymorphisms in the second chromosomal interval wherein each single nucleotide polymorphism is detected by a different marker.
[65] The process according to [57]-[64] including the following steps
   Provision of at least one plant, its tissue, a seed or at least one cell thereof
   Extraction of DNA, preferably genomic DNA from the at least one plant, its tissue, a seed or from the at least one cell thereof and
   Performing the detection step as defined in any of [57]-[64] on the extracted DNA.
[66] The process or method according to [48]-[65] wherein the detection step includes a polymerase chain reaction (PCR).
[67] The process or method according to [48]-[65] or [112] or [113] involving a polymerase chain reaction (PCR) wherein the PCR involves two allele-specific forward primers (or their use) and wherein the detection step involves fluorescence resonant energy transfer (FRET) wherein the presence, absence or kind of the fluorescence is determined by a sensor. The sensor signal may be turned into electronically transmittable and/or electronically storable data representing the detection of the presence of the nucleic acid molecule or the nucleotide sequence according to [1]. In addition, the electronically transmittable and/or electronically storable data may be stored on a computer readable medium. The kind of the fluorescence may be its color/wave length or the specific dye responsible for the fluorescence (like FAM or HEX).
[68] The process according to [67] furthermore involving one common reverse primer or involving the use of one common reverse primer. Examples for common primers are given in Tab. 1b.
[69] The process according to anyone of [67], [68] [112] and [113] wherein the determination of the fluorescence by the sensor is an end-point fluorescent read.
[70] The process according to anyone of [67] or [69], [112] and [113] wherein each of the two allele-specific forward primers comprise a unique tail sequence that undergoes chemical binding with a specific FRET cassette during the PCR.
[71] A method for identifying a plant, preferably a plant of the species Beta vulgaris or a plant of the subspecies Beta vulgaris subsp. vulgaris, that is resistant or tolerant to Cercospora, characterized in that the method includes the following steps:
   Provision of at least one plant, its tissue, seed or at least one cell thereof
   Extraction of DNA, preferably genomic DNA from the at least one plant, its tissue or from the at least one cell thereof and
   Detecting the presence or absence of the nucleic acid molecule or the nucleotide sequence according to [1] in the extracted DNA, preferably by a marker as given in Tab. 1b or by a marker pair as disclosed in [48], by a pair of oligonucleotides according to [20], [21], [72] or [73] or [76], a set of three oligonucleotides according to [74] or [75] or a molecular marker or a set of molecular markers according to [84].
   Optionally selecting the plant comprising the nucleic acid molecule or the nucleotide sequence according to [1] in the extracted DNA.
[72] A pair of oligonucleotides suitable for use as primer in a PCR which are able to hybridize to a genomic interval which comprises and is flanked by marker s4p1395s01 and s4p0421s01.

Preferably the genomic interval comprises and is flanked by a marker pair selected from the list consisting of: sxi0123s02 and s4p0238s01, s4p1396s01 and sxh1195s02, s4p1398s01 and s4p0234s01, s4p1462s01 and s4p0232s01, s4p1464s01 and s4p0323s01, s4p0044s01 and s4p0322s01, s4p0056s01 and s4p0320s01, s4p0058s01 and sxh0942s04, s4p0059s01 and sxh1834s05, s4p1564s01 and s4p0317s01, s4p1998s01 and s4p4308s01, s4p1343s01 and s4p4305d01, s4p1408s01 and sxh0678s01, s4p1565s01 and s4p4301s01, s4p1409s01 and s4p8772s01, s4p1411s01 and s4p4295s01. Most preferably the genomic interval comprises and is flanked by a marker pair selected from the list consisting of: s4p1343s01 and s4p0421s01, s4p1408s01 and s4p0238s01, s4p1565s01 and sxh1195s02 s4p1409s01 and s4p0234s01, s4p1411s01 and s4p0232s01, s4p1414s01 and 94p0323s01, s4p1485s01 and s4p0322s01, s4p0257s01 and s4p0320s01, s4p0258s01 and sxh0942s04, s4p0260s01 and sxh1834s05, sxh0876s05 and s4p0317s01, s4p0262s01 and s4p4308s01, s4p0263s01 and s4p4305d01. The genomic interval may be the genomic interval of a plant of the species Beta vulgaris or subspecies Beta vulgaris subsp. vulgaris.
[73] A pair of oligonucleotides suitable for use as primer in a PCR which are able to hybridize to the nucleic acid sequence according to SEQ ID No. 74 or SEQ ID No. 75 or to a sequence that is at least 99% identical to the sequence according to SEQ ID No. 74 or SEQ ID No. 75 over the whole length of the sequence according to SEQ ID No. 74 or SEQ ID No. 75.
[74] A set of three oligonucleotides suitable for use as primer in a PCR involving two forward primers and a reverse primer wherein each primer has a different nucleotide sequence and wherein the reverse primer and only one of the two forward primers are able to hybridize to a genomic interval which comprises and is flanked by marker s4p1395s01 and s4p0421s01. Preferably the genomic interval comprises and is flanked by a marker pair selected from the list consisting of: sxi0123s02 and s4p0238s01, s4p1396s01 and sxh1195s02, s4p1398s01 and s4p0234s01, s4p1462s01 and s4p0232s01, s4p1464s01 and s4p0323s01, s4p0044s01 and s4p0322s01, s4p0056s01 and s4p0320s01, s4p0058s01 and sxh0942s04, s4p0059s01 and sxh1834s05, s4p1564s01 and s4p0317s01, s4p1998s01 and s4p4308s01, s4p1343s01 and s4p4305d01, s4p1408s01 and sxh0678s01, s4p1565s01 and s4p4301s01, s4p1409s01 and s4p8772s01, s4p1411s01 and s4p4295s01. Most preferably the genomic interval comprises and is flanked by a marker pair selected from the list consisting of: s4p1343s01 and s4p0421s01, s4p1408s01 and s4p0238s01, s4p1565s01 and sxh1195s02 s4p1409s01 and s4p0234s01, s4p1411s01 and s4p0232s01, s4p1414s01 and s4p0323s01, s4p1485s01 and s4p0322s01, s4p0257s01 and s4p0320s01, s4p0258s01 and sxh0942s04, s4p0260s01 and sxh1834s05, sxh0876s05 and s4p0317s01, s4p0262s01 and s4p4308s01, s4p0263s01 and s4p4305d01.
[75] A set of three oligonucleotides suitable for use as primer in a PCR involving two forward primers and a reverse primer wherein each primer has a different nucleotide sequence and wherein the reverse primer and only one of the two forward primers are able to hybridize to a sequence according to SEQ ID No. 74 or SEQ ID No. 75 or to a sequence that is at least 99% identical to the sequence according to SEQ ID No. 74 or SEQ ID No. 75 over the whole length of the sequence according to SEQ ID No. 74 or SEQ ID No. 75.
[76] The pair or set of oligonucleotides according to [72]-[75] wherein each oligonucleotide comprises or consists of 15-40 nucleotides, preferably 17-30 nucleotides, more preferably 19-25 nucleotides.
[77] A process for the quantification of the level of resistance or tolerance of a plant to Cercospora including the following steps:
   Providing at least one first plant or at least one seed of the first plant comprising the nucleic acid molecule or the nucleotide sequence according to [1] and at least one second plant or at least one seed of the at least second plant lacking the nucleic acid molecule or the nucleotide sequence according to [1]
   Cultivate the plants or allow the seeds to germinate and cultivate the germinated plants wherein a) the conditions of the cultivation allow the interaction with Cercospora or b) the plants are inoculated with Cercospora
   Determine the infested surface of at least one leaf of each of the two plants.
   Optionally compare the level of resistance or tolerance of the first plant with the level of resistance or tolerance of the second plant and/or determine a rating score for the first and or second plant selected from Table 1A.
[78] The process according to [77] wherein the cultivation step is replaced by the inoculation of a leaf of the first plant and a leaf of the second plant with or in a solution comprising Cercospora.
[79] A method for reducing the application of fungicidal agrochemicals including the following steps:
   I) Provide seeds or seed stock comprising the nucleic acid molecule or the nucleotide sequence according to [1].
   II) Plant or drill the seeds or the seed stock to allow the germination of the seeds or the seed stock.
   III) Cultivate the germinated plants derived by step II)
   IV) Harvest the plants cultivated during step III) or harvest plant parts like storage organs, taproots or harvest seeds of the plants cultivated during step III)
   wherein the application of fungicidal agrochemicals during step II) and or step II) is reduced or avoided.
[80] The method according to [79] wherein the fungicidal agrochemical is an agrochemical which is effective against Cercospora wherein these agrochemicals may include those which contain one or more of the following fungicides: epoxiconazole, kresoxim-methyl, thiophanate methyl, mancozeb and those fungicides effective against Cercospora mentioned elsewhere herein.
[81] The method according to [79] or [80] wherein the application of fungicidal agrochemicals during step II) and or step III) is reduced or avoided in comparison to the cultivation ofplants lacking the nucleic acid molecule according to [1] under the same or comparable conditions as the plants in step III).
[82] Use of a marker or a molecular marker for the identification and/or selection of a plant comprising the nucleic acid molecule or nucleotide sequence according to [1].
[83] The use according to [82] wherein the marker or molecular marker is used in a process or method according to [48]-[71].
[84] A molecular marker or a set of molecular markers—preferably encompassing two or three molecular markers—suitable for use in a process or method according to [48]-[71].
[85] A method for the expression of the nucleic acid molecule or nucleotide sequence according to [1] comprising the following steps:
   i) Introduction of the nucleic acid molecule or nucleotide sequence according to [1] in one or more cells
   ii) Cultivating the one or more cells under conditions which allow the proliferation of the one or more cells and/or which allow the expression of the nucleic acid molecule or nucleotide sequence according to [1]
[86] The method according to [85] wherein the method is a method for in vitno expression.
[87] The method according to [85] or [86] wherein the introduction in step i) is by transformation, transfection, electroporation or infiltration.
[88] The method according to [85]-[87] wherein the introduction of the nucleic acid molecule or nucleotide sequence is the introduction of a vector, plasmid or transfer DNA (tDNA).
[89] The method according to [85]-[88] wherein the nucleic acid molecule or nucleotide sequence is part of an expression cassette and is operably linked to a promoter which is active, partly active or inducible in the one or more cells of i). The expression cassette may be the expression cassette according to [5].
[90] The method according to [89] wherein the promoter has at least 95% sequence identity to the 35S promoter derived from Cauliflower mosaic virus or is the 35S promoter derived from Cauliflower mosaic virus.
[91] The method according to [90] wherein the 35S promoter derived from Caulhflower mosaic virus comprises or consists of a nucleic acid sequence according to SEQ ID No. 225.
[92] The method according to [85]-[91] wherein the one or more cells are plant cells.
[93] The method according to [85]-[89] wherein the one or more cells are microorganisms preferably bacteria or fungal cells like yeast.
[94] The plant according to [7]-[9] characterized in that the plant furthermore comprises a nucleic acid molecule which encodes a polypeptide that is able to convey a resistance to the pathogen Beet Necrotic Yellow Vein Virus BNYVV in a plant in which the polypeptide is expressed, characterised in that the nucleic acid molecule comprises a nucleotide sequence selected from
   a) a nucleotide sequence that encodes a polypeptide having an amino acid sequence according to SEQ ID NO: 227 or SEQ ID NO: 228,
   b) a nucleotide sequence comprising the encoding sequence of the DNA sequence according to SEQ ID NO: 226,
   c) a nucleotide sequence that hybridises with the complementary sequence of the nucleotide sequence according to a) or b) under stringent conditions,
   d) a nucleotide sequence that encodes a polypeptide which is derived by substitution, deletion and/or addition of one or more amino acids of the amino acid sequence encoded by the nucleotide sequence according to a) or b) from a polypeptide encoded by the nucleotide sequence according to a) or b),
   e) a nucleotide sequence that encodes a polypeptide which has an amino acid sequence at least 90%, preferably at least 95%, most preferably 99% identical to an amino acid sequence encoded by the nucleotide sequence according to a) or b), or
   f) a nucleotide sequence that encodes at least one nucleotide-binding domain (NBS) corresponding to amino acid positions 168-227 of SEQ ID NO: 227 or corresponding to amino acid positions 182-241 of SEQ ID NO: 228, at least one leucine-rich domain (LRR) corresponding to amino acid positions 591-613 of SEQ ID NO: 227 or corresponding to amino acid positions 605-627 of SEQ ID NO: 228 and/or at least one internal repetitive domain (IR) corresponding to amino acid positions 1013-1072 of SEQ ID NO: 227 or corresponding to amino acid positions 1027-1086 of SEQ ID NO: 228.
[95] The plant according to [7]-[9] wherein the plant is obtainable from the seeds deposited with the NCIMB, Aberdeen, UK under access number NCIMB 43646.
[96] The seed or descendent according to [10] wherein the seed or descendent is obtainable from the deposition with the NCIMB, Aberdeen, UK under access number NCMB 43646.
[97] The plant according to [7]-[9], [94] and [95] furthermore characterized in that the plant comprises SEQ ID NO. 182 or is detectable by marker s4p8772s01.
[98] A process for the breeding of a Cercospora resistant plant of the species Beta vulgaris including the following steps:
   i) Obtaining seeds from the deposition with the NCIMB, Aberdeen, UK under access number NCIMB 43646 or offspring of the deposited seeds.
   ii) Cultivate the seeds of step i) under conditions which allow the growth of plants
   iii) Cross the plants resulting from step ii) with a plant of the species Beta vulgaris. The method may optionally include a further step of
   iv) Determine the presence or absence of the nucleic acid molecule or the nucleotide sequence according to [1] in the offspring resulting from step iii) by a method or process described herein like for example a method or process according to [48]-[71].
[99] Use of electronically transmittable and/or electronically storable data derived from a process according to anyone of [49], [50], [69], [70] and [104]-[113] for determining the estimated breeding value of a plant in a population of plants.
[100] The use according to [99] wherein the estimated breeding value is at least partly dependent on Cercmpora resistance and wherein the population of plants comprises 1000 or less plants.
[101] The plant according to one of [7]-[9] or a seed or descendant of the plant according to [10] wherein the nucleic acid molecule or the nucleotide sequence according to [1] is comprised or integrated as an introgression.
[102] A storage organ of the plant according to one of [7]-[9] or [101]. The storage organ may be a taproot, especially a beet body.
[103] A leaf or leaves of the plant according to one of [7]-[9] or [101], wherein the plant may be a swiss chard or a plant of the genus Spinacia.
[104] A method for identifying a plant that is resistant or tolerant to Cercospora, characterized in that the method includes the following steps:
   (i) detection of the presence or absence of a nucleotide sequence selected from the group consisting of
      (a) a nucleotide sequence that encodes a polypeptide having an amino acid sequence according to SEQ ID No. 3;
      (b) a nucleotide sequence that comprises the DNA sequence according to SEQ ID No. 2;
      (c) a nucleotide sequence that comprises a DNA sequence according to SEQ ID No. 1 or SEQ ID No. 53;
      (d) a nucleotide sequence that hybridizes with a complementary sequence of the nucleotide sequence according to (a), (b), or (c) in 4×SSC (Saline-Sodium Citrate) at 65° C., and subsequent repeated washing in 0.1×SSC at 65° C. for approximately 1 hour in total;
      (e) a nucleotide sequence that encodes a polypeptide which has an amino acid sequence that is at least 90% identical to an amino acid sequence according to SEQ ID No. 3;
      (f) a nucleotide sequence that is at least 90% identical to a DNA sequence according to SEQ ID No. 1 or SEQ ID No. 2;
   (ii) detection of the presence or absence of the polypeptide which is encoded by the nucleotide sequence as defined in step (i) in the plant or a portion of the plant; and/or
   (iii) detection of at least one marker locus in the nucleotide sequence as defined in step (1) or in a cosegregating region,
   wherein the cosegregating region is a genomic region which cosegregates with the Cercospora resistance conferred by the polypeptide, or with the nucleic acid molecule or the nucleotide sequence and wherein the cosegregating region comprises and is flanked by marker s4p1395s01 and s4p0421s01.
[105] The method according to [104], characterized in that the method includes the further step of:
   (iv) selection of the Cercospora-resistant plant.
[106] The method according to [104] or [105] wherein the detection in step (i) or (iii) is based on at least one polymorphism or a single nucleotide polymorphism.
[107] The method according to [106] furthermore characterized in that
   a) said at least one polymorphism or single nucleotide polymorphism is genetically linked to the nucleotide sequence or has a recombination frequency of 10% or less to the nucleotide sequence,
   b) said at least one polymorphism or single nucleotide polymorphism is located within 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less of the nucleotide sequence,
   c) said at least one polymorphism or single nucleotide polymorphism is detectable in the seeds deposited with the NCIMB, Aberdeen, UK under access number NCIMB 43646 or
   d) said at least one polymorphism or single nucleotide polymorphism is part of the cosegregating region,
   wherein the nucleotide sequence is the nucleotide sequence defined in step (i) of [104] and wherein the cosegregating region is the cosegregating region defined in step (iii) of [104].
[108] The method according to anyone of [104]-[107] wherein step (i) and/or step (iii) furthermore includes the following steps which are concluded by the detection:
   a) Provision of at least one plant, its tissue, a seed or at least one cell thereof and
   b) Extraction of DNA, preferably genomic DNA from the at least one plant, its tissue, the seed or from the at least one cell thereof.
   This means for example:
   A method for identifying a plant that is resistant or tolerant to Cercospora, characterized in that the method includes the following steps:
      ia) Provision of at least one plant, its tissue, a seed or at least one cell thereof and
      ib) Extraction of DNA, preferably genomic DNA from the at least one plant, its tissue, the seed or from the at least one cell thereof and
      ic) detection of the presence or absence of a nucleotide sequence as defined in step (i) of [104]
      ii) detection of the presence or absence of the polypeptide which is encoded by the nucleotide sequence as defined in step (i) in the plant or a portion of the plant; and/or
      iiia) Provision of at least one plant, its tissue, a seed or at least one cell thereof and
      iiib) Extraction of DNA, preferably genomic DNA from the at least one plant, its tissue, the seed or from the at least one cell thereof and
      iiic) detection of at least one marker locus in the nucleotide sequence as defined in step (1) or in a cosegregating region,
   wherein the cosegregating region is a genomic region which cosegregates with the Cercospora resistance conferred by the polypeptide, or with the nucleic acid molecule or the nucleotide sequence and wherein the cosegregating region comprises and is flanked by marker s4p1395s01 and s4p0421s01.
[109] The method according to [104]-[108] involving the use of at least two oligonucleotides.
[110] The method according to [109] wherein the oligonucleotides are suitable for use as primer in a PCR and are able to hybridize to a genomic interval which comprises and is flanked by marker s4p1395s01 and s4p0421s01 or by a marker pair as disclosed in [48]. Preferably the oligonucleotides may hybridize during a PCR with a genomic template comprising the nucleotide sequence as defined in step (i) of [104] in such a way/in such a distance that the resulting amplificate is comprises up to 2000 bp, preferably up to 1500 bp, more preferably 1000 bp, most preferably up to 500 or 200 or up to 100 bp.
[111] The method according to anyone of the preceding claims wherein the plant is a plant of the genus Beta, Spinacia, Glycine, Daucus or Pastinaca.
[112] The method according to anyone of [104]-[111] involving a PCR in step (i) and/or (iii) wherein the PCR involves two allele-specific forward primers and wherein the detection step involves fluorescence resonant energy transfer and wherein the presence, absence or kind of the fluorescence is determined by a sensor wherein optionally a sensor signal is produced. The sensor signal may be turned into electronically transmittable and/or electronically storable data representing the detection of the presence of the nucleic acid molecule or the nucleotide sequence according to [1]. In addition, the electronically transmittable and/or electronically storable data may be stored on a computer readable medium. The determination of the fluorescence by the sensor may be an end-point fluorescent read.
[113] The method according to [112] involving furthermore one common reverse primer. Preferably the reverse primer and the allele-specific forward primers may hybridize during a PCR with a genomic template comprising the nucleotide sequence as defined in step (i) of [104] in such a way/in such a distance that the resulting amplificate is comprises up to 2000 bp, preferably up to 1500 bp, more preferably 1000 bp, most preferably up to 500 or 200 or up to 100 bp. The primer may be oligonucleotides for example the oligonucleotides mentioned in [109].
[114] A plant of the genus Beta, Spinacia, Glycine, Daucus or Pastinaca comprising a nucleic acid molecule encoding a polypeptide that is able to confer resistance to Cercospora in a plant in which the polypeptide is expressed, characterized in that the nucleic acid molecule comprises a nucleotide sequence which is selected from the group consisting of:
   (a) a nucleotide sequence that encodes a polypeptide having an amino acid sequence according to SEQ ID No. 3;
   (b) a nucleotide sequence that comprises the DNA sequence according to SEQ ID No. 2;
   (c) a nucleotide sequence that comprises a DNA sequence according to SEQ ID No. 1 or SEQ ID No. 53;
   (d) a nucleotide sequence that hybridizes with a complementary sequence of the nucleotide sequence according to (a), (b), or (c) in 4×SSC at 65° C., and subsequent repeated washing in 0.1×SSC at 65° C. for approximately 1 hour in total;
   (e) a nucleotide sequence that encodes a polypeptide which has an amino acid sequence that is at least 90% identical to an amino acid sequence according to SEQ ID No. 3;
   (f) a nucleotide sequence that is at least 90% identical to a DNA sequence according to SEQ ID No. 1 or SEQ ID No. 2;
   wherein the plant of the genus Beta or Spinacia furthermore comprises an endogenous allele encoding an epsp synthase having at position 179 an amino acid different from proline. According to a specific embodiment this epsp synthase is a mutated epsp synthase and the mutation has been generated by a mutative reagent, radiation, gene editing, site-specific point mutation, zinc finger nucleases, TALENS or by in vitro culturing of the respective cells and subsequent selection by the use of a herbicide as glyphosate.
[115] The plant according to [114], further characterized in that the plant is a hybrid plant.
[116] The plant according to [114] or [115], further characterized in that the plant is a double haploid plant.
[117] The plant according to anyone of [114] to [116], further characterized in that the resistance against Cercospora is dominant.
[118] The plant according to anyone of [114] to [117], further characterized in that the nucleic acid molecule or nucleotide sequence is comprised as an introgression.
[119] The plant according to anyone of [114] to [118], further characterized in that the nucleic acid molecule or nucleotide sequence is homozygous.
[120] The plant according to anyone of [114] to [119], further characterized the plant has tolerance to glyphosate.
[121] The plant according to anyone of [114] to [120], further characterized in that the nucleic acid molecule or nucleotide sequence is comprised as a transgene.
[122] The plant according to anyone of [114] to [121], further characterized in that the nucleic acid molecule or nucleotide sequence is obtainable from the seeds deposited with the NCIMB, Aberdeen, UK under access number NCIMB 43646.
[123] The storage organ or a leaf of the plant according to anyone of [114] to [122].
[124] A pelleted seed of the plant according to anyone of [114] to [122], characterized in that the seed comprises the nucleic acid molecule and the endogenous allele.
[125] The plant according to anyone of [114] to [122], the storage organ or leaf according to claim 13 or the pelleted seed according to claim 14, wherein the epsp synthase having at position 179 an amino acid different from proline, comprises an amino acid sequence selected from
   i) the sequence of SEQ ID NO: 223
   ii) the sequence i) having at position 179 an amino acid different from serine and proline, or
   iii) a sequence having an identity of at least 90% to the sequence of i) or ii) over the entire length of the sequence.
[126] The method according to anyone of [104]-[113] wherein the cosegregating region is a cosegregating region as defined in [48].
[127] The method according to anyone of [109]-[113] wherein the at least two oligonucleotides are oligonucleotides as defined in [20], [21], [72] or [73].
[128] A mixture of a pelleting mass and a sugar beet plant seed characterized in that the sugar beet plant seed is a seed of the plant according to anyone of [114] to [122], characterized in that the seed comprises the nucleic acid molecule and the endogenous allele.

First, some of the terms used in this application are explained in detail in the following:

What is understood by “rating score” in the sense of the present invention is a qualitative assessment of the resistance to a Cercospora infestation that is represented using a scale from 1 to 9 (with 1=strong resistance and 9=no resistance).

TABLE 1A
9-level resistance rating for Cercospora

| Rating | | |
|---|---|---|
| score | Leaf phenotype | Whole plant phenotype |
| 1 | Healthy leaf | Healthy leaf, whole |
| 3 | Diseased leaf, spots on the | Whole plant, beginning of |
| | outer leaves | disease, spots on the outer |
| | | leaves |
| 5 | Diseased leaf, merging of | Whole plant, advanced disease, |
| | the spots into dying areas | merging of the spots into dying |
| | | areas |
| 7 | Diseased leaf, large part of | Whole diseased plant, large |
| | the leaf brown and dead, only | portions of the outer leaves |
| | lower lamina is still alive | are dying off |
| 9 | Discased leaves, lamina and | Whole diseased plant, outer |
| | petiole are dead and dried | leaves have died, inner leaves |
| | | with severe damage, strong new |
| | | leaf growth |

The genus Cercospora encompasses various species, e.g., the species Cercospora arachidicola, Cercospora ariminiensis, Cercospora asparagi, Cercospora bertoreae, Cercospora beticola, Cercospora bizzozeriana, Cercospora canescens, Cercospora carotae, Cercospora chenopodii, Cercospora cistinearum, Cercospora cladosporioides, Cercospora diazu, Cercospora dulcamarae, Cercospora erysimi, Cercospora hayii, Cercospora kikuchii, Cercospora malvacearum, Cercospora malvicola, Cercospora medicaginis, Cercospora oryzaem, Cercospora personata, Cercospora plantaginis, Cercospora ricinella, Cercospora setariae, Cercospora unamunoi, Cercospora volae, or Cercospora zeae-maydis.

In conjunction with the specification of a length of a nucleotide sequence, the term, “approximately,” means a deviation by +/−200 base pairs—preferably, by +/−100 base pairs, and, particularly preferably, by +/−50 base pairs.

A cosegregating region is a genomic region which is in most of the cases or always inherited together with the genomic region, locus, gene, polymorphism or single nucleotide polymorphism to which it is genetically coupled, genetically linked or adjacent or close by. For example the genetic linkage may be inherited in at least 95%, 96%, 97%, 98% or 99% to the next generation or the cosegregating region and the genomic region, locus, gene, polymorphism or single nucleotide polymorphism to which it is genetically coupled, genetically linked or adjacent or close have at distance of at most 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less. The presence of the cosegregating region is indicative or diagnostic for the corresponding genomic region, locus, gene, polymorphism or single nucleotide polymorphism. Cosegregating regions may be detected/identified by the corresponding methods, markers and oligonucleotides included herein for example in the seeds deposited with the NCIMB, Aberdeen, UK under access number NCIMB 43646. In context of the present invention the cosegregating region may be especially a genomic region in a plant of the genus Beta, Spinacia, Glycine, Daucus or Pastinaca.

A “plant” comprises plants of different genera including but not limited to Beta, Spinacia, Glycine, Daucus and Pastinaca.

A “plant of the genus Beta” belongs to the amaranth family (Amaranthaceae). Numbering among these plants are plants of the species Beta macrocapa, Beta vulgaris, Beta lomatogona, Beta macrorhiza, Beta corolliflora, Beta trigya, and Beta nana. A plant of the species Beta vulgaris is, in particular, a plant of the subspecies Beta vulgaris subsp. vulgaris. For example, numbering among these are Beta vulgaris subsp. vulgaris var. altissima (sugar beet in a narrower sense), Beta vulgaris ssp. vulgaris var. vulgaris (chard), Beta vulgaris sap. vulgaris var. conditiva (beetroot/red beet), Beta vulgaris ssp. vulgaris var. crassa/alba (fodder beet). It is noted that the nucleic acid according to the invention does not naturally occur in sugar beet, chard, beetroot, or fodder beet, but may be introduced into these via human action.

A “plant of the genus Spinacia” belongs to the amaranth family (Amaranthaceae). This genus especially encompasses Spinacia oleracea which is also known as spinach.

A “plant of the genus Glycine” belongs to the family of Fabaceae. This genus especially encompasses Glycine max which is also known as soybean.

A “plant of the genus Daucus” belongs to the family of Apiaceae. This genus especially encompasses Daucus carota and Daucus carota subsp. sativus which is also known as carrot.

A “plant of the genus Pastinaca” belongs to the family of Apiaceae. This genus especially encompasses Pastinaca sativa which is also known as parsnip.

A “functional fragment” of a nucleotide sequence means a segment of a nucleotide sequence which has a functionality identical or comparable to that of the complete nucleotide sequence from which the functional fragment originates. As such, the functional fragment may possess a nucleotide sequence which is identical or homologous to the total nucleotide sequence over a length of at least 50%, 55%, 60%, 65%, 70%, 75%, 80%, 85%, 90%, 92%, 94% 96%, 97%, 98%, or 99%. This also explicitly encompasses the range of 90-100%. Furthermore, a “functional fragment” of a nucleotide sequence may also mean a segment of a nucleotide sequence which modifies the functionality of the entire nucleotide sequence, e.g., in the course of post-transcriptional or transcriptional gene silencing. As such, the functional fragment of a nucleotide sequence may comprise at least 14, 15, 16, 17, 18, 19, 20, 21, 22, 23, 24, or 25—preferably, at least 30, 35, 40, 45, 50, 60, 70, 80, 90, 100, 120, or 140, and, particularly preferably, at least 160, 180, 200, 250, 300, 350, 400, 450, 500, 600, 700, 800, 900, or 1,000—successive nucleotides of the total nucleotide sequence. This also explicitly encompasses the range of 21 to 50 nucleotides.

A “functional part” of a protein means a segment of a protein, or a section of the amino acid sequence, that encodes the protein, wherein the segment may exert functionality identical or comparable to that of the entire protein in a plant cell. Over a length of at least 50%, 55%, 60%, 65%, 70%, 75%, 80%, 85%, 90%, 92%, 94% 96%, 97%, 98%, or 99%, a functional part of a protein has an amino acid sequence that is identical or, with consideration of conservative and semi-conservative amino acid exchanges, similar to the protein from which the functional part originates.

The term, “heterologous,” means that the introduced polynucleotide originates from a cell or an organism with a different genetic background, of the same species or a different species, or is homologous to the prokaryotic or eukaryotic host cell, but is then located in a different genetic environment and thus differs from a corresponding polynucleotide that is possibly naturally present. A heterologous polynucleotide may be present in addition to a corresponding endogenous gene.

In the sense of the invention, what is understood by a “homolog” is a protein of the same phylogenetic origin; what is understood by an “analog” is a protein which exerts the same function, but has a different phylogenetic origin; what is understood by an “ortholog” is a protein from a different species that exerts the same function; and what is understood by a “paralog” is a protein that has appeared within a species due to duplication, wherein this copy either retains the same protein function, alters its expression template, but not the function, changes its protein function, or divides up the original gene function between both copies.

What is to be understood by “hybridizing” or “hybridization” is a process in which a single-stranded nucleic acid molecule binds to a nucleic acid strand that is complementary to the greatest possible extent, i.e., forms base pairs with this. Standard methods for hybridization are described in, for example, Sambrook et al., Molecular Cloning: A Laboratory Manual, 3rd ed., Cold Spring Harbor Laboratory Press, Cold Spring Harbor, N Y, 2001. What is preferably understood by this is that at least 60%—more preferably, at least 65%, 70%, 75%, 80%, or 85%, and, particularly preferably, 90%, 91%, 92%, 93%, 94%, 95%, 96%, 97%, 98%, or 99%—of the bases of the nucleic acid molecule form a base pairing with the nucleic acid strand that is complementary to the greatest possible extent. The possibility of such an annealing depends upon the stringency of the hybridization conditions. The term, “stringency,” relates to the hybridization conditions. High stringency is present when a base pairing is made more difficult; low stringency is present if a base pairing is made easier. For example, the stringency of the hybridization conditions depends upon the salt concentration or ionic strength and the temperature. In general, the stringency may be increased by increasing the temperature and/or decreasing the salt content. What are to be understood by “stringent hybridization conditions” are those conditions given which a hybridization predominantly occurs only between homologous nucleic acid molecules. The term, “hybridization conditions,” thereby relates not only to the conditions prevailing in the actual addition of the nucleic acids, but also to the conditions prevailing in the following washing steps. For example, stringent hybridization conditions are conditions under which, predominantly, only those nucleic acid molecules hybridize that have at least 70%—preferably, at least 75%, at least 80%, at least 85%, at least 90%, or at least 95%—sequence identity. Stringent hybridization conditions are, for example: hybridization in 4×SSC at 65° C., and subsequent repeated washing in 0.1×SSC at 65° C. for approximately 1 hour in total. A hybridization preferably occurs under stringent conditions.

Sequences showing a certain identity level to a starting sequence may show the given identity level over the whole length of the starting sequence. This is applicable towards nucleotide sequences as well as amino acid sequences.

In relation to a nucleic acid in the form of a double-stranded DNA, “complementary” nucleotide sequence means that the second DNA strand complementary to the first DNA strand has the nucleotides that correspond to the bases of the first strand, in accordance with the base pairing rules. A complementary sequence is, preferably, entirely complementary to the counter-sequence, and thus preferably has the same length.

What is understood by an “isolated nucleic acid molecule” is a nucleic acid molecule extracted from its natural or original environment. The term also encompasses a synthetically-produced nucleic acid molecule. What is understood by an “isolated polypeptide” is a polypeptide extracted from its natural or original environment. The term also encompasses a synthetically-produced polypeptide.

A “molecular marker” or “marker” is a nucleic acid that is polymorphic in a plant population and is used as a reference or orientation point. Depending on the technical context the term “marker” may be related to a specific genomic position which is detectable by a corresponding “molecular marker” wherein the “molecular marker” in most cases is sequentially compatible to the genomic position. A marker for the detection of a recombination event should be suitable for monitoring differences or polymorphisms within a plant population. Such a marker is thus able to detect and differentiate between various allelic states (alleles). The term, “molecular marker,” also relates to nucleotide sequences which are complementary or at least largely complementary or homologous to genomic sequences—for example, nucleic acids which are used as probes or primers. These differences at the DNA level are to be found as markers and are, for example, polynucleotide sequence differences, e.g., SSR's (simple sequence repeats), RFLP's (restriction fragment length polymorphisms), FLP's (fragment length polymorphisms) or SNP's (single nucleotide polymorphisms). The markers may be derived from genomic or expressed nucleic acids, e.g., spliced RNA, cDNA, or ESTs, and may also relate to nucleic acids that are used as probes or primer pairs and as such are suitable for amplifying a sequence fragment using PCR-based methods. Markers that describe genetic polymorphisms (between parts of a population) may be detected using well-established methods from the prior art (An Introduction to Genetic Analysis, 7th edition, Griffiths, Miller, Suzuki, et al., 2000). For example, among these are DNA sequencing, PCR-based, sequence-specific amplification, verification of RFLP's, verification of polynucleotide polymorphisms by means of allele-specific hybridization (ASH), detection of amplified variable sequences of the plant genome, detection of a 3SR (self-sustained sequence replication), detection of SSR's, SNP's, RFLP's, or AFLP's (amplified fragment length polymorphisms). Furthermore, the methods for detection of EST's (expressed sequence tags) and SSR markers derived from EST sequences and RAPD (randomly amplified polymorphic DNA) are also known. Depending upon the context, the term, “marker,” in the description may also mean a specific chromosome position in the genome of a species where a specific marker (SNP, for example) may be found.

Markers also include synthetic oligonucleotides that may be connected with one or more detection molecules, wherein the detection molecules may be used for a detection reaction or the generation of a signal within the scope of a verification method. Synthetic oligonucleotides also include labeled primers. Synthetic oligonucleotides and labeled primers are artificial compounds, do not occur in nature, and cannot be isolated from nature. The production of such compounds is explained further below.

A “single nucleotide polymorphism” or SNP is a genetic variation between two samples of DNA wherein at least one nucleotide between the two samples is different. In most cases the samples belong two the same species and the comparison or alignment of the two samples is performed on the basis of homologues genomic regions. SNPs may cause allelic variations but not all SNPs need to occur within a functional genomic element like a gene. SNPs can be used two differentiate between for example different genotypes/haplotypes or may be used to screen and select for the presence of absence of a functional genomic element like for example a specific gene or its allelic variant. Due to genetic linkage SNPs need not to be within the functional genomic element which is to be selected. Examples for SNPs in the sense of the invention are given in Tab. 1b, especially they are included in the common primer sequences. The detection or identification of SNPs may occur by a PCR involving two different forward primers and one common revers primer. The technical details for such a detection or identification may be derived from passages [67]-[70] above.

A “promoter” is a non-translated, regulatory DNA sequence, typically upstream of a coding region, which contains the binding point for the RNA polymerase and initiates the transcription of the DNA. A promoter additionally contains other elements that act as a regulator gene for gene expression (for example, cis-regulatory elements). A “core or minimal promoter” is a promoter that has the basic elements which are needed for transcription initiation (for example, TATA box and/or initiator).

A “pathogen” means an organism that, in interactions with a plant, leads to disease symptoms in one or more organs in the plant. For example, animal, fungal, bacterial, or viral organisms or oomycetes number among these pathogens.

What is to be understood by a “pathogenic infection” is the earliest point in time at which a pathogen interacts with a plant host tissue. In this sense, “infestation” means the occurrence of contact between pathogen and host. With an anchorage of a pathogen at a host, e.g., of a fungal spore on a leaf surface of a plant, mechanisms of pathogen detection and signal relaying begin in the plant host cell. In the case of Cercospora beticola, conidia are formed in humid, warm weather and transferred to neighboring plants by rain and wind. New infections most often show individual leaf spots first at the physiologically older outer leaves. These are most often quite clearly delimited from the healthy leaf tissue by a brown ring. The brown conidia carriers of the fungus in the middle part of the spots may be observed with the aid of a magnifying glass (rating score 3). The number of these brown spots increases rapidly, wherein the sporocarps initially overlap even smaller dead areas (rating score 5). In the further course of the disease, which now also spans to the inner leaves, dying-off of the outer leaves finally occurs for the first time (rating score 7), and, then, of practically all leaves (rating score 9). Course of disease and symptom severity are strongly dependent upon the site and on the annually fluctuating weather conditions.

Plant “organs” means, for example, leaves, shoot, stem, roots, hypocotyl, vegetative buds, meristems, embryos, anthers, ovula, seeds, or fruits. “Plant parts” include, but are not limited to, the shoot or the stalk, leaves, blossoms, inflorescence, roots, fruits, and seeds, as well as the pollen. The term, “plant parts,” also means an association of multiple organs, e.g., a blossom or a seed, or a part of an organ, e.g., a cross-section through the plant shoot. Plant “tissues” are, for example, callus tissue, storage tissue, meristematic tissue, leaf tissue, shoot tissue, root tissue, plant tumor tissue, or reproductive tissue, as well as the cambium, parenchyma, vascular tissue, sclerenchyma, and epidermis. However, the tissue is not limited to this listing. For example, what are to be understood by plant “cells” are, for example, isolated cells having a cell wall or aggregates thereof, or protoplasts.

“Recombination frequency” means that between some genomic elements like single nucleotide polymorphisms, genes, loci, QTL or genetic regions a crossing over occurs during meiosis. A recombination frequency of 1% is equal to 1 cM. It is possible to determine the recombination frequency by the generation of meiosis maps.

“Resistance gene according to the invention” is a nucleotide molecule or nucleic acid sequence as described under passage [1] above. The gene may be genetically linked to cosegregating regions.

“Variety” means a plant grouping within a single botanical taxon of the lowest known rank, which grouping, irrespective of whether the conditions for the grant of a breeder's right are fully met, can be—defined by the expression of the characteristics resulting from a given genotype or combination of genotypes,
distinguished from any other plant grouping by the expression of at least one of the said characteristics and
considered as a unit with regard to its suitability for being propagated unchanged.

In conjunction with the present invention, the term, “regulatory sequence,” relates to a nucleotide sequence which influences the specificity and/or the expression strength, e.g., in that the regulatory sequence confers a defined tissue specificity. Such a regulatory sequence may be located upstream of the transcription initiation point of a minimal promoter, but also downstream thereof e.g., in a transcribed, but not translated, leader sequence or within an intron.

The term, “resistance,” is to be understood broadly and covers the range of the protection from a retardation up to a complete blocking of the development of the disease. One example of an important pathogen is Cercospora beticola. A resistant plant cell of the invention or resistant plant of the invention preferably achieves a resistance to Cercospora beticola. A resistance to a pathogen is to be equated to a resistance to the disease which this pathogen causes; for example, a resistance to Cercospora beticola is also a resistance to leaf spot disease. For example, an increase in the resistance can be measured via a reduced fungal biomass on the host plant; for this, the fungal DNA may be determined with the aid of quantitative PCR in comparison to the plant DNA in the infested plant tissue. An additional approach to the measurement of resistance is optical rating, wherein rating scores of 1 (not susceptible) to 9 (very susceptible) are awarded.

“Transgenic plant” relates to a plant into whose genome is integrated at least one polynucleotide. It may thereby be a heterologous polynucleotide. The polynucleotide is, preferably, stably integrated, which means that the integrated polynucleotide is stably preserved in the plant, is expressed, and also may be stably passed on to the descendants. The stable introduction of a polynucleotide into the genome of a plant also includes the integration into the genome of a plant of the preceding parental generation, wherein the polynucleotide may be stably passed on further. The term, “heterologous,” means that the introduced polynucleotide originates from a cell or an organism with a different genetic background, of the same species or a different species, or is homologous to the prokaryotic or eukaryotic host cell, for example, but then is located in a different genetic environment and thus differs from a corresponding polynucleotide that is possibly naturally present. A heterologous polynucleotide may be present in addition to a corresponding endogenous gene.

“Raw material for industrial sugar production” means plant material which can be fed into a sugar production facility which is specialized in the extraction of sugar from sugar beets. Such raw material is typically the beet body (taproot) of the harvested sugar beet. To ensure the conformity with the extraction process the beet body needs to have sufficient mass, volume and a conical shape so that the raw material can be mechanically cut into shreds (beet strips). These beet strips maximize the surface area for sugar extraction and should have a low content of Sodium, Potassium and Nitrogen to allow an efficient extraction. After the extraction remaining beet pulp is pressed, dried and used as animal feed.

“Saccharose concentration” is expressed as percentage of the fresh weight of the root.

“Monogerm” means that a seed grows into exactly one plant whereas a polygerm or multigerm seed (also called “seed ball”) grows into several plants.

“Bolting” is the production of a flowering stem (or stems) on a sugar beet in a natural attempt to produce seeds and reproduce. Bolting is triggered in sugar beet due to vernalization, i.e. a chilling stress which might occur e.g. during overwintering. However, commercially grown sugar beets are harvested before bolting as the bolting process and subsequent seed setting reduces the saccharose content in the beet body.

“Introgression” means that a nucleotide sequence has been transferred into the genome of a plant wherein this nucleotide sequence originates from a plant that does not belong to the same species or subspecies. This can for example mean that a nucleotide sequence deriving from a plant of the subspecies Beta vulgaris maritima has been transferred into a plant of the subspecies Beta vulgaris vulgaris.

Designs and embodiments of the present invention are described by way of example with reference to the pending sequences and figures.

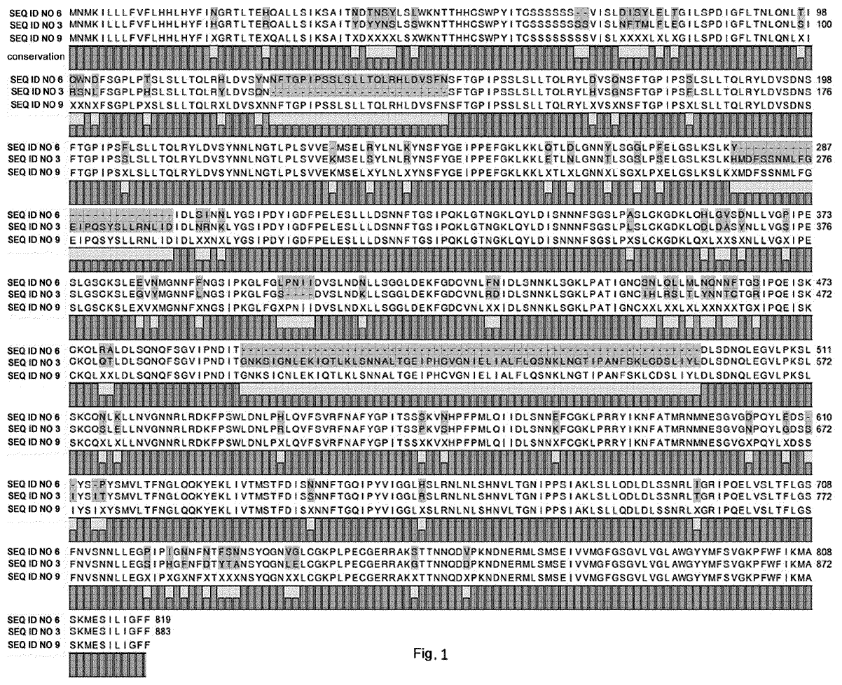

FIG. 1: Protein sequence alignment between the resistant protein (protein which confers Cercospora resistance in a plant) and the sensitive protein (protein which does not confer Cercospora resistance in a plant). The polymorphisms are highlighted in gray.

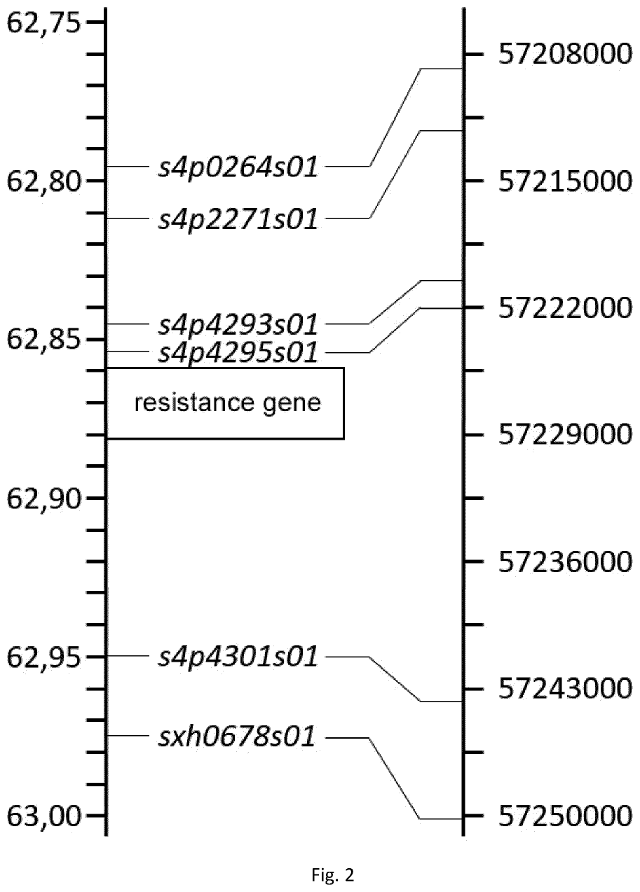

FIG. 2: Genetic (left part) an physical (right part) map of the genomic region including the resistance gene showing the positions of some most important markers.

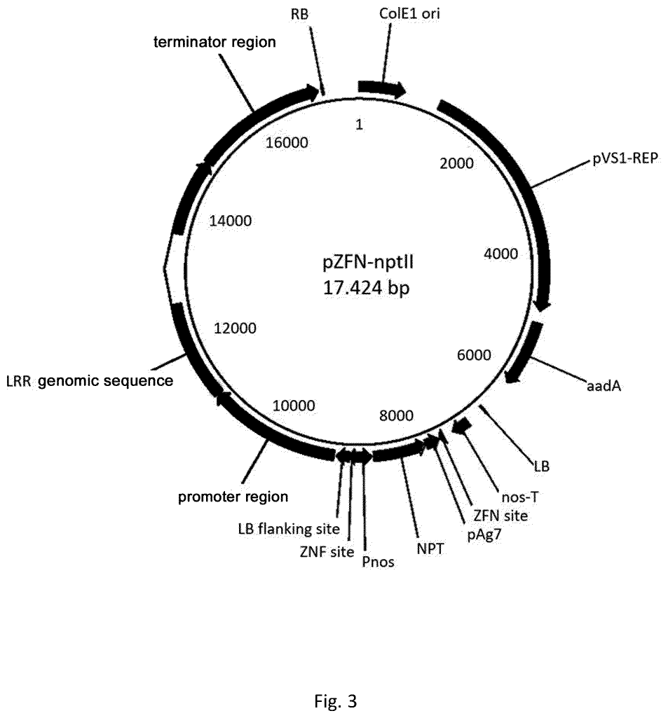

FIG. 3: Vector map of the vector pZFN-nptII including the LRR region.

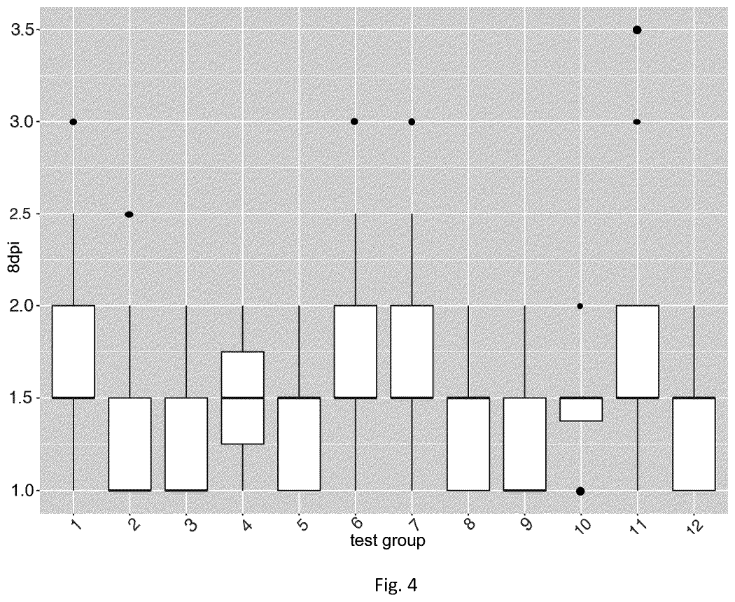

FIG. 4: Statistical box-plot evaluation of the data generated eight days post infection during the transgenic verification of the resistance gene.

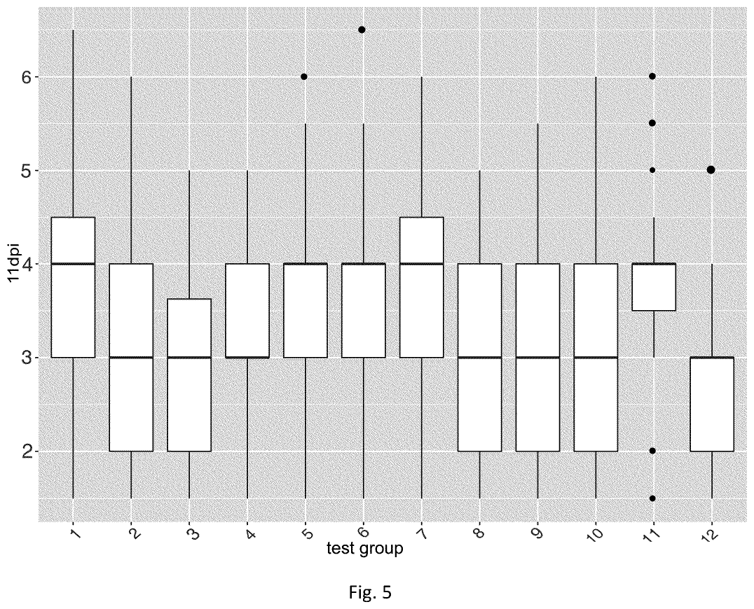

FIG. 5: Statistical box-plot evaluation of the data generated eleven days post infection during the transgenic verification of the resistance gene.

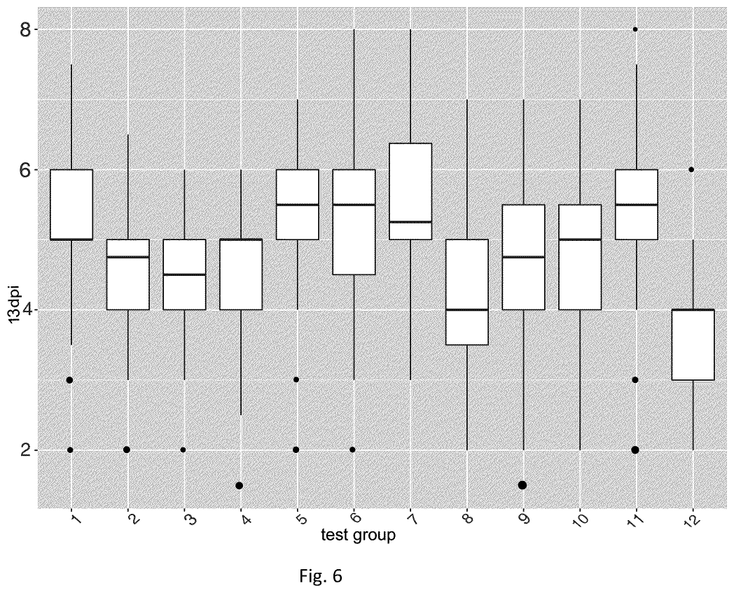

FIG. 6: Statistical box-plot evaluation of the data generated eight days post infection during the transgenic verification of the resistance gene.

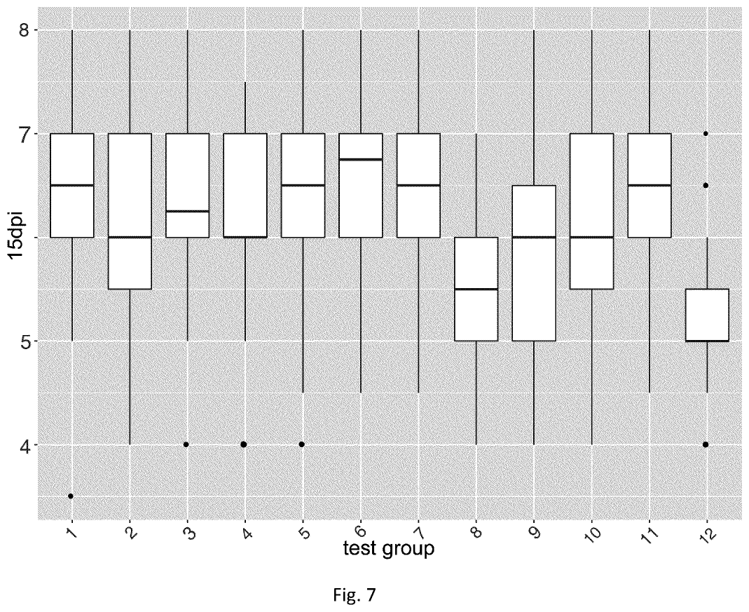

FIG. 7: Statistical box-plot evaluation of the data generated fifteen days post infection during the transgenic verification of the resistance gene.

### DETAILED DESCRIPTION OF THE INVENTION

The present invention relates to a nucleic acid molecule that is able to confer a resistance to Cercospora in a plant—in particular, in Beta vulgaris subsp. vulgaris—in which the polypeptide which is encoded by the nucleic acid molecule is expressed. According to a preferred embodiment of the invention, the pathogen is the fungus Cercospora beticola, which is among the most important and destructive leaf pathogens of sugar beets, beetroot, and chard, among others, and may cause crop losses of over 40%. The fungus produces the secondary metabolite cercosporin, which reacts with oxygen in the presence of light and leads to the formation of reactive oxygen species (ROS). The ROS cause massive cell damage in the leaf tissue of the infested plant that visible in the form of necrosis.

The present invention is based upon the genetic fine mapping, identification, isolation, and characterization of a gene or of a gene locus that originates from the donor Beta vulgaris subsp. maritima, whose presence in a plant—in particular, in Beta vulgaris subsp. vulgaris—correlates with or is causative of the resistance of the plant concerned to Cercospora leaf spot disease. Initial material was a Beta vulgaris subsp. maritima population which was developed from 37 Beta vulgaris subsp. maritima accessions from different sources. Seeds of a Beta vulgaris subsp. vulgaris plant (sugar beet cultivar 6626) comprising the resistance according to the invention have been deposited before the filing with the National Collection of Industrial Food and Marine Bacteria (NCIMB), Aberdeen, UK under access number NCIMB 43646. Especially a plant of the genus Beta, a plant of the species Beta vulgaris or a plant of the subspecies Beta vulgaris subsp. vulgaris or a sugar beet plant comprising the resistance gene according to the invention may be derived from the deposited seeds.

The nucleotide and amino acid encoding sequence of the nucleic acid molecule according to the invention is characterized by numerous polymorphisms, which differentiates the NPS-LRR gene identified according to the invention from the “sensitive” variant of the gene, i.e., the variant of the gene that does not confer resistance to Cercospora. Examples of polymorphisms are presented in FIG. 1.

The nucleic acid molecule according to the invention may be an isolated nucleic acid molecule. It is preferably DNA, and, particularly preferably, cDNA (coding DNA). The polypeptide which is encoded by the nucleic acid molecule according to the invention preferably confers a resistance to the pathogen Cercospora beticola, which causes the plant disease Cercospora leaf spot. Furthermore, the polypeptide which is encoded by the nucleic acid molecule according to the invention confers—in particular, in a plant of the genus Beta—a resistance to this pathogen. The plant is preferably a plant of the species Beta vulgaris—particularly preferably, a plant of the subspecies Beta vulgaris subsp. vulgaris; among these are, for example, the cultivars sugar beet, beetroot, fodder beet, chard, and Swiss chard.

In one embodiment of the present invention, the nucleic acid molecule according to the invention comprises a nucleotide sequence that encodes a polypeptide with an amino acid sequence according to SEQ ID No. 3 and/or the coding DNA sequence according to SEQ ID No. 2. Furthermore, the present invention provides a nucleotide sequence that comprises the DNA sequences according to SEQ ID No. 1 and SEQ ID No. 53.

The gene identified according to the invention is a resistance gene/protein of the type NBS-LRR, which is characterized by specific structural motifs. The general structure of such resistance proteins in plants has already been well-examined (Martin et al., Annual Review Plant Biology 54 (2003), 23-61). However, the principle of the structural embodiment—in particular, of what is known as the LRR domain, which applies as a potential detection domain for most unknown pathogenic effectors—is unpredictable, and the functional background of the resistance genes i.e., the genetic structure, is generally largely unknown. The identification of a Cercospora resistance-conferring gene or protein solely on the basis of the known structural motif is, consequently, impossible. Furthermore, the sequence region has turned out to be a highly repetitive region that contains, among other things, tandem repeats with very high sequence homology, which makes the development of diagnostic markers, as well as the assembly of sequence data, especially difficult.

With the aid of the setup of a population of over 4,000 dividing descendants and the development of special recombination screens, the target region was reduced, and thus ever further isolated, via analysis of informative recombinants (genotypical and phenotypical) in a series of resistance tests. This genetic mapping, as well as the creation of physical maps accompanied by WHG sequencing (“whole genome sequencing”), comparative BAC (Bac-by-Bac) sequencing, and bioinformatic analyses, led to the identification of three recombinant genotypes that confirmed the resistance gene (1 recombinant in the neighboring gene, on the one hand, and 2 recombinants in the neighboring gene, on the other). In light of particular requirements, the inventors placed the highly repetitive structure in the target region, which, among other things, contains tandem repeats with very high sequence homology, which made the marker development, and thus the identification of informative recombinants, enormously more difficult. The following steps were particularly decisive for the location of the genetic structure of the resistance gene:
development of the markers s4p0264s01, s4p2271s01, sxh0678s01, s4p4293s01, s4p4295s01, s4p4301s01 (see Table 1B).
Fine mapping coupled with intensive phenotyping. The phenotypes were verified with 90-180 descendants per plant in a greenhouse test, and with intensive statistical methods (for example, t-test, power analysis, etc.).
BAC clone identification and sequencing from BAC pools of the resistant genotype.
Sequence evaluation, as well as sequence and protein comparison between RR (i.e., resistant) and ss (i.e., sensitive) genotypes; an unambiguous assembly of the RR and ss sequence data was thereby not always possible, due to the sequence complexity.

TABLE 1B
Marker in the target region relating to sensitive genotype,
resistant genotype and consensus sequence.

| | Sequences: sensitive/resistant/ | Position on | Position on |
|---|---|---|---|
| Marker | consensus | genetic map [cM] | physical map [bp] |
| s4p1395s01 | SEQ ID No. 100/SEQ ID No. 101/ | 56,35582927 | 55312395 |
| | SEQ ID No. 102 | | |
| sxi0123s02 | SEQ ID NO. 103/SEQ ID NO. 104/ | 56,47317509 | 55357303 |
| | SEQ ID NO. 105 | | |
| s4p1396801 | SEQ ID NO. 106/SEQ ID NO. 107/ | 56,61163027 | 55409550 |
| | SEQ ID NO. 108 | | |
| s4p1398s01 | SEQ ID NO. 109/SEQ ID NO. 110/ | 56,78339709 | 55473271 |
| | SEQ ID NO. 111 | | |
| s4p1462s01 | SEQ ID NO. 112/SEQ ID NO. 113/ | 56,84738427 | 55496705 |
| | SEQ ID NO. 114 | | |
| s4p1464s01 | SEQ ID NO. 115/SEQ ID NO. 116/ | 56,87553455 | 55506972 |
| | SEQ ID NO. 117 | | |
| s4p0044s01 | SEQ ID NO. 118/SEQ ID NO. 119/ | 58,84432555 | 56160463 |
| | SEQ ID NO. 120 | | |
| s4p0056s01 | SEQ ID NO. 121/SEQ ID NO. 122/ | 59,85754673 | 56456734 |
| | SEQ ID NO. 123 | | |
| s4p0058s01 | SEQ ID NO. 124/SEQ ID NO. 125/ | 60,00588436 | 56498250 |
| | SEQ ID NO. 126 | | |
| s4p0059s01 | SEQ ID NO. 127/SEQ ID NO. 128/ | 60,00633209 | 56498372 |
| | SEQ ID NO. 129 | | |
| s4p1564s01 | SEQ ID NO. 130/SEQ ID NO. 131/ | 60,00815936 | 56498883 |
| | SEQ ID NO. 132 | | |
| s4p1998s01 | SEQ ID NO. 133/SEQ ID NO. 134/ | 60,02385255 | 56503259 |
| | SEQ ID NO. 135 | | |
| s4p1343s01 | SEQ ID NO. 136/SEQ ID NO. 137/ | 60,42647909 | 56613667 |
| | SEQ ID NO. 138 | | |
| s4p1408s01 | SEQ ID NO. 139/SEQ ID NO. 140/ | 60,42651091 | 56613673 |
| | SEQ ID NO. 141 | | |
| s4p1565s01 | SEQ ID NO. 142/SEQ ID NO. 143/ | 60,54596773 | 56645847 |
| | SEQ ID NO. 144 | | |
| s4p1409s01 | SEQ ID NO. 145/SEQ ID NO. 146/ | 60,547695 | 56646306 |
| | SEQ ID NO. 147 | | |
| s4p1411s01 | SEQ ID NO. 148/SEQ ID NO. 149/ | 60,86562555 | 56730683 |
| | SEQ ID NO. 150 | | |
| s4p1414s01 | SEQ ID NO. 151/SEQ ID NO. 152/ | 60,99547782 | 56764641 |
| | SEQ ID NO. 153 | | |
| s4p1485s01 | SEQ ID NO. 154/SEQ ID NO. 155/ | 61,54654291 | 56905647 |
| | SEQ ID NO. 156 | | |
| s4p0257s01 | SEQ ID NO. 157/SEQ ID NO. 158/ | 62,42235182 | 57120242 |
| | SEQ ID NO. 159 | | |
| s4p0258s01 | SEQ ID NO. 160/SEQ ID NO. 161/ | 62,42346318 | 57120506 |
| | SEQ ID NO. 162 | | |
| s4p0260s01 | SEQ ID NO. 163/SEQ ID NO. 164/ | 62,59136591 | 57160401 |
| | SEQ ID NO. 165 | | |
| sxh0876s05 | SEQ ID NO. 166/SEQ ID NO. 167/ | 62,730303 | 57193142 |
| | SEQ ID NO. 168 | | |
| s4p0262s01 | SEQ ID NO. 169/SEQ ID NO. 170/ | 62,79537191 | 57208387 |
| | SEQ ID NO. 171 | | |
| s4p0263s01 | SEQ ID NO. 172/SEQ ID NO. 173/ | 62,79557191 | 57208429 |
| | SEQ ID NO. 174 | | |
| s4p0264s01 | SEQ ID No. 54/SEQ ID No. 55/SEQ ID No. 10 | 62,79590373 | 57208510 |
| s4p2271s01 | SEQ ID No. 56/SEQ ID No. 57/SEQ ID No. 11 | 62,81185523 | 57212240 |
| s4p4288s01 | SEQ ID NO. 175/SEQ ID NO. 176/ | 62,81950148 | 57214020 |
| | SEQ ID NO. 177 | | |
| s4p4290d01 | SEQ ID NO. 178/SEQ ID NO. 179/ | 62,83393842 | 57217392 |
| | SEQ ID NO. 180 | | |
| s4p4293s01 | SEQ ID No. 58/SEQ ID No. 59/SEQ ID No. 12 | 62,84491806 | 57219956 |
| s4p4295s01 | SEQ ID No. 60/SEQ ID No. 61/SEQ ID No. 13 | 62,85399055 | 57222060 |
| s4p8772s01 | SEQ ID NO. 181/SEQ ID NO. 182/ | | |
| | SEQ ID NO. 183 | | |
| s4p4301s01 | SEQ ID No. 62/SEQ ID No. 63/SEQ ID No. 14 | 62,94635089 | 57243521 |
| sxh0678s01 | SEQ ID No. 64/SEQ ID No. 65/SEQ ID No. 15 | 62,97474964 | 57250119 |
| s4p4305d01 | SEQ ID NO. 184/SEQ ID NO. 185/ | 62,98035341 | 57251418 |
| | SEQ ID NO. 186 | | |
| s4p4308s01 | SEQ ID NO. 187/SEQ ID NO. 188/ | 63,00151327 | 57256313 |
| | SEQ ID NO. 189 | | |
| s4p0317s01 | SEQ ID NO. 190/SEQ ID NO. 191/ | 63,16585209 | 57294143 |
| | SEQ ID NO. 192 | | |
| sxh1834s05 | SEQ ID NO. 193/SEQ ID NO. 194/ | 63,26778918 | 57317441 |
| | SEQ ID NO. 195 | | |
| sxh0942s04 | SEQ ID NO. 196/SEQ ID NO. 197/ | 63,31312755 | 57327765 |
| | SEQ ID NO. 198 | | |
| s4p0320s01 | SEQ ID NO. 199/SEQ ID NO. 200/ | 63,49891636 | 57369798 |
| | SEQ ID NO. 201 | | |
| s4p0322s01 | SEQ ID NO. 202/SEQ ID NO. 203/ | 63,52934236 | 57376639 |
| | SEQ ID NO. 204 | | |
| s4p0323s01 | SEQ ID NO. 205/SEQ ID NO. 206/ | 63,79248973 | 57435390 |
| | SEQ ID NO. 207 | | |
| s4p0232s01 | SEQ ID NO. 208/SEQ ID NO. 209/ | 64,19409409 | 57523548 |
| | SEQ ID NO. 210 | | |
| s4p0234s01 | SEQ ID NO. 211/SEQ ID NO. 212/ | 64,32903091 | 57552769 |
| | SEQ ID NO. 213 | | |
| sxh1195s02 | SEQ ID NO. 214/SEQ ID NO. 215/ | 64,32903091 | 57552771 |
| | SEQ ID NO. 216 | | |
| s4p0238s01 | SEQ ID NO. 217/SEQ ID NO. 218/ | 64,36171473 | 57559821 |
| | SEQ ID NO. 219 | | |
| s4p0421s01 | SEQ ID NO. 220/SEQ ID NO. 221/ | 65,86730118 | 57873806 |
| | SEQ ID NO. 222 | | |

The compounds provided in Table 1B can be used as molecular markers according to the invention. All markers are suitable to detect genetic material comprising the resistance gene according to the invention (especially by the identification of genetically linked polymorphisms like single nucleotide polymorphisms) and to trace the inheritance of the genetic material from a source plant to the offspring of the source plant via marker assisted selection (MAS). In this regard the markers disclosed herein can be highly useful in transmitting the resistance gene according to the invention from one subspecies to a different subspecies. The markers disclosed herein can be furthermore used for genetic mapping and the tracing of recombination as a consequence meiosis. It seems that the resistance gene is located close to or in between the physical positions 57219956 bp and 57243521 bp. Markers disclosed herein which are located very close to this region are highly suitable for the detection of the resistance gene. Markers disclosed herein which are in larger distance to the resistance gene still show a strong genetic linkage to the resistance gene but can furthermore be used to detect rare recombination events and genetic exchange in the flanking areas of the resistance gene. This can be useful for example in backcrossing approaches and the transmittance of the resistance gene into a different genetic background. The markers disclosed herein and especially those which are in larger distance to the resistance gene are especially useful when the resistance gene is transmitted to the offspring as part of a cosegregating region. The resistance gene including cosegregating regions may also be transmitted from one subspecies to a different subspecies. The successful transmittance can be confirmed by the markers disclosed herein for example by the detection of the resistance gene or the cosegregating region. The markers disclosed herein can also be used to reduce the cosegregating regions in a backcrossing program involving several generations while keeping the resistance gene according to the invention in the offspring. Further markers for the detection of the resistance gene according to the invention can be deduced from the data disclosed herein. Especially those markers can be deduced which are located in a genomic interval that is flanked by markers s4p0421s01 and s4p1395s01 or which are flanked by those marker pairs which are given in passage [48] above. The deduction of further markers is facilitated by the genomic material comprised by the seeds which have been deposited with the NCIMB, Aberdeen, UK under access number NCIMB 43646. This genomic material allows for example the alignment of genetic material encoding the resistance according to the invention with genetic material lacking the resistance gene according to the invention. The genetic material lacking the resistance may be homologues or of the same physical position (compare Tab. 1b) as the genetic material encoding the resistance according to the invention. Those alignments may be used for the identification of polymorphisms like single nucleotide polymorphisms. On the basis of polymorphisms additional markers can be created for the detection of the identified polymorphisms, especially for those polymorphisms which are genetically linked to the resistance gene according to the invention. Alignments and detection of polymorphisms on the basis of alignment usually involve the use of computers and the storage of data on computer readable media. It is also suggested to convert the signal produced by molecular markers during the analysis of genetic material into electronically transmittable and/or electronically storable data which may be processed by the help of a computer. Such computer-based process may involve the determination or calculation of the estimated breeding value of a plant. The estimated breeding value may encompass the level of resistance or tolerance towards Cercospora. More information on the estimation of breeding values may be derived from U.S. Pat. No. 8,321,147B2.

Analyses yielded that the LRR gene has a moderate protein homology to the Cf-2 resistance protein from the tomato (UNIPROT|Q41397_SOLP1P. Cf-2.1)(sequence identity 322/830=38%). In fact, the identified Cercospora resistance-conferring protein is the best sugar beet protein homolog to the Cf-2 tomato resistance protein. The Cf-2 resistance protein from the tomato confers a resistance to Cladosporium fulvum—a type of black mold fungus (U.S. Pat. No. 6,287,865 B1)—via interaction with the avirulence protein Avr2 from C. fulvum. This leads to the activation of the plant immune defense against the pathogen; see Dixon et al., 1996 (Dixon, Mark S., et al., “The tomato Cf-2 disease resistance locus comprises two functional genes encoding leucine-rich repeat proteins.” Cell 84.3 (1996): 451-459). Due to the sequence homology between the Cf-2 gene and the identified LRR gene, it is to be assumed—but without thereby being bound to one theory—that a similar defense mechanism forming the basis of Cercospora resistance also occurs in the case of the sugar beet. However, a different mechanism is not to be precluded, due to the moderate sequence homology.

Furthermore, substitutions, deletions, insertions, additions, and/or any other change may be introduced into the nucleotide sequence according to the invention that, alone or in combinations, do in fact change the nucleotide sequence, wherein the modified nucleotide sequence may, however, perform the same function as the initial sequence. The present case deals with the coding of an amino acid sequence which confers resistance to Cercospora leaf spot disease. In a further embodiment, the invention therefore includes a nucleotide sequence that encodes a polypeptide which represents a derivative of the polypeptide which is encoded by the nucleotide sequence according to the invention, or which includes the amino acid sequence according to the invention. A derived amino acid sequence which has at least one substitution, deletion, insertion, or addition of one or more amino acids, wherein the functionality of the encoded polypeptide/protein is preserved, represents a derivative of the polypeptide. Substitutions, deletions, insertions, additions, and/or any other change, either solely or in combinations, that do in fact change the nucleotide sequence, but perform the same function as the initial sequence, may thereby be introduced into the nucleotide sequence using conventional methods that are known in the prior art, e.g., via site-directed mutagenesis, PCR-mediated mutagenesis, transposon mutagenesis, genome editing, etc.

The substitution of one amino acid by a different amino acid having the same or equivalent or similar chemical/physical properties is referred to as a “conservative substitution” or “semi-conservative substitution.” Examples of physical/chemical properties of an amino acid are, for example, hydrophobia or the charge. Which amino acid substitution represents a conservative or semi-conservative substitution is known to the person skilled in the art. Moreover, general expertise allows the person skilled in the art to recognize, identify, and detect which amino acid deletions and additions are harmless to the functionality of the resistance protein, and at which positions these are possible. The person skilled in the art is aware that, in the case of the present NBS-LRR protein for modifications of the amino acid sequence (substitutions, deletions, insertion, or additions of one or more amino acids), the functionality, in particular, of the conserved domains must be preserved, and that therefore only limited preceding modifications are possible in these domains.

The invention thus includes a functional fragment of the nucleotide sequence according to the invention. The term, “fragment,” thereby includes genes with a nucleotide sequence sufficiently similar to the aforementioned nucleotide sequence. The term, “sufficiently similar,” means that a first nucleotide sequence or amino acid sequence has a sufficient or minimum number of identical or equivalent nucleotides or amino acid groups relative to a second nucleotide sequence or a second amino acid sequence.

With regard to the amino acid sequence, after modification via an aforementioned method, this also has a common structural domain and/or possesses common functional activity. Nucleotide sequences or amino acid sequences that have an identity of at least approximately 70%, at least approximately 75%, at least approximately 80%, at least approximately 85%, at least approximately 90%, at least approximately 91%, at least approximately 92%, at least approximately 93%, at least approximately 94%, at least approximately 95%, at least approximately 96%, at least approximately 97%, at least approximately 98%, at least approximately 99%, or at least approximately 100% with the nucleotide sequence or amino acid sequence according to the invention are defined here as being sufficiently similar. This also explicitly encompasses the range of 90% to 100%. For the functional fragments, a sufficient similarity is established if the nucleotide sequence or amino acid sequence generally has the same property as the previously-named nucleotide sequence or amino acid sequence of the present invention. Those nucleotide sequences which encode a derivative or for a derived amino acid sequence are generated either directly or indirectly (for example, via amplification or replication steps) from an initial nucleotide sequence which corresponds to the nucleotide sequence according to the invention over the entire length, or at least in part.

Accordingly, the present invention includes a nucleotide sequence that is able to hybridize, under stringent conditions, with a nucleotide sequence complementary to a nucleotide sequence according to the invention or to the nucleotide sequence that encodes the amino acid sequence according to the invention.

In a further embodiment, the nucleic acid molecule according to the invention is characterized in that, after expression in a plant, it already, on its own, confers a dominant resistance effect against a pathogen—preferably, against Cercospora beticola—or that it encodes for a polypeptide that is able to confer a dominant resistance effect against Cercospora. In a preferred embodiment, the nucleic acid molecule or the polypeptide confers a resistance effect of at least one rating score—preferably, of at least two rating scores, and, particularly preferably, of three to four rating scores. Such a gene that already, on its own, confers such a strongly pronounced resistance to Cercospora in a plant, or that encodes a polypeptide that is able to confer such a pronounced resistance, is not known from the prior art. As was already described above, in previously available varieties on the market, the Cercospora resistance is transmitted via many resistance genes having little effect, and a disadvantage of such varieties is that their development is very slow and expensive due to the complicated transmission, and that such varieties have a markedly poorer crop yield relative to normal varieties, in the absence of an infestation. Among other things, this may be linked to the epigenetic interaction of some resistance genes with genes that are responsible for sugar production, which leads to reduced fitness of the plants, in the absence of the pathogen.

The inventors could thus for the first time provide a Cercospora resistance gene that may be used for markedly simplified breeding. Via the incorporation of this gene in elite lines, it is now possible to very quickly develop very high-yield varieties with a high Cercospora resistance. Accordingly, in the framework of the present invention there are provided for the first time a sugar beet plant, a chard plant, a red beet or beetroot plant, a fodder beet plant having the resistance according to the invention against Cercospora beticola and thus being encompassed by the present invention. As the listed plants are all cultivated plants, crops or plants which are suitable for the agricultural cultivation and which have the resistance according to the invention, are part of the invention. Especially such crops are part of the invention which comprise a subterrestrial storage organ usable as food, raw material or industrial source of sugar and which comprise the resistance according to the invention are a further aspect of the present invention. The storage organ can be for example the sugar containing beet body of the sugar beet, the consumable beet body of the red beet or the feedable beet body of the fodder beet. The subterrestrial storage organ can sum up to more than 50% and for the sugar beet even to more than 70% of the total mass of the full-grown plant. Furthermore, also seeds or seeding material of these plants are part of the invention. The seeds or the seeding material can be technically treated as described further below. Part of the invention are also plants of the genus Spinacia, Glycine, Daucus and Pastinaca comprising the resistance gene according to the invention. Especially plants of the species Spinacia oleracea and their varieties comprising the resistance gene according to the invention are included. The storage organ may be a taproot, especially a beet body.

The resistance gene according to the invention may be under control or may be operably linked to a promoter according to SEQ ID No. 7. The resistance gene may be also under control or may be operably linked to a nucleic acid molecule comprising a promoter according to SEQ ID No. 7. Sequences having 90%, 95% or 99% sequence identity over the whole length of the sequence to SEQ ID No. 7 are encompassed in this respect.

In this context, the invention also includes a nucleic acid that encodes the protein according to SEQ ID No. 3, wherein, in a specific embodiment, the nucleic acid according to SEQ ID No. 1 is excluded.

Furthermore, the present invention relates to a recombinant and/or heterologous DNA molecule that comprises the sequences of the nucleic acid molecule according to the invention. This DNA molecule, furthermore, preferably has a regulatory sequence. It may thereby be operatively linked with this regulatory sequence or be under the influence of this regulatory sequence. This regulatory sequence is preferably a promoter sequence and/or other sequences of transcription or translation control elements—for example, cis-elements. The regulatory sequence, which controls the expression of a gene that includes the nucleic acid molecule according to the invention, is preferably a sequence that is able to confer or modulate the expression, as a result of a pathogenic infection. This promoter is preferably able to control the expression of the DNA sequence specifically in leaves of the plant. The regulatory sequence may be heterologous to the expressing sequence. Such an approach has the advantage that the person skilled in the art may better adjust the expression rate of the expressing sequence, the tissue in which the expression occurs, and the point in time at which the expression occurs, in that he selects that regulatory sequence which is best suited to the respective use case. The heterologous DNA sequence preferably includes a nucleotide sequence which encodes a component of the plant pathogen defense (example: resistance genes (R-genes) or genes which encode enzymes involved in signal transfer, such as kinases or phosphatases, and for G-protein, or which encode a pathogenic effector (what are known as avirulence genes (avr))). The heterologous DNA sequence may be one of the DNA sequences according to the invention. The heterologous DNA sequence may also additionally encode further components of the plant pathogen defense. The heterologous DNA sequence may therefore be designed such that a polycistronic mRNA is created after its transcription.

The present invention furthermore also relates to a polypeptide which can be encoded by the nucleic acid molecule according to the invention and a functionally and/or immunologically active fragment thereof as well as an antibody that specifically binds to the polypeptide or to its fragment. The polypeptide particularly preferably has an amino acid sequence according to SEQ ID No. 3. The recombinant production of proteins, polypeptides, and fragments is familiar to the person skilled in the art (Sambrook et al., Molecular Cloning: A Laboratory Manual, 3rd ed., Cold Spring Harbor Laboratory Press, Cold Spring Harbor, N Y, 2001, or Wingfield, P. T., 2008, Production of Recombinant Proteins, Current Protocols in Protein Science, 52:5.0:5.0.1-5.0.4). Polyclonal or monoclonal antibodies to the protein according to the invention may be produced by the person skilled in the art according to known methods (E. Harlow et al., editor, Antibodies: A Laboratory Manual (1988)). The production of monoclonal antibodies, as well as of Fab and F(ab′)₂ fragments that are also useful in protein detection methods, may be performed via various conventional methods (Goding, Monoclonal Antibodies: Principles and Practice, pp. 98-118, New York: Academic Press (1983)). The antibodies may then be used for the screening of expression cDNA libraries in order to identify identical, homologous, or heterologous genes by means of immunological screening (Sambrook et al., Molecular Cloning: A Laboratory Manual, Cold Spring Harbor Laboratory Press, Cold Spring Harbor, N Y, 1989, or Ausubel et al., 1994, “Current Protocols in Molecular Biology.” John Wiley & Sons), or may be used for western blot analyses. In particular, the present invention relates to antibodies that selectively detect a polypeptide encoded by the Cercospora resistance-conferring allele according to the invention, and essentially do not detect the polypeptide encoded by the correspondingly sensitive allele, i.e., that they detect less, by a factor of 2—preferably, a factor of 5, and, more preferably, a factor or 10 or more—of the polypeptide encoded by the correspondingly sensitive allele than the polypeptide encoded by the Cercospora resistance-conferring allele according to the invention.

In a preferred embodiment, the antibody according to the invention is characterized in that it is a synthetic polypeptide which does not occur in nature.

Furthermore, the antibodies according to the invention may be linked with a fluorescent dye in order to be usable in an immunohistochemical method, for example, and evoke an antibody coloration. The fluorescent dye may be fluorochrome. The antibodies according to the invention may also be present linked with other signaling molecules. Among these are, for example, biotin, radioisotopes, reporter enzymes such as alkaline phosphatase, or oligonucleotides.

An additional subject matter of the invention is vectors or expression cassettes that include the nucleic acid molecule or the recombinant DNA molecule according to the invention—possibly under control of regulatory elements and, in particular, under control of functional regulatory elements in plants, as well as negative and/or positive selection markers. The vector backbone is thereby heterologous to the nucleic acid molecule according to the invention, which means that such a vector does not occur in nature and cannot be isolated from nature. The vector is a plasmid, a cosmid, a phage or an expression vector, a transformation vector, shuttle vector or cloning vector; it may be double-stranded or single-stranded, linear or circular; or it may transform a prokaryotic or eukaryotic organism either via integration into its genome or extrachromosomally. The nucleic acid molecule or DNA molecule according to the invention in an expression vector or expression cassette is, preferably, operatively linked with one or more regulatory sequences which allow the transcription and, optionally, the expression in a prokaryotic or eukaryotic cell; (Sambrook et al., Molecular Cloning: A Laboratory Manual, 3rd ed., Cold Spring Harbor Laboratory Press, Cold Spring Harbor, N Y, 2001). These regulatory sequences are preferably promoters or terminators—in particular, a transcription initiation starting point, a ribosome binding location, an RNA-processing signal, a transcription termination location, and/or a polyadenylation signal. For example, the nucleic acid molecule is here under the control of a suitable promoter and/or a terminator. Suitable promotors may be constitutive promotors (example: 35S promoter from the “Cauliflower mosaic virus” (Odell et al., Nature 313 (1985), 810-812); those promoters which are pathogenically inducible are especially suitable (example: PR1 promoter from parsley (Rushton et al., EMBO J. 15 (1996), 5,690-5,700)). Particularly suitable pathogenically-inducible promoters are synthetic or chimeric promoters which do not occur in nature, are composed of multiple elements, and contain a minimal promoter, and have at least one cis-regulatory element upstream of the minimal promoter, which at least one cis-regulatory element serves as a binding location for special transcription factors. Chimeric promoters ar designed according to the desired requirements and are induced or repressed via different factors. Examples of such promoters are found in WO 00/29592, WO 2007/147395, and WO 2013/091612. For example, a suitable terminator is the nos-terminator (Depicker et al., J. Mol. Appl. Genet. 1(1982), 561-573). Suitable promoters and terminators may also be the native promoter and the native terminator, whose DNA sequences are reproduced in SEQ ID Nos. 7 and 8. The vectors or expression cassettes additionally contain for conventional indicator/reporter genes or resistance genes for the detection of the transfer of the desired vector or DNA molecule/nucleic acid molecule, and for selection of the individuals that contain these, since a direct detection via the expression of the gene is for the most part rather difficult. Since the nucleic acid molecule according to the invention here itself encodes for a polypeptide which confers resistance to Cercospora leaf spot disease, it is not essential for the expression in plant cells to provide an additional resistance gene; however, it is recommended, in order to allow a rapid selection.

Examples of indicator/reporter genes are, for example, the luciferase gene and the gene encoding green fluorescent protein (GFP). These, furthermore, also allow tests for the activity and/or regulation of a promoter of the gene. Examples of resistance genes—especially, for plant transformations—an the neomycin phosphotransferase gene, the hygromycin phosphotransferase gene, or the gene encoding phosphinothricin acetyltransferase. Additional positive selection markers may be enzymes which provide the transformed plant a selection advantage over the non-transformed plant—in particular, a nutrient advantage, e.g., the mannose-6-phosphate isomerase or the xylose isomerase. However, this does not preclude additional indicator/reporter genes or resistance genes known to the person skilled in the art. In a preferred embodiment, the vector is a plant vector. Furthermore, the expression cassette may be present as integrated into a plant genome.

In a further aspect, the present invention relates to cells that include the vectors, recombinant DNA molecules, and/or nucleic acid molecules according to the invention. A cell in the sense of the invention may be a prokaryotic (for example, bacterial) or eukaryotic cell (for example, a plant cell or a yeast cell). The cell is preferably an agrobacterium such as Agrobacterium tumefaciens or Agrobacterium rhizogenes, an Escherichia coli cell, or a plant cell; the plant cell is particularly preferably a cell of a plant of the genus Beta, the species Beta vulgaris, or the subspecies Beta vulgaris subsp. vulgaris. The cell may also be present as a culture. The invention also consequently covers a cell culture which contains such cells. The cell culture is preferably a pure culture or an isolate that contains no cells of another type.

Known to the person skilled in the art are both numerous methods, such as conjugation or electroporation, with which he may introduce the nucleic acid molecule according to the invention, the recombinant DNA molecule, and/or the vector or the expression cassette of the present invention into an agrobacterium, and methods such as diverse transformation methods (biolistic transformation, agrobacterium-mediated transformation) with which he may introduce the nucleic acid molecule according to the invention, the DNA molecule, and/or the vector of the present invention into a plant cell (Sambrook et al., Molecular Cloning: A Laboratory Manual, 3rd ed., Cold Spring Harbor Laboratory Press, Cold Spring Harbor, N Y, 2001.

Furthermore, the present invention preferably relates to a Cercospora-resistant plant—preferably, a plant of the species Beta vulgaris subsp. vulgaris or a portion thereof—that contains the nucleic acid molecule according to the invention which confers the Cercospora resistance. The Cercospora-resistant plant may contain the nucleic acid molecule according to the invention as a transgene or as an endogene. Within the scope of the invention, for the first time, plants of the subspecies Beta vulgaris subsp. vulgaris were produced which contain the nucleic acid molecule according to the invention. The invention here also includes plants of the subspecies Beta vulgari subsp. vulgaris which contain the nucleic acid molecule according to the invention as an endogene.

A portion may thereby be a cell, a tissue, an organ, or a combination of multiple cells, tissues, or organs. A combination of multiple organs is, for example, a blossom or a seed. A Cercospora-resistant plant of the present invention preferably shows a higher resistance to Cercospora—in particular, Cercospora beticola—than a corresponding plant that does not contain the nucleic acid molecule according to the invention (control plant). The control plant ideally has the identical genotype as the transgenic plant, and has been cultured under identical conditions, but does not contain the resistance-conferring nucleic acid molecule. The level of the resistance, e.g., to Cercospora beticola, may be qualitatively established in plants of the genus Beta by determining rating scores. A higher resistance manifests in an improvement in the resistance by at least one rating score, by at least two rating scores, and, preferably, by at least three or more rating scores.

A plant cell or plant or portion thereof of the present invention that contains the nucleic acid molecule according to the invention—in particular, a plant of the genus Beta—preferably shows a higher resistance to a pathogen—in particular, to Cercospora beticola—than a corresponding plant cell or plant or portion thereof that does not contain the nucleic acid molecule according to the invention, or contains a sensitive allelic variant of the nucleic acid molecule. The level of the resistance, e.g., to Cercospora beticola, may be qualitatively established in plants of the genus Beta by determining rating scores. A higher resistance manifests in an improvement in the resistance by at least one rating score, by at least two rating scores, and, preferably, by at least three or more rating scores.

In the case of a transgenic plant cell, or plant or portion thereof, this comprises the nucleic acid molecule or DNA molecule according to the invention as a transgene or the vector or the expression cassette of the present invention. Such a transgenic plant cell or plant or portion thereof is, for example, one that is transformed—preferably, stably—with the nucleic acid molecule, DNA molecule according to the invention, or with the vector or the expression cassette of the present invention. In a preferred embodiment, the nucleic acid molecule is operatively linked with one or more regulatory sequences which allow the transcription and, optionally, the expression in the plant cell. The total structure made up of the nucleic acid molecule according to the invention and the regulatory sequence(s) then represents the transgene. Such regulatory sequences are, for example, a promoter or a terminator. Numerous functional promoters and terminators that are applicable in plants are known to the person skilled in the art.

The invention also includes a vacuole of the cell according to the invention, and the content substances stored therein.

Furthermore, the invention also relates to the cell extract from a cell—preferably, from a plant cell, particularly preferably, from a cell of Beta vulgaris, and, especially preferably, from a cell of one of the following crops: sugar beet, chard, or beetroot. No plant can be regenerated from the cell extract

Likewise encompassed by the invention is a plant genome containing the nucleic acid according to the invention.

The sugar concentration from the cell extract may thereby be increased relative to a cell that is not a cell according to the invention, but that belongs to the same species or crop. This applies, in particular, under the conditions when infested by Cercospora.

Also encompassed by the invention is the use of the cell extract for the production of sugar (saccharose) or for the production of juice—preferably, beetroot juice.

Likewise encompassed by the invention is the sugar—in particular, saccharose—contained in the cells according to the invention and their vacuoles.

An additional aspect of the invention is seed stock comprising seeds that contain the nucleic acid according to the invention. The nucleic acid according to the invention may be present transgenically or endogenously. The seed stock and the seeds may be technically treated. The invention thus also comprises technically-treated seed stock and technically-treated seeds. The various embodiments of technically-treated seed stock are explained in detail in the following whereby the term seed stock also includes seeds: Technically-treated seed stock may be present in polished form. The outermost layer of the seed is thereby removed, so that the seed assumes a more rounded form. This is helpful in sowing, where an optimally uniform shape leads to a uniform distribution of the seed stock grains. Technically-treated seed stock furthermore encompasses pelleted seed stock. The seed stock is thereby embedded in a pelleting mass that protects the seed stock contained therein and leads to a larger mass, such that the pelleted seed stock shows a greater resistance capability with regard to wind drift and is thus less susceptible to being blown away by the wind, and, at the same time, a more precise positioning during sowing is enabled. In a preferred embodiment of the invention, all pelleted seed stock grains of a batch or unit designated for sale have essentially the same shape and the same mass. Deviations of 5% in diameter and mass are possible. However, the deviations preferably do not exceed 1%. As one of the main components, the pelleting mass may contain for example a mineral compound such as clay, bentonite, kaolin, humus and/or peat, for example. It is possible to add an adhesive material like polyacylamide. Additional possible components are cited in U.S. Pat. No. 4,067,141. Moreover, the pelleting mass may contain additional chemical agents that positively influence the cultivation in practice. These may here be substances that are counted among fertilizing agents. These include compounds rich of one or more of the following elements: Nitrogen, Phosphorus and Potassium (macronutrients). Therefore, the fertilizing ingredients may contain for example Nitrate nitrogen, Ammonium nitrogen, Magnesium Nitrate, Calcium Ammonium Nitrate, Mono Ammonium Phosphate, Mono Potassium Phosphate and Potassium Nitrate. Furthermore, pelleting mass may contain fungicides, insecticides, and/or antifeedants. The fungicides may be thiram and/or hymexazol and/or other fungicides. The insecticide may be a substance from the neonicotinoid group. The substance from the neonicotinoid group is preferably imidacloprid (ATC Code: QP53AX17) and/or clothianidin (CAS number 210880-92-5). Furthermore, the insecticide may also be cyfluthrin (CAS number 68359-37-5), beta-cyfluthrin or tefluthrin. It is worth mentioned that the compound included in the dressing or pelleting mass are taken up by the plant and show systemic effect thereby providing suitable protection of the whole plant. Plants resulting from pelleted seed including one or more pesticides therefore differ from naturally occurring plants and show better performance under biotic stress conditions. In this context the invention also encompasses a mixture of a pelleting mass and a seed according to the invention. Furthermore, the invention also encompasses a method for producing a pelleted seed according to the invention comprising the following steps:
a) providing a plant seed comprising the nucleic acid according to the invention,
b) embedding the plant seed in a pelleting mass, and
c) allow the pelleting mass to dry or drying the pelleting mass, wherein the plant seed may be optionally a primed or pregerminated plant seed or the plant seed may be allowed to be primed during step b). Optionally, the method can be concluded by additional step d)
d) Pack up the embedded plant seed as derived by step c) in a packing. Examples of suitable packings are given herein elsewhere. The plant seed may for example be a seed of the plant genus Beta as described herein including sugar beet or red beet.

The seed pellet may also comprise beneficial bacteria. Preferably the bacteria are in a vital status and can undergo proliferation. Symbiotic bacteria can have diverse positive effects on the germination of the seed including increased vigor, increased germination speed and better resistance towards stress. According to a preferred embodiment microorganisms of the species Pseudomonas spp. are incorporated in the seed pellets. Further strains of different bacteria may be added. A possible method for improving the viability of microorganisms comprises the steps of:
a) suspending one or more pro-cultures of microbial cells in polymeric substance solution,
b) immobilizing said microbial cells by dropping the solution of step a) into multivalent ion solution thereby obtaining polymeric particles,
c) cultivating the encapsulated microorganisms in said polymeric particles for at least 12 hours, preferably for at least 24 hours in liquid media until an increase of the cell density of at least 2 to 10 log is obtained,
d) collecting the polymeric particles, and e) drying the polymeric particles.

Pelleted seeds comprising the resistance gene according to the invention and comprising furthermore microorganisms which have been obtained by the method disclosed in this paragraph are part of the invention. Furthermore, the microorganisms may be encapsulated and/or may be embedded in a extracellular matrix comprising naphthazarin.

In this context technically-treated seed stock encompasses also the dressed seed stock. The invention may be applied with any form of dressed seed stock. Dry dressing, wet dressing, and suspension dressing are thus also encompassed. The dressing may thereby also contain at least one dye (coloring), such that the dressed seed stock may be quickly differentiated from undressed seed stock, and, furthermore, good visibility in the environment is ensured after sowing. The dressing may also contain those agrochemicals which are described in the context of the pilling mass. The invention includes thus such dressed seed stock whereby the dressing contains at least one anti-feedant such as an insecticide and/or at least one fungicide. Optionally, so called electronic dressing (dressing by application of electric energy) may be applied. Electronic dressing is not a dressing in the strict sense of the word but is very suitable to destroy plant pathogens which adhere to the seed or seed stock before planting the seed or seed stock. It is also beneficial that seeds or seed stock which have only been treated by use of electronic dressing (without using agrochemicals) can be fed to animals in case more seed or seed stock is available than needed to till a field.

An additional form of technically-treated seed stock is encrusted seed stock. What is known as coating is also spoken of in this context as well as of seed stock treated with a coating. The difference to pelleted seed stock is that the seed grains retain their original shape, wherein this method is especially economical. The method is described in EP 0 334 258 A1, for example. An additional form of technically-treated seed stock is sprouted or primed seed stock. Sprouted seed stock is pretreated via a pro-germination, whereas primed seed stock has been pretreated via a priming (“germination”). Pre-germinated and primed seed stock have the advantage of a shorter emergence time. The point in time of the emergence after sowing is, at the same time, more strongly synchronized. This enables better agrotechnical processing during cultivation and especially during the harvest, and, additionally, increases the yield quantity. In pro-germination, the seed stock is germinated until the radicle exits the seed stock shell, and the process is subsequently stopped. In the priming, the process is stopped before the radicle exits the seed stock shell. Compared to pre-germinated seed stock, seed stock that has been subjected to a priming is insensitive to the stress of a re-drying and, after such a re-drying, has a longer storage life in comparison to pre-germinated seed stock, for which a re-drying is generally not advised. In this context, technically pre-treated seed stock also includes primed and re-dried seed stock. The process of pre-germination is explained in U.S. Pat. No. 4,905,411 A. Various embodiments of priming are explained in EP 0 686 340 A1. In addition to this, it is also possible to simultaneously pill and prime seed stock in one process. This method is described in EP 2 002 702 B1. Primed seed stock which is moreover pelleted, is encompassed by the present invention.

The technically-treated seed stock may additionally be furnished with one or more of the herbicide resistances explained above. This allows a further-improved agrotechnical cultivation, since the technically-treated seed stock may be deployed on a field that has previously been treated with weed killer, and that therefore is weed-free.

In addition to this, the invention also encompasses a mixture containing the seed stock according to the invention or the seeds according to the invention, and a dressing mass as defined above. The dressing mass is thereby preferably embodied as a pelleting mass, as defined above.

With storage of seed stock according to the invention, storage conditions are preferably to be chosen that do not negatively affect the stability or storage life of the seed stock. Fluctuations in humidity may, especially, have a disadvantageous effect here. Part of the invention is a method for the storage of the seed stock in a bag or container that is via simultaneously water-repellent and breathable. Such a bag or container may be designed as a carton or packing. Such a carton or packing may optionally possess an inner vapor barrier. If the carton or packing is designed as a duplex carton, its stability increases. A container, bag, carton or packing comprising the seed stock according to the invention, or technically-treated seed stock according to the invention, is likewise a part of the invention. It is likewise part of the invention to store seed stock according to the invention or technically-treated seed stock according to the invention in such a bag, container, box, packing or carton.

The present invention also encompasses varieties comprising the resistance gene according to the invention. Furthermore, plants, seeds and seedstock of such a variety are included. The seeds and seedstock of such a variety may be subject to a technical treating as described herein (e.g. pelleting). Suitable sugar beet varieties for the introduction of the resistance gene are for example BTS 7300 N, BTS 2045, BTS 3750, DAPHNA, KORTESSA KWS or SABATINA KWS. Sugar beet plants of the named varieties are also examples of hybrid sugar beet plants. Suitable red beet varieties for the introduction of the resistance gene are for example Jolie, Scarlett (PV-9503) or Diaz wherein Jolie and Diaz are also examples of hybrid red beet plants. Suitable Swiss Chard varieties for the introduction of the resistance gene are for example Fluence, Ion or Tesla/PV-9022. Suitable varieties of Spinacia oleracea (spinach) for the introduction of the resistance gene are for example PV-9210, PV-1194 or La Paz/PV-1237. Hybrid plants take advantage from the heterosis effect.

In one embodiment, the plant according to the invention is a hybrid plant or a double haploid plant. Hybrid plants and double haploid plants do not occur in nature and cannot be isolated from nature. In a further embodiment of the plant according to the invention, the nucleic acid molecule according to the invention is present in heterozygous or homozygous form. In the case of a hybrid plant, the nucleic acid molecule may also be present in hemizygous form. The invention also encompasses hybrid seeds and double haploid seeds which contain a nucleic acid according to the invention or a polypeptide according to the invention.

A further embodiment of the present invention comprises a plant—preferably, of the species Beta vulgaris—that is characterized in that the resistance to Cercospora in this plant is further increased. For example, this may be realized by means of “gene stacking,” i.e., the resistance is increased using this dose effect. For this, the plants according to the invention that contain the Cercospora resistance-conferring allele are over-transformed with this resistance allele in order to increase the amount of the transcription of the gene in the plant. An alternative approach includes the gene editing/site-directed mutagenesis or TILLING-mediated modification of the native promoter of the resistance-conferring allele, in order to increase its expression rate, or the modification of the resistance-conferring LRR gene allele itself, in order to increase its activity or stability. Such a method for increasing the activity by means of modification of a resistance gene is described in WO 2006/128444 A2, for example, and may be performed by means of the techniques known to the person skilled in the at. An additional approach may include the fusion of the nucleic acid molecule according to the invention with a heterologous promoter that exhibits a higher activity in comparison to the native promoter—in particular, after Cercospora infection.

An additional embodiment of the present invention relates to a sugar beet plant or a portion thereof or a pelleted seed of such a plant which is harvestable before bolting because no bolting of the sugar beet plant occurs during the first 10, 11, 12, 13, 14 or 15 months after germination and the development of a beet body is finished during this period. Suitable varieties to create a sugar beet plant according to this paragraph by introduction of the resistance according to the invention are for example DAPHNA, KORTESSA KWS or SABATINA KWS.

In one embodiment of the present invention the sugar beet plant or a portion thereof or a pelleted seed of such a plant has a genome allowing the development of a beet body having a mass summing up to at least 50%, 60%, 70%, 80% or even 90% of the total mass of the full-grown plant. Suitable varieties to create a sugar beet plant according to this paragraph by introduction of the resistance according to the invention are for example DAPHNA, KORTESSA KWS or SABATINA KWS. In another embodiment of the present invention the sugar beet plant or a portion thereof or a pelleted seed of such a plant has a genome allowing the development of a beet body having a minimum mass of 200 g, 250 g, 300 g, 350 g, 400 g, 450 g or 500 g and a maximum mass of 1000 g, 1100 g, 1200 g, 1300 g, 1400 g, 1500 g, 1600 g, 1700 g, 1800 g, 1900 g or even 2000 g via photosynthesis. Suitable varieties to create a sugar beet plant according to this paragraph by introduction of the resistance according to the invention are for example DAPHNA, KORTESSA KWS or SABATINA KWS.

An additional embodiment of the present invention is directed to a sugar beet plant or a portion thereof or a pelleted seed of such a plant wherein the genome of the sugar beet plant allows development of a beet body having a saccharose concentration of at least 10%, 11%, 12%, 13%, 14%, 15%, 16%, 17%, 18%, 19% or even 20%. Suitable varieties to create a sugar beet plant according to this paragraph by introduction of the resistance according to the invention are for example DAPHNA, KORTESSA KWS or SABATINA KWS.

In one embodiment of the present invention the sugar beet plant or a portion thereof or a pelleted seed of such a plant includes at least one, at least two, at least three, at least four, at least five, at least ten, at least twenty or even at least thirty mutation(s) relative to SEQ ID No. 1, 2 or 4.

The method for the production of an organism which comprises a mutated version of the nucleic acid molecule according to the above given embodiment [1] and/or a mutated version of a promoter comprising a nucleic acid sequence selected from the group consisting of (a) SEQ ID NO: 7, (b) a nucleotide sequence, which hybridizes under stringent conditions with a sequence which is complementary to the sequence according to (a), and (c) a nucleotide sequence which is at least 70% identical to a sequence according to SEQ ID NO: 7, wherein the method includes the following steps:
(I) Provision of an organism or a cell comprising the nucleic acid molecule and/or the promoter
(II) Increase of the mutation rate of the organism or the cell or mutagenesis of the organism or the cell
(III) Phenotypic selection of an organism, which as a result of a mutation exhibits an altered resistance or altered resistance level towards Cercospora beticola or Genotypic selection of an organism or a cell which comprises a mutation in the nucleic acid molecule and/or the promoter wherein the mutation has been created via step (II) and optionally
(IV) Regeneration of the organism from the cell obtained via step (III).

The organism can be a plant. Preferably the plant is a Beta vulgaris. However, it is also possible to use unicellular organisms as bacteria. The bacterium can be E. coli. If the organism is a plant then the method can be applied in vivo as well as in vitro. If the organism is a plant and the method is applied in vitro, a cell culture of the plant may be established and the increase of the mutation rate or the mutagenization may occur in the cell culture. The increase of the mutation rate encompasses for example the application of mutagenic agents like for example 5-bromouracil or ethylmethane sulfonate (EMS) or the application of physical mutagens like ionizing radiation or UV light. The mutagenization encompasses also the targeted mutagenesis. The targeted mutagenesis can be achieved by precise methods as gene editing (as explained further below). The regeneration of organism out of cells is explained in various standard references of the cell biology. The regeneration of plants is for example explained in the standard reference “Plant biotechnology: comprehensive biotechnology, second supplement” (Michael W. Fowler, Graham Warren, Murray Moo-Young—Pergamon Press—1992). The regeneration of Beta vulgaris out of the cell culture is described in Lindsey & Gallois “Transformation of sugarbeet (Beta vulgaris) by Agrobacterium tumefaciens.” Journal of experimental botany 41.5 (1990): 529-536.

These references also describe how plant cell cultures are established. As explained further above the mutated version of the nucleic acid molecule respectively the promoter characterizes themselves preferably due to the expression rate of the resistance imparting nucleic acid molecule which is increased by the mutation. Such an effect can also rely on the presence of several mutations. For example, it is possible to introduce two, three, four, five or more mutations in the promoter or the nucleic acid molecule.

By the introduction of mutations thus more resistance imparting protein can be built in the cell or the protein has a better effect. Thereby, the resistance in comparison to a control plant comprising the unaltered nucleic acid according to the invention can be increased for example by at least 1, 2, 3, 4, 5 or more percent. The increase can be measured as explained further below. Moreover, the resistance due to the mutation or mutations can be increased by at least one rating score. The determination of rating scores is explained elsewhere herein. Furthermore, the resistance protein can impart—as a result of the mutations—an altered effect and in some circumstances can exhibit effect against such pathogens which have adapted themselves to the initial resistance mechanism. In this context the invention encompasses also such mutated variants of the nucleic acids according to the invention and mutated variants of the protein according to the invention. Preferably the invention encompasses such variants which do not occur in nature and cannot be isolated from nature to make sure that the pathogen had no opportunity to adapt itselfto such variants. The above described method for the production of an organism which comprises a mutated version of the nucleic acid molecule may furthermore include a further step, in which those organisms or respectively plants are identified, which have a further increased resistance due to the mutation or mutations. If an increase of resistance has taken place may be determined by the herein explained rating scores or the measuring of the resistance level.

Besides the above described method for the production of organisms which comprise a mutated version of the nucleic acid molecule or of the promoter it is also possible to modify the according nucleic acids chemically in an isolated state to achieve the desired effects (as for example those which are described above). The advantage of this approach is that the compounds can be edited even more precisely. For this purpose, the following method is offered:

Production of a chemically modified nucleic acid molecule according to the above given embodiment [1] and/or a chemically modified promoter comprising a nucleotide sequence which is chosen from
(a) SEQ ID NO: 7;
(b) a nucleotide sequence which hybridizes under stringent conditions to a nucleotide sequence according to (a);
(c) a nucleotide sequence which is at least 70% identical to a sequence according to SEQ ID NO: 7;
wherein the method comprises the following steps:
(I) Provision of the nucleic acid molecule as stated above in isolated form
(II) chemical modification of the nucleic acid molecule or the promoter by one of the following steps:
(IIa) Mutagenization
(IIb) Gene editing
(IIc) Restriction and ligation respectively insertion or deletion.

Furthermore, chemical modifications can be generated by such approaches as stated elsewhere herein in context of allelic variants. The gene editing given under step (II) above is equal to the term “Genome-Editing”. Optionally the chemically modified nucleic acid molecule or the chemically modified promoter can be subsequently introduced into a cell or can be stably integrated. With the help of such a cell, the chemically modified nucleic acid molecule and the modified promoter can be propagated in context of the cell proliferation. They can be subsequently isolated in vast number and expression analyses may be performed. Expression analyses are especially suitable when the chemical modification concerns the promoter. It is possible to harvest the cells and to isolate the chemically modified resistance protein for chemical analyses. If the cell which comprises the chemically modified nucleic acid molecule or the modified promoter is a plant cell, a complete plant may be regenerated out of this cell. The approaches described within this passage can be performed subsequently to the above given method for the production of a modified form of the nucleic acid molecule and/or a modified promoter and the obtained variants are also a part of this invention. Moreover, a plant comprising the chemically modified nucleic acid molecule or the modified promoter are also part of the invention. Thus, the invention is also related to a plant obtained by this method. Furthermore, the invention relates also to the chemically modified nucleic acid molecules obtained by this method and to the encoded polypeptides. These compounds may be optimized versions of the original (not modified) compounds, wherein the resulting resistance level—as explained further above—may be increased by at least by 1, 2, 3, 4, 5, or more percent or may be increased by at least one rating score. In this regard the method for the production of a chemically modified nucleic acid molecule is also a method for the optimization of the nucleic acid molecule. The method for optimization may furthermore contain an additional step, in which those modified variants of the nucleic acid molecule are identified which lead in comparison to the unamended variants to an increased resistance in a plant.

In a further embodiment, the plant of the present invention additionally, transgenically or endogenously, comprises a second nucleic acid molecule at a different position in the genome, which encodes a polypeptide that is able to confer a resistance to Cercospora in the plant in which the polypeptide is expressed. For example, one or more of the resistance genes or resistance loci that are described in the prior art may—insofar as they are not already present in the initial genotype—be introduced into the present plant by means of crossing, transformation, homology-directed repair, or homologous recombination in the plant. Among these are, for example, the rhizomania resistance RZ1 (Lewellen, R. T., I. O. Skoyen, and A. W. Erichsen, “Breeding sugar beet for resistance to rhizomania: Evaluation of host-plant reactions and selection for and inheritance of resistance.” 50th Winter Congress of the International Institute for Sugar Beet Research, Brussels (Belgium), Feb. 11-12, 1987. IIRB. Secretariat General, 1987), or the rhizomania resistance RZ3 (WO 2014/202044) wherein the embodiment concerning RZ3 is explained under passage [94] in more detail.

The present invention additionally relates to a method for increasing the resistance to Cercospora in a plant of the species Beta vulgaris, wherein the increase in the resistance takes place without the resistance-conferring gene according to the invention, in comparison to an isogenic plant. The increase in the resistance may take place via integration of the nucleic acid molecule according to the invention into the genome of at least one cell of a plant of the species Beta vulgaris, as well as possible regeneration of a plant from the plant cell. The integration may take place both by means of sexual crossing, e.g., with one of the aforementioned Beta vulgaris subsp. maritima and subsequent selection, or by means of homology-directed repair or homologous recombination. The two latter methods cited are preferably supported by site-directed nucleases which may be selected from, but are not limited to, the following: CRISPR nuclease, including Cas9, CasX, CasY, or Cpf1 nuclease, TALE nuclease, zinc finger nuclease, meganuclease, Argonaut nuclease, restriction endonuclease, including FokI or a variant thereof, recombinase, or two, site-specific, nicking endonucleases. The introduction of the resistance-conferring gene by means of CRISPR-mediated homologous recombination in Beta vulgaris subsp. vulgaris is shown in Example 1.

Moreover, the invention encompasses also a method of producing an agronomically sugar beet plant of the genus Beta that displays improved resistance to Cercospora beticola, the method comprising introgressing into said plant a chromosomal interval that confers the improved resistance to Cercospora beticola, wherein the chromosomal interval maps to a position between a sequence represented by a marker selected from the group consisting of s4p4293s01 and s4p4295s01
and a sequence represented by a marker selected from the group consisting of s4p4301s01 and sxh0678s01,
characterized in that the chromosomal interval comprises a nucleotide sequence encoding a polypeptide that is able to confer resistance to Cercospora beticola in a plant in which the polypeptide is expressed wherein the nucleotide sequence is selected from
(a) a nucleotide sequence encoding a polypeptide having an amino acid sequence according to SEQ ID No. 3;
(b) a nucleotide sequence that comprises the DNA sequence according to SEQ ID No. 2;
(c) a nucleotide sequence that comprises a DNA sequence selected from the group consisting of SEQ ID No. 1 or SEQ ID No. 53;
(d) a nucleotide sequence that hybridizes to a nucleotide sequence which is complementary to the nucleotide sequence according to (a), (b), or (c), under stringent conditions;
(e) a nucleotide sequence encoding a polypeptide which, via substitution, deletion, and/or addition of one or more amino acids of the amino acid sequence, differs from a polypeptide encoded by the nucleotide sequence according to (a), (b), or (c);
(f) a nucleotide sequence encoding a polypeptide which has an amino acid sequence that is at least 70% identical to an amino acid sequence according to SEQ ID No. 3;
(g) a nucleotide sequence that is at least 70% identical to a DNA sequence according to SEQ ID No. 1 or SEQ ID No. 2;

An alternative approach includes the increase in the expression of the nucleic acid molecule according to the invention in the plant. This may take place via modification of the native promoter, wherein the modification preferably takes place by means of gene editing or site-directed mutagenesis which is mediated via site-directed nucleases, and, optionally, repair models. Examples of such nucleases have already been cited above. The increase in the expression of the nucleic acid molecule according to the invention may likewise take place via fusion of the nucleic acid molecule with a heterologous promoter, which exhibits a higher activity in comparison to the native promoter—in particular, after Cercospora infection. The fusion may likewise take place via site-directed nuclease and repair models, but also by means of direct insertion after double-strand break.

As has already been mentioned above, a method for increasing the Cercospora resistance, may also result in the increase in the activity and/or stability of the polypeptide according to the invention, via modification of the nucleotide sequence of the nucleic acid molecule according to the invention. Such a method for increasing the activity by means of modification of a resistance gene is described in WO 2006/128444 A2, for example, and may be performed by means of the techniques known to the person skilled in the art. This approach is explained in detail further below.

Alternatively, a Cercospora-resistant genotype may be produced from a Cercospora-sensitive genotype by means of random or directed mutagenesis of the nucleic acid sequence of the sensitive gene, and thus the Cercospora resistance may be increased. Examples of polymorphisms which differentiate the sensitive allele from the resistant allele are presented in FIG. 1.

For example, the sensitive allele may be modified via gene mutation by means of TALE nucleases (TALEN's) or zinc finger nucleases (ZFN's), as well as CRISPR/Cas systems, which—among other things—are described by way of example in WO 2014/144155 A1 (Engineering plant genomes using CRISPR/Cas systems) and in Osakabe & Osakabe, Plant Cell Physiol., 56 (2015), 389-400. This may also be achieved via use of the method designated as TILLING (Targeted Induced Local Lesions in Genomes), wherein it is described, e.g., in the German patent application DE 10 2013 101 617, how point mutations are caused in the sensitive gene, and plants are subsequently selected that exhibit a suitable, i.e., resistance-conferring, mutation, e.g., a barley resistant to yellow mosaic virus; see DE 10 2013 101 617 on pp. 4, 8, and 12, in paragraphs [0014], [0026], and [0038]. The TILLING method is also described in detail in the publication by Henikoff et al. (Henikoff et al., Plant Physiol. 135, 2004, 630-636).

These methods preferably lead to an improvement in the resistance by at least one rating score—particularly preferably, to an improvement in the resistance by at least two, three, or more rating scores. After mutagenesis of the plant cells and subsequent regeneration of plants from the mutagenized plant cells, or mutagenesis of plants, the plants may then be identified that exhibit one or more mutations, as depicted in FIG. 1, in an endogenous nucleic acid molecule. In this context the already mentioned plant according to the invention may be characterized by that the resistance is increased by at least one rating score, preferably by at least two or more rating scores. Alternatively, the resistance of the plants according to the invention may be increased for example by at least 1, 2, 3, 4, 5 or more percent in comparison to a control plant, which does not comprise the nucleic acid according to the invention. The increase can be measured by inoculation of respectively one healthy leaf with an isolate of the pathogen and the determination of the infested surface after 15 days. A reduce of 5% of the infested surface corresponds to an increase of the resistance of 5%. Further parameters for the conduction of the measuring can be derived from the below given embodiment “resistance rest”.

An additional embodiment of the present invention is a method for producing a Cercospora-resistant plant, which may take place via transformation of a plant cell with the nucleic acid molecule according to the invention, the recombinant DNA molecule, or with the vector or the expression cassette, and regeneration of the transgenic plant from the transformed plant cell (see Example 2), as well as, as described above, by means of random or targeted mutagenesis of the nucleic acid sequence of the sensitive gene to generate a Cercospora-resistant genotype, or via crossing and selection, e.g., with one of the aforementioned Beta vulgaris subsp. maritima. Vectors or expression cassettes, as well as methods for transforming plants, have already been described above.

The method for production of a Cercospora-resistant plant alternatively includes, as described above, the introduction of a site-directed nuclease and a repair matrix into a cell of a plant of the species Beta vulgaris, wherein the site-directed nuclease is able to generate at least one double-strand break of the DNA in the genome of the cell—preferably, upstream and/or downstream of a target region—and the repair matrix comprises the nucleic acid molecule according to the invention. The method furthermore includes the cultivation of this cell under conditions that allow a homology-directed repair or a homologous recombination, wherein the nucleic acid molecule is incorporated from the repair matrix into the genome of the plant. Furthermore, the regeneration of a plant from the modified plant cell is encompassed (see Example 1).

In a preferred embodiment, the target region is an allelic variant of the nucleic acid molecule according to the invention, wherein the allelic variant encodes a polypeptide which does not confer resistance to Cercospora. In a further preferred embodiment, this allelic variant comprises a nucleotide sequence that encodes a polypeptide with an amino acid sequence according to SEQ ID No. 6 and/or comprises the encoded DNA sequence according to SEQ ID NO: 5 or the genomic DNA sequence according to SEQ ID No. 4.

As described in connection with the nucleic acid molecule according to the invention, substitutions, deletions, insertions, additions, and/or any other change may be introduced that, either alone or in combinations, do in fact change the nucleotide sequence, but perform the same function as the initial sequence—here, the nucleotide sequence of the allelic variant of the nucleic acid molecule according to the invention. Therefore, in a further embodiment, the invention includes a nucleotide sequence that encodes a polypeptide which represents a derivative of the polypeptide which is encoded by the allelic variant of the nucleic acid molecule according to the invention, or which comprises the amino acid sequence of the allelic variant of the nucleic acid molecule according to the invention. A derived amino acid sequence which has at least one substitution, deletion, insertion, or addition of one or more amino acids, wherein the functionality of the encoded polypeptide/protein is preserved, represents a derivative of the polypeptide. The nucleotide sequence, using conventional methods that are known in the prior art, e.g., via site-directed mutagenesis, PCR-mediated mutagenesis, transposon mutagenesis, genome editing, etc., substitutions, deletions, insertions, additions, and/or any other change, either solely or in combinations with the gene, may thereby be introduced, which do in fact change the nucleotide sequence, but perform the same function as the initial sequence.

With regard to the amino acid sequence, after modification via an aforementioned method, this also has a common structural domain and/or possesses common functional activity. Nucleotide sequences or amino acid sequences that at least approximately 800, at least approximately 85%, at least approximately 90%, at least approximately 91%, at least approximately 92%, at least approximately 93%, at least approximately 94%, at least approximately 95%, at least approximately 96%, at least approximately 97%, at least approximately 98%, at least approximately 99%, or at least approximately 100% identical to the nucleotide sequence or amino acid sequence of the cited allelic variant of the nucleic acid molecule according to the invention are defined here as being sufficiently similar. Accordingly, the present invention includes a nucleotide sequence that is able to hybridize, under stringent conditions, with a nucleotide sequence that is complementary to a nucleotide sequence of the allelic variant of the nucleic acid molecule according to the invention or to the nucleotide sequence that encodes the corresponding amino acid sequence.

In a further preferred embodiment, the method according to the invention is characterized in that the double strand break occurs in an allelic variant of the nucleic acid molecule according to embodiment [1] or that the at least one double strand break occurs at a position which is at least 10,000 base pairs upstream or downstream of the allelic variant, wherein the allelic variant codes for a polypeptide which does not impart a resistance towards Cercospora.

For the person skilled in the art, it is obvious that numerous, different sensitive sequences may occur that derive from the nucleic acid molecule according to the invention, but do not confer resistance to Cercospora, such that the sequences listed above (SEQ ID Nos. 4, 5, and 6) should only be considered as an example of sequences, and the present invention is not limited to the aforementioned allelic variant of the nucleic acid molecule according to the invention. Such an allelic variant can comprise a nucleotide sequence, which is selected from:
(a) a nucleotide sequence encoding a polypeptide having an amino acid sequence according to SEQ ID No. 6;
(b) a nucleotide sequence that comprises the DNA sequence according to SEQ ID No. 5;
(c) a nucleotide sequence that comprises a DNA sequence according to SEQ ID No. 4;
(d) a nucleotide sequence that hybridizes with the complementary sequence according to (a), (b), or (c), under stringent conditions;
(e) a nucleotide sequence encoding a polypeptide which, via substitution, deletion, and/or addition of one or more amino acids of the amino acid sequence, differs from a polypeptide encoded by the nucleotide sequence according to (a), (b), or (c);
(f) a nucleotide sequence encoding a polypeptide which has an amino acid sequence that is at least 70%, 75%, 80%, 85%, 90%, 95%, 96%, 97%, 98% or 99% identical to an amino acid sequence according to SEQ ID No. 6;
(g) a nucleotide sequence that is at least 70%, 75%, 80%, 85%, 90%, 95%, 96%, 97%, 98% or 99% identical to a DNA sequence according to SEQ ID No. 4 or SEQ ID No. 5;

As described above, with quantitative heredity of QTL, not only is the desired resistance to often introduced into the plant, but, rather, also often unwanted features such as, for example, reduced yield, due to the inheritance of additional genes that are not linked with the positive feature of the resistance. This increasingly occurs if, as in the case of Cercospora resistance, the resistance is inherited in previously available cultivars via many resistance genes with small effect. Therefore, in a preferred embodiment, the introduction of the nucleic acid molecule according to the invention, which already shows, on its own, a dominant resistance effect, or of the vector or the expression cassette, is not linked with the introduction of unwanted features, wherein the yield is, preferably, not negatively affected. Furthermore, encompassed by the invention is the plant that is obtained via such a method.

Although the QTL analyses with that have previously been known from the prior art could detect actual QTL's, the underlying genomic regions that had shown a QTL effect also mediated the disadvantages described above, which is why “linkage drag” is also discussed in this context. At the same time, the QTL's and the effects connected therewith were not described uniformly in the respective prior art, and merely mediated a weak effect, such that the utilization of these results in the breeding of Cercospora-resistant plants was possible to only a limited extent, and was largely uncertain. Targeted breeding and controlled integration of the resistance gene into the gene pool of the sugar beet are now enabled by means of the identification of the resistance gene described herein. This ensures the breeding and generation of entirely new Cercospora-resistant cultivars that exhibit a high resistance to the pathogen, without negatively affecting the sugar yield.

The present invention likewise relates to methods/processes for the identification or detection, and optionally the selection and/or provision, of a plant of the species Beta vulgaris that is resistant to the pathogen Cercospora, characterized in that the method includes a step of the detection of the presence and/or of the expression of a nucleic acid molecule according to the invention or of the polypeptide according to the invention in the plant or a sample/portion thereof. The presence and/or the expression of a nucleic acid molecule according to the invention, or of the polypeptide according to the invention, may be tested by means of standard methods known to the person skilled in the art, e.g., by means of PCR, RT-PCR, or western blot. Preferably during the PCR the oligonucleotides or primers hybridize with a genomic template comprising the nucleotide sequence as defined in step (i) of [104] in such a way/in such a distance that the resulting amplificate is comprises up to 2000 bp, preferably up to 1500 bp, more preferably 1000 bp, most preferably up to 500 or 200 or up to 100 bp. The plant to be identified/detected and optionally selected and/or provided may be a plant comprising the resistance gene according to the invention and furthermore comprising a nucleotide sequence according to SEQ ID NO. 182 and/or the resistance allele according to marker s4p8772s01 or may be a plant obtainable from the seeds deposited with the NCIMB, Aberdeen, UK under access number NCIMB 43646. The methods and processes according to this paragraph may also be used to exclude or disregard plants which do not carry the resistance gene according to the invention. This is especially useful for example in breeding programs where some but not all of the offspring plants include the resistance.

Furthermore, the identification method according to the invention also includes the detection of the nucleic acid molecule according to the invention by means of detection of at least one polymorphism between resistant and sensitive sequences, i.e., between the sequences of the nucleic acid molecule according to the invention and the sequences of the allelic variant of the nucleic acid molecule according to the invention that is described above, using molecular markers that detect one or more polymorphisms. As has already been described above, it is obvious to the person skilled in the art that numerous sensitive sequences exist, i.e., numerous sequences that encode the allelic variant of the nucleic acid molecule according to the invention. One of these is presented by way of example in the sequence comparison with the nucleotide sequence of the nucleic acid molecule according to the invention in FIG. 1. A preferred embodiment of the method according to the invention consequently includes the detection of at least one polymorphism that is presented in FIG. 1 using molecular markers which detect the polymorphisms—in particular, diagnostic polymorphisms. This detection preferably occurs using at least one molecular marker per polymorphism—in particular, per diagnostic polymorphism. It is known to the person skilled in the art which marker techniques are to be applied to detect a corresponding polymorphism, and how molecular markers for this are constructed (see Advances in Seed Science and Technology Vol. I, Vanangamudi et al., 2008). Furthermore, the present invention encompasses molecular markers which describe or detect a polymorphism according to FIG. 1, such as the use of a molecular marker for detection of a polymorphism according to FIG. 1. It is thereby also possible to use markers that do not differentiate between various polymorphisms, as long as the markers are able to detect such a polymorphism as it occurs in the nucleic acid molecule according to the invention, but is not contained the sensitive allelic variant.

Alternatively, or additionally, the identification method according to the invention includes a step of detecting at least one marker locus in the nucleotide sequence of the nucleic acid molecule according to the invention or in a cosegregating regions thereof. Preferably the cosegregating region is a genomic region in Beta vulgaris which cosegregates with the Cercospora resistance conferred by the polypeptide according to the present invention, or with the nucleic acid molecule according to the present invention, more preferably the cosegregating region comprises and is flanked by markers sxh067&01 and s4p0264s01, by markers s4p4301s01 and s4p2271s01, by markers s4p4301s01 and s4p4293s01, or by markers s4p4301s01 and s4p4295s01. The detection may thereby take place via a method step in which at least one marker or at least one primer pair binds at the locus according to SEQ ID No. 74 or 75—preferably, at the locus according to SEQ ID No. 76 or 77—and, optionally as a result of this, a signal is generated, e.g., a fluorescence signal or a sequence amplificate. Thus, alternatively or additionally the cosegregating region may comprise a sequence according to SEQ ID NO 74 and/or 75, or SEQ ID NO: 76 and/or 77. Furthermore, the preceding identification methods also represent methods for selection of a plant which exhibits the resistance to Cercospora according to the invention. The method for selection includes a concluding step of selecting a resistant plant.

In this context, the present invention also includes the development or production of molecular markers that are suitable for detecting the aforementioned polymorphisms between the nucleic acid molecule according to the invention (resistant allele) and the sensitive allelic variant, wherein the markers are preferably suitable for detecting the polymorphisms presented in FIG. 1 or the construction of hybridization probes that specifically bind to the nucleotide sequence of the nucleic acid molecule according to the invention, or the production of a pair of nucleic acid molecules that is suitable for amplifying, in a PCR, a region that is specific to the nucleic acid molecule according to the invention, and thus for detecting these in a plant or plant cell.

The invention preferably includes a method for producing oligonucleotides of at least 15, 16, 17, 18, 19, or 20—preferably, at least 21, 22, 23, 24, or 25, particularly preferably, at least 30, 35, 40, 45, or 50, and, especially preferably, at least 100, 200, 300, 500 or 1,000—nucleotides in length that specifically hybridize with a nucleotide sequence of the nucleic acid molecule according to the invention or the nucleic acid molecule that is complementary thereto, or a pair of nucleic acid molecules—preferably, in the form of oligonucleotides—that is suitable for attachment as a forward and reverse primer to a region that is specific to the nucleic acid molecule according to the invention, and for amplifying this in a polymerase chain reaction (PCR), or that is suitable for hybridization as a forward and reverse primer to a region in the Beta vulgaris genome that, in Beta vulgaris, has a cosegregation with the Cercospora resistance conferred by the polypeptide according to the invention or with the nucleic acid molecule according to the invention. An example for suitable primers for the detection of a resistance-mediating nucleotide sequence according to the invention are given by SEQ ID NO 98 and SEQ ID NO 99. These two sequences build a primer pair which can by used in the PCR. The invention also includes a kit comprising oligonucleotides or molecular markers according to the invention.

The method for the production of oligonucleotides initially includes: the comparison of the nucleotide sequence of the nucleic acid molecule according to the invention with the nucleotide sequence of the corresponding nucleic acid molecule that does not confer resistance or of the sensitive allelic variant, which preferably has a nucleotide sequence according to SEQ ID No. 4 or 5; the identification of the sequence differences between the two nucleotide sequences; and the generation of nucleic acid molecules—here, meaning oligonucleotides—that specifically bind to the nucleic acid molecule according to the invention, but not to the nucleic acid molecule that does not mediate resistance.

Furthermore, the oligonucleotide according to the invention may be connected to a fluorescent dye in order to generate a fluorescence signal, e.g., under excitation via light of the corresponding wavelength. The fluorescent dye may be fluorochrome. The oligonucleotides according to the invention may be coupled with other compounds that are suitable for generating a signal. Such oligonucleotides do not occur in nature and also cannot be isolated from nature. The following is executed to produce such marked oligonucleotides: DNA may be marked bio-orthogonally. For this, DNA may be marked in vivo or in vitro with nucleoside analogs, which, for example, may subsequently be coupled with a fluorophore per Staudinger reaction. In addition to this, DNA may also be chemically provided with fluorophores. Oligonucleotides may be marked via a phosphoramidite synthesis with fluorophores that, for example, are used in QPCR, DNA sequencing, and in situ hybridization. Furthermore, DNA may be generated enzymatically in the course of a polymerase chain reaction with fluorescent nucleotides, or be marked with a ligase or a terminal deoxynucleotidyl transferase. DNA may also be detected indirectly via a biotinylation and fluorescent avidin. For couplings, fluorescein, fluorescent lanthanides, gold nanoparticles, carbon nanotubes, or quantum dots, among other things, are used as fluorophores. One of the most commonly used fluorescent substances is FAM (carboxyfluorescein). Consequently, oligonucleotides and, in particular, primers that possess a FAM marking are encompassed by the invention. FAM is preferably present as 6-FAM, wherein—depending upon the desired wavelength of the emission and excitation—other FAM variants, e.g., 5-FAM, may, however, also be used. Examples of additional fluorescence markers are AlexaFluor, ATTO, Dabcyl, HEX, Rox, TET, Texas Red, and Yakima Yellow. Depending upon the field of use, the oligonucleotides may be furnished with modifications of the bases or of the sugar phosphate spine. Among these are, among others, amino-dT, azide-dT, 2-aminopurine, 5-Br-dC, 2′-deoxyinosine (INO), 3′-deoxy-A, C, G, 5-Met-dC, 5-OH-Met-dCN6-Met-dA, and others.

Furthermore, the present invention also relates to a marker chip (“DNA chip”, “assay” or microarray) which contains at least one oligonucleotide according to the invention that is suitable for detection. The marker chip is suitable for application in one or more detection methods according to the invention.

The invention likewise includes a method for production of the protein according to the invention. The method includes the provision or cultivation of a cell culture which contains the SEQ ID No. 2, and the subsequent expression of the protein encoded by SEQ ID No. 2.

Furthermore, the present invention also relates to a Cercospora-resistant plant or a portion thereof which was identified, and, if applicable, selected, via a method as described in the preceding. In particular, the present invention relates to a population of plants comprising plants that are available according to one of the methods according to the invention as described in the preceding, and that preferably are resistant to Cercospora leaf spot disease or Cercospora infestation, and are characterized by the presence ofa nucleic acid molecule according to the invention. The population preferably has at least 10—preferably, at least 50, more preferably, at least 100, particularly preferably, at least 500, and, particularly in agricultural farming, preferably at least 1,000—plants. The proportion of plants in the population that do not carry the nucleic acid molecule according to the invention and/or are susceptible to Cercospora leaf spot disease is preferably below 25% —preferably, below 20%, more preferably, below 15%, even more preferably, 10%, and, in particular, preferably below 5%, if present at all.

With the fine mapping described above, the position of the Cercospora resistanco-conferring gene in the genome of Beta vulgaris subsp. maritima could be determined, and the gene itself and the surrounding sequence regions could be identified. This in turn represents the basis for the development of DNA hybridization probes or genetic markers in the target region, with the aid of which the Cercospora resistance-mediating gene could be detected, or could be differentiated from the gene that does not confer resistance.

DNA hybridization probes may be derived from the sequence of the Cercospora resistance-conferring gene and be used for the screening of genomic and/or cDNA banks of the desired organism. The probes may be used to amplify identified homologous genes via the known process of polymerase chain reaction (PCR), and to check whether the Cercospora resistance-conferring gene is present endogenously in an organism, or has been successfully introduced as heterologous genetic element.

The person skilled in the art may here resort to customary hybridization, cloning, and sequencing methods, which, for example, are listed in Sambrook et al., Molecular Cloning: A Laboratory Manual, 3rd ed., Cold Spring Harbor Laboratory Press, Cold Spring Harbor, N Y, 2001. The person skilled in the art may also synthesize and use oligonucleotide primers to amplify sequences of the Cercospora resistance-conferring gene. In order to achieve a specific hybridization, such probes should be specific and have at least a length of 15 nucleotides—preferably, at least 20 nucleotides. A detailed guide to hybridization of nucleic acids may be found in Tijssen, Laboratory Techniques in Biochemistry and Molecular Biology—Hybridization with Nucleic Acid Probes, Part 1, Chapter 2, “Overview of principles of hybridization and the strategy of nucleic acid probe assays.” Elsevier, New York (1993); and in Current Protocols in Molecular Biology, Chapter 2, Ausubel et al., eds., Greene Publishing and Wiley lnterscience. New York (1995).

Therefore, a nucleic acid molecule of at least 15, 16, 17, 18, 19, or 20—preferably, at least 21, 22, 23, 24, or 25, particularly preferably, at least 30, 35, 40, 45, or 50, and, especially preferably, at least 100, 200, 300, 500, or 1,000—nucleotides in length is the subject matter of the present invention, wherein this nucleic acid molecule specifically hybridizes with a previously-described nucleotide sequence according to the invention that comprises the Cercospora resistance-conferring gene. This also explicitly encompasses the range of 15 to 35 nucleotides.

The present invention thus also relates to markers as oligonucleotides—in particular, primer oligonucleotides. These comprise a nucleic acid molecule of at least 15 nucleotides in length that specifically hybridizes with a nucleotide sequence defined as in the preceding.

In particular, the present invention encompasses a pair of nucleic acid molecules—preferably, in the form of oligonucleotides or a kit containing this pair of oligonucleotides—that is suitable for hybridization as a forward and reverse primer to a region that is specific to the nucleic acid molecule according to the invention, and for amplifying this in a polymerase chain reaction (PCR), or that is suitable as a forward and reverse primer for hybridization to a region in the Beta vulgaris genome that, in Beta vulgaris, exhibits a cosegregation with the Cercospora resistance conferred by the polypeptide according to the invention, or with the nucleic acid molecule according to the invention.

The following advantages for the breeding and development of new resistant plant lines of the genus Beta may also be achieved via the present invention. Sequence information, as well as the identified polymorphisms which allow a differentiation between resistant and susceptible alleles of the disclosed gene, i.e., between the alleles that confer a Cercospora resistance and the alleles that are not capable of conferring this resistance, make possible the marker development directly in the gene, as described above, as well as in the regions situated upstream and downstream, which represents an important facilitation for the plant breeder—in particular, with regard to the development of optimized elite lines without “linkage drag.” Moreover, knowledge about the sequential structure may be used for the identification of additional resistance genes—in particular, against Cercospora—which are homologous or orthologous, for example.

Therefore, the present invention also encompasses a method for the identification of additional nucleic acid molecules encoding polypeptides or additional proteins that are able to confer a resistance to Cercospora in a plant in which the polypeptide is expressed. The person skilled in the art may thereby use databases, employing suitable search profiles and computer programs for the screening for homologous sequences or for sequence comparisons. Moreover, by means of conventional molecular biology techniques, the person skilled in the art may himself derive additional DNA sequences encoding Cercospora resistance proteins, and use these within the scope of the present invention. For example, suitable hybridization probes may be derived from the sequence of the nucleic acid molecule according to the invention and be used for the screening of genomic and/or cDNA banks of the desired organism. The person skilled in the art may here resort to customary hybridization, cloning, and sequencing methods, which, for example, are listed in Sambrook et al., Molecular Cloning: A Laboratory Manual, 3rd ed., Cold Spring Harbor Laboratory Press, Cold Spring Harbor, N Y, 2001. Using known sequences, the person skilled in the art may also synthesize and use oligonucleotide primers to amplify sequences of Cercospora resistance-conferring nucleic acid molecules.

In one embodiment, the present invention therefore encompasses a method for the identification of a nucleic acid molecule which encodes a polypeptide that is able to confer a resistance to Cercospora in a plant of the species Beta vulgaris in which the polypeptide is expressed. The method thereby includes the comparison of the amino acid sequence of the polypeptide according to the invention which, in Beta vulgaris subsp. vulgaris, confers a Cercospora resistance with amino acid sequences from a sequence database, or with sequences of allelic variants of the polypeptide according to the invention in genotypes of the species Beta vulgaris. Furthermore, the method according to the invention includes the identification of an amino acid sequence or of an allelic variant that is at least 80% identical to the amino acid sequence of the polypeptide according to the invention, as well as the introduction of a nucleic acid molecule encoding the identified amino acid sequence or allelic variant in a plant of the species Beta vulgaris; expression of the nucleic acid molecule in the plant; and, optionally, subsequent verification of the resistance to Cercospora.

As described in the preceding, additional Cercospora resistance-conferring proteins or their coding genes, i.e., homologs, analogs, and orthologs, that are at least 70%—preferably, at least 80%, particularly preferably, at least 90%, especially preferably, at least 95%, or even 98%—identical to the amino acid sequence of the polypeptide which is encoded by the nucleic acid molecule according to the invention may be identified via classical bioinformatic approaches (database searches and computer programs for screening for homologous sequences).

The term, homolog(s), thereby means that the genes concerned (from two different plant species) have essentially the same function and a common ancestor, and therefore typically show a significant identity in their nucleic acid or coded amino acid sequences. However, there are also many genes that are homologous to one another, without protein sequences resulting in a meaningful paired alignment. In contrast to this, the term, analog(s), describes genes or proteins that (likewise) have an identical or similar function, but are not created from the same structure, i.e., have no common ancestor. In this case, often, no significant identity can be established in their nucleic acid or encoded amino acid sequence, or, in the best case, in specific functional domains.

In the context of genome sequencing, homologs are, for annotation, more finely classified. The terms, orthology and paralogy, have been introduced for this. Orthologs are genes that are connected via a speciation event. Paralogs are genes that trace back to a duplication event.

A gene is, then, fundamentally a homolog or analog or ortholog in the sense of the present invention if it is able to confer Cercospora resistance in a plant. To check, methods, which have already been described in the preceding, known to the person skilled in the art are used, e.g., the amplification of the identified homolog or analog or ortholog by means of PCR, cloning in expression vectors, introduction into the target plant or plant cell, and checking the resistance.

As described above, the usage disclosed here of the resistant gene allele in cis- or transgenic approaches opens up the possibility of new resistant species of the genus Beta which, using the dose effect, exhibit a higher resistance, or in which a resistance break may be avoided and the resistance development optimized via the stacking of the disclosed gene with other resistance genes. Modifications of the gene by means of tilling or targeted engineering to optimize the codon selection for an increased expression or for the development of new or modified resistance alleles are also possible. According to a preferred embodiment the codon-optimized sequences or the modified resistance alleles are not occurring in nature but are artificial. An example of a modified genomic sequence is provided by SEQ ID No. 94 in which the codon at position 16-18 is modified but the encoded amino acid sequence is unchanged and corresponds to SEQ ID No. 3. An example of a modified cDNA sequence is provided by SEQ ID No. 95 in which the codon at position 55-57 is modified but the encoded amino acid sequence is unchanged and corresponds to SEQ ID No. 3. SEQ ID No. 94 and SEQ ID No. 95 are also examples for hybridizing sequences. An example of a modified resistance conferring allele is given by the amino acid sequence according to SEQ ID No. 96 in which the amino acid valine has been replaced with the amino acid leucine at position 209. The amino acid sequence according to SEQ ID No. 96 is encoded by the modified cDNA according to SEQ ID No. 97. These sequences do not occur in nature but are artificial. When replacing amino acids in for example the resistance-mediating Sequence according to SEQ ID No. 3 it is recommended to exchange amino acids within the following groups:
a) glycine, alanine, valine, leucine, isoleucine
b) serine, cysteine, selenocysteine, threonine, methionine
c) phenylalanine, tyrosine, tryptophan
d) histidine, lysine, arginine
e) aspartate, glutamate, asparagine, glutamine.

The present invention also relates to the use in a plant of the identified Cercospora resistance-conferring gene allele in a genetic or molecular stack with other genetic elements which may confer agronomically advantageous properties. The economic value of cultivated plants may thereby be markedly increased, in that, for example, the yield performance is increased in comparison to plants that possess the same genetics, but have not been furnished with the nucleic acid according to the invention. Furthermore, new crop areas for a plant may be opened up that were not previously accessible to the cultivation of this plant due to biotic factors such as strong pathogen pressure. In particular, the present invention relates to the use of the identified Cercospora resistance-conferring gene allele in methods for controlling an infestation with the pathogen Cercospora beticola in the agricultural or horticultural cultivation of plants of the genus Beta, e.g., encompassing the identification and selection of plants of the genus Beta with the aid of one of the methods described in the preceding and/or the cultivation of the plants so selected or descendants thereof. The present invention thus includes a method for the cultivation of plants of the species Beta vulgaris, including, in a first step, the provision of Cercospora-resistant plants of the species Beta vulgaris according to the invention, or the production of plants of the species Beta vulgaris with the aid of the production method according to the invention, or the identification and selection of plants of the species Beta vulgaris with the aid of the identification method according to the invention that has been described in the preceding; and including, in a second step, the cultivation of the plants from the first step, or the deployment of seed stock of the plants from the first step, or the raising of plants from the first step. The cultivation method thereby counteracts an infestation of the cultivated plants by Cercospora. The cultivation method may be part of a method for producing sugar. The method for the production of sugar includes the steps of the cultivation method, and additionally, as a penultimate step, the harvesting of the cultivated plants, and, as a last step, the extraction of sugar from the aforesaid plants. The methods for identification or detection, the marker and oligonucleotides as described herein may be for example used on the seeds deposited with the NCIMB, Aberdeen, UK under access number NCIMB 43646 or may be used on a plant resulting from the deposited seeds. Furthermore, they may be used a) on a plant which is the offspring of the deposited seeds or b) on a plant into which the resistance gene according to the invention has been transferred by one of the methods described herein (e.g. transformation etc.) or c) for the discrimination of a plant which comprises a resistance gene according to the invention and a plant lacking the resistance gene of the invention.

The cultivation method may also be part of a method for producing seed stock. The method for the production of seed stock includes the steps of the cultivation method, and additionally, as a penultimate step, the vernalization of the cultivated plants, and, as a last step, the extraction of seeds from the aforesaid plants.

The extracted seeds may optionally be pelleted, in order to obtain pelleted seed stock of the species Beta vulgaris. In this instance, it is a method for the production of pelleted seed stock.

Moreover, the method for the production of seed stock may be designed as a method for the production of Cercospora-resistant seed stock. The method for the production of Cercospora-resistant seed stock includes the steps of the method described above for the production of seed stock, and additionally, as a last step, the verification of the nucleic acid according to the invention according to a method described herein in at least one of the extracted seeds—preferably, in at least 0.1% or in at least 1% of the extracted seeds. The verification is particularly preferably implemented so that the seed remains germinable. This means that the extraction of the DNA required for verification from the seed does not neutralize the germinability of the seed. In such an instance, the verification of the nucleic acid according to the invention may have taken place in an especially large proportion of all extracted seeds. For example, the verification may take place in at least 2%—preferably, at least 3%, particularly preferably, at least 4%—of all extracted seeds.

The plants according to the invention, their cells, or seeds or seed stock according to the invention may possess additional, agronomically advantageous properties, or be furnished with such. One example is the tolerance or resistance to an herbicide such as glyphosate, glufosinate, or ALS inhibitors. The tolerance to glyphosate or an ALS-inhibitor herbicide is preferred. A specific embodiment of the glyphosate resistance is disclosed in U.S. Pat. No. 7,335,816 B2. Such a glyphosate resistance is, for example, available from seed stock stored at the NCIMB, Aberdeen (Scotland, UK), under the access number, NCIMB 41158 or NCIMB 41159. Such seeds may be used in order to obtain a glyphosate-tolerant sugar beet plant. The glyphosate resistance may also be introduced into other species of the genus Beta via crossing.

In the context of glyphosate resistance the invention thus also encompasses plants, their cells, or seeds or seed stock, characterized in that these contain the nucleic acid according to the invention, and furthermore in that
a) a DNA fragment of the genomic DNA of the plant, portions, or seeds thereof may be amplified via polymerase chain reaction with a first primer that has the nucleotide sequence of SEQ ID No. 81, and a second primer that has the nucleotide sequence of SEQ ID No. 82, wherein the DNA fragment is at least 95%—preferably, 100%—identical to the nucleotide sequence of SEQ ID No. 83, and/or
b) a DNA fragment of the genomic DNA of the plant, portions, or seeds thereof may be amplified via polymerase chain reaction with a first primer that has the nucleotide sequence of SEQ ID No. 84, and a second primer that has the nucleotide sequence of SEQ ID No. 85, wherein the DNA fragment is at least 95% identical—preferably, 100% identical—to the nucleotide sequence of SEQ ID No. 86, and/or
c) a DNA fragment of the genomic DNA of the plant, portions, or seeds thereof may be amplified via polymerase chain reaction with a first primer that has the nucleotide sequence of SEQ ID No. 87, and a second primer that has the nucleotide sequence of SEQ ID No. 88, wherein the DNA fragment is at least 95% identical—preferably, 100% identical—to the nucleotide sequence of SEQ ID No. 89.

In certain embodiments, the wild type Beta vulgaris epsp synthase has an amino acid sequence as provided in NCBI reference sequence XP_010692222.1. This wild type epsp synthase may be mutated or modified (for example by one of the means for mutation or modification as disclosed herein) for conferring resistance to glyphosate. Such a mutation may create an endogenous allele encoding an epsp synthase having at position 179 an amino acid different from proline. Preferably the amino acid different from proline is serine. Plants comprising a mutated epsp synthase and the resistance gene according to the invention are encompassed herein. This includes plants comprising the resistance gene of the invention and furthermore a nucleic acid molecule encoding an epsp synthase having at position 179 an amino acid different from proline and comprising an amino acid sequence selected from
i) the sequence of SEQ ID NO: 223
ii) the sequence i) having at position 179 an amino acid different from serine and proline, or
iii) a sequence having an identity of at least 90%, more preferable 95%, most preferably 99% to the sequence of i) or ii)
over the entire length of the sequence.

In this context the mutated EPSP synthase may be for example encoded by a nucleic acid sequence according to SEQ ID No. 224. Further technical details on the use of mutated EPSP synthase may be derived from WO2020064687 (A1). A plant comprising the resistance gene according to the invention and expressing a mutated EPSP synthase may furthermore comprise a resistance towards one or more ALS inhibitor herbicide as described herein. A plant comprising the resistance gene according to the invention and expressing a mutated EPSP synthase may be a non-transgenic plant. The mutated EPSP synthase in the context of this invention is an artificial compound which does not occur in nature. According to a specific embodiment the plant comprising the mutated EPSP synthase and the resistance gene according to the invention is not a product of an essentially biological process.

A specific embodiment of the ALS-inhibitor herbicide resistance is disclosed in the document, WO2012/049268 A1. For example, such an ALS-inhibitor herbicide resistance is available from a deposit of NCIMB, Aberdeen, UK, under the number NCIMB 41705. Furthermore, such an ALS-inhibitor resistance may be produced via tilling or site-directed mutagenesis, e.g., via gene editing, such as through the use of CRISPR/Cas, CRISPR/Cpf1, TALENS or zinc finger nucleases. The invention thus also encompasses plants, their cells, or seeds or seed stock, characterized in that these contain the nucleic acid according to the invention, and furthermore in that these exhibit a mutation in an endogenous acetolactate synthase gene, wherein the acetolactate synthase gene encodes an acetolactate synthase protein which, as a result of the mutation at position 569, has a different amino acid than tryptophan. As a result of the mutation, the amino acid at position 569 is preferably alanine, glycine, isoleucine, leucine, methionine, phenylalanine, proline, valine, or arginine. Position 569 is preferably defined via the position 569 of SEQ ID No. 90. Furthermore, the specific sequence of the mutated acetolactate synthase gene SEQ ID No. 91 is preferred. The mutated sequence of the acetolactate synthase gene, or the sequence according to SEQ ID No. 91, does not occur in nature and cannot be isolated from nature. Furthermore, the mutation may be present both heterozygously and homozygously in the plants, their cells or seeds, or the seed stock. We recommend the homozygous presence of the mutation, since this promotes a more stable or more intensive phenotypical occurrence of the resistance.

Numerous additional herbicides and their applicability are known to the person skilled in the art from the prior art. He may resort to the prior art in order to achieve knowledge of which genetic elements are to be used in what manner in order to implement a corresponding tolerance in plants.

Moreover, an herbicide tolerance has the synergistic effect that the occurrence of weeds is reduced via the use of herbicides. This is advantageous in combating Cercospora, because it is known that the conidia (asexual spores) or the pseudostroma (mycelium) of Cercospora beticola can survive for up to 2 years on plant material.

A further example of an agronomically advantageous property is an additional pathogen resistance, wherein pathogens may be insects, viruses, nematodes, bacteria, or fungi, for example. For example, a broad pathogen defense for a plant may be achieved via combination of different pathogen resistances/tolerances, since genetic elements may exhibit additive effects among one another. For example, numerous resistance genes for this are known to the person skilled in the art as genetic elements. For example, US20160152999A1 discloses an RZ resistance gene against the disease Rhizomania. This disease is caused by the agent, “Beet Necrotic Yellow Vein Virus.” Several disease resistances contained in one plant have synergistic effects upon one another. If a plant is infested for the first time by a pathogen, its immune system is normally weakened, and the epidermis as an outer barrier is often damaged, such that the probability of further infections is increased. An additional example of an agronomically advantageous property is cold tolerance or frost tolerance. Plants which exhibit this property may already be sown earlier in the year, or may remain in the field longer, which may lead to increased yields, for example. Here, the person skilled in the art may also resort to the prior art to find suitable genetic elements. Additional examples of agronomically advantageous properties are water usage efficiency, nitrogen usage efficiency, and yield. Genetic elements which may be used to confer such properties might be found in the prior art.

Furthermore, numerous modifications for pathogen defense are known to the person skilled in the art. In addition to the families of the R-genes that are often described, the Avr/R approach, the Avr gene complementation (WO 2013/127379), the autoactivation of an R-gene (WO 2006/128444), or the HIGS (host-induced gene silencing) approach (e.g., WO2013/050024) may be advantageously used. In particular, the autoactivation of an R-gene might be important to the present invention. For this, a nucleic acid is to be created that encodes an autoactivated resistance protein for generation of a resistance to pathogens in plants. This nucleic acid then has only a limited portion of an NBS-LRR resistance gene, such as the wb-R-gene, which extends downstream from the 5′ end of the coding region of the NBS-LRR resistance gene to the beginning of the coding for the NBS domain of the NBS-LRR resistance gene.

In this context, a method is also encompassed which contains the step of the removal of that region of the nucleic acid according to the invention which encodes the N-tenminal region and which begins with the p-loop in the NBS domain, and extends up to the end of the N-terminal region.

The resistance proteins that are encoded for by such shortened nucleic acids are generally autoactivated, in that these resistance proteins trigger an immune reaction in the plant, even in the absence of the associated pathogen, and thus increase the base immunity of the plant. Furthermore, such a shortened nucleic acid according to the invention, and the polypeptide that is encoded by this, are encompassed.

Furthermore, the invention also includes the use of the Cercospora resistance-conferring gene allele, identified with a method described above, for combination with one of the preceding modifications, or with a genetic element described in the preceding which may convey in a plant one or more agronomically advantageous properties.

In addition to relating to the plant according to the invention, the present invention also relates to seeds or descendants, or to an organ, a plant part, a tissue, or a cell thereof in the production of products that are typically produced from sustainable raw materials, such as foodstuffs and animal feed—preferably, sugar or syrup (molasses), wherein the molasses is also used for industrial applications, e.g., in alcohol production or as a growing medium for the production of biotechnological products, in the production of materials or substances for the chemical industry, e.g., refined chemicals, pharmaceuticals or precursors thereof, diagnostics, cosmetics, bioethanol, or biogas. An example of the use of sugar beet as a biogenic raw material in biogas plants is described in the application DE 10 2012 022 178 A1; see, for example, paragraph 10.

The following examples explain the invention, but without limiting the subject matter of the invention. Unless indicated otherwise, standard molecular biology methods have been used; see, for example, Sambrook et al., Molecular Cloning: A Laboratory Manual, 3rd ed., Cold Spring Harbor Laboratory Press, Cold Spring Harbor, N Y, 2001, Fritsch et al., Cold Spring Harbor Laboratory Press: 1989; Mayer et al., Immunochemical Methods in Cell and Molecular Biology, eds., Academic Press, London, 1987, and Weir et al., Handbook of Experimental Immunology, Volumes I-TV, Blackwell, eds., 1986.

Some of the most important sequences according to the invention are explained in detail in the following:
SEQ ID No. 1: genomic DNA sequence of the Cercospora resistance-conferring gene from Beta vulgaris subsp. maritima.
SEQ ID No. 2: cDNA sequence of the Cercospora resistance-conferring gene as it does not occur in nature.
SEQ ID No. 3: amino acid sequence of the Cercospora resistance-conferring protein as it is encoded by SEQ ID No. 1 or SEQ ID No. 2.
SEQ ID No. 4: genomic DNA sequence of the sensitive Variant of the Cercospora resistance-conferring gene
SEQ ID No. 5: cDNA of the sensitive Variant of the Cercospora resistance-conferring gene
SEQ ID No. 6: Amino acid sequence of the sensitive Variant of the Cercospora resistance-conferring gene
SEQ ID No. 7: native promoter of the Cercospora resistance-conferring gene from Beta vulgaris subsp. maritima.
SEQ ID No. 8: native terminator of the Cercospora resistance-conferring gene from Beta vulgaris subsp. maritima.
SEQ ID No. 53: sequence of the locus from Beta vulgaris subsp. maritima containing the Cercospora resistance-conferring gene according to SEQ ID No. 1.

### EXAMPLES

### Example 1: Introduction of the Resistance-Conferring Gene by Means of CRISPR-Mediated Homologous Recombination in Beta vulgaris subsp. vulgaris

Design and Selection of the crRNA:

Suitable crRNA's for Cpf1-mediated induction of double-strand breaks have been designed with the aid of CRISPR RGEN Tools (Park J., Bae S., and Kim J.-S. Cas-Designer: A web-based tool for choice of CRISPR-Cas9 target sites. Bioinformatics 31, 4014-4016 (2015); Bae S., Park J., and Kim J.-S. Cas-OFFinder: A fast and versatile algorithm that searches for potential off-target sites of Cas9 RNA-guided endonucleases. Bioinformatics 30, 1473-1475 (2014)). For this, suitable protospacers were sought within the genomic DNA sequences having a length of 500-1,300 bp which flank the 5′- and 3′-end of the Cercospora resistance gene from Beta vulgaris subsp. maritima. In order to ensure the functionality of the endonuclease Cpf1 from Lachnospiraceae bacterium ND2006 (Lb), protospacers having a length of 24 nt were selected whose genomic binding sequence at the 5′-end was flanked by an essential protospacer adjacent motif (PAM) having the sequence 5′-TTTV-3′ (V=G or C or A). Suitable protospacers were selected according to the predetermined quality criteria of the tool and reconciled as to potential off-targets with a reference genome of B. vulgaris subsp. vulgaris. For the continuing tests, crRNA's, exclusively, were selected that, in addition to the actual target sequence, have at most 15 identical bases with a functional PAM. Since the first 18 nt of the protospacer are essential to the detection and cutting of the target sequence, an unwanted cutting within other genomic sequences could be precluded in this way (Tang, X., L. G. Lowder, T. Zhang, A. A. Malzahn, X. Zheng, D. F. Voytas, Z. Zhong, Y. Chen, Q. Ren, Q. Li, E. R. Kirkland, Y. Zhang, and Y. Qi (2017), “A CRISPR-Cpf1 system for efficient genome editing and transcriptional repression in plants.” Nat Plants 3: 17018). In this way, four potential crRNA's at the 5′-flanking region (5′crRNA #1-4) and three crRNA's at the 3′-flanking region (3′crRNA #1-3) of the resistance gene could be identified (see Table A).

TABLE A
Selected target sequences within the 5′- and 3′-flanking DNA
sequences of the resistance gene in B. vulgaris. The PAM is underlined.

| Name of the | Genomic target sequence with | Binding at the +/− |
|---|---|---|
| crRNA | 5′-flanking PAM (underlined) | strand |
| 5′crRNA#1 | TTTATTTCGATTTCGATTCTTGGATTAT | − |
| | (SEQ ID No. 16) | |
| 5′crRNA#2 | TTTCAACCCAGTATCCTTATCCGTCACT | − |
| | (SEQ ID No. 17) | |
| 5′crRNA#3 | TTTATTTAAACATGATACGTATCATATT | + |
| | (SEQ ID No. 18) | |
| 5′crRNA#4 | TTTAAACATGATACGTATCATATTGAGT | + |
| | (SEQ ID No. 19) | |
| 3′crRNA#1 | TTTGTGGGTGGGTGGTTTTCACGTGTGT | − |
| | (SEQ ID No. 20) | |
| 3′crRNA#2 | TTTCCCCTCCCTTTGCCGCTGCGAAGTT | − |
| | (SEQ ID No. 21) | |
| 3′crRNA#3 | TTTCTTCTTCTTGCTTCCACCATAACAC | − |
| | (SEQ ID No. 22) | |

Cloning of the genetic elements: For the cloning of the cpf1-expression cassette and the crRNA-expression cassette, first, a detection sequence of the restriction enzyme of BbsI that prevents cloning was removed from the target vector pZFNnptII via introduction of a point mutation (T to G). The mutagenesis was performed with a mutagenesis kit according to the specification of the manufacturer, using two mutagenesis primers (see Table B).

TABLE B
Mutagenesis primer used to introduce a point
mutation (T to G, underlined) to remove the BbsI
detection sequence.

| Name | Sequence 5′→3′ |
|---|---|
| Mutagenesis | TCAGTGCAGCCGTCGTCTGAAAACGACA |
| primer 1 | (SEQ ID No. 23) |
| Mutagenesis | TGTCGTTTTCAGACGACGGCTGCACTGA |
| primer 2 | (SEQ ID No. 24) |

For the expression of the Lbcpf1 gene in B. vulgaris, a DNA sequence codon-optimized for A. thaliana, with 5′-flanking PcUbi promoter sequence from Petroselinum crispum (SEQ ID No. 79) and a 3′-flanking 3A terminator sequence from Pea sp. as a DNA fragment, was synthetically produced. The restriction interface (HindIII) that is relevant to cloning within the Lbcpf1-coding sequence (CDS) [SEQ ID No. 78] were removed via the introduction of a silent mutation (base exchange, without modifying the amino acid sequence), in order to avoid an unintended cutting within the coding region. The codon optimization was performed with the aid of the GeneArt algorithm from Invitrogene/ThermoScientific. In order to enable the transport of the Cpf1 in the cell nucleus, the coding sequence of the nucleus location signal (NLS) of the SV40 was integrated into the cpf1 CDS at the 5′-end, and the NLS of the nucleoplasmin was integrated at the 3′-end. For the ligation in the binary target vector pZFNnptII (FIG. 2), the expression cassette was flanked by two HindIII restriction interfaces and subsequently ligated to pZFNnptII_LbCpf1. The successful insertion of the PcUbi::Cpf1::TPea expression cassette was verified by means of sequencing, wherein the binding regions of the primers used for the sequencing were situated both in the flanking vector regions and within the expression cassette (see Table C).

TABLE C
Primer used for the sequencing of the PeUbi::Cpf1::TPea
expression cassette integrated into pZFNnptII

| Name | Sequence 5′→3′ |
|---|---|
| pSeq_CRBM_F1 | SEQ ID No. 25 |
| pSeq_CRBM_R1 | SEQ ID No. 26 |
| pSeq_CRBM_F2 | SEQ ID No. 27 |
| pSeq_CRBM_R2 | SEQ ID No. 28 |
| pSeq_CRBM_F3 | SEQ ID No. 29 |
| pSeq_CRBM_R3 | SEQ ID No. 30 |
| pSeq_CRBM_F4 | SEQ ID No. 31 |
| pSeq_CRBM_R4 | (SEQ ID No. 32) |

After transcription into the plant cell, the crRNA's should be cut out via two flanking ribozymes. For this, the precursor crRNA was flanked by the coding sequences of a hammerhead ribozyme and an HDV ribozyme (Tang, X., L. G. Lowder, T. Zhang, A. A. Malzahn, X. Zheng, D. F. Voytas, Z. Zhong, Y. Chen, Q. Ren, Q. Li, E. R. Kirkland, Y. Zhang and Y. Qi (2017), “A CRISPR-Cpf1 system for efficient genome editing and transcriptional repression in plants.” Nat Plants 3:17018).

For a perfect ligation of the individual protospacers at the coding sequence of the crRNA repeat, two bbsI detection sequences were integrated between crRNA repeat and HDV ribozyme, wherein the overhangs that were used for the cloning were adapted accordingly. In order to ensure an identical expression strength of the cpf1 and the crRNA's, the crRNA ribozyme cassette was bounded, at the 5′-end, by the PcUbi promoter sequence and, at the 3′-end, by [a/the] 3A terminator sequence. For the later ligation in the target vector pZFNnptII Cpf1, the crRNA expression cassette was flanked by two PstI interfaces and ordered as a synthetic DNA fragment. The protospacers were synthesized as complementary oligonucleotides and annealed according to a standard protocol. The 24-bp-long DNA fragment that was generated in this way was flanked by the 4-nt overhangs that are relevant to the ligation (see Table D).

TABLE D
Sequence of oligonucleotides that were used for the
generation of short 24-bp protospacers. The 4-nt overhangs
that are used for the ligation are the respective four
first nucleotides of each listed sequence.

| Name of the crRNA | Sequence 5′→3′ |
|---|---|
| 5′crRNA#1 | SEQ ID No. 33 |
| | SEQ ID No. 34 |
| 5′crRNA#2 | SEQ ID No. 35 |
| | SEQ ID No. 36 |
| 5′crRNA#3 | SEQ ID No. 37 |
| | SEQ ID No. 38 |
| 5′crRNA#4 | SEQ ID No. 39 |
| | SEQ ID No. 40 |
| 3′crRNA#1 | SEQ ID No. 41 |
| | SEQ ID No. 42 |
| 3′crRNA#2 | SEQ ID No. 43 |
| | SEQ ID No. 44 |
| 3′crRNA#3 | SEQ ID No. 45 |
| | SEQ ID No. 46 |

The efficiency of the four crRNA's was tested by means of agrobacteria-mediated gene transfer in leaves of B. vulgaris. The pZFNtDTnptII plasmid was co-transformed in order to check the transformation efficiency. The transformation of the leaf explant took place via vacuum infiltration according to a standard protocol. The fluorescence of the tDT was chocked after six days by means of fluorescence microscopy, and leaf explants with heterogeneous fluorescence were discarded. Ten days after infiltration took place, the leaf explants were quick-frozen in liquid nitrogen, pestled, and the genomic DNA was isolated by means of the CTAB method (Clarke, Joseph D., “Cetyltrimethyl ammonium bromide (CTAB) DNA miniprep for plant DNA isolation.” Cold Spring Harbor Protocols 2009.3 (2009): pdb-prot5177). The efficiency of the individual crRNA's was determined by an external service provider, using the frequency of the inserted editions (e.g., insertions, deletions, or base exchange) in comparison to unedited sequences in the genomic DNA, by means of NGS.

As a synthetic DNA construct, the most efficient crRNA's—5′crRNA #3 and 3′crRNA #1—with the previously described ribozymes, promoter, and terminator sequences, were ordered as reverse-oriented expression cassettes. The entire DNA construct was flanked by two PstI restriction interfaces for cloning in the target vector pZFNnptII_LbCpf1. After insertion of the crRNA's has taken place, the LbCpf1 and crRNA expression cassettes were ligated from the vector pZFNnptII_LbCpf1_crRNA into the pUbitDTnptII vector via HindIII.

As a repair template which should be integrated into the genome of B. vulgaris via homologous recombination, the resistance gene expression cassette was flanked, at the 5′-end, by the 5′crRNA #3 and, at the 3′-end, by the 3′crRNA #1 binding sequence. This enabled the excision of the resistance gene expression cassette from the plasmid via Cpf1. The entire DNA template was synthesized as an 87,326-bp-long synthetic DNA fragment (SEQ ID No. 80) and used directly in the vector backbone for the transformation. The resistance gene plasmid and the pUbitDTnptII_LbCpf1_crRNA plasmid were introduced into B. vulgaris callus cultures with the aid of a gene cannon.

The transformation efficiency was determined using the transient tDT fluorescence, one day after the transformation, by means of fluorescence microscopy. The callus cultures were cultivated on shoot induction medium without selection pressure (without Kanamycin), and the regenerated shoots were subsequently checked for the site-directed integration of the resistance-conferring resistance gene cassette. For this, the genomic DNA was isolated by means of CTAB. The integration of the resistance-conferring gene was amplified by means of PCR using the primers pCRBM_F1 according to SEQ ID No. 47 and pCRBM_R1 according to SEQ ID No. 48 (see Table E), and the PCR products were subsequently sequenced with both primers. Shoots, in which the successful insertion of the expression cassette could be verified in this manner, were identified in the following analyses of the integration site of the resistance gene. In order to verify the insertion within the desired target sequence in the genome, the flanking regions of the resistance gene expression cassette were amplified by means of PCR. The binding of a primer here took place within the resistance gene DNA sequence; the binding of the second primer took place outside of the 5′- or 3′-flanking homologous region of the inserted expression cassette (see Table E). The amplified DNA sequences were sequenced using the same primers, and the integration at the desired location was confirmed in this way. In order to preclude the binding of the primers pCRBM_F1 (SEQ ID No. 47), pCRBM_R1 (SEQ ID No. 48), pCRBM_R2 (SEQ ID No. 50) and pCRBM_F3 (SEQ ID No. 51) in sequence-similar regions of the genome, all primer sequences were compared beforehand with the B. vulgaris genome. For the primer pCRBM_F3 (SEQ ID No. 51), it was not possible to select the nucleotide sequence such that a binding to the wild-type sequence could be precluded. Therefore, the 3′-flanking region was amplified in all shoots that tested positive for the resistance gene, and the site-specific insertion was verified exclusively via the subsequent sequencing. The generated PCR product thereby differs by 18 bp from the wild-type sequence. In order to enable the complete sequencing of the amplified sequences, the PCR products were additionally sequenced via a third primer with a binding location within the amplified sequence (pCRBM_S2, pCRBM_S3; see Table E). In order to preclude the nonspecific binding of the primers pCRBM_F1 (SEQ ID No. 47), pCRBM_R1 (SEQ ID No. 48) and pCRBM_R2 (SEQ ID No. 50) within the wild-type genome, the nucleotide sequences were compared with an internal reference genome of B. vulgaris. The primers were additionally tested by means of PCR for the binding in genomic sequences of B. vulgaris wild-type plants.

In order to preclude the integration of the resistance gene in other regions of the genome, a targeted amplification of the target location was performed (Targeted Locus Amplification, TLA).

TABLE E
Primer used to verify the insertion of the resistance gene
expression cassette at the desired integration site.

| | Sequence | Size of the | | |
|---|---|---|---|---|
| Name | 5′→3′ | PCR product | | Binding |
| pCRBM_F1 | SEQ ID No. 47 | 450 | bp | within the resistance gene |
| | | | | expression cassette |
| pCRBM_R1 | SEQ ID No. 48 | | | within the resistance gene |
| | | | | expression cassette |
| pCRBM_F2 | SEQ ID No. 49 | 1,140 | bp | up-strand of the 5′-flanking |

| | | | |
|---|---|---|---|
| | | | homologous region |
| pCRBM_R2 | SEQ ID No. 50 | | within the resistance gene |
| | | | promoter sequence |
| pCRBM_S2 | SEQ ID No. 66 | | |
| pCRBM_F3 | SEQ ID No. 51 | 1,280 | within the resistance gene |
| | | | terminator sequence |
| pCRBM_R3 | SEQ ID No. 52 | | down-strand of the 3′- |
| | | | flanking homologous region |
| pCRBM_S3 | SEQ ID No. 67 | | |

In addition to the verification and the successful insertion of the resistance gene expression cassette into the genome of B. vulgaris, the unwanted integration of plasmid DNA was also checked. For this, genomic DNA, in which the verification had already yielded a successful insertion of the resistance gene at the desired target site, was checked for the presence of plasmid DNA by means of PCR. Sequence regions within the cpf1, the crRNA ribozyme cassette, and the tDT were thereby amplified using the primers listed in Table F, and subsequently sequenced.

TABLE F
Primers used to verify stably-integrated, plasmid-specific sequences
in the genome of the regenerated B. vulgaris shoots

| | Sequence | Size of the | |
|---|---|---|---|
| Name | 5′→3′ | PCR product | Binding |
| pSeq_LbCpf1_F4 | SEQ ID No. 68 | 214 | Cpf1 |
| pSeq_LbCpf1_R3 | SEQ ID No. 69 | | |
| pSeq_Ribozyme_F | SEQ ID No. 70 | 172 | crRNA ribozyme |
| pSeq_Ribozyme_R | SEQ ID No. 71 | | cassette |
| pSeq_tDT_F | SEQ ID No. 72 | 400 | tDT |
| pSeq_tDT_R | SEQ ID No. 73 | | |

### Example 2: Introduction of the Resistance-Conferring Gene as a Transgene by Means of Gene Transformation in Beta vulgaris Subsp. vulgaris

The transgenic approach to the production of Cercospora-resistant plants served not only for the alternative validation of the LRR gene as the resistance-conferring gene, but also as a means of producing transgenic resistance events that confers a novel Cercospora resistance or improve already existing Cercospora resistances.

The binary vector pZFN-nptII-LRR was generated by means of the following standard cloning procedures: Within the T-DNA of this vector, the cDNA of the resistance gene according to SEQ ID NO 2 was cloned together with its native promoter sequence. The T-DNA furthermore included the neomycin phosphotransferase II (nptII) gene, which confers resistance to a bandwidth of aminoglycoside antibiotics such as kanamycin or paromomycin. These antibiotic resistances were used for the selection of the transgenic plant cells and tissues. The NOS promoter and the pAG7 terminator flanked the nptII gene. The backbone of the binary vector furthermore contained the colE1 and the pVS1 origin for the plasmid replication in Escherichia coli or Agrobacterium tumefaciens. The aadA gene confers streptomycin/spectinomycin resistance for bacteria selection. The pZFN-nptII-LRR plasmid was transformed in agrobacterium strain AGL-1 by means of standard procedure.

The transformation of the sugar beets took place according to Lindsey & Gallois (1990), “Transformation of sugarbeet (Beta vulgaris) by Agrobacterium tumefaciens.” Journal of experimental botany 41.5, 529-536.). For this, “micropropagated shoots” of genotype 04E05B1DH5, which did not carry the resistance gene according to the invention, were used as starting material. Shoots were multiplied in the corresponding medium according to Lindsey & Gallois (1990). In order to induce as many meristems as possible, the “shoots” were transferred into a different medium (see Lindsey & Gallois (1990)) and incubated in darkness for several weeks at approximately 30° C. Agrobacterium strain AGL-1 with vector pZFN-nptlI-LRR (FIG. 3) was cultured in an additional medium (see Lindsey & Gallois (1990)), additionally provided with corresponding antibiotics for selection. Sections of meristematic tissue based upon the shoot to be treated were incubated with agrobacterium for several hours in an additional medium (see Lindsey & Gallois (1990)). Plant explants and agrobacteria were co-cultivated in darkness for at least 2 days in medium (see Lindsey & Gallois (1990)), and inoculated explants were subsequently incubated in darkness for approximately 2 weeks in an additional medium (see Lindsey & Gallois (1990)). The explants were thereupon further propagated in an additional medium (see Lindsey & Gallois (1990)) and sub-cultivated, in order to enable the selection of the transgenic tissue. In order to conclude the selection phase and to reduce the extent of chimera formation, green “shoots” were transferred to medium H, and all were propagated for 2 weeks. Leaf material was then extracted from the green, growing “shoots” and examined by means of PCr for the presence of the transgene. Suitable “shoots” were rooted in medium I and subsequently transferred to a greenhouse for production of T1 seed stock. Furthermore, leaf material derived from these “shoots” was used to analyse the expression of the transformed resistance gene.

### Analysis of the Expression Level

RNA was isolated from the leafes of the in vitro “shoots” and used within an qRT-PCR. The qRT-PCR was performed according to Weltmeier et al. 2011 (s. background of invention). Measured values were normalized against the reference gene PLT3_075_F09 (s. Weltmeier et al. 2011). The expression was determined by the use of the following primer sequences:

| | Size [No. | | Size of amplification |
|---|---|---|---|
| Sequence | nucleotides] | Tₘ [C. °] | product [No. nucleotides] |
| SEQ ID No. 92 | 21 | 59.8 | 170 |
| SEQ ID No. 93 | 21 | 58.9 | 170 |

Resistance Test in Sugar Beet after Inoculation with Cercospora Beticola Under Greenhouse Conditions:

A pure Cercospora beticola culture with a known high virulence was propagated on vegetable juice agar in Petri dishes (9 cm diameter) at 20° C. under near-ultraviolet (NUV) light. After 14 days, the surface of the agar on which the mold was grown was flooded with 10 ml of sterile water per Petri dish, and the conidia and mycelium fragments were carefully scraped off with the aid of a subject carrier. An inoculum density of 20,000 conidia/mycelium fragments per ml, plus 0.1% TWEEN 20, was used to inoculate the plants. At the point in time of the inoculation, the plants had been cultivated for 8 to 9 weeks under greenhouse conditions. The top side and underside of the leaves were treated with the inoculum. The plants were subsequently incubated for 5 to 7 days at 25° C., 18 h/6 h light/dark, and approximately 100% humidity. The first Cercospora symptoms on the sugar beet leaves occurred after 12 to 14 hours. An assessment of the symptoms of the individual plants was performed regularly, with the assistance of the assessment of the rating scores shown in Tab. 1A. The results are shown below.

TABLE G
Results of transgenic verification of the function of the
resistance gene according to the invention in transformed plants;

| | | | | | | | Expression level |
|---|---|---|---|---|---|---|---|
| | | | | | | | of the resistance |
| | Number of | Average rating score | | | | | gene according to |
| Test group | Individuals | 8 dpi | 11 dpi | 13 dpi | 15 dpi | Function | the invention |
| 1 | 57 | 1.60 | 3.82 | 5.24 | 6.46 | negative control | 0.0 |
| 2 | 38 | 1.28 | 3.07 | 4.42 | 6.04 | transgenic validation | 4.6 |
| 3 | 60 | 1.26 | 2.83 | 4.38 | 6.20 | transgenic validation | 12.5 |
| 4 | 45 | 1.50 | 3.40 | 4.62 | 6.18 | no validated | 0.0 |
| | | | | | | expression in vitro | |
| 5 | 60 | 1.44 | 3.62 | 5.29 | 6.38 | transgenic validation | 2.6 |
| 6 | 57 | 1.68 | 3.69 | 5.30 | 6.52 | transgenic validation | 3.3 |
| 7 | 66 | 1.58 | 3.84 | 5.43 | 6.57 | transgenic validation | 2.9 |
| 8 | 60 | 1.31 | 2.81 | 4.19 | 5.48 | transgenic validation | 11.3 |
| 9 | 72 | 1.25 | 3.07 | 4.61 | 5.97 | transgenic validation | 26.8 |
| 10 | 60 | 1.44 | 3.26 | 4.77 | 6.00 | transgenic validation | 10.7 |
| 11 | 57 | 1.60 | 3.83 | 5.48 | 6.64 | transgenic validation | 4.4 |
| 12 | 72 | 1.34 | 2.62 | 3.75 | 5.19 | resistant source plant | not determined |
| mean value | 58.66 | 1.44 | 3.32 | 4.8 | 6.13 | | |
| overall | | | | | | | |
| LSD value | — | 0.17 | 0.47 | 0.48 | 0.4 | | |

LSD = least significant difference;
dpi = days post infection

Results of the Transgenic Validation of the Resistance Gene According to the Invention (s. Table G)

Test group 1 represents a negative control. The genotype is the same as for test groups 2 to 11 but no transformation has taken place. Therefore, no expression could be detected. Test group 4 has been transformed but no expression could be detected. Test groups 2, 3 and 5 to 11 represent transformants which carry the resistance gene according to the invention only due to the transformation. Test group 12 represents a breeding line comprising the resistance gene according to the invention in a non-transgenic version. The rating scores of all lines has been established after inoculating the plant material with Cercospora beticola as described above. Test group 12 shows the highest resistance which is indicated by a final value of 5.19.

The transgenic lines showed a rating score according to the following table:

TABLE H
Rating scores of the transgenic lines of table G

| | 8dpi | 11dpi | 13dpi | 15dpi |
|---|---|---|---|---|
| mean value | 1.42 | 3.33 | 4.87 | 6.2 |
| transgenic | | | | |
| validation | | | | |
| mean value | 1.315 | 2.99 | 4.48 | 5.91 |
| transgenic | | | | |
| validation for | | | | |
| lines having an | | | | |
| expression | | | | |
| level >10 | | | | |

Table H shows that the rating scores for transgenic validation groups only. First the mean value for all transgenic test groups (excluding group 4) are shown. Below the rating scores only for those transgenic lines which showed an expression level of at least 10 (groups 3, 8, 9, 10; s. Table G) are given. Here, the final rating score is 5.91. That is a significantly higher resistance than the negative control of group 1 which has a rating score of only 6.46 (least significant difference=0.4; s. Table G). The best transgenic test group (group 8) shows an even better resistance due to a rating score is 5.48 (s. Table G).

It is worth mentioning that the expression level of transgenic insertions may be influenced by the integration locus. As the expression level was measured in vitro the actual expression level under infection conditions could be higher—especially under when the resistance gene is under control of a pathogen inducible promoter.

### Statistical Evaluation of the Results of the Transgenic Validation

TABLE I
statistic clustering

| test | cluster | cluster | cluster | cluster |
|---|---|---|---|---|
| group | 8dpi | 11dpi | 13dpi | 15dpi |
| 1 | ab | a | a | ab |
| 2 | e | de | bc | cd |
| 3 | e | ef | c | bcd |
| 4 | bc | bcd | bc | bcd |
| 5 | cd | abc | a | abc |
| 6 | a | ab | a | ab |
| 7 | ab | a | a | a |
| 8 | de | ef | c | e |
| 9 | e | de | bc | d |
| 10 | cd | cd | b | d |
| 11 | ab | a | a | a |
| 12 | de | f | d | e |

Table I shows a statistical evaluation of the rating scores contained in Table G. Each letter symbolizes the allocation to a statistical group. For example, it is evident that after the final evaluation (15 dpi) test group 8 (transgenic verification) is in the same cluster as test group 12 (resistant source) but in a different cluster than test group 1 (negative control). According to this test group 8 is significantly different from test group 1 but not significantly different than test group 12.

In addition, a box-plot analysis has been performed. The illustration of the box-plots is available from FIG. 4-7.

### Example 3: Production of a Resistant Sugar Beet Plant According to the Invention on the Basis of Genetic Material Accessed from Beta vulgaris supsp. maritima

The process described hereafter was based on pooling wild beet material to generate a Cercospora resistance genepool. The Beta vulgaris subsp. maritima accessions used as starting material for the breeding program are listed in the following table.

TABLE 2
Accessions of Beta vulgaris subsp. maritima and their resistance rating scores towards Cercospora used in a breeding program;
the first 4 columns on the right present the accession numbers wherein a certain accession may have different accession numbers
depending on the deposition facility

| | | | | Resistance rating score for Cercospora | | | | | | | | | | |
|---|---|---|---|---|---|---|---|---|---|---|---|---|---|---|
| | | | | GRIN studies | | | | | | | | | | |
| | | | | | | SUGARBEET_ | SUGARBEET_ | SUGARBEET_ | SUGARBEET_ | SUGARBEET_ | SUGARBEET_ | | | |
| Accession denomination | | | | SUGARBEET_ | SUGARBEET_ | CERCOSPORA_ | CERCOSPORA_ | CERCOSPORA_ | CERCOSPORA_ | CERCOSPORA_ | RICHARDSON_ | IDBB | | |
| USDA | | DEU | | CERCO_ | CAC_ | 1989_ | 1992_ | 1994_ | 1997_ | 1999_ | 2010_ | studies | | |
| GRIN | IDBBNR | 001 | IPK | 1 | WBITALY | RUPPEL | RUPPEL | RUPPEL | RUPPEL | PANELLA | TEST1910 | 202 | 237 | 238 |
| PI 120704 | 5191 | | | 1 | | | | | 9 | | | | | |
| PI 169020 | 5265 | | | 1 | | | | | 8 | | | | | |
| PI 169023 | 5268 | | | 1 | | | | | 7 | | | | | |
| PI 169030 | 5274 | | | 1 | | | | | 8 | | | | | |
| PI 546536 | 9703 | | | | | | 3 | | 8 | | | | | |
| PI 546539 | 9706 | | | | | | 3 | | 6 | | | | | |
| PI 518303 | 5797 | | | | | | | | | 4 | 2 | | | |
| PI 518303 | 5797 | | | | | | | | | 4 | 2 | | | |
| PI 546534 | 9701 | | | | | | 3 | | | | 8 | | | |
| PI 590763 | 4587 | | | | | | | | | 4 | | | | |
| PI 590766 | 4591 | | | | | | | | | 4 | | | | |
| PI 109038 | 5160 | | | 1 | | 5 | | | 7 | | | | | |
| | | 28894 | BETA | | | | | | | | | | | |
| | | | 1521 | | | | | | | | | | | |
| | 2195 | 32375 | BETA | | | | | | | | | 2 | | |
| | | | 1429 | | | | | | | | | | | |
| | 3555 | 48819 | | | | | | | | | | | | |
| | | 64088 | BETA | | | | | | | | | | | |
| | | | 2157 | | | | | | | | | | | |
| | 8535 | 58260 | BETA | | | | | | | | | | | |
| | | | 1987 | | | | | | | | | | | |
| | 3358 | 36542 | BETA | | | | | | | | | | | |
| | | | 1228 | | | | | | | | | | | |
| | 3744 | 51437 | BETA | | | | | | | | | | 6 | |
| | | | 1083 | | | | | | | | | | | |
| | 6071 | 54762 | BETA | | | | | | | | | | | |
| | | | 1655 | | | | | | | | | | | |
| | 2649 | 54832 | BETA | | | | | | | | | | | |
| | | | 992 | | | | | | | | | | | |
| | 7103 | 57737 | BETA | | | | | | | | | | | |
| | | | 1127 | | | | | | | | | | | |
| | 8634 | 62120 | BETA | | | | | | | | | | | |
| | | | 1057 | | | | | | | | | | | |
| | 8635 | 62121 | BETA | | | | | | | | | | | |
| | | | 1447 | | | | | | | | | | | |
| | 8636 | 62122 | BETA | | | | | | | | | | | |
| | | | 1014 | | | | | | | | | | | |
| | 8637 | 62123 | BETA | | | | | | | | | | | |
| | | | 1432 | | | | | | | | | | | |
| | 8638 | 62124 | BETA | | | | | | | | | | | |
| | | | 1090 | | | | | | | | | | | |
| | 8640 | 62126 | BETA | | | | | | | | | | | |
| | | | 1377 | | | | | | | | | | | |
| | 8642 | 62128 | BETA | | | | | | | | | | | |
| | | | 1558 | | | | | | | | | | | |
| | 8643 | 62130 | BETA | | | | | | | | | | | |
| | | | 1348 | | | | | | | | | | | |
| | 8644 | 62131 | BETA | | | | | | | | | | | |
| | | | 1610 | | | | | | | | | | | |
| | 2212 | 28931 | BETA | | | | | | | | | | | |
| | | | 1666 | | | | | | | | | | | |
| | 3546 | 48810 | BETA | | | | | | | | | | | |
| | | | 1304 | | | | | | | | | | | |
| PI 504196 | | | | | 3 | | | | 7 | | 8 | | | |
| PI 546409 | | | | | | | | 3 | | | | | | |
| | 3401 | 45516 | BETA | | | | | | | | | | | |
| | | | 2174 | | | | | | | | | | | |
| PI 504245 | 5726 | | | | | | | | | | 8 | | | |
| | Accession denomination | | | | Resistance rating score for Cercospora | | | | | | | | | |
| | USDA | | DEU | | IDBB studies | | | | | | | | | |
| | GRIN | IDBBNR | 001 | IPK | 244 | 248 | 249 | 250 | 252 | 254 | 259 | 263 | 270 | 288 |
| | PI 120704 | 5191 | | | | | | | | | | | | |
| | PI 169020 | 5265 | | | | | | | | | | | | |
| | PI 169023 | 5268 | | | | | | | | | | | | |
| | PI 169030 | 5274 | | | | | | | | | | | | |
| | PI 546536 | 9703 | | | | | | | | | | | | |
| | PI 546539 | 9706 | | | | | | | | | | | | |
| | PI 518303 | 5797 | | | | | | | | | | | | |
| | PI 518303 | 5797 | | | | | | | | | | | | |
| | PI 546534 | 9701 | | | | | | | | | | | | |
| | PI 590763 | 4587 | | | | | | | | | | | | |
| | PI 590766 | 4591 | | | | | | | | | | | | |
| | PI 109038 | 5160 | | | | | | | | | | | | 8 |
| | | | 28894 | BETA | 3 | | | | | 1 | | | | |
| | | | | 1521 | | | | | | | | | | |
| | | 2195 | 32375 | BETA | | | | | | | | | | |
| | | | | 1429 | | | | | | | | | | |
| | | 3555 | 48819 | | | | 4 | | | 4 | | | | |
| | | | 64088 | BETA | | | | | | | | | | |
| | | | | 2157 | | | | | | | | | | |
| | | 8535 | 58260 | BETA | | | | | | | | | | |
| | | | | 1987 | | | | | | | | | | |
| | | 3358 | 36542 | BETA | 6 | | | | | 3 | | | | |
| | | | | 1228 | | | | | | | | | | |
| | | 3744 | 51437 | BETA | | | | | | | 7 | | | |
| | | | | 1083 | | | | | | | | | | |
| | | 6071 | 54762 | BETA | | | | | | | | 2 | | |
| | | | | 1655 | | | | | | | | | | |
| | | 2649 | 54832 | BETA | | 8 | | | | | | 5 | | |
| | | | | 992 | | | | | | | | | | |
| | | 7103 | 57737 | BETA | | | | | | | 3 | | | |
| | | | | 1127 | | | | | | | | | | |
| | | 8634 | 62120 | BETA | | | | | 5 | | | | | |
| | | | | 1057 | | | | | | | | | | |
| | | 8635 | 62121 | BETA | | | | | 1 | | | | | |
| | | | | 1447 | | | | | | | | | | |
| | | 8636 | 62122 | BETA | | | | | 3 | | | | | |
| | | | | 1014 | | | | | | | | | | |
| | | 8637 | 62123 | BETA | | | | | 3 | | | | | |
| | | | | 1432 | | | | | | | | | | |
| | | 8638 | 62124 | BETA | | | | | 2 | | | | | |
| | | | | 1090 | | | | | | | | | | |
| | | 8640 | 62126 | BETA | | | | | 2 | | | | | |
| | | | | 1377 | | | | | | | | | | |
| | | 8642 | 62128 | BETA | | | | | 2 | | | | | |
| | | | | 1558 | | | | | | | | | | |
| | | 8643 | 62130 | BETA | | | | | 4 | | | | | |
| | | | | 1348 | | | | | | | | | | |
| | | 8644 | 62131 | BETA | | | | | 2 | | | | | |
| | | | | 1610 | | | | | | | | | | |
| | | 2212 | 28931 | BETA | 2 | | | | | 7 | | | | |
| | | | | 1666 | | | | | | | | | | |
| | | 3546 | 48810 | BETA | | | | | | 3 | 3 | | | |
| | | | | 1304 | | | | | | | | | | |
| | PI 504196 | | | | | | | | | | | | | |
| | PI 546409 | | | | | | | | | | | | | |
| | | 3401 | 45516 | BETA | | | | 3 | | | | | | |
| | | | | 2174 | | | | | | | | | | |
| | PI 504245 | 5726 | | | | | | | | | | 1 | | |

(USDA GRIN = US Department of Agriculture Germplasm Resources Information Network;
IDBBNR = International Database for Beta;
DEU001 = Plant Genetic Resource Collection;
IPK = Leibnitz-Institut für Pflanzengenetik und Kulturpflanzenforschung)

As it is apparent from Tab. 2 the accessed genetic material had shown previously a non-uniform resistance level against Cercospora and the degree of resistance varied throughout the different studies. For example, the accession “PI 120704” showed a score of 1 in one study and a score of 9 in another study. As this publicly available data seemed to be unreliable, seed material from the accessions has been planted and the resulting plants were screened phenotypically for Cercospora resistance. About 150 partial resistant plants have been selected. However, as the seen resistance in each plant could have been the result of a plenty of genes all having a small contribution the chances to identify a single gene suitable for establishing a resistance or increasing the resistance level in a measurable manner was limited. It was decided to cross the about 150 resistant plants among each other using an open pollination scenario. This approach also allowed for the generation of recombinations within the genetic material. Crossing and selection have been repeated for several generations to improve the resistance level. The best descendants have been cloned and prepared for a genetic mapping approach. The mapping of the herein described resistance was coupled with intensive phenotyping. With the aid of the setup of a population of over 4,000 dividing descendants and the development of special recombination screens, the target region was reduced, and thus ever further isolated, via analysis of informative recombinants (genotypical and phenotypical) in a series of resistance tests. This genetic mapping, as well as the creation of physical maps accompanied by WHG sequencing (“whole genome sequencing”), comparative BAC (Bac-by-Bac) sequencing, and bioinformatic analyses, led to the identification of three recombinant genotypes that confirmed the resistance gene (1 recombinant in the neighboring gene, on the one hand, and 2 recombinants in the neighboring gene, on the other). In light of particular requirements, the inventors placed the highly repetitive structure in the target region, which, among other things, contains tandem repeats with very high sequence homology, which made the marker development, and thus the identification of informative recombinants, enormously more difficult. The following steps were particularly decisive for the location of the genetic structure of the resistance gene:
development of the markers s4p0264s01, s4p2271s01, sxh0678s01, s4p4293s01, s4p4295s01, s4p4301s01 (see Table 1B).
Fine mapping coupled with intensive phenotyping. The phenotypes were verified with 90-180 descendants per plant in a greenhouse test, and with intensive statistical methods (for example, t-test, power analysis, etc.).
BAC clone identification and sequencing from BAC pools of the resistant genotype.
Sequence evaluation, as well as sequence and protein comparison between RR (i.e., resistant) and ss (i.e., sensitive) genotypes; an unambiguous assembly of the RR and ss sequence data was thereby not always possible, due to the sequence complexity.

In the framework of the breeding program, the Beta vulgaris subsp. maritima derived resistance was crossed with an elite sugar beet line. Several back crossings via marker assisted selection allowed to transfer the resistance gene in established sugar beet germplasm. Surprisingly, no undesired effects towards sugar yield etc. could be observed. Subsequently, a proof of concept for the resistance gene within sugar beet has been established via transformation and the generation of sugar beets which were transgenic for the resistance gene (s. above). After this successful proof of concept the generated sugar beet germplasm comprising the resistance gene could be used for the generation of a Cercospora resistant sugar beet variety.

### Example 4: Screening the Starting Accessions for the Identified Resistance Gene

After the resistance gene has been identified the genetic source material (accessions according to Tab. 2) was screened by the help of markers to identify the accession which carried the resistance gene. The number of the analyzed plants per accession was dependent on the availability of seeds and is given in the table below.

TABLE 3
Number of plants per accession analyzed for the
presence of the identified resistance gene

| Accession denomination | | | | Plants |
|---|---|---|---|---|
| USDA GRIN | IDBBNR | DEU001 | IPK | [No.] |
| PI 120704 | 5191 | | | 40 |
| PI 169020 | 5265 | | | 40 |
| PI 169023 | 5268 | | | 18 |
| PI 169030 | 5274 | | | 18 |
| PI 546536 | 9703 | | | 50 |
| PI 546539 | 9706 | | | 34 |
| PI 518303 | 5797 | | | 2 |
| PI 518303 | 5797 | | | 16 |
| PI 546534 | 9701 | | | 40 |
| PI 590763 | 4587 | | | 12 |
| PI 590766 | 4591 | | | 28 |
| PI 109038 | 5160 | | | 23 |
| | | 28894 | BETA 1521 | 40 |
| | 2195 | 32375 | BETA 1429 | 40 |
| | 3555 | 48819 | | 14 |
| | | 64088 | BETA 2157 | 12 |
| | 8535 | 58260 | BETA 1987 | 40 |
| | 3358 | 36542 | BETA 1228 | 40 |
| | 3744 | 51437 | BETA 1083 | 31 |
| | 6071 | 54762 | BETA 1655 | 40 |
| | 2649 | 54832 | BETA 992 | 12 |
| | 7103 | 57737 | BETA 1127 | 40 |
| | 8634 | 62120 | BETA 1057 | 18 |
| | 8635 | 62121 | BETA 1447 | 40 |
| | 8636 | 62122 | BETA 1014 | 40 |
| | 8637 | 62123 | BETA 1432 | 37 |
| | 8638 | 62124 | BETA 1090 | 11 |
| | 8640 | 62126 | BETA 1377 | 40 |
| | 8642 | 62128 | BETA 1558 | 40 |
| | 8643 | 62130 | BETA 1348 | 40 |
| | 8644 | 62131 | BETA 1610 | 40 |
| | 2212 | 28931 | BETA 1666 | 36 |
| | 3546 | 48810 | BETA 1304 | 40 |
| PI 504196 | | | | 37 |
| PI 546409 | | | | |
| | 3401 | 45516 | BETA 2174 | 40 |
| PI 504245 | 5726 | | | 40 |

Each of the given plants of each accession has been screened by the use of 572 SNP markers which were located 5′ as well as 3′ to the resistance gene. Due to the large amount of markers a haplotype pattern could be derived. However, none of the accessions used as starting material showed the haplotype of the line CRBM which carried the identified resistance gene. Most similarities have been found to the accession 48819 (DEU001 denomination)/3555 (IDBBNR) (s. Tables 2 and 3). The following table shows an extract of the entire marker analysis including positions 5′ and 3′ of the resistance gene.

TABLE 4
Comparison of a resistant line according to the invention and
14 plants of accession 48819 via SNP marker analysis

| | | s4e5 | s4p2 | Is4p2 | s4p4 | s4p4 | sxh6 | sxh3 | s4p4 | s4p8 | |
|---|---|---|---|---|---|---|---|---|---|---|---|
| | s4e562 | 628s | 272s | 273s | 291s | 293s | 2648 | 116s | 295s | 772s | |
| Marker | 8s03 | 02 | 01 | 01 | 01 | 01 | 01 | 01 | 01 | 01 | |
| Pos | 62.81 | 62.82 | 62.83 | 62.83 | 62.84 | 62.84 | 62.84 | 62.84 | 62.85 | 62.86 | |
| start | | | | | | | | | | | |
| CRBM | A | A | A | A | A | A | G | C | A | T | * |
| comprising | | | | | | | | | | | |
| SEQ ID NO: 1 | | | | | | | | | | | |
| 48819_1 | A | G | T | C | G | G | G | C | T | A | |
| 48819_2 | A | G | T | C | G | G | G | C | T | A | |
| 48819_3 | A | G | T | C | G | G | G | C | A/T | A/T | |
| 48819_4 | A | G | T | C | G | G | G | C | A/T | A/T | |
| 48819_5 | A | G | T | C | G | G | G | C | A/T | T | |
| 48819_6 | A | G | T | A/C | G | A | G | C | A/T | A/T | |
| 48819_7 | A | G | T | C | G | G | A/G | A/C | NA | A | |
| 48819_8 | A | G | T | A/C | G | G | G | A/C | NA | A | |
| 48819_9 | A | G | T | C | G | G | G | A/C | A | A/T | |
| 48819_10 | A | G | T | C | G | G | G | C | A | T | |
| 48819_11 | A | G | T | C | G | G | G | C | A | T | |
| 48819_12 | A | G | T | C | G | G | G | C | A/T | T | |
| 48819_13 | A | G | T | A/C | G | A | G | C | A/T | A/T | |
| 48819_14 | A | G | A/T | A/C | G | G | G | C | T | A | |
| | | s4p8 | | | | | | | | | |
| | | 783s | s4p430 | sxh067 | s4p227 | s4p430 | s4p430 | s4e971 | s4e789 | s4p430 | s4p430 |
| | Marker | 01 | 1s01 | 8s01 | 6s01 | 5d01 | 6301 | 4s01 | 5s01 | 7801 | 9s01 |
| | Pos | 62.94 | 62.94 | 62.97 | 62.97 | 62.98 | 62.98 | 62.98 | 62.98 | 62.99 | 63.01 |
| | start | | | | | | | | | | |
| | CRBM | C/T | A | A | G | del | G | G | C | C | T |
| | comprising | | | | | | | | | | |
| | SEQ ID NO: 1 | | | | | | | | | | |
| | 48819_1 | C | A | A | G | ins | G | G | C | C | T |
| | 48819_2 | C | A | A | G | ins | G | G | C | C | T |
| | 48819_3 | C | A/T | A/C | A/G | ins | A/G | C/G | C/G | C/T | C/T |
| | 48819_4 | C | A/T | A/C | A/G | ins | A/G | C/G | C/G | C/T | C/T |
| | 48819_5 | C | T | A/C | A | ins | A | C/G | G | C/T | C |
| | 48819_6 | C | A/T | C | G | ins | G | G | C | C | C/T |
| | 48819_7 | C | T | C | G | ins | G | G | C | C | C/T |
| | 48819_8 | C | A/T | A/C | G | ins | G | G | NA | C | C |
| | 48819_9 | C | T | C | A/G | ins | A/G | C/G | G | C/T | C |
| | 48819_10 | C | T | C | A | ins | A | C | G | T | C |
| | 48819_11 | C | T | C | A | ins | A | C | G | T | C |
| | 48819_12 | C | T | A/C | A | | A | C/G | G | C/T | C |
| | 48819_13 | C | A/T | C | A/G | ins | A/G | C/C | C/G | C/T | C/T |
| | 48819_14 | C | A/T | A/C | G | ins | A/G | C/C | C/G | C/T | C/T |

(del = deletion,
ins = insertion,
Pos start = starting position of molecular marker on genetic strand,
* = Position of resistance gene according to SEQ ID NO 1)

The results of the marker analysis (as exemplified by the data given in Tab. 4) show that the resistance gene according to the invention could not be traced back to one of the accessions according to Tab. 2. Even plants of the accession 48819 which shared the strongest marker overlap with the resistant line according to the invention had significant differences. Noticeable, was the detection of a deletion within the resistant line whereas accession 48819 showed a deletion at the same position. This could be an indication that a significant genetic restructuring at this locus took place during the generation of the resistance gene according to the invention. This assumption would also explain why it was not possible to trace back the resistance gene back to the starting material of the breeding program.

### Example 5: Creation of Cercospora Resistant Seed Stock

The generated sugar beet germplasm comprising the resistance gene (outcome of Example 3) could be used for the generation of a Cercospora resistant sugar beet variety. For this purpose the gene was transmitted via crossing into a DH parent line which was crossed with a DH parent line originating from the other hybrid breeding pool. The result was a hybrid variety comprising the resistance towards Cercospora according to the present invention. The seeds of the variety were separated from each other (singularized), cleaned and polished. Afterwards, the seeds have been subjected to priming and pelleting as described in EP2002702A1. The resulting seed stock has been filed in a packaging made of cardboard which comprised an interlayer as vapor barrier. The resulting seed stock was suitable for sowing, growing, harvesting and subsequent industrial sugar production.

1. A method for identifying a plant that is resistant or tolerant to Cercospora, characterized in that the method includes the following steps:
(i) detection of the presence or absence of a nucleotide sequence selected from the group consisting of:
(a) a nucleotide sequence that encodes a polypeptide having an amino acid sequence according to SEQ ID NO: 3;
(b) a nucleotide sequence that comprises the DNA sequence according to SEQ ID NQ; 2;
(c) a nucleotide sequence that comprises a DNA sequence according to SEQ ID NQ; 1 or SEQ ID NO: 53;
(d) a nucleotide sequence that hybridizes with a complementary sequence of the nucleotide sequence according to (a), (b), or (c) in 4×SSC at 65° C., and subsequent repeated washing in 0.1×SSC at 65° C. for approximately 1 hour in total;
(e) a nucleotide sequence that encodes a polypeptide which has an amino acid sequence that is at least 90% identical to an amino acid sequence according to SEQ ID NQ; 3;
(f) a nucleotide sequence that is at least 90% identical to a DNA sequence according to SEQ ID NO: 1 or SEQ ID NO: 2;
(ii) detection of the presence or absence of the polypeptide which is encoded by the nucleotide sequence as defined in step (i) in the plant or a portion of the plant; and/or
(iii) detection of at least one marker locus in the nucleotide sequence as defined in step (1) or in a cosegregating region,
wherein the cosegregating region is a genomic region which cosegregates with the Cercospora resistance conferred by the polypeptide, or with the nucleotide sequence and wherein the cosegregating region comprises and is flanked by marker s4p1395s01 and s4p0421s01.

2. The method according to claim 1, wherein the method includes a further step of:
(iv) selection of the Cercospora-resistant plant.

3. The method according to claim 1 wherein the detection in step (i) or (iii) is based on at least one polymorphism or single nucleotide polymorphism.

4. The method according to claim 1, wherein the detection in step (i) or (iii) is based on at least one polymorphism or single nucleotide polymorphism and wherein:
a) said at least one polymorphism or single nucleotide polymorphism is genetically linked to the nucleotide sequence or has a recombination frequency of 10% or less to the nucleotide sequence,
b) said at least one polymorphism or single nucleotide polymorphism is located within 2562 kbp, 2300 kbp, 2100 kbp, 1900 kbp, 1700 kbp, 1500 kbp, 1300 kbp, 1100 kbp, 900 kbp, 700 kbp, 500 kbp, 300 kbp, 100 kbp, 50 kbp, 25 kbp, 10 kbp, 5 kbp or 1 kbp or less of the nucleotide sequence,
c) said at least one polymorphism or single nucleotide polymorphism is detectable in the seeds deposited with the NCIMB, Aberdeen, UK under access number NCIMB 43646 or
d) said at least one polymorphism or single nucleotide polymorphism is part of the cosegregating region,
wherein the nucleotide sequence is the nucleotide sequence defined in step (i) of claim 1 and wherein the cosegregating region is the cosegregating region defined in step (iii) of claim 1.

5. The method according to claim 1, wherein step (i) and/or step (iii) further includes the following steps which are concluded by the detection:
a) provision of a plant, its tissue, a seed, or a cell thereof and
b) extraction of DNA, preferably genomic DNA from the plant, its tissue, the seed or from the cell thereof.

6. The method according to claim 1 involving the use of at least two oligonucleotides.

7. The method according to claim 6, wherein the oligonucleotides are suitable for use as primers in a polymerase chain reaction (PCR) and are able to hybridize to a genomic interval which comprises and is flanked by marker s4p1395s01 and s4p0421s01.

8. The method according to claim 1, wherein the plant is a plant of the genus Beta or a plant of the genus Spinacia.

9. The method according to claim 1 involving a PCR in step (i) and/or (iii) wherein the PCR involves two allele-specific forward primers and wherein the detection step involves fluorescence resonant energy transfer and wherein the presence, absence or kind of the fluorescence is determined by a sensor.

10. The method according to claim 9 involving one common reverse primer.

11. A plant of the genus Beta or Spinacia comprising a nucleic acid molecule encoding a polypeptide that is able to confer resistance to Cercospora in a plant in which the polypeptide is expressed, wherein the nucleic acid molecule comprises a nucleotide sequence which is selected from the group consisting of:
(a) a nucleotide sequence that encodes a polypeptide having an amino acid sequence according to SEQ ID NO: 3;
(b) a nucleotide sequence that comprises the DNA sequence according to SEQ ID NO: 2;
(c) a nucleotide sequence that comprises a DNA sequence according to SEQ ID NO: 1 or SEQ ID NO: 53;
(d) a nucleotide sequence that hybridizes with a complementary sequence of the nucleotide sequence according to (a), (b), or (c) in 4×SSC at 65° C., and subsequent repeated washing in 0.1×SSC at 65° C. for approximately 1 hour in total;
(e) a nucleotide sequence that encodes a polypeptide which has an amino acid sequence that is at least 90% identical to an amino acid sequence according to SEQ ID NO: 3; and
(f) a nucleotide sequence that is at least 90% identical to a DNA sequence according to SEQ ID NO: 1 or SEQ ID NO: 2;
wherein the plant of the genus Beta or Spinacia furthermore comprises an endogenous allele encoding an epsp synthase having at position 179 an amino acid different from proline.

12. The plant according to claim 11, further characterized by a feature or a combination of features selected from the group consisting of: the plant is a hybrid and/or a double haploid plant; the resistance against Cercospora is dominant the nucleic acid molecule or nucleotide sequence is comprised as an introgression or is comprised homozygous; and the plant has tolerance to glyphosate.

13. A storage organ or a leaf of the plant according to claim 11.

14. A pelleted seed of the plant according to claim 11, wherein the seed comprises the nucleic acid molecule and the endogenous allele.

15. The plant according to claim 11, wherein the epsp synthase having an amino acid different from proline at position 179, comprises an amino acid sequence selected from the group consisting of:
i) the sequence of SEQ ID NO: 223;
ii) the sequence i) having an amino acid different from serine and proline at position 179; and
iii) a sequence having an identity of at least 90% to the sequence of i) or ii) over the entire length of the sequence.

16. The storage organ or leaf according to claim 13, wherein the epsp synthase having an amino acid different from proline at position 179, comprises an amino acid sequence selected from the group consisting of:
i) the sequence of SEQ ID NO: 223;
ii) the sequence i) having an amino acid different from serine and proline at position 179; and
iii) a sequence having an identity of at least 90% to the sequence of i) or ii) over the entire length of the sequence.

17. The pelleted seed according to claim 14, wherein the epsp synthase having an amino acid different from proline at position 179, comprises an amino acid sequence selected from the group consisting of:
i) the sequence of SEQ ID NO: 223;
ii) the sequence i) having an amino acid different from serine and proline at position 179; and
iii) a sequence having an identity of at least 90% to the sequence of i) or ii) over the entire length of the sequence.


SEQUENCE LISTING (228 sequences)

SEQ ID NO: 1 - DNA, 3720 bp, Beta vulgaris
atgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcggtcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggtaagaaagtatattaaacttgttacttttgaaaatattcgctctagtttttgtttcagttggtccattctcactttgtattattgaaatatatcccaaaaaagtaaatataattatataaaagaatcttgctaaaaataatatgaattatttttgtatgtgcaaaataatgtacaaatctaactaatttgttgtggataataatattaattgtgtgaaatagtaaatgtgtggagatatataactttatttatcatattcactcaggtttttaggtatttattatgagttttgcattggagatatccaacttgacaatagtatttttgtaatataccaatatataaagattactgtacataaccaaaatgtatacttttcttatttttataaacttatatattcctcttctttgtatttatcacaacattttttatacccttttgcctcatattaatagcaacacttataatttatttatttactttttatttcttggtctataacctcatctacccacatatgacacaccctataaaggacccacatgattaaccaaaatatacaaatatcttcaatgaaattaactttaacactaatatgataaaaatcatgtcccgctttttatcctctaactaagactctgcataaaggtatattgcaattaatatgagatggaagaggtataataattatatgatcaaattcctggattgaaaaataaatatgagattaaaagtggtatgtttttggttaaaagaaactatccataaagtatgtttttggttaaaagaaactatgcaacataccaatcaaatgtttatacgcttacaatttatgtaccacttttttgtcattgtttttctattgtttgccatacgtacgttactaaatcatgttgtcttttcacattttaactaacaataaattactattgatacaccaaaaaaatctatgagcattggagtacgttgtttgatagaagcttcgtgctattatttcttgtcaaagaatttcatatctcaatatcttctaatttaacaatctaacgaaatttttttgacccaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctga

SEQ ID NO: 2 - DNA, 2652 bp, Artificial Sequence
  cDNA sequence of the Cercospora resistance-conferring gene
atgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcggtcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctga

SEQ ID NO: 3 - protein, 883 aa, Beta vulgaris
MNMKILLLFVFLHHLHYFIHGRTLTERQALLSIKSAITYDYYNSLSSWKNTTHHCSWPYITCSSSSSSSSVISLNFTMLFLEGILSPDIGFLTNLQNLSIRSNLFSGPLPHSLSLLTQLRYLDVSQNSFTGPIPSSLSLLTQLRYLHVSGNSFTGPIPSFLSLLTQLRYLDVSDNSFTGPIPSSLSLLTQLRYLDVSYNNLNGTLPLSVVEKMSELSYLNLRYNSFYGEIPPEFGKLKKLETLNLGNNTLSGSLPSELGSLKSLKHMDFSSNMLFGEIPQSYSLLRNLIDIDLNRNKLYGSIPDYIGDFPELESLLLDSNNFTGSIPQKLGTNGKLQYLDISNNNFSGSLPLSLCKGDKLQDLDASYNLLVGSIPESLGSCKSLEGVYMGNNFLNGSIPKGLFGSDVSLNDKLLSGGLDEKFGDCVNLRDIDLSNNKLSGKLPATIGNCIHLRSLTLYNNTCTGRIPQEISKCKQLQTLDLSQNQFSGVIPNDITGNKSICNLEKIQTLKLSNNALTGEIPHCVGNIELIALFLQSNKLNGTIPANFSKLCDSLIYLDLSDNQLEGVLPKSLSKCQSLELLNVGNNRLRDKFPSWLDNLPRLQVFSVRFNAFYGPITSSPKVSHPFPMLQIIDLSNNKFCGKLPRRYIKNFATMRNMNESGVGNPQYLGDSSIYSITYSMVLTFNGLQQKYEKLIVTMSTFDISSNNFTGQIPYVIGGLRSLRNLNLSHNVLTGNIPPSIAKLSLLQDLDLSSNRLTGRIPQELVSLTFLGSFNVSNNLLEGSIPHGFNFDTYTANSYQGNLELCGKPLPECGERRAKGTTNNQDDPKNDNERMLSMSEIVVMGFGSGVLVGLAWGYYMFSVGKPFWFIKMASKMESILIGFF

SEQ ID NO: 4 - DNA, 4748 bp, Beta vulgaris
ttactatgaacaataccctaatatcattaggttttccccttctctctcctaagtgccaaactgccaaccccctcccatctttatttcaataagagcaccattaaattattgtgtaacaaagaccattattttaagatcactaataaggttgctctaattattcctagaattctagtgaaaaaagaaagataaaagatgaacatggggtgatgactgatgactgagagacaacagacaacacttggttgagttgatatttgacgcaaagacttggcgtgttggaaggttcattacacattttatccaagtcaactttgaagtcttcttagctagagactaatagagtgaacgtgttggaaggttcatgttcatgacattataaaagtaataatagtgaaatttcacaaagtatttataaacccaggacagactcaagagctctacttattatattagtgaaaaacaaacatacacacgacaataacacaacataaacaataatgaacatgaaaatcctccttttgtttgtcttccttcatcacctccactacttcatcaatggcagaacactaacagaacatcaagctttactaagtatcaaatctgccattactaatgatacgaatagctatctctccttatggaaaaacacaacacaccactgcagttggccatacatcacttgctcctcctcttcttcttctgtcatttctctcgatatctcctacttagagctcaccggaattctctcccctgatataggcttcctcaccaacctccaaaacctcactattcaatggaacgatttttctggccccctccccacttctctctctctcctcacccaactccgccatctcgacgtttcctacaacaatttcacaggtccaatcccatcttctctctctctcctcacccaactccgccatctcgacgtttccttcaacagtttcacaggtccaatcccatcttctctctctctcctcacccaactccgctatctcgacgtttcccaaaacagtttcacaggtccaatcccatcttctctctctctcctcacccaactccgctatctcgacgtttccgacaacagtttcacaggtccaatcccatcttttctctctctcctcacccaactccgctatctagacgtttcctacaacaatctaaatggcactcttcccttatcggtcgttgagatgtcggaactcaggtaccttaaccttaagtataactctttctacggtgagattccaccggagtttgggaaacttaagaagcttcaaacattggatcttggtaacaactatctttctgggggtcttccatttgagttgggttcattaaagagtttgaaatatattgatcttagtataaacaatttatatgggagtatacctgattatattggagattttccggagttggaatcacttttattagactcgaataacttcacagggagtatcccacaaaagttaggtacaaacgggaagttgcaatatctagatataagtaacaacaattttagtgggagtttgccagcaagtctttgcaaaggagacaaactccaacatttgggagtatccgataatttgttggttgggccaattcctgagagtttgggaagttgcaagtcacttgaagaagtgaacatgggaaataatttctttaacgggtcgattcctaagggcttgtttggcctcccaaacattattgatgtttcactcaatgacaatcttcttagcggaggtctcgatgagaaatttggtgattgtgttaatcttttcaacattgatctctctaataataagctatcagggaagttacctgcgactattggaaactgttctaatcttcagttgttgatgcttaatcagaataacttcaccggaagtatccctcaagagattagcaagtgtaagcagctacgggccctcgatctcagccaaaatcagttctctggtgtgatacccaatgatattacaggtaagaaagtatattaaacttgttacttttgaaaatattcgctctagttttctttcagttggtccattctcacttttgcattattgaaatatatccctaaaaaagtaaatgtaattatataaaagaatcttgctcaaaataatatgaattatttttgtatgtgcaaaataatgtacaatctaactaatttgttgtgaaaaataatataattgtgtgaaatagtaaatgtgtggagatatataactttatttatcatattcactaagggttttaggtattttactatgacttttgcattatggagatatccaacttgacaatagtatttttgtaatatacttcctccgtttctaaataagtgcaacatttacatagtgtttactattcacagtttaaactttaattagctttggtgatttacattttaggaaaaacatagtcatgtgggatcttattagattcgtctgaatgtgaatttttttaatatcaactttttataatttttacttattgacaattgaagatattaatggttaaaataatgcattggcaaacgtgcaaacaagaaatgttgcacttatttagaaacggaggaagtatcatatatgaagattattgtacataacacttttcttatttttataaactatatattcttcttctttgtatttatcacaacactttttatatctttgcctcatattaatggcaacacttttaatttatctatttactttttatttcttggtctatagcccatttacatacttatgacacacctcataaaggacccacacgattaaccaaaatatacaaatatcttcaatgaaattaacttcaatactaatatgataaaaatcatgccccgctgtttatcctcatcctaagactctgcataaaattattatttcttgtccatacttaatcatgttgtgttttcacattttaactaataataaattacaattgatacaccaaaaaactctatgagcattgggtatgttgtttgatagaagcttcatgctattatttcttgtcaaagaatttcatatctcgatatcttctataccatctaacgaacaattattttctgcaggaaacaaaaccatttgcaattttgaagaaattaaattacttgatttatcaaacaatattttgaccggtgaaatccctcgttgtcttggaaatactagtactcaactcgaaacattatttcttcaatcaaacaaactgaacggtaccatacccgcaaacttctcaaagttatgtgattcattgatgtatctagatcttagtgacaatcaactcgaaggagttctacctaagtcattgtccaaatgtcaaaatttgaaactcctaaatgtcgggaacaacaggctaagagataaatttccctcatggctagacaacctcccacatctccaagttttcagtgtgcgtttcaatgccttctacggtcctataactagctcatcaaaggttaatcacccatttcctatgctacaaattatcgacctatctaacaatgagttttgtggcaagttgccaagaagatatatcaaaaattttgcaaccatgcgcaatatgaatgagtctggtgttggggatccacagtacctggaggactcatatagtccgtactctatggtattgacattcaatgggttacaacaaaaatatgaaaagcttattgtgacgatgtcgacctttgatatatccaacaacaactttactggacagattccatatgttatagggggattacactcacttcgtaaccttaatctctcgcataatgtcttaaccgggaacattcctccatcaattgcaaaattgtctttgcttcaggatttggacctttcatcaaacagacttattggtcgtatccctcaagaattagttagtttaacatttcttgggagcttcaatgtttcgaacaatctattggaggggcctatacctattggtaacaacttcaatacattctcgaataattcataccaggggaatgtcggattgtgtggaaaaccattacctgagtgtggagaaagaagggcaaaaagcaccactaataatcaagatgttcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctgaccaacaatttgttagccgatgaagagcatcaaaaccaaaaaaacaaaaaaattgagtaatatgcatgagtgtgaccttgttttccaaagtttagcattactattagtgtctcaattcataataataaaaaaattagcttgttcaagatttgtattttattcaaagattttttatgtctcttgtgcttcttttatcttatatatattttttgtatggtttgtttttgtttaatattagtccctccgctcaaaatgatctttcacgcttgagattggcattaaggtcaagagatgttgctaagctttagaataaaaaaattccaaatgcatagagggaaagaaagcgagacaaaatgttggagaaggcagagtaaatgatgtgatggaggataaatagtagaagtgtgataccgaaagtttgaaaataataaggaattttatttcttgctggcacttcgttctagtacaggtttttggcccttcaaaatgcttataatgtag

SEQ ID NO: 5 - DNA, 2460 bp, Artificial Sequence
  cDNA sequence of the sensitive variant of the Cercospora resistance-mediating gene
atgaacatgaaaatcctccttttgtttgtcttccttcatcacctccactacttcatcaatggcagaacactaacagaacatcaagctttactaagtatcaaatctgccattactaatgatacgaatagctatctctccttatggaaaaacacaacacaccactgcagttggccatacatcacttgctcctcctcttcttcttctgtcatttctctcgatatctcctacttagagctcaccggaattctctcccctgatataggcttcctcaccaacctccaaaacctcactattcaatggaacgatttttctggccccctccccacttctctctctctcctcacccaactccgccatctcgacgtttcctacaacaatttcacaggtccaatcccatcttctctctctctcctcacccaactccgccatctcgacgtttccttcaacagtttcacaggtccaatcccatcttctctctctctcctcacccaactccgctatctcgacgtttcccaaaacagtttcacaggtccaatcccatcttctctctctctcctcacccaactccgctatctcgacgtttccgacaacagtttcacaggtccaatcccatcttttctctctctcctcacccaactccgctatctagacgtttcctacaacaatctaaatggcactcttcccttatcggtcgttgagatgtcggaactcaggtaccttaaccttaagtataactctttctacggtgagattccaccggagtttgggaaacttaagaagcttcaaacattggatcttggtaacaactatctttctgggggtcttccatttgagttgggttcattaaagagtttgaaatatattgatcttagtataaacaatttatatgggagtatacctgattatattggagattttccggagttggaatcacttttattagactcgaataacttcacagggagtatcccacaaaagttaggtacaaacgggaagttgcaatatctagatataagtaacaacaattttagtgggagtttgccagcaagtctttgcaaaggagacaaactccaacatttgggagtatccgataatttgttggttgggccaattcctgagagtttgggaagttgcaagtcacttgaagaagtgaacatgggaaataatttctttaacgggtcgattcctaagggcttgtttggcctcccaaacattattgatgtttcactcaatgacaatcttcttagcggaggtctcgatgagaaatttggtgattgtgttaatcttttcaacattgatctctctaataataagctatcagggaagttacctgcgactattggaaactgttctaatcttcagttgttgatgcttaatcagaataacttcaccggaagtatccctcaagagattagcaagtgtaagcagctacgggccctcgatctcagccaaaatcagttctctggtgtgatacccaatgatattacagatcttagtgacaatcaactcgaaggagttctacctaagtcattgtccaaatgtcaaaatttgaaactcctaaatgtcgggaacaacaggctaagagataaatttccctcatggctagacaacctcccacatctccaagttttcagtgtgcgtttcaatgccttctacggtcctataactagctcatcaaaggttaatcacccatttcctatgctacaaattatcgacctatctaacaatgagttttgtggcaagttgccaagaagatatatcaaaaattttgcaaccatgcgcaatatgaatgagtctggtgttggggatccacagtacctggaggactcatatagtccgtactctatggtattgacattcaatgggttacaacaaaaatatgaaaagcttattgtgacgatgtcgacctttgatatatccaacaacaactttactggacagattccatatgttatagggggattacactcacttcgtaaccttaatctctcgcataatgtcttaaccgggaacattcctccatcaattgcaaaattgtctttgcttcaggatttggacctttcatcaaacagacttattggtcgtatccctcaagaattagttagtttaacatttcttgggagcttcaatgtttcgaacaatctattggaggggcctatacctattggtaacaacttcaatacattctcgaataattcataccaggggaatgtcggattgtgtggaaaaccattacctgagtgtggagaaagaagggcaaaaagcaccactaataatcaagatgttcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctga

SEQ ID NO: 6 - protein, 819 aa, Beta vulgaris
MNMKILLLFVFLHHLHYFINGRTLTEHQALLSIKSAITNDTNSYLSLWKNTTHHCSWPYITCSSSSSSVISLDISYLELTGILSPDIGFLTNLQNLTIQWNDFSGPLPTSLSLLTQLRHLDVSYNNFTGPIPSSLSLLTQLRHLDVSFNSFTGPIPSSLSLLTQLRYLDVSQNSFTGPIPSSLSLLTQLRYLDVSDNSFTGPIPSFLSLLTQLRYLDVSYNNLNGTLPLSVVEMSELRYLNLKYNSFYGEIPPEFGKLKKLQTLDLGNNYLSGGLPFELGSLKSLKYIDLSINNLYGSIPDYIGDFPELESLLLDSNNFTGSIPQKLGTNGKLQYLDISNNNFSGSLPASLCKGDKLQHLGVSDNLLVGPIPESLGSCKSLEEVNMGNNFFNGSIPKGLFGLPNIIDVSLNDNLLSGGLDEKFGDCVNLFNIDLSNNKLSGKLPATIGNCSNLQLLMLNQNNFTGSIPQEISKCKQLRALDLSQNQFSGVIPNDITDLSDNQLEGVLPKSLSKCQNLKLLNVGNNRLRDKFPSWLDNLPHLQVFSVRFNAFYGPITSSSKVNHPFPMLQIIDLSNNEFCGKLPRRYIKNFATMRNMNESGVGDPQYLEDSYSPYSMVLTFNGLQQKYEKLIVTMSTFDISNNNFTGQIPYVIGGLHSLRNLNLSHNVLTGNIPPSIAKLSLLQDLDLSSNRLIGRIPQELVSLTFLGSFNVSNNLLEGPIPIGNNFNTFSNNSYQGNVGLCGKPLPECGERRAKSTTNNQDVPKNDNERMLSMSEIVVMGFGSGVLVGLAWGYYMFSVGKPFWFIKMASKMESILIGFF

SEQ ID NO: 7 - DNA, 1998 bp, Beta vulgaris
  promoter (1)..(1998): native promoter of the Cercospora resistance-conferring gene Beta vulgaris subsp. maritima
gagcatagtgagtgcaaaagccatggaagctagattaaaaaggccatcattctaagttagacaattggaaacaacatcgagatacacgtacacataagggctgctcttctctattactccctctgttcctaatcatttgcttttttagcgggttccaaaggcctatgtttgaccactaatatatttaaattaaaactggtgatatatattaaaagaaaattatgatgaatttaacaaaaaccatatatgttatgtccttttttttcctatattaatgaatttttacagtcaaagttggtgaactttgacccaaaaaaagaaatggagcaaaaaaaaaaaaaaaaaaaaaaaactagggacaatgagtaacatttttatctatgtctttttaatatgaatatacgtaacaaattctgcaaaaatagagatagcaactaataacacgcatgaaaatgacaagttatattatacctttttttctcaatatatgaatatacgtaacaaattaactccagtagtttttagtaaaactattagattattgtgtaacatatactctggaaatagtactaagatccattacaatctttattgagaaatttcctcatgtaccccctgaggtttggcgtaatttccaaatacccctcatatttgaggaatttctcaaataccctgatgtttttgtttagactcaaaatacctttactatggacagtaccctaatgtcattaagttttccccttctctctccccaattttctctctcctcccattcccccacccactacccactgcccactgccaagtaggggtgtaagtggattggactggattggactttgccaaattcaaatccagtccaaagttttttggactcgagaaattgagtccaagtccgatccaaatattttttgagtccagtccaatctagtccgataattttttcttgagtccgaatccagtccagtccagtccgattattatatcttttttcccgatttaggttcaatgattcacaacattttttgagatgcttgagcatttgacatctgattcaattatcaatatccacaaataagattgaaagcttaaattaaagtaaaatactatgaataaaaagttgaattagatgcttaccttgatctaagttgagaggaagcatagagactgagaattaatctgagggacaaatagagaatgcgagagtcgagacagtgaggtagaaagaaaatgaagagtaagaggaagtgagtattaaggactgaggagtaaagtaagatagaattagttggctactagcctactaatgcagtattgctagtataatttacttatttaacaaatggagctaagtgcaatagtttagcgccaattgacatatttagagagagaaggctgaaaaatccaatatttttaaaatagtatcattatttttaatatatacattatatataaaaatatttttggactggactggacatattggactccaaagggatgagtccaaatccagacaaaaaatatttggacttgaaaatttaagtccgagtccagtccgaaaaattttcagtccaatccagtccgacaaatttggactggactggattggactctgaacttttcgtagtccgcttacacccctactgccaagtgccaaactgccaacccccttttggttgagttgatatttgacgcaaagacttggcgtgttggaaggttcattacacattttatccaagtcaactttgaagtcttcttagctagagactagagtgaacgtgttggaaggttcattacacattttatccaatcaaactttgaagtcttcttagctagagactagagtgaacgtgttggaaggttcatgttcatgacattataaaagtaataatagtgaaatttcacaaagtatttataaacccaggacagactcaagagctctacttattattagtgaaaaacaaacatacacacgacaataacacaacataaacaata

SEQ ID NO: 8 - DNA, 2000 bp, Beta vulgaris
  terminator (1)..(2000): native terminator of the Cercospora resistance-conferring gene from Beta vulgaris subsp. maritima
ccaacaatttgttagccgatgaagagcatcaaaaccaaaaaaaacaaaaaaaattgattaatatgcatgagtgtgaccttgttttccaaagtttagcattactattagtgtctcaattcataataataaaaaaattagcttgttcaagatttgtatttttattcaaagattttttttgtctcttgtgcttcttttatcttatatatattttttgtatggtttgtttttgtttaatattagtccctccgctcaaaatgatctttcacgcttgagattggcattaaggtcaagagatgttgctaagctttagaataaaaaaattccaaatgcatagagggaaagaaagcgagacaaaatgttggagaaggcagagtaaatgatgtgatggaggataaatagtagaagtgtgataccgaaagtttgaaaataataaggaattttatttcttgctggcactttgttctagtacaggtttttagcccttcaaaatgtttataatgtagagtcaaaattaatatccttaactagtttttaagtccgggttatatcctagatattaataatattcatttattagtaacattttattttataaatataatactaagcattatttggtttgctggttaagactttagtgtatatctatttctttttttttttattgtatgcgtgtttacataaactaaagactataagggatagtaccacgtggcgcagttccttgcttaggaacgtcttttaatatattaactagtatttgggcccgggcgttgctccgggttggtattgtgtttccgaacatgatgtgcagtttttcccattcccactaaaatatataaaggaaaactcaacatttaaaagatacaaatataataatatggacacttaaaacatgattaaaagttgattgagatggtaattgtgtcatgttataatagtaagaggttgcctaattgaggttgaggtggtggagtagtggtatcgcttcccatctgttatccctgaggtataaggatcaaacctcataggactcatttgagtaatttcccatatcctcctctcaaatgagtccttttcatctgacaaaaaaaaagagtctaattttaaattaaaattagacgatcttttataaaatcggcactttctgcacataggtcacaatttttttgtttctatctctctgctttctttaatttcacagtctccaactctccatcaacatcttacttattttagaatagatgatgtatggtagtattaaatggtaaagtactaaagctcctataatacacagaagcttacatagtatagattcgtacatgagacaaggttacaatatactttctccgttctttttatattacaataattactattttaagtagtttcacatctattgtaacaattccaattttgttatagaaagcaactttaataattgacaatattgcccttactttatcttattaaaaccatcattaattactcactttctcttataaaattgcttttattttctaaggatgatttctctcctattctagttaattaaagagttacttttgtgctaaactgctcatttattccaaatccttaaaaattgtgtccaaacgtattgttgtaatataaaaagaacagaggtactattagtttgaataaattttgatcagattaggtcacctttagggggcgtttggttaggggtattctggaaagggtaagggaatcaacttacttaattcccttacttgttgtttgtttgctcaatttaatgattccctttacccaccccttactcccaaagtcctttactctcattctccccaccccccaaggtttcacttaccctttcttgattcatcattgaccatatctttgaccacccaactaccaccaccacttgaccacctaatcacctaaccacctaattacccaaccactattaccacccaacccctccacctgcccaccaatcggcaccataactgcccaaccgt

SEQ ID NO: 9 - protein, 911 aa, Beta vulgaris
  PEPTIDE (1)..(911): Consensus sequence of Fig. 1
  VARIANT (20)..(20): N or H
  VARIANT (27)..(27): H or R
  VARIANT (39)..(39): N or Y
  VARIANT (41)..(41): T or Y
  VARIANT (42)..(42): N or Y
  VARIANT (42)..(42): S or N
  VARIANT (43)..(43): Y or S
  VARIANT (47)..(47): L or S
  VARIANT (75)..(75): D or N
  VARIANT (76)..(76): I or F
  VARIANT (77)..(77): S or T
  VARIANT (78)..(78): Y or M
  VARIANT (80)..(80): E or F
  VARIANT (82)..(82): T or E
  VARIANT (99)..(99): T or S
  VARIANT (101)..(101): Q or R
  VARIANT (102)..(102): W or S
  VARIANT (104)..(104): D or L
  VARIANT (111)..(111): T or H
  VARIANT (121)..(121): H or Y
  VARIANT (126)..(126): Y or Q
  VARIANT (171)..(171): D or H
  VARIANT (174)..(174): Q or G
  VARIANT (184)..(184): S or F
  VARIANT (208)..(208): F or S
  VARIANT (246)..(246): K or R
  VARIANT (265)..(265): Q or E
  VARIANT (268)..(268): D or N
  VARIANT (277)..(277): G or S
  VARIANT (280)..(280): F or S
  VARIANT (290)..(290): Y or H
  VARIANT (318)..(318): S or N
  VARIANT (319)..(319): I or R
  VARIANT (321)..(321): N or K
  VARIANT (376)..(376): A or L
  VARIANT (386)..(386): H or D
  VARIANT (388)..(388): G or D
  VARIANT (389)..(389): V or A
  VARIANT (391)..(391): D or Y
  VARIANT (397)..(397): P or S
  VARIANT (410)..(410): E or G
  VARIANT (412)..(412): N or Y
  VARIANT (418)..(418): F or L
  VARIANT (429)..(429): L or S
  VARIANT (440)..(440): N or K
  VARIANT (457)..(457): F or R
  VARIANT (458)..(458): N or D
  VARIANT (478)..(478): S or I
  VARIANT (479)..(479): N or H
  VARIANT (481)..(481): Q or R
  VARIANT (482)..(482): L or S
  VARIANT (484)..(484): M or T
  VARIANT (486)..(486): N or Y
  VARIANT (487)..(487): Q or N
  VARIANT (489)..(489): N or T
  VARIANT (490)..(490): F or C
  VARIANT (493)..(493): S or R
  VARIANT (505)..(505): R or Q
  VARIANT (506)..(506): A or T
  VARIANT (605)..(605): R or Q
  VARIANT (607)..(607): K or E
  VARIANT (629)..(629): H or R
  VARIANT (648)..(648): S or P
  VARIANT (651)..(651): N or S
  VARIANT (666)..(666): E or K
  VARIANT (692)..(692): D or N
  VARIANT (697)..(697): E or G
  VARIANT (705)..(705): P or T
  VARIANT (733)..(733): H or R
  VARIANT (733)..(733): N or S
  VARIANT (748)..(748): H or R
  VARIANT (785)..(785): I or T
  VARIANT (811)..(811): P or S
  VARIANT (814)..(814): I or H
  VARIANT (816)..(816): N or F
  VARIANT (819)..(819): N or D
  VARIANT (821)..(821): F or Y
  VARIANT (822)..(822): S or T
  VARIANT (823)..(823): N or A
  VARIANT (823)..(823): N or A
  VARIANT (830)..(830): V or L
  VARIANT (831)..(831): G or E
  VARIANT (847)..(847): S or G
  VARIANT (854)..(854): V or D
MNMKILLLFVFLHHLHYFIXGRTLTEXQALLSIKSAITXDXXXXLSXWKNTTHHCSWPYITCSSSSSSSSVISLXXXXLXLXGILSPDIGFLTNLQNLXIXXNXFSGPLPXSLSLLTQLRXLDVSXNNFTGPIPSSLSLLTQLRHLDVSFNSFTGPIPSSLSLLTQLRYLXVSXNSFTGPIPSXLSLLTQLRYLDVSDNSFTGPIPSXLSLLTQLRYLDVSYNNLNGTLPLSVVEKMSELXYLNLXYNSFYGEIPPEFGKLKKLXTLXLGNNXLSGXLPXELGSLKSLKXMDFSSNMLFGEIPQSYSLLRNLIDIDLXXNXLYGSIPDYIGDFPELESLLLDSNNFTGSIPQKLGTNGKLQYLDISNNNFSGSLPXSLCKGDKLQXLXXSXNLLVGXIPESLGSCKSLEXVXMGNNFXNGSIPKGLFGXPNIIDVSLNDXLLSGGLDEKFGDCVNLXXIDLSNNKLSGKLPATIGNCXXLXXLXLXXNXXTGXIPQEISKCKQLXXLDLSQNQFSGVIPNDITGNKSICNLEKIQTLKLSNNALTGEIPHCVGNIELIALFLQSNKLNGTIPANFSKLCDSLIYLDLSDNQLEGVLPKSLSKCQXLXLLNVGNNRLRDKFPSWLDNLPXLQVFSVRFNAFYGPITSSXKVXHPFPMLQIIDLSNNXFCGKLPRRYIKNFATMRNMNESGVGXPQYLXDSSIYSIXYSMVLTFNGLQQKYEKLIVTMSTFDISXNNFTGQIPYVIGGLXSLRNLNLSHNVLTGNIPPSIAKLSLLQDLDLSSNRLXGRIPQELVSLTFLGSFNVSNNLLEGXIPXGXNFXTXXXNSYQGNXXLCGKPLPECGERRAKXTTNNQDXPKNDNERMLSMSEIVVMGFGSGVLVGLAWGYYMFSVGKPFWFIKMASKMESILIGFF

SEQ ID NO: 10 - DNA, 201 bp, Beta vulgaris
agagcagattggcatacttrtgaatattctcactggctattaaattctcagaagaaaaatcaacaccaagattatgacatgcttgtgcaaagacacacccrgtcatgaatgcatcatagccagcttcatgcttagccccagagttccaatttgaggayctgcaagaaaacatgggagtaagatggtttcacataaaacatg

SEQ ID NO: 11 - DNA, 201 bp, Beta vulgaris
gggtttcttcgaagtttgattttgttacatttttcaaagagaaattagttgttgatgttgaataatgatgataagtagttagggttcgtagtaaggtggasgaragagaaaatggcgtcactctgayragcttcttcattttgttcttcttccttagctctgttttcagtcactgcgccattttttttttaaaaggaagat

SEQ ID NO: 12 - DNA, 134 bp, Beta vulgaris
caagcacaaaatcaaataatgagaatcacactatccaaagaaaatttccatccacatttatccaacacarttatctctcttttacacccaaattatgtcaaccaaaaacastaaaacaagtgagtgcagtagct

SEQ ID NO: 13 - DNA, 134 bp, Beta vulgaris
taagtaaaaagtggtaaaagaattaccaaaarcgcacaraataaattaattagytggatwtaactawttaacctattccttttttctgtcgctataactacttttgcttaacttattgatggtttgatcgttga

SEQ ID NO: 14 - DNA, 150 bp, Beta vulgaris
ttataatgtagagtcaaaattaatatccttaactagtttttaagtccgggttatatcctagatattwataatattcatttattagtaacattttattttataaatataatactaagcattatttggtttgctggttaagactttagtgta

SEQ ID NO: 15 - DNA, 201 bp, Beta vulgaris
acatctacactgggagactgataaggacgtttgcagatgtcaagtatgggaatcatcatctaacatgggtggagattgtgtacaatgttatttcattcatmgtggcaataattaccattgttgcgtttactgtatatgccaagagagccttcgaagaacttaagagggcagaagctaaggaggatcgagaagaagaaacct

SEQ ID NO: 16 - DNA, 28 bp, Artificial Sequence
  genomic target sequence with 5′-flanking PAM (4 bp) 5′crRNA # 1
tttatttcgatttcgattcttggattat

SEQ ID NO: 17 - DNA, 28 bp, Artificial Sequence
  genomic target sequence with 5′-flanking PAM (4 bp) 5′crRNA # 2
tttcaacccagtatccttatccgtcact

SEQ ID NO: 18 - DNA, 28 bp, Artificial Sequence
  genomic target sequence with 5′-flanking PAM (4 bp) 5′crRNA # 3
tttatttaaacatgatacgtatcatatt

SEQ ID NO: 19 - DNA, 28 bp, Artificial Sequence
  genomic target sequence with 5′-flanking PAM (4 bp) 5′crRNA # 4
tttaaacatgatacgtatcatattgagt

SEQ ID NO: 20 - DNA, 28 bp, Artificial Sequence
  genomic target sequence with 5′-flanking PAM (4 bp) 3′crRNA # 1
tttgtgggtgggtggttttcacgtgtgt

SEQ ID NO: 21 - DNA, 28 bp, Artificial Sequence
  genomic target sequence with 5′-flanking PAM (4 bp) 3′crRNA # 2
tttcccctccctttgccgctgcgaagtt

SEQ ID NO: 22 - DNA, 28 bp, Artificial Sequence
  genomic target sequence with 5′-flanking PAM (4 bp) 3′crRNA # 3
tttcttcttcttgcttccaccataacac

SEQ ID NO: 23 - DNA, 28 bp, Artificial Sequence
  Mutagenesis primer 1
tcagtgcagccgtcgtctgaaaacgaca

SEQ ID NO: 24 - DNA, 28 bp, Artificial Sequence
  Mutagenesis primer 2
tgtcgttttcagacgacggctgcactga

SEQ ID NO: 25 - DNA, 20 bp, Artificial Sequence
  pSeq_CRBM4_F1
agcgcaacgcaattaatgtg

SEQ ID NO: 26 - DNA, 20 bp, Artificial Sequence
  pSeq_CRBM4_R1
gatgaagctgaggtagtacc

SEQ ID NO: 27 - DNA, 21 bp, Artificial Sequence
  pSeq_CRBM4_F2
aggaaggttagcaagctcgag

SEQ ID NO: 28 - DNA, 20 bp, Artificial Sequence
  pSeq_CRBM4_R2
tctcgtcgaccttctggatg

SEQ ID NO: 29 - DNA, 21 bp, Artificial Sequence
  pSeq_CRBM4_F3
atgctgagtacgatgacatcc

SEQ ID NO: 30 - DNA, 22 bp, Artificial Sequence
  pSeq_CRBM4_R3
tagacctgcttctcaaccttca

SEQ ID NO: 31 - DNA, 20 bp, Artificial Sequence
  pSeq_CRBM4_F4
accactcactcctcgataag

SEQ ID NO: 32 - DNA, 21 bp, Artificial Sequence
  pSeq_CRBM4_R4
aacgacaatctgatcgggtac

SEQ ID NO: 33 - DNA, 28 bp, Artificial Sequence
  Oligonucleotide fw for the generation of short 24-bp protospacers (5′crRNA # 1)
agattttcgatttcgattcttggattat

SEQ ID NO: 34 - DNA, 28 bp, Artificial Sequence
  Oligonucleotide rev for the generation of short 24-bp protospacers (5′crRNA # 1)
ggccataatccaagaatcgaaatcgaaa

SEQ ID NO: 35 - DNA, 28 bp, Artificial Sequence
  Oligonucleotide fw for the generation of short 24-bp protospacers (5′crRNA # 2)
agataacccagtatccttatccgtcact

SEQ ID NO: 36 - DNA, 28 bp, Artificial Sequence
  Oligonucleotide rev for the generation of short 24-bp protospacers (5′crRNA # 2)
ggccagtgacggataaggatactgggtt

SEQ ID NO: 37 - DNA, 28 bp, Artificial Sequence
  Oligonucleotide fw for the generation of short 24-bp protospacers (5′crRNA # 3)
agattttaaacatgatacgtatcatatt

SEQ ID NO: 38 - DNA, 28 bp, Artificial Sequence
  Oligonucleotide rev for the generation of short 24-bp protospacers (5′crRNA # 3)
ggccaatatgatacgtatcatgtttaaa

SEQ ID NO: 39 - DNA, 28 bp, Artificial Sequence
  Oligonucleotide fw for the generation of short 24-bp protospacers (5′crRNA # 4)
agataacatgatacgtatcatattgagt

SEQ ID NO: 40 - DNA, 28 bp, Artificial Sequence
  Oligonucleotide rev for the generation of short 24-bp protospacers (5′crRNA # 4)
ggccactcaatatgatacgtatcatgtt

SEQ ID NO: 41 - DNA, 28 bp, Artificial Sequence
  Oligonucleotide fw for the generation of short 24-bp protospacers (3′crRNA # 1)
agattgggtgggtggttttcacgtgtgt

SEQ ID NO: 42 - DNA, 28 bp, Artificial Sequence
  Oligonucleotide rev for the generation of short 24-bp protospacers (3′crRNA # 1)
ggccacacacgtgaaaaccacccaccca

SEQ ID NO: 43 - DNA, 28 bp, Artificial Sequence
  Oligonucleotide fw for the generation of short 24-bp protospacers (3′crRNA # 2)
agatccctccctttgccgctgcgaagtt

SEQ ID NO: 44 - DNA, 28 bp, Artificial Sequence
  Oligonukleotid rev fuer die Generierung kurzer 24-bp Protospacer (3′crRNA#2)
ggccaacttcgcagcggcaaagggaggg

SEQ ID NO: 45 - DNA, 28 bp, Artificial Sequence
  Oligonucleotide fw for the generation of short 24-bp protospacers (3′crRNA # 3)
agatttcttcttgcttccaccataacac

SEQ ID NO: 46 - DNA, 28 bp, Artificial Sequence
  Oligonucleotide rev for the generation of short 24-bp protospacers (3′crRNA # 3)
ggccgtgttatggtggaagcaagaagaa

SEQ ID NO: 47 - DNA, 24 bp, Artificial Sequence
  Primer pCRBM4_F1
cacattttatccaatcaaactttg

SEQ ID NO: 48 - DNA, 23 bp, Artificial Sequence
  Primer pCRBM4_R1
ccttcgagaaataacatggtgaa

SEQ ID NO: 49 - DNA, 25 bp, Artificial Sequence
  Primer pCRBM4_F2
gtacagtgacggataaggatactgg

SEQ ID NO: 50 - DNA, 25 bp, Artificial Sequence
  Primer pCRBM4_R2
ttagtggtcaaacataggcctttgg

SEQ ID NO: 51 - DNA, 23 bp, Artificial Sequence
  Primer pCRBM4_F3
agtaagaggttgcctaattgagg

SEQ ID NO: 52 - DNA, 23 bp, Artificial Sequence
  Primer pCRBM4_R3
ttgccgctgcgaagttccctctc

SEQ ID NO: 53 - DNA, 42480 bp, Beta vulgaris
aaatgatacaggggtatatttgactctatgaatttcagaaatctaatcaaatttgctaagcttccaatgattctactaagccctacaaattacaagaattagttactttcatctctctgtcggcttcagaaccagaagtgtacaatatcttgtcaaacaaactctgcttagaggagctctttcgatcatcttttttcgatttggaagttcccggtgataggattgacattgctgttttctcggtcaattcttctggatcttggttctgtccatctatctctggctccattaatctggtcttccaattaattccgatagcctcagcttgctctgcaaacaagacctttgagatcggggagctgcagatatccttataaacttcataaccagcagcacaggttttcccaccttccaacaactttgataaaggatgtaggagagagatagaatcatcactcgtttctaacctatccttcaaggcaaggaagttaacagccaagtctgccttactaaactgaacaaatactgcagtttcatccaagttatagatgcaagcaactgagtatataccaaacactttacagatgcattgttttatgtcacttgccttgagttttggggagaatccccaaatcaaaactatgttaggatgcaaaatgttaagaaacctccttttagctgaactgataacgggaatttcattcatatcaccagtgcttagattgatcacatctccactgttccaactaagatagagcagattggcatacttgtgaatattctcactggctattaaattctcagaagaaaaatcaacaccaagattatgacatgcttgtgcaaagacacacccggtcatgaatgcatcatagccagcttcatgcttagccccagagttccaatttgaggacctgcaagaaaacatgggagtaagatggtttcacataaaacatgtgtagaagtgcagtgaacactggcgaaaacaatctaattttacgaattcattcactcactcagcttcaaattaagtttcccctttatttagggtgccccaaaaagatacactcttctgtttaccttctctctccaagcgaccaatcttttctctcttctccaacatcgttttctttttctctctctacccactatccattttgtcctcctacatttgataactattcttaatctccaagaaaatccaatgtgtgaaataattacgggacagggagtatacagaagcagcccccttgccaatatagtttacaaattaccctcagaattaggcttacctttcccaaaggagcaataaattcaaacaaatctaaaaggtacaaggcattaagtgccgaacctcatgtcatcaacctggacctccaccttcacacatggatgtacaccaccattagaggattgtccagaggctatctcagggcacaacagagaaaatgctgaggccaatgacgtgctggctttattcaagaatttttgaaggctcgtgtctgcattcaaaagtattttcgtgtcgacaacatgaggaaaatacttgtggatctcgagaacaaactcttcaacagttgatggaagaggaccaaagaatttatggtaaatatgtgccatatctgcaaaaaattataatggataagatgacaagaaaagatactaggaaggccttcaagtacaaatattatatcatgatgctggacgaccgatgctcccacaattatgtttgttaccaaatgcttcgaaggataattactaaattatgtgaatggtggttaccaagtgtcccggaccatgcaataacttctcctttcagtgaccaacaagaagaagacgtacctaaaaagcaattgtgacctacaattagcttcttttcagcagcgagaaggtcaaggacatgccggaaacctgcagctgcttttattttgcgagttgcttgctggtgagacccatacttcacctcctcctacaagaacaaacagaacaatcacacatgcagaaagttccccacataccaagttgctgtctgctaaacactgaaactaacttatctctacaaacaatgaaggaagttcctcaccagaaggttgatcttatcattgtcagattctacaaaaacaataagcttctgcaagatggcactgccgtcatgagcacacacaaaaacaagatccttgaagtgcttccttgtaacctgtaattgcagatcattagtatatattcaagatgttataaatttattgaaaagcagcgtctaaaacaataaaagtcatgcttaaggcatagagcgatagagcatagacacttcagagtttaataagagcaaatactccaggagaacataaatatatttcatatcacaaatcctagtaccaactggcaacggctaactgccaatttatgtactgctcaaaaaggccaagcatctaaaagatggcttaaaagtcggattttataagaaagtcgtcacatgattgctattacattgacatatcaaaggtcaaatgctgaaatttggttcagcttgatatatattaagcatacaaacgatacgttgacaagaaagcctaacaagacatgaagcatcaggcacataacattcaaaagattaccaattcaatcaacttcagctgatgagaagtaaatccattcaatcgaagagcaggacgcatactaaaaaatatggtttcaaattgttgtttagattcaacagcacttggaagctcggcatttctgttctgtagcaacatatcatgccaatcactgagccgaattttcatccgttcggagaataaaatatcagctacattgcccaaaggtaaatctctaacttcattagaatatgcccatttcccgttatatactgaattcaagcgactcaaagcctcgtcttcctgtcgtctagataaataagacacgcctgagattatacaatgtgtaaatttaccacaataatgacatcatactgacaaaatctcaaacaaatagttctaataaagtcatgttatctgaaatttctatgaataggaaattgaacaaaaccttcatgcttttttccaactaaaattgacatcttctacattaccttcatgtatgcatgcattgaagtcaaactggtattttgccaagaagtcaatcgaagttgtttggcacaggaattcatatgatgggccatcagtgggaagctcttgacgtggaaatatataaaaattatgcctgacaatgaaccattgtaaaattattagatggagtatctctatttattgtttacagccaattgagcttttaacaattactataggtagtgtttggaaacttgtatttcatttcaaataatggaattgaaatctggaatttaaagtttgtatttcaattcctaatcactgttttgtaaagggggtttgatagaagagagagaaatagaggtttaatggaggagagagaaaaagtgtgggtttactaaaaaaaagagaaataaatattagaaagtgtgggtttactcatagagttgggatatgtatgaggagagaattttcaaatgccaaggtaatagcttgaatgacaaatttaataatttcaaattccatgtcatccaaacaatagatttcatccaaatccaagatttgaaatgaaatcttgctatccaaacatatcataaattaattagtaatttagacttgctttctgctgcacttacttatggaaataattttacttcagtccttaaataacccgcaatttacatcaaaggcactaatataaacacctagttacgaaatggaaatatcagatatacctgtaaaagtaaagaaacaaaaatacaaccctgagcatgaaggtatccttcaaaagtgcaatatctgcatacttagaaccggaattagaagtgcgaatgcagacaataaccatcccaggatcagaaacgtccaagaaagttgagattcatcatccattctctttagcaaatttatgaactctaatatataaatcatacccccccccccatccaaaagcaattgtcaagctgcctgaacccctcataatttaggatacaacaaagtaatcctaaaagaccctttacaatactagtactcgggtatttccacaatcttctcatcattgaatccaaagcattgcatttgaagaaatcaaatcataatccattactatattagagcaaaatctatgtcattatagtattggagagcaagtatgactattacccctttacactaggcaaaacacattgtcacaatgctaacttagtcattaaccaatatcaatatgggactgtggatattcataaaatcgaagtttttcgcttgctcataaactatctttcattccagcacagtacaagagagaaaagacagcattttcatacacttctttctttagttcaaattcacacagcagcaaaaaattcacttcttcatagctttagctcagcaaacaaagcacaaagcatgcaattactctcacacatagcacaccaaaaaaacaaaaaccactaaaaattcacacaaaaaaaaccaacaaaaattccatcgcaatttcaacaatcaaaacaatcttctaagttaaaaagagagataaagatgagaagaaaaactaacggatgagcaacgaaggaattcttcgaagaatcccatcgaaatggacaaacaccaaattgaacaacggcgaatttctcagcagaatctttcattttaaggtatcgaacatcgtgccgatcaaactcgaacgattcgcgccaaggtgagcttgtaattccagtcatttcgagatcaatggcgacaaaatcggcagattttacatgcgtagtgaggtcaattagggtttcttcgaagtttgattttgttacatttttcaaagagaaattagttgttgatgttgaataatgatgataagtagttagggttcgtagtaaggtggaggaaagagaaaatggcgtcactctgacaagcttcttcattttgttcttcttccttagctctgttttcagtcactgcgccatttttttaaaaaaaaggaagatgaacaaagcaaatattgaacccaaattttgtaattttggcccactttatatgtacccctccgtttcaaaatatggagcacgccgcacacacgacatttagggtcgaattttgaacattcttcaagatgatctaatggtataatctctataatttatatgtggcatattataataagagttttatgaagtcaaaaagtggatgtcatatatttaatgcatggtaagtttttcctaaatctgtatactagggtaacatacatatgttgacttgaagtatatataattcttgtagtataaatatggctttggccataagtagtaatacacaacaactagaaaaattgaaatcagtccactgttatcttgtactctataattttctgtttccttttgtttcgcaacaaagacatatttgtggtgaaagataattttcgtaaattgaatgacttatattttgaaataaagagagtattaggtaaggttacgtgcttttcgcttgaatttgttagacctcaaatgtatatgtgattagaacggattggctctagtttttattttatagaagtatatatgcatttttcttagagcacactcgaaattactttcggatagatatattcgggaaaaaaagaggttgaagggaagttcatcaataattatggtaaaggaaaaaggacatcgttacaattctaaattctagataggatgtgatgataatccaaaagtcatctgaaaaactaaacaagtccaagatgctaatgattcgagtagagattgaatgagtgaccctaaggattgtcaaccctcttattctaacgtgtgtaaaagaattgacaactctaagagttactcaaacatttttcgattcgagtggttaatataccaatttgaaactattgacaggagttattttaatgagtataatggtcaatggagcactgaattccatctcacatagtcacatatttcatctcaagttctgatgatttcaaacattgaaaaaagatgatacaagcaattaattcctagggaaacatattgtggttttcatggatacaagagtgagaataaatcaaaacttaggctctaacatttcttttctctactagtaattgctaattatatcaattcaattgtcagtgtaatcagttaatcaccaaatctcttgtatagtcagtaaactatacactgtttagtcctctggattttgcccggtcgaattatgcagcataaccaaactttgaagtttagtacttcctttgcacccaagttagcttcacggcccctgccttctggtggatggtcaccctatgctttgagcattctctgcaatgcgcacgatattcaatgagaacgtcgccttgaaaatctaaattgcaactaaaaattagattgaaatgaaacccacaagagttgtttttctgagtagttggtgtagaattcacaagtcttgctccattgtttgaagatatgaagacaataatgtgctatgtaaagtgcagccgctagctaacagtggaagtggaaacttgatcattttacactcgcacaagcgaaagctcggctgacgttgcaaactgaagaaaaacctctcaaaccaattcgacttttgctcaaagttgcaaactaaagaaaaaggctgaatgcaaagcaagttcaccaatgaacaatagatcggtgttggcctgaggccacatcaagtgaagttgcctaattgcggccctctcatctgttcacaggaatcattttccatatagaatcactccaaaataaaagagcaaagctgcaccagatgcagaagcataactttcaagacaactgatgacagataaatagcaaaagaatgcttaagaaatgatcaaaattgaatggctctggaattacctcatcagctgattttcctttctctctatctctctatctctttactcgtctatggagctaccacatcacatggcgtttcatatgctttctgccgtcgaactagacgtgcagcaaaagctccatccattgaatgcttcactgggcatgagcgataaaacccatcttcagttaaaaagtcagatggaacatatctgcttacagaatctctttggaagtcctatcacccaacaaagaatatattaaaatagagaaggagaaaagaacgtatctatctgtcagcatccatatgaggtggaaactaggagtactatataaagccagtgcagtagctcctaccggatgtctaagaaggaaggcagaaaccctatcttcgttttcttcaagatcaatggagcaggtactgtacacaagcacgccatctggtttgaccagcctgtagatgttaaacaatcccacagacaaaagggaataatatgagtgaaacaagtcaacagggggaaataaccaataattctaggactgtcaaactcaagctcttcaaaaacaaagatagctcttaatctcacttgcaagcagcatcgaacagctcgtcctgcaacttctttagctcttccatatcctctgactttctattccaacgcaaatccggcctcttcagcaaaaatataaagttggaacaaggctcttagatacaagaactgaaaaaccttcgacatataatggactctatcaagggcacaatgacaaattctaaacatgagcatgtatatcaataaaatactaagaaccctttcaatggtactgctagaaggtttattgctacactttttagtacaccatctataggttttatagtaccatcaaaatggttcatggtgccataagaaaattttatgtatttatggtactatctaccatatctaattttctctgtaaaaatgtatttgtagatagagaccacgagttcctcttttagatactgacttttttttttctacatgatggccaacagacttctcaaacaaaaagaaaaagaaaatatttagataatatgagcaacaaaatagcaaccacctacttttgatagtacacccaggcccgaacaaggaacatctaaaagaactttatcaaacttcgaagtgttgctgtcctgaaaagaatagaaagtaactgcttcaacaaagaagaagaggcagaaagcaaagctagtacgcattttgcaatgacttactgaaaaggagcgaagatcagcatggatgcaagtgatcacattatcaacacgctgcagcttggctgtttcttcaagtatccgtaaccgacctttatttatgtccattgctgatatcatacctgaaaagctacacatttagaatgcagaaccagcatcattggtagttaagttatcactataccttggccattcaagcgagatgccatgaagagtgtcttccctccaggagcagcacagcaatcaatgatgtgatcaccaggctgtggatccagaacagaaacagctagacctgcagcgaagtatagatgtaaacttgggttgggctgtcacattttttcacatcttatcttcctttctattctttcaaaactgaggagaaatggttgggatttctataaacgtgagaaaaatggcatcagattagatggttttactgcatgaaaaaaattgaatgtgtttcggcatcacattactacaaggtcaaaagcactatctttgaaaatgtaggacataatgggacagagatgtgctgacctgcactctcatcctggactgagcataaaccttcttttagaagtccagtttgtatcacaatctaggatatgagaaaacaactcaagatgtaattgctcctaagatatcaatcatttcataataaacataaaagttattattacaagacagcacctgcatcccacttctgatgcagacaaagtcatccaaatgcaaggaaggctcatgcgggacctgcggacaaagtgttgtgatgcgcatagatatcgaaagaaggccctgtatagcactaatgagataagattcagtaaccttcagcatgttgagcttcacaacaaggtcatctcgagttaatccttttgcaatattggccctagtaccaagaaaaccatatgtattaacaagagaaaagtggcatagggatctttatgacttaggtaagcagttggcaattagagagaataaaacccccaaacctcaagctgaaactcggaacactattgttccacatcatcaatttgatagctccttcttgcccaagatacttggtccaccgtcttaccatccactgaaaggaaggtattaaaagggagaaaagactcgtcagcatagaaaattgtacatcttaaattttagaagtatagcaccatcttcaggcatcagtcaacgtaaataaataccacatctacaaatagaaccatactttctggacagtcgggatcatgagcacagacaatactgcatgattattgcctcgtattctcatgtatacaagtatatgtaacattaaatagcagtatttcttgagaaactcaccacgggatgggaataagttgtagcaagggcacgtgcttgtgaacgatcatcaccctccaatttgggtacaggaagggagtcattatcctataaagagaaacagcttttgttttcaaccatatcaagacaaacagtttattaaactataaacaacaacaatacacatgcacacacctactgggaacaagatatatactactgataagtattttctgattgaagaaaaaaaatctcatttatttgcaaatatagatttaatgacaagaaagctttgaaccttaaggaaaactagctttcggaggatcccattcaccatgtttcctgcgcctggtctaagagcatacttggcaagattcacattctgcaatataatccaacagtaagaacacgacatggatttagactcaagtctctgaacctatagaacaagtaaaattagatcttatctcatttgacaatttaaaattagatagtgcaatattctgcagttataagacttcatgtgtgcatactgcacaagtcatcttaaaggtgttattaaagctttaattgccatttgacatccccttgctcaactttagcatgtttttaggctacaacaatacgcactgtctacatggacatacaaattacaagcgtatggaaaagcaataagcgcaaggaagtcttcagccagaaactctctatgagtccaacaatatgcaactaaatatccaagtaccgtgaatgagtaagaactaacctcgtcaacaacagcatatggtggcatttccagtttcacaatctcatagcatccaatcctgaggatctaaaattaaagataaatcaatacacaacatatgatatgggtcggagcgtatataacaagtatagcaactacatttgaacagataacagcctttgagacaataaggaactccgacattccagtatatgccagatttcatatctttagctctaaattgccacgcaaaatgttattgggcaatatacctgtagcaggagaggttccatgttcctaaaggagctttcatcatggcatgaagaaacaataagataatccagatattttctccaacgaattgaaccaccaacaatgtcagtgacctacaaagacaagttgtcaacttaaaacttttgaagcgtcatttcacttctgtagaccaatacaaaagctactactgctttacatcataaaacctttagtccttaggttcatctgattggcaaaaaaggtccagatgcaagaaaagcaagtagctgtaatgctgtattatatcagcattattcagaacagaataataaatatctacagattttgggtggaagcttgatgatagagtatctccacaaagagaactcgcttgagtcccaactcccaaatctacttttttggagtcacattatcagtcattttttctggactcttataggaatagtgtgctatgtaatgatttatggagcaggggcatttcatgaatagctttataagttagtatgggtgtcttggggaataagttaaagggttagttagagggaagaagtacaacatatatatagagcttttgtaagaagggtggttatgttgaaaatagatgagaaattgggtgagctcatagtagttcaatttggactttgggagagaattaagcctcttgaaagcttgaatatcatttacatttgttgtttttactcttattaatcaaccaaagttcattttcttcctttaatttctccattttagcactatgatttgtccaagctaagtgatttcttagcatagtgcacagtgtagtatatcggagaactcatttgagtcctgaaaggtcccacaagttacatttttcctactactacttgcaccaaaacaataagcatcattaagacattgtcactggtccttcttaggttcttttggaggggattcctcagatgggggaggcacccatgaaggaacatgttaccaagcaatgggacaatgcaaaatgcaccaatacagtagcttcacttcattgattgcatctatgtcacggaaaactgaagaaagaagcaacacctcaactttatccaggacagatatccactaacctaggatgcaagcttgagactatttagcaattgcctctgggatattaaatcagattacgactatatttctacagttattgcttaagaaaaaggtacgatttgaagcttgggaagaaagagaacaagagtaaaagaccaatctgagatctctttcatccaggtctctggtgcgaaatccaagagtcctctcaacatattccatctcattgttccctgaaccctttcctctctcatttagaagatcagcgaaggcaccaccaaactctatccgcatcaatctcacagcagccactggatttacacatgaaagcaaaccaggagaaccataaaaatcacaacaaacttcctgatagcctactcactagcatcaaccattgtgttcagcctaaaatgagcggctgttttcaattgaacagcaacttacatggaccactgcataaaagtgatttcttaatccagacaaacaaaaatgtttacttcaaccaactgaatttgcatcagctcattagtgatttgacaagttctaatttatgtatcaacaaacaagaccatatagctaggaaacaagaggcttaggctaagcttaatgcgtgaacaatgttagatttcaacctatcagcactgtggataactgcaaactgcgacttaaataaggaagataaaggaactgaatatgcaatttcaaggtgctcagcatttgaatcaacagttacttcagataattcagaacataaaagatttgaacattctaaggctacctcatgattgcaagcaatgttacctgattcgctaaccctcacaagccacaagccaaagaagcaatttggtaaatggttcatggtacaactgttcgcttttggactaatctaacaatactaggtggtaaattatgttcccatatctattaccataatgtacagcaaattaggcagcactaattccaaatgacccaacaaaaaaagaggaagaaaatccaaaaattcaagccaacatatgcactaaaattacaagcacaaaatcaaataatgagaatcacactatccaaagaaaatttccatccacatttatccaacacaattatctctcttttacacccaaattatgtcaaccaaaaacactaaaacaagtgagtgcagtagcttcacatcaaagaatatcaatcacaaacaccacataataaaatttcaactcctgcccaaacaaaaaaaatataaagaaaaaaaaacagcaaaatttcaaagataaaatagaaaaaaaaaaatcaaaatacagggggaaaaaaagtaaatttaccagctctatgaggcgaaacctgcaaattcagcttctgggttttctctgaaatatcaagcacaataaccagcaattaaaaaaaattataaataaaattaaaaagaaaagattgataattaaaatcaaaagagagcaatttaaagcacaatcctttttttaccattttttctgggaggaagagcatccttcgttttgggtttagacgaaaaaaatgagagttgttgtatttgtgcgcatgagtgatcattgctggaaatgaaagtgggaaagtggtaaatgagtgctttgtgaaattgggttttgaggaaaagtagaaagaagaagaagggtcgatgtcagagaagagagagagtggatggaaagtagtgatgattgcctccattgttgccggtgaagtgagctttctgcaaatatttcactggactagttttttttagcagataacgctaaaacagagaaagatgttcggttaattttaatttttggacatttaaatgactattcaatatgtttcaaccttttttttttaaaacaaaggaacaatactagtattagattacgttaatgtttagtacatccaatacttatgtgtgtttgacctaacttaaaatcgtaagttgtttaaaatgtcggtgtcttgtttttaagagatatcatacttactatctttggtttttactcttccattgttaacagaaactgtatttatttgggtaaggggtttgagtgaattcctgtaagtatgagaaagttttgagtgaagcaagagaaagagagaagaaaggaacttcgagtgaagattgagagaaacaacagttagtgggaactgttgttgggaacttgagtttaggagctcaggttgtaccccgagagaattaataggtttgtaacagagtcggtggcctattatagtggaaagtttgagtcaaaatccattgtggccgatgtcgtttcttcttattgggcctaggaagtttttcctcgctaaaatttcctgtgttcccattgtgtgttccttagctagctttcaattccgcaaaaagttacgtttattctctcactataattcacccccctcttatagtgctcatattatacaacaattgatatcaaagcaggaactctaaaaatacagaaatcatgttgagttcaagatcttggaaaatatgaatactacagaaaaactggaagaaaggtactctactcagagaccaccgatgttcaatggcaaattctacacaaactggaagaactgaatgaagatcttcatcaaagccgacaaatatcaggtttgtagaatcatagaggcaggcgattttgaagtcactaccactaatgacacatatgaggtaattcctaaattcataactcatttcgataaagtatatttcgaaaagttggaaattaacgttcttgctattaaactgcttcattgtggtcttagacctcatgaacacaatcatgtcatgggatgcaaaatcgcaaaacaaatttgggatcttcttgaagtcactcatgaaggtacgggtaaagttaagagatcaaaaatcgatcttttaatgaatcaatatgaactttttcaaatgaaatataaggagtccactcaagagatgtttacacgctttactaatactattaatgagctaacctctcttggaaaagaaattacatatgatgaacaggtaagaaaggtcccaaggatcgttggatggctaaggttacgccttacaaaaaactaaggactttacgaagttcaatccggaacaacttactggctcccttatgactcacgagctacacttggacactgagaatggtgacttgtccaaacagaagtcgattgccttgaaagccatttttgtcataccgtcaattaattaagtaaaaagtggtaaaagaattaccaaaaacgcacaaaataaattaattagttggatataactaattaacctattccttttttctgtcgctataactacttttgcttaacttattgatggtttgatcgttgaatccaagttttctccacccacaaagatattatagactttactttaaaaggtacgataaataatgtttaatcaggtatgcatcaaccttgaaattattaatttattaagatcaaattatgcatatttatattaaacgtacaggacttgtgcacaatccatggatgatattgtagattttgttgtaaaggagttagggacaaatgatgttgaattaagaatgatgaggaacaacattgaggtacctaatggcatacaagattatgtggtaacaaaggtgaagaagttggttgtaccaggcaatacagcagcggcaagccatatataggatgagctaccatacccttatgttgtgaactattgtcaccaccaacaagacattggtcattacgacatcactttagttgaggaatgataaacctctttttgctagatatttgcaaacatctagcagataaagaggaataaaacactatttatatttcatgaacactatttgttagttgcatgaacactatttttagttacacgaacactagttttagtagcatcatgaacactattttttagcatcggaattttcacgactactttttggtttgactgacactctgcaattttcgagataactttttggtgatatgggtcccatgaaatagaagatttatatttcatgaacactatttgttagttgcatgaacaatatttttagttacacgaacactagttttagtagcatgaacactattttttagcatcggaatctttgcgactactttttggtttgactgacactttgcaattttcgagataacttttttgtttgactgacaactatttcctatatatattgacagttttacccctgttagatgtttgcaaacatctagcaaaaagaggtttatcattcctccactttagttagcccaacctccagtaacgccatccagaccactgtcgtttgtcactacgacacttacgcttggcaaccctatgtcctagcccttcgatacctcgatatccgtccgggcaatgtccccagtttgtcacttctctgccattaatgacatattttggagtatcaaacccaactccaagtatatatcgcaacatggctcagtaaagagagtcatataatcatgacgtagtttctatatgccatcctacgtagtatcttgtaacatgaataacagcctggtttgcaggttgatggtacatggtataaattggtattactccctccggtctttattagtttaatcctttcttttgtacagagttataggagaaataatattgtgggtcatagaaggaaagagaaattattattttatgttaaagttgaatgtatgtgtgatgaaaagttagtagtcccatttcaaaatagaaaaaaaaaaggtaaactaataagggacatcccaaaaaggaatacgggtaaactaataaatatccatgcaggttgttggtacatggtacatgaagccgtccaaaaccttcaaaagcagtaagtcctgctgctatgccatattcaaatattcaactccaaaaaaaaaaaaaaaaaaaatcaaaaatccgcttttcagcgaaaatataggaaataatccaagaatcgaaatcgaaataaagtcatgatgcaagtttggagagctgaagttacactatatcggagtacttactcaaatgttgattagtactccgtgcgtttgaagtaaagtcacatatggagtagttccaagctaggttgtacagtgacggataaggatactgggttgaaaaggtgaacgtcgagatttatacgtgtatttatttaaacaggatacgtatcatattgggttctcatacgcgtaccagctgtgacttagaaaaattaaccacgctatataggttccaagccctcatgattaccttttcatagtgtaaatttcatgtagttgaatggtgggaatccaatcacaaaaacactgcaggtaatggaaatgttccaactttttccaagcattttaaaataagacatgtgattactaattagggcgtgttcggcaacagtaattgtggtgatagtttttagctgtgagagtagttgttagctgtgctattagcttttagtggttggtgtgtagctgttagctgttagatgtccaagtagcggtgtaaaatattgatgttcggtaaaagaagctgtcaaagtagctgtctaagaataactagttaaaaattcaaataaaactttaacatataatttatacaccactaaaagctacccaaaagctacaaattgtagcttttgacaaacactactaaaacactacttgtaccactaaaagctacttacaccactatcttgccaaacactcttattttttctaattagtgttttgacctagtcaagacactaaaagctacttaaaaagcttgtgccgaacatgccaattctgaaccaaggaacaaactataacaaaaaagtgctatgtgaaacttttgtaggcaacagaagtaaggcatttttggaatgtactaacaaatccgtattaagacttgtacatgaaaattaccgtggtaacatttacccacacttcctcattcacgtactccgattcattcttataagggcataaccgcataaggcacatcaagatccatgtatctaatagtttaatttgcctctgtgtttctgtattaacaatgagcatagtgagtgcaaaagccatggaagctagattaaaaaggccatcattctaagttagacaattggaaacaacatcgagatacacgtacacataagggctgctcttctctattactccctctgttcctaatcatttgcttttttagcgggttccaaaggcctatgtttgaccactaatatatttaaattaaaactggtgatatatattaaaagaaaattatgatgaatttaacaaaaaccatatatgttatgtccttttttttcctatattaatgaatttttacagtcaaagttggtgaactttgacccaaaaaaagaaatggagcaaaaaaaaaaaaaaaaaaaaaaaactagggacaatgagtaacatttttatctatgtctttttaatatgaatatacgtaacaaattctgcaaaaatagagatagcaactaataacacgcatgaaaatgacaagttatattatacctttttttctcaatatatgaatatacgtaacaaattaactccagtagtttttagtaaaactattagattattgtgtaacatatactctggaaatagtactaagatccattacaatctttattgagaaatttcctcatgtaccccctgaggtttggcgtaatttccaaatacccctcatatttgaggaatttctcaaataccctgatgtttttgtttagactcaaaatacctttactatggacagtaccctaatgtcattaagttttccccttctctctccccaattttctctctcctcccattcccccacccactacccactgcccactgccaagtaggggtgtaagtggattggactggattggactttgccaaattcaaatccagtccaaagttttttggactcgagaaattgagtccaagtccgatccaaatattttttgagtccagtccaatctagtccgataattttttcttgagtccgaatccagtccagtccagtccgattattatatcttttttcccgatttaggttcaatgattcacaacattttttgagatgcttgagcatttgacatctgattcaattatcaatatccacaaataagattgaaagcttaaattaaagtaaaatactatgaataaaaagttgaattagatgcttaccttgatctaagttgagaggaagcatagagactgagaattaatctgagggacaaatagagaatgcgagagtcgagacagtgaggtagaaagaaaatgaagagtaagaggaagtgagtattaaggactgaggagtaaagtaagatagaattagttggctactagcctactaatgcagtattgctagtataatttacttatttaacaaatggagctaagtgcaatagtttagcgccaattgacatatttagagagagaaggctgaaaaatccaatatttttaaaatagtatcattatttttaatatatacattatatataaaaatatttttggactggactggacatattggactccaaagggatgagtccaaatccagacaaaaaatatttggacttgaaaatttaagtccgagtccagtccgaaaaattttcagtccaatccagtccgacaaatttggactggactggattggactctgaacttttcgtagtccgcttacacccctactgccaagtgccaaactgccaacccccttttggttgagttgatatttgacgcaaagacttggcgtgttggaaggttcattacacattttatccaagtcaactttgaagtcttcttagctagagactagagtgaacgtgttggaaggttcattacacattttatccaatcaaactttgaagtcttcttagctagagactagagtgaacgtgttggaaggttcatgttcatgacattataaaagtaataatagtgaaatttcacaaagtatttataaacccaggacagactcaagagctctacttattattagtgaaaaacaaacatacacacgacaataacacaacataaacaataatgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcggtcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggtaagaaagtatattaaacttgttacttttgaaaatattcgctctagtttttgtttcagttggtccattctcactttgtattattgaaatatatcccaaaaaagtaaatataattatataaaagaatcttgctaaaaataatatgaattatttttgtatgtgcaaaataatgtacaaatctaactaatttgttgtggataataatattaattgtgtgaaatagtaaatgtgtggagatatataactttatttatcatattcactcaggtttttaggtatttattatgagttttgcattggagatatccaacttgacaatagtatttttgtaatataccaatatataaagattactgtacataaccaaaatgtatacttttcttatttttataaacttatatattcctcttctttgtatttatcacaacattttttatacccttttgcctcatattaatagcaacacttataatttatttatttactttttatttcttggtctataacctcatctacccacatatgacacaccctataaaggacccacatgattaaccaaaatatacaaatatcttcaatgaaattaactttaacactaatatgataaaaatcatgtcccgctttttatcctctaactaagactctgcataaaggtatattgcaattaatatgagatggaagaggtataataattatatgatcaaattcctggattgaaaaataaatatgagattaaaagtggtatgtttttggttaaaagaaactatccataaagtatgtttttggttaaaagaaactatgcaacataccaatcaaatgtttatacgcttacaatttatgtaccacttttttgtcattgtttttctattgtttgccatacgtacgttactaaatcatgttgtcttttcacattttaactaacaataaattactattgatacaccaaaaaaatctatgagcattggagtacgttgtttgatagaagcttcgtgctattatttcttgtcaaagaatttcatatctcaatatcttctaatttaacaatctaacgaaatttttttgacccaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctgaccaacaatttgttagccgatgaagagcatcaaaaccaaaaaaaacaaaaaaaattgattaatatgcatgagtgtgaccttgttttccaaagtttagcattactattagtgtctcaattcataataataaaaaaattagcttgttcaagatttgtatttttattcaaagattttttttgtctcttgtgcttcttttatcttatatatattttttgtatggtttgtttttgtttaatattagtccctccgctcaaaatgatctttcacgcttgagattggcattaaggtcaagagatgttgctaagctttagaataaaaaaattccaaatgcatagagggaaagaaagcgagacaaaatgttggagaaggcagagtaaatgatgtgatggaggataaatagtagaagtgtgataccgaaagtttgaaaataataaggaattttatttcttgctggcactttgttctagtacaggtttttagcccttcaaaatgtttataatgtagagtcaaaattaatatccttaactagtttttaagtccgggttatatcctagatattaataatattcatttattagtaacattttattttataaatataatactaagcattatttggtttgctggttaagactttagtgtatatctatttctttttttttttattgtatgcgtgtttacataaactaaagactataagggatagtaccacgtggcgcagttccttgcttaggaacgtcttttaatatattaactagtatttgggcccgggcgttgctccgggttggtattgtgtttccgaacatgatgtgcagtttttcccattcccactaaaatatataaaggaaaactcaacatttaaaagatacaaatataataatatggacacttaaaacatgattaaaagttgattgagatggtaattgtgtcatgttataatagtaagaggttgcctaattgaggttgaggtggtggagtagtggtatcgcttcccatctgttatccctgaggtataaggatcaaacctcataggactcatttgagtaatttcccatatcctcctctcaaatgagtccttttcatctgacaaaaaaaaagagtctaattttaaattaaaattagacgatcttttataaaatcggcactttctgcacataggtcacaatttttttgtttctatctctctgctttctttaatttcacagtctccaactctccatcaacatcttacttattttagaatagatgatgtatggtagtattaaatggtaaagtactaaagctcctataatacacagaagcttacatagtatagattcgtacatgagacaaggttacaatatactttctccgttctttttatattacaataattactattttaagtagtttcacatctattgtaacaattccaattttgttatagaaagcaactttaataattgacaatattgcccttactttatcttattaaaaccatcattaattactcactttctcttataaaattgcttttattttctaaggatgatttctctcctattctagttaattaaagagttacttttgtgctaaactgctcatttattccaaatccttaaaaattgtgtccaaacgtattgttgtaatataaaaagaacagaggtactattagtttgaataaattttgatcagattaggtcacctttagggggcgtttggttaggggtattctggaaagggtaagggaatcaacttacttaattcccttacttgttgtttgtttgctcaatttaatgattccctttacccaccccttactcccaaagtcctttactctcattctccccaccccccaaggtttcacttaccctttcttgattcatcattgaccatatctttgaccacccaactaccaccaccacttgaccacctaatcacctaaccacctaattacccaaccactattaccacccaacccctccacctgcccaccaatcggcaccataactgcccaaccgtcgcccaatcaagccacccaaccggcaccataaccgcccaaccaagccacccaaccggcaccagaaattgtaccaagctacccacacacgtgaaaaccacccacccacaagccctagaaaaaatggaagaatcgagagaaagggaggggagagaaaagatgcagcgactagaaggggagggggaggatgtgacggcaaggggagagggaacttcgcagcggcaaagggaggggaaacgtcgcgtcggcaaagggctaaggtggaattgacggggttgcagcaacaaggggagggcatggagacgtcgtaaccgcaaggggaggggcagcggcagtggaactggggtggagaggggtagtggcggcactagggtgtgggagaggtggcgggggatatcaagagaggggggatatggtggtgttatggtggaagcaagaagaagaaagaggaaagacaatgtactaaccaaacaacacattaaatctaagggttttggtttcctttccccatctacccctttcttgattccattccctttacccctttacaaccaaactcccccttagtttttactacttataaccttcaattttggctgttttttgtgacattttttacttctccgagcctggtcatattttctcccgaaacatttcgaggaaagtcgaagtgacttgtgaagttgtgcgggtgcttggcaccatttgtgttgcctcgaaaagcatctgaataccccatttattcctttctcctgaaacccaaaattacctcgcaataaacgaaaagatatccatatatttgttccaagccacatgactcctttccaacgacctcccatgtgaccatgtccttagaaggcatcccgtggcgttcgaagctcggacccccggaaagtccgaaagtgtgtattataactttcaattttggctgtttttgggatattttttacttcttcgggccttgtcatattttctctcgaaacattcataggattgtcaatgtgacttgtaagttgtaacgttgcacgggtgcttggcacaatttgcattgcctcgaaaagcctctgaacaccccatttgttcatttctcgtgaaatccaaaattgcctcgaaaaaaacgtaaaggcatccacatattcgttccaagccacataactcatttccaatgacctcccatagagtccgtagctcggaccccaggaaagtccaaaaacgtgtactataaccttcaattttggctgtttttgggacatgtttggacttcaccggcctggtcatattatcttccgaagcattcctacaaaatccgacgagactagtaacgttgttacgcgggtgcttgacaccatatgtgttgccttagaaagcctttaaacaccccatttgttcatttttcgtgaaacccaaaattgtcccgaaatgaacataaatgcatccatgtattcgttgcaagccacatgatttctttccaatgacctcccatatccttaggaggcatgcatcatgtggcgttcggcgagcgggtctcgggaaagtccgaaagcctgtgttataaccttcaattttggctatttttgggacatttttggcctttttcaagcgtgttcatattttctcccgaagcattcctaggttaggcgatgtgacttgtaaagcgtgggtacttggcaccattttctttgcctcgaaaagtctttgagcaccacatttgttcatttctcgtgaaattcaaaattgcctcgaaatgaacgtaaagacattcacatattcattccaagccacacatgactcctttccaatgacctcccaagcccctaggagtcgtcccgtggcgttcggatccggagctcgggcccccgagaatgtccgaaaccgtgtattatgaccttcaatttttgctgtttttggaacattttttgacttctctgggctggtcatattttctcccgaaacatttgtaggactaccgacgtgacttgtaatgttgcgtgggtgcttggcacaatttgcattgcctcgaaaaacctttaaacaccgcatttgttcatttctcgtgacacccaaaactgcctcgaaatgaacgtaaaggcatccatatattcgtttcatgccacatgactcctttccactgacctcccatgtccctagaaagcaccccatatccgaaagcttgtattataaccttcaattttggctgtttttgggacacttggactttttcggttcgttcatattttctctcgaaatgttcctagaaaaggtgacgtgagttgtaacgttgcgcgggtacatggaaccatttgccttgcctcgaaaaacctctgaacaccgcatttgttcatttctcgtgaaactcataattacctcaaaatgaacgtaaatgcatccatatattttttccaagccacttgactcttatccaatgacattctatgtccttagaaggcactgcttgtcgtccataattcgggccagggaaatgtatgaaagtgtgtattataaccttcaattttggctgtttttgagacaattttttacttctccgggactggtcatattttctcccgaaaaaatacttcgagtgccgacgtgacttgtaacgtcgcgcggatgcttgacaccatttgtgttacctcgaaaagcctttgaacaccacatttgttcatttctcgtgaaacccaaaattgcctcgaaatgaacgtaaaggcatccacatatttgttccaagccacatgactcatttccaattctctcccatgtccctaggaggcatcccgtggcgttcggagctcggaccctgggaaagtccgaaagcgtgtattataaccttcaattttggctgtttttgggtcattttttgacgtctcttggcttggtcatattttgtgccgaaacattcccaggattgccgacttgacttgtaacattgctcgagtgcttggcacaatttgcattgcctcaaaaagactctaaacaccccatttgttcatttctcgggaaacccaaaattacctcgaaatgaacgtaaaggcatccacatattcgttccatgccacatgactcttttccaatgacctcccatgtccctaggaggcatcccatggcattcggagctcgaacactgggaaagtccgaaagcgtgtattgtaaccttcaattttggttgtttgtgggacatttttgggcttctccgggcctggccatattttctcccgaaacgttccttggaaagccgaagtgagttgtaacattgcacgggtgtttggcaccattagtgttgcctcgaaaagcctttaaccaacccatttgttcatttctcgtgaaacctaaaactgcctcgaaatgaacgtaaatgcatccacatattcgttccaagccacatgactcctttccaatgaccttccaggcccctaggagtcatcttgtggcgtttggagctcagtccccggtaaagtctgaaagcgtgtattataaccttcaattttggttgtttttaagacattatttgacttctccgggactgggcatattatctcccgaaacattactaggagtgccgacgtgacttgtaacgccgcgtgggtgcttggcgcaattgtgttgcctcgaaaagccattgaacacccccatttgttcatttctcgagaaacccaaaattgcctcgaaatgaatgtaaaggcatcgacatattcattccaagccacatggctcatttccaatgacctcccatatccctaggtgtacaccccatttgtctgatgttataatagcaagaggtcacgggttcaaatcttgttacaagctaattttacttttgttaattgacatgacttatgtacacattggacaattatagtggagtaacaaaggtgacatgtgacgcgtatacattatcacacacgtcttttaatatatttgtatagatctagatttaagagtaatttttttaatgcgcaatacttggccaatttcttctgtatcaaatcataggtctttggttggttcataagagtaaagaccaaaataataatctgaactgcaaaaattttctccaagagttaaaagtttgtataagttagattaaaaaaattaatgacatatgatgtagttggacattaaatatgtaagtttagaagtaattgtgttaacataaaaaaagattcgattataacataaaaactaaagaaacacaaaggcgccgtacaacaatcaatattacccaagtcccctcattaatattaagggatgacctagctcgtacatatttaattatctttgaaaattcgttgttcagacttgctagttgctattctatatttgtatattcattaatcaatttttcaatatgtgagcatttacattttaaactagagcaaatattgtctcttttactattttgttgttgtcaaattttcaaaaataaattgctcaaatacttttcctagtgacataaaaaatagagcaaataatcaaacagtagcagacccaggaacttttacataatgtagacggcataatgtgttaatttttgcttcttttttctaatatcatccaataacacaattctgcttctattagtttgtagtttcagatgatgatacccaaacaataagaccaagcaacaaattgataagattttgcttctctttcttccacttggtgtaactgtaacagctttgaagtttaacttcagtaatcagttgcatatttggcatatgatcaaaacaatcaaattattatgtatggaaaagcaaaaaacttccaggtttccatctgaacaaggaggccaagagggtggaagcaagcaaggatatatgatcataaaatcctatgaatatgatgtacaaaccttttctactgcaattaggtaacctaaatgataccacctaggaacagcaacaacttatttacagcactaaacctaaatcaggttaaagttaatcagaccaccatgtatctgggtggtctctcgagggaaagcgtctccatctgtatccgggtaacagaggtttcttcttctcgatcctccttggcttctgccctcttaagttcttcgaaggctctcttggcatatacagtaaacgcaacaatggtaattattgccactatgaatgaaataacattgtacacaatctccacccatgttagatgatgattcccatacttgacatctgcgaacgtccttatcagtctcccactgcaaatgaatgctatcagcgtcaatattcgagataccaactcatttaactattgaattgccaaaaacagatatctttgaccatatatttgttactaaaaataacgattgataatgtgaaactatcactgatagatttaaaagaacttttataaaagtatagtttctctaatgtataactgcagaaaatagaatggggtagacaaatgaagtaattgttttgaagaatgcaaaaggtcaattcagtaatacttttatacgtgattgggggaagcattaaaaatcccttctaagataaagatgacctcattggcaatggaatcgacatccacagacccttgcattagaacagagtggaagtttctgtgaacttacgtgtagatgtaaagaaaagcttctggcaccatccctgcaattgatccccatagataaggccaaaacgtcatacttgtcaccacaactgcgtagttgaagatagtatagggaaatggtgaaaccctaaagagtgccaccacgcggaactgatgaaaccagctaccttcggcagcaagcctaagcatagcagccttatccggccatctttgcaaccattgctaacaaggtacaaaaacataaacattgtggacttaattagacaagaaagttaaattaaaatcaacattagataatcaataaatcaaatgtaagcagggaacatatttcttacatggattctatcccggaagagcaatccaagtaaatagggaagaatcattccaatagtagttccaaccatgattatcacaaaaccaagaccataaccaaagatcatgcctgcaagccacatggatgggccagaaggaatcagaaatacagggaagattgctagggaagtaacaaggaccacagcaagaaccggacggccaaaggcagtggcttcccattgcatcattggaacaagaacctgcagagaaagtaccaaaaactttgaggcaaaaatttcctgcttgtatattgcaaaaagtagtacagcgaaggcattccgtgcagaatggcttatagattggaaatacggagaacaatgcaactataagcacaggcccatctcttgacttttgggacaataacatggacccccagattgatttataagttctcacaccatagctagattttgttggaactttcataaatcatagtgacataagtatagcataatattcatgccttcgacagaagttttcgcatatggtaaggctactattgaaaaaattcccttgtgtttgaagtacgcataaaaatatctagtggcagtcaaccaaataaaacattctaggagtccctcaaaaaattaaagagtcatcagttcagaagactttaatatcaatactttctattatccgggtttggcatgcagtaaatttcatgagaaaaggaaaaatcagctatttgattatataaggaactaattcggatgtatcactaagctttccatcgactggaacatcgggagctagtctccaatactcgtcaaggatctaacataaacatcttctccgcaatcaaaaagccaaggtcacatacatctaggcctctgtctcattctgatggcatggtatgatgcaagttagacaacactattatttggcagatgacacttaggggtctaatatttaagctcattcaagataatcaagtaatcaagttcaatctcaaggtttcagttgcgctaaaaaatgtaatacttggctcattcagaattagtttgttgaagctggttggtatttgcttcatttgttaatggaaccaggctcataaacaagctttcattaggctaaacttatttaacaaaatcaaaagcttaatactataatttttgataggatttcttttgggcagttatacatgagtaatgaacaagctctacacaatcttttttaatgaacaagctttaatcgagctagggtacgttctattcaacttattggacctgaacttattggaacttatctgaactgaacttattgaacctgaactgaacttattggaacttattaaacctgattggacctgattcaacttattggacctgattgaacctgattggaacttattggacctgattgaacctgattgaccttattggaccttattggaacttattgaccctgattgaaacttattagaccttattggacctgattgaaacttattagaccttattgaacctgattgaaacttatttgaccttattagacaaaaacattattattattattgttattattattattattattattattattattattattattattattattattattattattgttaacctgattgataacatttatatctttcatagttattagtaacgaaaacatgttatctctagttattcaaagacgaattgcaaaatattgtaataataataataataatatattattattattattattgttaaccttaattatttgaccatgattataatattattcaatagcaatatgaataatcaaataatagacaataatacaagtataatactatacattgtggtactttaataaaaaaattctaataataacataatcagctaatagtaatatgaataataaaataatagacataatacaaataaataataaaataatagacataatacagataaataataaaataatttacactaatacaagtataatactatataatcattgtggtactttaattaaaattctaataataacataatccgctaatagtgatatgaaattatgaataacaaaatagtggacaataatacaaatgtttattaaacattgactatttggaccttattggaccttattagacctgattggaacttattggaccttattagacctgattggaacttattgcacctgattggaacttattacacctgattggaacttattgcacctgattggaacttattgcacctgattggaacttattgcacttattagaccttattgcaacttatctgaacttatctgaacttattggacctgaaacttaattttttaagttgaacagaacgcacccctagtatccacgaacatagttagttgttcatcgacaagggtgttaattccttgactataaaaaaaatatctgctaatatgtcctccataccatgtcttgatctgattcccaaaatcacgtgttttcgtgtctggtgaccacgttgctagacatggaagacaggtctaattgttcagtttcaagtcaggttgattaaacatatgttagcaatatacaatcattattagtcaaactaattcaactcgggtttggtttgattcaggttatgtcgaggatcaggtccaaatcgggttaatccttccaggtcaaatatatctaagtctgttttgccaaagtctactttttgtatccgtgtccatgctaaatgacaaacaaaaagcagcttttaccaagctcgaatcagatttgttcgcttaaagagtcacttcgctcatttacagcaacaattaaaggacaaaacattgtccattcaactacttacggatattaacttattggcaactgctagcgtaataaggcaatcaacagcactcggcctcaataatgaacctacaaggagtccaatgaccaatacaaattatcactggcatcatctagcacgacaatctcttaactctaagagtctaagtgccttgacatacaaaagtattccttttaaaagtacccccgtgtggatattctgccaagcaaatgcaatcgatacacccaattagggcttttccattatgagtcctcagagcctcagattgtaaaacaggtcagtaaaagaggaaaatagtatttgattcttttgctaaacccttggatataagaatggtgacttgtattgtcacgccaagcttctttcataaaagctgatcatattattatatgagagttctgagtttcaaggtccgcattcgatctaactagacatcacttccaattaaagttgagaaacgaaactaggtgtcctctttgtttcccaaaggtgaactttagatacttattataagcatattttgttatgaatcgggctaaggagagggctactcttggtattgcataattagttaattacttagtagtagcttgaggaataaggaagcaagtaagttagaggaaagagtatgaaaatctgctataaagtgaggagaggagggatagaaggataatcacaaaattattgagttaactttggttttagttgcttaggttgggagtgtccagccactcgaatgtcttgggactgtaaacaccattgttcatgatctaattgcatcaatattacaattaactcatttctcttcttatccatattcatcttcttacaatcacaactatttccagatcatccatccaaatcttcatccacttgccttagtttctactccagatttcagtctattacaaattgatttctacaatatgtcaattcatcacaaattatcatgttttctgaacaaaagttcactgtttcaggacaaatacagaaagaactactttgatgcttagaacagatatattgtaaaattgtattcggaatttgggatacaactggagaagatatgaataaataggcattcagggagctcagaaaaacagaccgtgccatatggtgctctgctgcataacaggaaataatggataaagtatgaataacgttataacttcttaaaaacctagatgacaagtattttggttgctttttattattggtaggcaaggagaatactcaacaacagtttagccttaaactgcttcttatttctcctcttcccctttttcctgatgatttggggttgtcactcagttcttttacctctcatttccaggtactttagagttatattacacaaaggattgcaagagaagaacaggtcgccctggcatgcactcagaaagtatacgacccttcacaggaaatgtggtgctccaagacttatatctcaggctctcatgagtcatgtcaaggaccatctttaatcatttgtattctaggtttctcaggcgatgcggtgtgctggtgtgtctctccctcccacttgagtgtgtgtattgtttgtgcccctaagtttttatcttaacaatcactactagtcaattagtcattaccaaccctacccacctctcttgttactgttgttcttggagatatttcatatatgtcagcttagaacttatattacgtttcttattacatattctcttaagctcgcgcacatactctgtgatcgaagggatccatattagttatcttttagtggagttgttgtgaaaaaagactgcatagaaaaattaagatagctcatagttgtaaatgtaattgaacttttagattgatagccttgaggctgcttgcattgaaccaaccaaattcagccaggctagtctatgcctctttggtgtcacctggtaggttgaatttgtgtagctgtagttctacaagagactgatttaaaaatgttttcgcactgaaacagcttaaaccacaaaacaggaaagtgcagaacaaactccagaaaatggtgcagaacataccttctcaaaaaggaaaggaactccccattttaacagtacgaggacaactgctacagcactaatggaggagatcaagattttgatccaccagatgaaggattctgatcttgtttcagcctgagaatgtaaggttgaagcttcaggcctctttgtaatagcagatgtcaccagactaacaaattcactgtcgtcttgcatagcaggcccaacatctatgtcatgcttagttagctccattgaatttggcatctccaagagatctcaagagctgcccaaaaagacggtacaatattatgagcatacatgacatgatgacaacccataaagaatatcataacctgtcacattttttattcaaagttcaacagccctcttacaacatgattgagaatggaggggaagagagagagagttggtctcagacattgatcacataatcatttcaattagttttaaaggtgctcatgaaatagaactagtgtcttaagctggagacttctgtatttttcatggttttagattatcaatcatattcttagaatctttgatctctagaactctttcctttcctcccaatattttttccactttgtcttttgttaattacggcttcgctgcaggcctgcaataaatcttttaaatttttacagatactatgtagagttgtatacataagctctaatctgaagacgattggtttcgatgctagttaatacaaataaatatattatggatataatatgcagtaaattgggccatgggcaccagggacaacttagacaagtatagtgcaactaccaggaaatttaagctgggtacctctgattcatcatgctggttgataatattattgcttccacaagtgttcgctacggctcaaccaaactaagtcacaactcacaagctgcacaacccaactgacaattatcgcctattgtctaagctatacattacattaccccaatgccacaacgtggctcacgcctaggcatggtaaggaagttcagatgtacgcagccttacccttttaataacaaagaggctgtttccaggtgacccttaaatcttaattgcaaacaccatctgctgcttcacataaataagcgacttcaaaattgtaaattaaagaatttgaatgcaaattgtgtgaaaaacaactccatcaagaatccattaagcacgctttactattagtatcaataataggaaacccttatatcccttttgacgaaggcacacatgcaacactaatgtgtccttataaacttcatgaaagtatatctctacgaaacccttttagtcttatgtgattctttaagtgtccaactgatgattggttacaaggtatttagcccaaagtagcatttcagagagatggtgtagaatgagtagcttataaaccgaggttgaggtgtaatcctaataaattaggaactaataccacaagagagatggacatgtagagatacaatatagtacagaataagattatttgaaatctttttaccagggaaactccagaggtgttccataaaacacaataccatataactgggagatcaatattttagattaaaaaatataaaaatctatttgggttgagtatatagttggttagtccaataatatataaatttataaggtggaggtcttcggtatatgacattccaaatttgagtatcaaatgatatatatggttttccatacttgaatcccttttcatgtactacctctgtttcaaattaatagttacacttacacttttcacgcatgccaatgcagaactttgaggacatatatctttagttttgtatttgtaaaaattataaaaagtacatattaataaaatacatattaatacgaatctaacaagatcccacatgactatgattttattcacgtataaatcacaaacgagggtcaaaatgcaattgtgaatagtgtaaaatgtcaaagtgtaactattaatttgaaacggaggtagtatgtgtttatgcaacacttttcctttttccctttttgctatttagtaatttatgtaaaatacttccattgacccaaaagttgggtgattatagtttacatctatcattattatttatcattactatagattattcaccattgtaatcaactttataaaagtatacacaggtaactcaggagtcaggggtgctgggccaaacacttttatagtttaaggtgaaaaatctcgagaatcttctcctgccacgcaaaatgagtgttcttccactttaaagatgttataacacttatcttaacctactattcgtaaataacacttatcttaacctactattcgtcaagacatacttgcttcatctcactaagaacgtcttagttttcatttgaaattcgtaccagaaagattcacttcaaatctatttatttttagataaattgttattaaaaacgacgaagaaacgtcagaggacaacaaatcctctaaactccaaattataagtgagtccaactatgttgacgtaaggtaattagagtatccataaaagccctggccgctttggcccacaaagcagcttagaatactacccaaccccaaatataatcaatcaggtgaggaagctcgcaacagatgcgagagttccactccaatcaaaggcaccagaacatagccatcgacatcttctcttctttaccccccttgaaaccaacagatcttaaggaagtccactagtgaacaaggacataaccactactcatgtggaatgccaatcagcctctgtcaaagggaagtccattagtgaacaaggacatacccactgctcaaggtagtcatgtggaaattggaatcccaatcagcctttgtcaaaaggaataagccacatcgcaatgaagaaaaaggtgcaaaccagatttattgcatctccaacacgacataaatatcgagaatgaggcctttactgacaaaggaactctggatttccaatttccactgagcattggactcagttgagaagtaattggtcttgctagattctgtttacgcacatactcttaatgataaataaatgtaacaggccaattggtctggaaaaaaacagttgataaaaggctagtttgggccttggggataaatataatctggtatgagttaataaatttctgtttaaggtaaagagaatgtgttatgtgggataatttaatcaagaaaatcttagtaagatggaggtagtctaacttccattcctcaaaatgtgtaattccttataaaatcagtcagcctctagatacatagttagcaaaaatggaaggtatagaagtgggggtgagggaagaggaaggaaagagaaccgcgatcaatcatattgttcgtgctcaagtttgagttgtgcctatagctagttagagtttgtctatttcattgtttttggtcagtgttcatattctgagtgtcatcgtgtttgggttctagaatgctccttttcctaatgtcgacatttctccactttactctagaaaaatgatctcattgtagccattccagcttcaattttaatggatactaagatccctttcaggaacaatgttaaggtagatgttagtgttttaacagccatgtggatgttagtgtctagaacgagtggtcaaaacactactagcctcaaaatattgtgatcagtctgaaaactctatgttagatggttgctttttttggtaggttcgcttgttttggggggttagctttgtttattttcttcacaatttgcccttaaacttttcacaaaatctacaattgaagattcttaaatagataacagacgtgtcagctacttcaacagctaattgtacgaaaaagttcagctaccttgaaaccaaaccactaacagctagtacagtttgtttctactattacatttatctaatataacagctagtatttagtccaacgatgtataatatcaatgaaatggaactaatctgtaaattggaccttaggcataagagtcgagttgagcaggtacactccaatcaccaagttatttaagcttaaaatgtctaacttccaatgctgtttgacgatactcattgccaagtgtttgttacagatcaaccaagcaaataaagcaacaagtgaacagctgcactagtacccaactgcgaattttcgtcgattgccaagtgcatgtctgggacacaataccatcatgtccatacccattaccttgcttagccagctatcgtaatccataacacataaaaaccaacaaagtcttgatagtttcacaaatcaaaatgttcacttttcattccaaccaaaacaagcaataaatctcttcatccatactcacaagaagaacaatctctcacactacccacttgattagtaaaaaccccaatcaaaaacaaaatccaacccacataaacaaatcaaatttagtaactacccataaactcaaaaacctcaaatcacaataccaataaaagagatatacaatcaatcaaaaaaaatacaacaacagctaaacaaataacatcataaactaaagttattcattttatttcctaactagagatcaattaagcagcataaaacaacatcactaattcaagttaataatcatcaaattctatactataaaacatacataccttaccaaaactacccagctgaaaattagggtagagctccagaaatcccggcgaaaaatccggtgagaaattcagctaaatttgaaaacttctttaggttaagtagtgtacacgatgaattgaagatttttacaagcatatgaaaatggtggttgaaattgaaatgggggtttttgaaaattgttgcgacgcgtaaaagtggaaaaaaaaaaggagagaatcaaagaaatgagcaagtttttgtaggtgggtttactgttgttgcttttgtttgtgcacattactgactattcttaattcttccatgcgtgtgggggtgaaggaattgttttcctaagttgtttagccacttcatagagtcattggatttgaataatctagggaataatgatcatgtgtttagtgtatctataaattataatttatgtatgtatattgtatatgtggtgaggcatagaggacaaggtctaagaggaatagaggattgtgagggagtgtttcatgcttttaagaatgatgagtcattgagtgtattaagttataagtagtatttgatcgagtagtaaagtttgtatcacgtaaatcagagtgataattaggaattgggatttgctcaagtggtgagttttcccatctttccgagcaaggtttctagggttcaattcctacctcaagcatttccttgggatttaaggggacggctcagaggaattcttcttaccaatattttaaaaaaaaaaaaattaagagtggtaatttagttcagatcctacctttatccggttcgaaacgacttcaagaaaaaaaaatccgacatcgtttaaaattttttacttccgactcatttaatccgcctccaactttgaaacaagtagtcttatttcttttatgttaagaaaatttgccaaaaaaaccctttttaaagtccagttttgcgaaaaaaaaaaaccttataaagcattctttgtgaaaacaaaccaaaaagtaaattatttttgcaaaatgaaacctaatctcatttttcggttttgaccatggacttttcgacattgaccacttctatttatcttcttcctccataatcacagcctagccaccactaccaacacctgccgctagcccccacaacctgcacccccacaacctccatccaccccctcaagcggcaaccccccttattcccatacgcggcaaccctacaccttatcctccacccccctccgcccttaccttttctcctctcccttcttccctccatcacccctccccactctcttctccctttgccccccatcgttgcaccacccataatccctctctgtaaccccctctcctcgcagctccccctccctcccagccaaggttgaaaaattacagaggcagtcgcatatggggatgggggactatcgtctaaggggtggagagagggtttgggggctgctggtgggggtggggtaggctgaatgtggtgggggctgagggtggggggtgaaggtggggctgcaggtcgggctggcggtatggagaaagaagggaaatagaagtggttaacaccggaaagtccatgatcaacaccgaaaaatgaaattaggtttcatcttgcaaaaataatttattactttttgatttgttttcgcaaagaatgctttataaggttttttcgcataacatttagacttttatcatccctcttagatttgacacatattatacgaattatactaaaaagactccttatagtaattcgactaatgttttattaaaatgaacctttagaataactcgggtaatat

SEQ ID NO: 54 - DNA, 201 bp, Beta vulgaris
agagcagattggcatacttrtgaatattctcactggctattaaattctcagaagaaaaatcaacaccaagattatgacatgcttgtgcaaagacacacccagtcatgaatgcatcatagccagcttcatgcttagccccagagttccaatttgaggayctgcaagaaaacatgggagtaagatggtttcacataaaacatg

SEQ ID NO: 55 - DNA, 201 bp, Beta vulgaris
agagcagattggcatacttrtgaatattctcactggctattaaattctcagaagaaaaatcaacaccaagattatgacatgcttgtgcaaagacacacccggtcatgaatgcatcatagccagcttcatgcttagccccagagttccaatttgaggayctgcaagaaaacatgggagtaagatggtttcacataaaacatg

SEQ ID NO: 56 - DNA, 201 bp, Beta vulgaris
gggtttcttcgaagtttgattttgttacatttttcaaagagaaattagttgttgatgttgaataatgatgataagtagttagggttcgtagtaaggtggacgaragagaaaatggcgtcactctgayragcttcttcattttgttcttcttccttagctctgttttcagtcactgcgccattttttttttaaaaggaagat

SEQ ID NO: 57 - DNA, 201 bp, Beta vulgaris
gggtttcttcgaagtttgattttgttacatttttcaaagagaaattagttgttgatgttgaataatgatgataagtagttagggttcgtagtaaggtggaggaragagaaaatggcgtcactctgayragcttcttcattttgttcttcttccttagctctgttttcagtcactgcgccattttttttttaaaaggaagat

SEQ ID NO: 58 - DNA, 134 bp, Beta vulgaris
caagcacaaaatcaaataatgagaatcacactatccaaagaaaatttccatccacatttatccaacacagttatctctcttttacacccaaattatgtcaaccaaaaacastaaaacaagtgagtgcagtagct

SEQ ID NO: 59 - DNA, 134 bp, Beta vulgaris
caagcacaaaatcaaataatgagaatcacactatccaaagaaaatttccatccacatttatccaacacaattatctctcttttacacccaaattatgtcaaccaaaaacastaaaacaagtgagtgcagtagct

SEQ ID NO: 60 - DNA, 134 bp, Beta vulgaris
taagtaaaaagtggtaaaagaattaccaaaarcgcacaraataaattaattagytggatwtaactatttaacctattccttttttctgtcgctataactacttttgcttaacttattgatggtttgatcgttga

SEQ ID NO: 61 - DNA, 134 bp, Beta vulgaris
taagtaaaaagtggtaaaagaattaccaaaarcgcacaraataaattaattagytggatwtaactaattaacctattccttttttctgtcgctataactacttttgcttaacttattgatggtttgatcgttga

SEQ ID NO: 62 - DNA, 150 bp, Beta vulgaris
ttataatgtagagtcaaaattaatatccttaactagtttttaagtccgggttatatcctagatatttataatattcatttattagtaacattttattttataaatataatactaagcattatttggtttgctggttaagactttagtgta

SEQ ID NO: 63 - DNA, 150 bp, Beta vulgaris
ttataatgtagagtcaaaattaatatccttaactagtttttaagtccgggttatatcctagatattaataatattcatttattagtaacattttattttataaatataatactaagcattatttggtttgctggttaagactttagtgta

SEQ ID NO: 64 - DNA, 201 bp, Beta vulgaris
acatctacactgggagactgataaggacgtttgcagatgtcaagtatgggaatcatcatctaacatgggtggagattgtgtacaatgttatttcattcatcgtggcaataattaccattgttgcgtttactgtatatgccaagagagccttcgaagaacttaagagggcagaagctaaggaggatcgagaagaagaaacct

SEQ ID NO: 65 - DNA, 201 bp, Beta vulgaris
acatctacactgggagactgataaggacgtttgcagatgtcaagtatgggaatcatcatctaacatgggtggagattgtgtacaatgttatttcattcatagtggcaataattaccattgttgcgtttactgtatatgccaagagagccttcgaagaacttaagagggcagaagctaaggaggatcgagaagaagaaacct

SEQ ID NO: 66 - DNA, 21 bp, Artificial Sequence
  Primer pCRBM4_S2
gtagttgaatggtgggaatcc

SEQ ID NO: 67 - DNA, 21 bp, Artificial Sequence
  Primer pCRBM4_S3
caatattgcccttactttatc

SEQ ID NO: 68 - DNA, 20 bp, Artificial Sequence
  Primer pSeq_LbCpf1_F4
accactcactcctcgataag

SEQ ID NO: 69 - DNA, 22 bp, Artificial Sequence
  SEQ 70 pSeq_LbCpf1_R3
tagacctgcttctcaaccttca

SEQ ID NO: 70 - DNA, 21 bp, Artificial Sequence
  Primer pSeq_Ribozyme_F
tgcagcggatccaaattactg

SEQ ID NO: 71 - DNA, 18 bp, Artificial Sequence
  Primer pSeq_Ribozyme_R
cctggtcccattcgccat

SEQ ID NO: 72 - DNA, 20 bp, Artificial Sequence
  Primer pSeq_tDT_F
ttacaagaagctgtccttcc

SEQ ID NO: 73 - DNA, 20 bp, Artificial Sequence
  Primer pSeq_tDT_R
gtactgttccacgatggtgt

SEQ ID NO: 74 - DNA, 19956 bp, Beta vulgaris
aaatgatacaggggtatatttgactctatgaatttcagaaatctaatcaaatttgctaagcttccaatgattctactaagccctacaaattacaagaattagttactttcatctctctgtcggcttcagaaccagaagtgtacaatatcttgtcaaacaaactctgcttagaggagctctttcgatcatcttttttcgatttggaagttcccggtgataggattgacattgctgttttctcggtcaattcttctggatcttggttctgtccatctatctctggctccattaatctggtcttccaattaattccgatagcctcagcttgctctgcaaacaagacctttgagatcggggagctgcagatatccttataaacttcataaccagcagcacaggttttcccaccttccaacaactttgataaaggatgtaggagagagatagaatcatcactcgtttctaacctatccttcaaggcaaggaagttaacagccaagtctgccttactaaactgaacaaatactgcagtttcatccaagttatagatgcaagcaactgagtatataccaaacactttacagatgcattgttttatgtcacttgccttgagttttggggagaatccccaaatcaaaactatgttaggatgcaaaatgttaagaaacctccttttagctgaactgataacgggaatttcattcatatcaccagtgcttagattgatcacatctccactgttccaactaagatagagcagattggcatacttgtgaatattctcactggctattaaattctcagaagaaaaatcaacaccaagattatgacatgcttgtgcaaagacacacccggtcatgaatgcatcatagccagcttcatgcttagccccagagttccaatttgaggacctgcaagaaaacatgggagtaagatggtttcacataaaacatgtgtagaagtgcagtgaacactggcgaaaacaatctaattttacgaattcattcactcactcagcttcaaattaagtttcccctttatttagggtgccccaaaaagatacactcttctgtttaccttctctctccaagcgaccaatcttttctctcttctccaacatcgttttctttttctctctctacccactatccattttgtcctcctacatttgataactattcttaatctccaagaaaatccaatgtgtgaaataattacgggacagggagtatacagaagcagcccccttgccaatatagtttacaaattaccctcagaattaggcttacctttcccaaaggagcaataaattcaaacaaatctaaaaggtacaaggcattaagtgccgaacctcatgtcatcaacctggacctccaccttcacacatggatgtacaccaccattagaggattgtccagaggctatctcagggcacaacagagaaaatgctgaggccaatgacgtgctggctttattcaagaatttttgaaggctcgtgtctgcattcaaaagtattttcgtgtcgacaacatgaggaaaatacttgtggatctcgagaacaaactcttcaacagttgatggaagaggaccaaagaatttatggtaaatatgtgccatatctgcaaaaaattataatggataagatgacaagaaaagatactaggaaggccttcaagtacaaatattatatcatgatgctggacgaccgatgctcccacaattatgtttgttaccaaatgcttcgaaggataattactaaattatgtgaatggtggttaccaagtgtcccggaccatgcaataacttctcctttcagtgaccaacaagaagaagacgtacctaaaaagcaattgtgacctacaattagcttcttttcagcagcgagaaggtcaaggacatgccggaaacctgcagctgcttttattttgcgagttgcttgctggtgagacccatacttcacctcctcctacaagaacaaacagaacaatcacacatgcagaaagttccccacataccaagttgctgtctgctaaacactgaaactaacttatctctacaaacaatgaaggaagttcctcaccagaaggttgatcttatcattgtcagattctacaaaaacaataagcttctgcaagatggcactgccgtcatgagcacacacaaaaacaagatccttgaagtgcttccttgtaacctgtaattgcagatcattagtatatattcaagatgttataaatttattgaaaagcagcgtctaaaacaataaaagtcatgcttaaggcatagagcgatagagcatagacacttcagagtttaataagagcaaatactccaggagaacataaatatatttcatatcacaaatcctagtaccaactggcaacggctaactgccaatttatgtactgctcaaaaaggccaagcatctaaaagatggcttaaaagtcggattttataagaaagtcgtcacatgattgctattacattgacatatcaaaggtcaaatgctgaaatttggttcagcttgatatatattaagcatacaaacgatacgttgacaagaaagcctaacaagacatgaagcatcaggcacataacattcaaaagattaccaattcaatcaacttcagctgatgagaagtaaatccattcaatcgaagagcaggacgcatactaaaaaatatggtttcaaattgttgtttagattcaacagcacttggaagctcggcatttctgttctgtagcaacatatcatgccaatcactgagccgaattttcatccgttcggagaataaaatatcagctacattgcccaaaggtaaatctctaacttcattagaatatgcccatttcccgttatatactgaattcaagcgactcaaagcctcgtcttcctgtcgtctagataaataagacacgcctgagattatacaatgtgtaaatttaccacaataatgacatcatactgacaaaatctcaaacaaatagttctaataaagtcatgttatctgaaatttctatgaataggaaattgaacaaaaccttcatgcttttttccaactaaaattgacatcttctacattaccttcatgtatgcatgcattgaagtcaaactggtattttgccaagaagtcaatcgaagttgtttggcacaggaattcatatgatgggccatcagtgggaagctcttgacgtggaaatatataaaaattatgcctgacaatgaaccattgtaaaattattagatggagtatctctatttattgtttacagccaattgagcttttaacaattactataggtagtgtttggaaacttgtatttcatttcaaataatggaattgaaatctggaatttaaagtttgtatttcaattcctaatcactgttttgtaaagggggtttgatagaagagagagaaatagaggtttaatggaggagagagaaaaagtgtgggtttactaaaaaaaagagaaataaatattagaaagtgtgggtttactcatagagttgggatatgtatgaggagagaattttcaaatgccaaggtaatagcttgaatgacaaatttaataatttcaaattccatgtcatccaaacaatagatttcatccaaatccaagatttgaaatgaaatcttgctatccaaacatatcataaattaattagtaatttagacttgctttctgctgcacttacttatggaaataattttacttcagtccttaaataacccgcaatttacatcaaaggcactaatataaacacctagttacgaaatggaaatatcagatatacctgtaaaagtaaagaaacaaaaatacaaccctgagcatgaaggtatccttcaaaagtgcaatatctgcatacttagaaccggaattagaagtgcgaatgcagacaataaccatcccaggatcagaaacgtccaagaaagttgagattcatcatccattctctttagcaaatttatgaactctaatatataaatcatacccccccccccatccaaaagcaattgtcaagctgcctgaacccctcataatttaggatacaacaaagtaatcctaaaagaccctttacaatactagtactcgggtatttccacaatcttctcatcattgaatccaaagcattgcatttgaagaaatcaaatcataatccattactatattagagcaaaatctatgtcattatagtattggagagcaagtatgactattacccctttacactaggcaaaacacattgtcacaatgctaacttagtcattaaccaatatcaatatgggactgtggatattcataaaatcgaagtttttcgcttgctcataaactatctttcattccagcacagtacaagagagaaaagacagcattttcatacacttctttctttagttcaaattcacacagcagcaaaaaattcacttcttcatagctttagctcagcaaacaaagcacaaagcatgcaattactctcacacatagcacaccaaaaaaacaaaaaccactaaaaattcacacaaaaaaaaccaacaaaaattccatcgcaatttcaacaatcaaaacaatcttctaagttaaaaagagagataaagatgagaagaaaaactaacggatgagcaacgaaggaattcttcgaagaatcccatcgaaatggacaaacaccaaattgaacaacggcgaatttctcagcagaatctttcattttaaggtatcgaacatcgtgccgatcaaactcgaacgattcgcgccaaggtgagcttgtaattccagtcatttcgagatcaatggcgacaaaatcggcagattttacatgcgtagtgaggtcaattagggtttcttcgaagtttgattttgttacatttttcaaagagaaattagttgttgatgttgaataatgatgataagtagttagggttcgtagtaaggtggaggaaagagaaaatggcgtcactctgacaagcttcttcattttgttcttcttccttagctctgttttcagtcactgcgccatttttttaaaaaaaaggaagatgaacaaagcaaatattgaacccaaattttgtaattttggcccactttatatgtacccctccgtttcaaaatatggagcacgccgcacacacgacatttagggtcgaattttgaacattcttcaagatgatctaatggtataatctctataatttatatgtggcatattataataagagttttatgaagtcaaaaagtggatgtcatatatttaatgcatggtaagtttttcctaaatctgtatactagggtaacatacatatgttgacttgaagtatatataattcttgtagtataaatatggctttggccataagtagtaatacacaacaactagaaaaattgaaatcagtccactgttatcttgtactctataattttctgtttccttttgtttcgcaacaaagacatatttgtggtgaaagataattttcgtaaattgaatgacttatattttgaaataaagagagtattaggtaaggttacgtgcttttcgcttgaatttgttagacctcaaatgtatatgtgattagaacggattggctctagtttttattttatagaagtatatatgcatttttcttagagcacactcgaaattactttcggatagatatattcgggaaaaaaagaggttgaagggaagttcatcaataattatggtaaaggaaaaaggacatcgttacaattctaaattctagataggatgtgatgataatccaaaagtcatctgaaaaactaaacaagtccaagatgctaatgattcgagtagagattgaatgagtgaccctaaggattgtcaaccctcttattctaacgtgtgtaaaagaattgacaactctaagagttactcaaacatttttcgattcgagtggttaatataccaatttgaaactattgacaggagttattttaatgagtataatggtcaatggagcactgaattccatctcacatagtcacatatttcatctcaagttctgatgatttcaaacattgaaaaaagatgatacaagcaattaattcctagggaaacatattgtggttttcatggatacaagagtgagaataaatcaaaacttaggctctaacatttcttttctctactagtaattgctaattatatcaattcaattgtcagtgtaatcagttaatcaccaaatctcttgtatagtcagtaaactatacactgtttagtcctctggattttgcccggtcgaattatgcagcataaccaaactttgaagtttagtacttcctttgcacccaagttagcttcacggcccctgccttctggtggatggtcaccctatgctttgagcattctctgcaatgcgcacgatattcaatgagaacgtcgccttgaaaatctaaattgcaactaaaaattagattgaaatgaaacccacaagagttgtttttctgagtagttggtgtagaattcacaagtcttgctccattgtttgaagatatgaagacaataatgtgctatgtaaagtgcagccgctagctaacagtggaagtggaaacttgatcattttacactcgcacaagcgaaagctcggctgacgttgcaaactgaagaaaaacctctcaaaccaattcgacttttgctcaaagttgcaaactaaagaaaaaggctgaatgcaaagcaagttcaccaatgaacaatagatcggtgttggcctgaggccacatcaagtgaagttgcctaattgcggccctctcatctgttcacaggaatcattttccatatagaatcactccaaaataaaagagcaaagctgcaccagatgcagaagcataactttcaagacaactgatgacagataaatagcaaaagaatgcttaagaaatgatcaaaattgaatggctctggaattacctcatcagctgattttcctttctctctatctctctatctctttactcgtctatggagctaccacatcacatggcgtttcatatgctttctgccgtcgaactagacgtgcagcaaaagctccatccattgaatgcttcactgggcatgagcgataaaacccatcttcagttaaaaagtcagatggaacatatctgcttacagaatctctttggaagtcctatcacccaacaaagaatatattaaaatagagaaggagaaaagaacgtatctatctgtcagcatccatatgaggtggaaactaggagtactatataaagccagtgcagtagctcctaccggatgtctaagaaggaaggcagaaaccctatcttcgttttcttcaagatcaatggagcaggtactgtacacaagcacgccatctggtttgaccagcctgtagatgttaaacaatcccacagacaaaagggaataatatgagtgaaacaagtcaacagggggaaataaccaataattctaggactgtcaaactcaagctcttcaaaaacaaagatagctcttaatctcacttgcaagcagcatcgaacagctcgtcctgcaacttctttagctcttccatatcctctgactttctattccaacgcaaatccggcctcttcagcaaaaatataaagttggaacaaggctcttagatacaagaactgaaaaaccttcgacatataatggactctatcaagggcacaatgacaaattctaaacatgagcatgtatatcaataaaatactaagaaccctttcaatggtactgctagaaggtttattgctacactttttagtacaccatctataggttttatagtaccatcaaaatggttcatggtgccataagaaaattttatgtatttatggtactatctaccatatctaattttctctgtaaaaatgtatttgtagatagagaccacgagttcctcttttagatactgacttttttttttctacatgatggccaacagacttctcaaacaaaaagaaaaagaaaatatttagataatatgagcaacaaaatagcaaccacctacttttgatagtacacccaggcccgaacaaggaacatctaaaagaactttatcaaacttcgaagtgttgctgtcctgaaaagaatagaaagtaactgcttcaacaaagaagaagaggcagaaagcaaagctagtacgcattttgcaatgacttactgaaaaggagcgaagatcagcatggatgcaagtgatcacattatcaacacgctgcagcttggctgtttcttcaagtatccgtaaccgacctttatttatgtccattgctgatatcatacctgaaaagctacacatttagaatgcagaaccagcatcattggtagttaagttatcactataccttggccattcaagcgagatgccatgaagagtgtcttccctccaggagcagcacagcaatcaatgatgtgatcaccaggctgtggatccagaacagaaacagctagacctgcagcgaagtatagatgtaaacttgggttgggctgtcacattttttcacatcttatcttcctttctattctttcaaaactgaggagaaatggttgggatttctataaacgtgagaaaaatggcatcagattagatggttttactgcatgaaaaaaattgaatgtgtttcggcatcacattactacaaggtcaaaagcactatctttgaaaatgtaggacataatgggacagagatgtgctgacctgcactctcatcctggactgagcataaaccttcttttagaagtccagtttgtatcacaatctaggatatgagaaaacaactcaagatgtaattgctcctaagatatcaatcatttcataataaacataaaagttattattacaagacagcacctgcatcccacttctgatgcagacaaagtcatccaaatgcaaggaaggctcatgcgggacctgcggacaaagtgttgtgatgcgcatagatatcgaaagaaggccctgtatagcactaatgagataagattcagtaaccttcagcatgttgagcttcacaacaaggtcatctcgagttaatccttttgcaatattggccctagtaccaagaaaaccatatgtattaacaagagaaaagtggcatagggatctttatgacttaggtaagcagttggcaattagagagaataaaacccccaaacctcaagctgaaactcggaacactattgttccacatcatcaatttgatagctccttcttgcccaagatacttggtccaccgtcttaccatccactgaaaggaaggtattaaaagggagaaaagactcgtcagcatagaaaattgtacatcttaaattttagaagtatagcaccatcttcaggcatcagtcaacgtaaataaataccacatctacaaatagaaccatactttctggacagtcgggatcatgagcacagacaatactgcatgattattgcctcgtattctcatgtatacaagtatatgtaacattaaatagcagtatttcttgagaaactcaccacgggatgggaataagttgtagcaagggcacgtgcttgtgaacgatcatcaccctccaatttgggtacaggaagggagtcattatcctataaagagaaacagcttttgttttcaaccatatcaagacaaacagtttattaaactataaacaacaacaatacacatgcacacacctactgggaacaagatatatactactgataagtattttctgattgaagaaaaaaaatctcatttatttgcaaatatagatttaatgacaagaaagctttgaaccttaaggaaaactagctttcggaggatcccattcaccatgtttcctgcgcctggtctaagagcatacttggcaagattcacattctgcaatataatccaacagtaagaacacgacatggatttagactcaagtctctgaacctatagaacaagtaaaattagatcttatctcatttgacaatttaaaattagatagtgcaatattctgcagttataagacttcatgtgtgcatactgcacaagtcatcttaaaggtgttattaaagctttaattgccatttgacatccccttgctcaactttagcatgtttttaggctacaacaatacgcactgtctacatggacatacaaattacaagcgtatggaaaagcaataagcgcaaggaagtcttcagccagaaactctctatgagtccaacaatatgcaactaaatatccaagtaccgtgaatgagtaagaactaacctcgtcaacaacagcatatggtggcatttccagtttcacaatctcatagcatccaatcctgaggatctaaaattaaagataaatcaatacacaacatatgatatgggtcggagcgtatataacaagtatagcaactacatttgaacagataacagcctttgagacaataaggaactccgacattccagtatatgccagatttcatatctttagctctaaattgccacgcaaaatgttattgggcaatatacctgtagcaggagaggttccatgttcctaaaggagctttcatcatggcatgaagaaacaataagataatccagatattttctccaacgaattgaaccaccaacaatgtcagtgacctacaaagacaagttgtcaacttaaaacttttgaagcgtcatttcacttctgtagaccaatacaaaagctactactgctttacatcataaaacctttagtccttaggttcatctgattggcaaaaaaggtccagatgcaagaaaagcaagtagctgtaatgctgtattatatcagcattattcagaacagaataataaatatctacagattttgggtggaagcttgatgatagagtatctccacaaagagaactcgcttgagtcccaactcccaaatctacttttttggagtcacattatcagtcattttttctggactcttataggaatagtgtgctatgtaatgatttatggagcaggggcatttcatgaatagctttataagttagtatgggtgtcttggggaataagttaaagggttagttagagggaagaagtacaacatatatatagagcttttgtaagaagggtggttatgttgaaaatagatgagaaattgggtgagctcatagtagttcaatttggactttgggagagaattaagcctcttgaaagcttgaatatcatttacatttgttgtttttactcttattaatcaaccaaagttcattttcttcctttaatttctccattttagcactatgatttgtccaagctaagtgatttcttagcatagtgcacagtgtagtatatcggagaactcatttgagtcctgaaaggtcccacaagttacatttttcctactactacttgcaccaaaacaataagcatcattaagacattgtcactggtccttcttaggttcttttggaggggattcctcagatgggggaggcacccatgaaggaacatgttaccaagcaatgggacaatgcaaaatgcaccaatacagtagcttcacttcattgattgcatctatgtcacggaaaactgaagaaagaagcaacacctcaactttatccaggacagatatccactaacctaggatgcaagcttgagactatttagcaattgcctctgggatattaaatcagattacgactatatttctacagttattgcttaagaaaaaggtacgatttgaagcttgggaagaaagagaacaagagtaaaagaccaatctgagatctctttcatccaggtctctggtgcgaaatccaagagtcctctcaacatattccatctcattgttccctgaaccctttcctctctcatttagaagatcagcgaaggcaccaccaaactctatccgcatcaatctcacagcagccactggatttacacatgaaagcaaaccaggagaaccataaaaatcacaacaaacttcctgatagcctactcactagcatcaaccattgtgttcagcctaaaatgagcggctgttttcaattgaacagcaacttacatggaccactgcataaaagtgatttcttaatccagacaaacaaaaatgtttacttcaaccaactgaatttgcatcagctcattagtgatttgacaagttctaatttatgtatcaacaaacaagaccatatagctaggaaacaagaggcttaggctaagcttaatgcgtgaacaatgttagatttcaacctatcagcactgtggataactgcaaactgcgacttaaataaggaagataaaggaactgaatatgcaatttcaaggtgctcagcatttgaatcaacagttacttcagataattcagaacataaaagatttgaacattctaaggctacctcatgattgcaagcaatgttacctgattcgctaaccctcacaagccacaagccaaagaagcaatttggtaaatggttcatggtacaactgttcgcttttggactaatctaacaatactaggtggtaaattatgttcccatatctattaccataatgtacagcaaattaggcagcactaattccaaatgacccaacaaaaaaagaggaagaaaatccaaaaattcaagccaacatatgcactaaaattacaagcacaaaatcaaataatgagaatcacactatccaaagaaaatttccatccacatttatccaacacaattatctctcttttacacccaaattatgtcaaccaaaaacactaaaacaagtgagtgcagtagcttcacatcaaagaatatcaatcacaaacaccacataataaaatttcaactcctgcccaaacaaaaaaaatataaagaaaaaaaaacagcaaaatttcaaagataaaatagaaaaaaaaaaatcaaaatacagggggaaaaaaagtaaatttaccagctctatgaggcgaaacctgcaaattcagcttctgggttttctctgaaatatcaagcacaataaccagcaattaaaaaaaattataaataaaattaaaaagaaaagattgataattaaaatcaaaagagagcaatttaaagcacaatcctttttttaccattttttctgggaggaagagcatccttcgttttgggtttagacgaaaaaaatgagagttgttgtatttgtgcgcatgagtgatcattgctggaaatgaaagtgggaaagtggtaaatgagtgctttgtgaaattgggttttgaggaaaagtagaaagaagaagaagggtcgatgtcagagaagagagagagtggatggaaagtagtgatgattgcctccattgttgccggtgaagtgagctttctgcaaatatttcactggactagttttttttagcagataacgctaaaacagagaaagatgttcggttaattttaatttttggacatttaaatgactattcaatatgtttcaaccttttttttttaaaacaaaggaacaatactagtattagattacgttaatgtttagtacatccaatacttatgtgtgtttgacctaacttaaaatcgtaagttgtttaaaatgtcggtgtcttgtttttaagagatatcatacttactatctttggtttttactcttccattgttaacagaaactgtatttatttgggtaaggggtttgagtgaattcctgtaagtatgagaaagttttgagtgaagcaagagaaagagagaagaaaggaacttcgagtgaagattgagagaaacaacagttagtgggaactgttgttgggaacttgagtttaggagctcaggttgtaccccgagagaattaataggtttgtaacagagtcggtggcctattatagtggaaagtttgagtcaaaatccattgtggccgatgtcgtttcttcttattgggcctaggaagtttttcctcgctaaaatttcctgtgttcccattgtgtgttccttagctagctttcaattccgcaaaaagttacgtttattctctcactataattcacccccctcttatagtgctcatattatacaacaattgatatcaaagcaggaactctaaaaatacagaaatcatgttgagttcaagatcttggaaaatatgaatactacagaaaaactggaagaaaggtactctactcagagaccaccgatgttcaatggcaaattctacacaaactggaagaactgaatgaagatcttcatcaaagccgacaaatatcaggtttgtagaatcatagaggcaggcgattttgaagtcactaccactaatgacacatatgaggtaattcctaaattcataactcatttcgataaagtatatttcgaaaagttggaaattaacgttcttgctattaaactgcttcattgtggtcttagacctcatgaacacaatcatgtcatgggatgcaaaatcgcaaaacaaatttgggatcttcttgaagtcactcatgaaggtacgggtaaagttaagagatcaaaaatcgatcttttaatgaatcaatatgaactttttcaaatgaaatataaggagtccactcaagagatgtttacacgctttactaatactattaatgagctaacctctcttggaaaagaaattacatatgatgaacaggtaagaaaggtcccaaggatcgttggatggctaaggttacgccttacaaaaaactaaggactttacgaagttcaatccggaacaacttactggctcccttatgactcacgagctacacttggacactgagaatggtgacttgtccaaacagaagtcgattgccttgaaagccatttttgtcataccgtcaattaattaagtaaaaagtggtaaaagaattaccaaaaacgcacaaaataaattaattagttggatataactaattaacctattccttttttctgtcgctataactacttttgcttaacttattgatggtttgatcgttgaatccaagttttctccacccacaaagatattatagactttactttaaaaggtacgataaataatgtttaatcaggtatgcatcaaccttgaaattattaatttattaagatcaaattatgcatatttatattaaacgtacaggacttgtgcacaatccatggatgatattgtagattttgttgtaaaggagttagggacaaatgatgttgaattaagaatgatgaggaacaacattgaggtacctaatggcatacaagattatgtggtaacaaaggtgaagaagttggttgtaccaggcaatacagcagcggcaagccatatataggatgagctaccatacccttatgttgtgaactattgtcaccaccaacaagacattggtcattacgacatcactttagttgaggaatgataaacctctttttgctagatatttgcaaacatctagcagataaagaggaataaaacactatttatatttcatgaacactatttgttagttgcatgaacactatttttagttacacgaacactagttttagtagcatcatgaacactattttttagcatcggaattttcacgactactttttggtttgactgacactctgcaattttcgagataactttttggtgatatgggtcccatgaaatagaagatttatatttcatgaacactatttgttagttgcatgaacaatatttttagttacacgaacactagttttagtagcatgaacactattttttagcatcggaatctttgcgactactttttggtttgactgacactttgcaattttcgagataacttttttgtttgactgacaactatttcctatatatattgacagttttacccctgttagatgtttgcaaacatctagcaaaaagaggtttatcattcctccactttagttagcccaacctccagtaacgccatccagaccactgtcgtttgtcactacgacacttacgcttggcaaccctatgtcctagcccttcgatacctcgatatccgtccgggcaatgtccccagtttgtcacttctctgccattaatgacatattttggagtatcaaacccaactccaagtatatatcgcaacatggctcagtaaagagagtcatataatcatgacgtagtttctatatgccatcctacgtagtatcttgtaacatgaataacagcctggtttgcaggttgatggtacatggtataaattggtattactccctccggtctttattagtttaatcctttcttttgtacagagttataggagaaataatattgtgggtcatagaaggaaagagaaattattattttatgttaaagttgaatgtatgtgtgatgaaaagttagtagtcccatttcaaaatagaaaaaaaaaaggtaaactaataagggacatcccaaaaaggaatacgggtaaactaataaatatccatgcaggttgttggtacatggtacatgaagccgtccaaaaccttcaaaagcagtaagtcctgctgctatgccatattcaaatattcaactccaaaaaaaaaaaaaaaaaaaatcaaaaatccgcttttcagcgaaaatataggaaataatccaagaatcgaaatcgaaataaagtcatgatgcaagtttggagagctgaagttacactatatcggagtacttactcaaatgttgattagtactccgtgcgtttgaagtaaagtcacatatggagtagttccaagctaggttgtacagtgacggataaggatactgggttgaaaaggtgaacgtcgagatttatacgtgtatttatttaaacaggatacgtatcatattgggttctcatacgcgtaccagctgtgacttagaaaaattaaccacgctatataggttccaagccctcatgattaccttttcatagtgtaaatttcatgtagttgaatggtgggaatccaatcacaaaaacactgcaggtaatggaaatgttccaactttttccaagcattttaaaataagacatgtgattactaattagggcgtgttcggcaacagtaattgtggtgatagtttttagctgtgagagtagttgttagctgtgctattagcttttagtggttggtgtgtagctgttagctgttagatgtccaagtagcggtgtaaaatattgatgttcggtaaaagaagctgtcaaagtagctgtctaagaataactagttaaaaattcaaataaaactttaacatataatttatacaccactaaaagctacccaaaagctacaaattgtagcttttgacaaacactactaaaacactacttgtaccactaaaagctacttacaccactatcttgccaaacactcttattttttctaattagtgttttgacctagtcaagacactaaaagctacttaaaaagcttgtgccgaacatgccaattctgaaccaaggaacaaactataacaaaaaagtgctatgtgaaacttttgtaggcaacagaagtaaggcatttttggaatgtactaacaaatccgtattaagacttgtacatgaaaattaccgtggtaacatttacccacacttcctcattcacgtactccgattcattcttataagggcataaccgcataaggcacatcaagatccatgtatctaatagtttaatttgcctctgtgtttctgtattaacaatgagcatagtgagtgcaaaagccatggaagctagattaaaaaggccatcattctaagttagacaattggaaacaacatcgagatacacgtacacataagggctgctcttctctattactccctctgttcctaatcatttgcttttttagcgggttccaaaggcctatgtttgaccactaatatatttaaattaaaactggtgatatatattaaaagaaaattatgatgaatttaacaaaaaccatatatgttatgtccttttttttcctatattaatgaatttttacagtcaaagttggtgaactttgacccaaaaaaagaaatggagcaaaaaaaaaaaaaaaaaaaaaaaactagggacaatgagtaacatttttatctatgtctttttaatatgaatatacgtaacaaattctgcaaaaatagagatagcaactaataacacgcatgaaaatgacaagttatattatacctttttttctcaatatatgaatatacgtaacaaattaactccagtagtttttagtaaaactattagattattgtgtaacatatactctggaaatagtactaagatccattacaatctttattgagaaatttcctcatgtaccccctgaggtttggcgtaatttccaaatacccctcatatttgaggaatttctcaaataccctgatgtttttgtttagactcaaaatacctttactatggacagtaccctaatgtcattaagttttccccttctctctccccaattttctctctcctcccattcccccacccactacccactgcccactgccaagtaggggtgtaagtggattggactggattggactttgccaaattcaaatccagtccaaagttttttggactcgagaaattgagtccaagtccgatccaaatattttttgagtccagtccaatctagtccgataattttttcttgagtccgaatccagtccagtccagtccgattattatatcttttttcccgatttaggttcaatgattcacaacattttttgagatgcttgagcatttgacatctgattcaattatcaatatccacaaataagattgaaagcttaaattaaagtaaaatactatgaataaaaagttgaattagatgcttaccttgatctaagttgagaggaagcatagagactgagaattaatctgagggacaaatagagaatgcgagagtcgagacagtgaggtagaaagaaaatgaagagtaagaggaagtgagtattaaggactgaggagtaaagtaagatagaattagttggctactagcctactaatgcagtattgctagtataatttacttatttaacaaatggagctaagtgcaatagtttagcgccaattgacatatttagagagagaaggctgaaaaatccaatatttttaaaatagtatcattatttttaatatatacattatatataaaaatatttttggactggactggacatattggactccaaagggatgagtccaaatccagacaaaaaatatttggacttgaaaatttaagtccgagtccagtccgaaaaattttcagtccaatccagtccgacaaatttggactggactggattggactctgaacttttcgtagtccgcttacacccctactgccaagtgccaaactgccaacccccttttggttgagttgatatttgacgcaaagacttggcgtgttggaaggttcattacacattttatccaagtcaactttgaagtcttcttagctagagactagagtgaacgtgttggaaggttcattacacattttatccaatcaaactttgaagtcttcttagctagagactagagtgaacgtgttggaaggttcatgttcatgacattataaaagtaataatagtgaaatttcacaaagtatttataaacccaggacagactcaagagctctacttattattagtgaaaaacaaacatacacacgacaataacacaacataaacaataatgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatccca

SEQ ID NO: 75 - DNA, 19206 bp, Beta vulgaris
ccaacaatttgttagccgatgaagagcatcaaaaccaaaaaaaacaaaaaaaattgattaatatgcatgagtgtgaccttgttttccaaagtttagcattactattagtgtctcaattcataataataaaaaaattagcttgttcaagatttgtatttttattcaaagattttttttgtctcttgtgcttcttttatcttatatatattttttgtatggtttgtttttgtttaatattagtccctccgctcaaaatgatctttcacgcttgagattggcattaaggtcaagagatgttgctaagctttagaataaaaaaattccaaatgcatagagggaaagaaagcgagacaaaatgttggagaaggcagagtaaatgatgtgatggaggataaatagtagaagtgtgataccgaaagtttgaaaataataaggaattttatttcttgctggcactttgttctagtacaggtttttagcccttcaaaatgtttataatgtagagtcaaaattaatatccttaactagtttttaagtccgggttatatcctagatattaataatattcatttattagtaacattttattttataaatataatactaagcattatttggtttgctggttaagactttagtgtatatctatttctttttttttttattgtatgcgtgtttacataaactaaagactataagggatagtaccacgtggcgcagttccttgcttaggaacgtcttttaatatattaactagtatttgggcccgggcgttgctccgggttggtattgtgtttccgaacatgatgtgcagtttttcccattcccactaaaatatataaaggaaaactcaacatttaaaagatacaaatataataatatggacacttaaaacatgattaaaagttgattgagatggtaattgtgtcatgttataatagtaagaggttgcctaattgaggttgaggtggtggagtagtggtatcgcttcccatctgttatccctgaggtataaggatcaaacctcataggactcatttgagtaatttcccatatcctcctctcaaatgagtccttttcatctgacaaaaaaaaagagtctaattttaaattaaaattagacgatcttttataaaatcggcactttctgcacataggtcacaatttttttgtttctatctctctgctttctttaatttcacagtctccaactctccatcaacatcttacttattttagaatagatgatgtatggtagtattaaatggtaaagtactaaagctcctataatacacagaagcttacatagtatagattcgtacatgagacaaggttacaatatactttctccgttctttttatattacaataattactattttaagtagtttcacatctattgtaacaattccaattttgttatagaaagcaactttaataattgacaatattgcccttactttatcttattaaaaccatcattaattactcactttctcttataaaattgcttttattttctaaggatgatttctctcctattctagttaattaaagagttacttttgtgctaaactgctcatttattccaaatccttaaaaattgtgtccaaacgtattgttgtaatataaaaagaacagaggtactattagtttgaataaattttgatcagattaggtcacctttagggggcgtttggttaggggtattctggaaagggtaagggaatcaacttacttaattcccttacttgttgtttgtttgctcaatttaatgattccctttacccaccccttactcccaaagtcctttactctcattctccccaccccccaaggtttcacttaccctttcttgattcatcattgaccatatctttgaccacccaactaccaccaccacttgaccacctaatcacctaaccacctaattacccaaccactattaccacccaacccctccacctgcccaccaatcggcaccataactgcccaaccgtcgcccaatcaagccacccaaccggcaccataaccgcccaaccaagccacccaaccggcaccagaaattgtaccaagctacccacacacgtgaaaaccacccacccacaagccctagaaaaaatggaagaatcgagagaaagggaggggagagaaaagatgcagcgactagaaggggagggggaggatgtgacggcaaggggagagggaacttcgcagcggcaaagggaggggaaacgtcgcgtcggcaaagggctaaggtggaattgacggggttgcagcaacaaggggagggcatggagacgtcgtaaccgcaaggggaggggcagcggcagtggaactggggtggagaggggtagtggcggcactagggtgtgggagaggtggcgggggatatcaagagaggggggatatggtggtgttatggtggaagcaagaagaagaaagaggaaagacaatgtactaaccaaacaacacattaaatctaagggttttggtttcctttccccatctacccctttcttgattccattccctttacccctttacaaccaaactcccccttagtttttactacttataaccttcaattttggctgttttttgtgacattttttacttctccgagcctggtcatattttctcccgaaacatttcgaggaaagtcgaagtgacttgtgaagttgtgcgggtgcttggcaccatttgtgttgcctcgaaaagcatctgaataccccatttattcctttctcctgaaacccaaaattacctcgcaataaacgaaaagatatccatatatttgttccaagccacatgactcctttccaacgacctcccatgtgaccatgtccttagaaggcatcccgtggcgttcgaagctcggacccccggaaagtccgaaagtgtgtattataactttcaattttggctgtttttgggatattttttacttcttcgggccttgtcatattttctctcgaaacattcataggattgtcaatgtgacttgtaagttgtaacgttgcacgggtgcttggcacaatttgcattgcctcgaaaagcctctgaacaccccatttgttcatttctcgtgaaatccaaaattgcctcgaaaaaaacgtaaaggcatccacatattcgttccaagccacataactcatttccaatgacctcccatagagtccgtagctcggaccccaggaaagtccaaaaacgtgtactataaccttcaattttggctgtttttgggacatgtttggacttcaccggcctggtcatattatcttccgaagcattcctacaaaatccgacgagactagtaacgttgttacgcgggtgcttgacaccatatgtgttgccttagaaagcctttaaacaccccatttgttcatttttcgtgaaacccaaaattgtcccgaaatgaacataaatgcatccatgtattcgttgcaagccacatgatttctttccaatgacctcccatatccttaggaggcatgcatcatgtggcgttcggcgagcgggtctcgggaaagtccgaaagcctgtgttataaccttcaattttggctatttttgggacatttttggcctttttcaagcgtgttcatattttctcccgaagcattcctaggttaggcgatgtgacttgtaaagcgtgggtacttggcaccattttctttgcctcgaaaagtctttgagcaccacatttgttcatttctcgtgaaattcaaaattgcctcgaaatgaacgtaaagacattcacatattcattccaagccacacatgactcctttccaatgacctcccaagcccctaggagtcgtcccgtggcgttcggatccggagctcgggcccccgagaatgtccgaaaccgtgtattatgaccttcaatttttgctgtttttggaacattttttgacttctctgggctggtcatattttctcccgaaacatttgtaggactaccgacgtgacttgtaatgttgcgtgggtgcttggcacaatttgcattgcctcgaaaaacctttaaacaccgcatttgttcatttctcgtgacacccaaaactgcctcgaaatgaacgtaaaggcatccatatattcgtttcatgccacatgactcctttccactgacctcccatgtccctagaaagcaccccatatccgaaagcttgtattataaccttcaattttggctgtttttgggacacttggactttttcggttcgttcatattttctctcgaaatgttcctagaaaaggtgacgtgagttgtaacgttgcgcgggtacatggaaccatttgccttgcctcgaaaaacctctgaacaccgcatttgttcatttctcgtgaaactcataattacctcaaaatgaacgtaaatgcatccatatattttttccaagccacttgactcttatccaatgacattctatgtccttagaaggcactgcttgtcgtccataattcgggccagggaaatgtatgaaagtgtgtattataaccttcaattttggctgtttttgagacaattttttacttctccgggactggtcatattttctcccgaaaaaatacttcgagtgccgacgtgacttgtaacgtcgcgcggatgcttgacaccatttgtgttacctcgaaaagcctttgaacaccacatttgttcatttctcgtgaaacccaaaattgcctcgaaatgaacgtaaaggcatccacatatttgttccaagccacatgactcatttccaattctctcccatgtccctaggaggcatcccgtggcgttcggagctcggaccctgggaaagtccgaaagcgtgtattataaccttcaattttggctgtttttgggtcattttttgacgtctcttggcttggtcatattttgtgccgaaacattcccaggattgccgacttgacttgtaacattgctcgagtgcttggcacaatttgcattgcctcaaaaagactctaaacaccccatttgttcatttctcgggaaacccaaaattacctcgaaatgaacgtaaaggcatccacatattcgttccatgccacatgactcttttccaatgacctcccatgtccctaggaggcatcccatggcattcggagctcgaacactgggaaagtccgaaagcgtgtattgtaaccttcaattttggttgtttgtgggacatttttgggcttctccgggcctggccatattttctcccgaaacgttccttggaaagccgaagtgagttgtaacattgcacgggtgtttggcaccattagtgttgcctcgaaaagcctttaaccaacccatttgttcatttctcgtgaaacctaaaactgcctcgaaatgaacgtaaatgcatccacatattcgttccaagccacatgactcctttccaatgaccttccaggcccctaggagtcatcttgtggcgtttggagctcagtccccggtaaagtctgaaagcgtgtattataaccttcaattttggttgtttttaagacattatttgacttctccgggactgggcatattatctcccgaaacattactaggagtgccgacgtgacttgtaacgccgcgtgggtgcttggcgcaattgtgttgcctcgaaaagccattgaacacccccatttgttcatttctcgagaaacccaaaattgcctcgaaatgaatgtaaaggcatcgacatattcattccaagccacatggctcatttccaatgacctcccatatccctaggtgtacaccccatttgtctgatgttataatagcaagaggtcacgggttcaaatcttgttacaagctaattttacttttgttaattgacatgacttatgtacacattggacaattatagtggagtaacaaaggtgacatgtgacgcgtatacattatcacacacgtcttttaatatatttgtatagatctagatttaagagtaatttttttaatgcgcaatacttggccaatttcttctgtatcaaatcataggtctttggttggttcataagagtaaagaccaaaataataatctgaactgcaaaaattttctccaagagttaaaagtttgtataagttagattaaaaaaattaatgacatatgatgtagttggacattaaatatgtaagtttagaagtaattgtgttaacataaaaaaagattcgattataacataaaaactaaagaaacacaaaggcgccgtacaacaatcaatattacccaagtcccctcattaatattaagggatgacctagctcgtacatatttaattatctttgaaaattcgttgttcagacttgctagttgctattctatatttgtatattcattaatcaatttttcaatatgtgagcatttacattttaaactagagcaaatattgtctcttttactattttgttgttgtcaaattttcaaaaataaattgctcaaatacttttcctagtgacataaaaaatagagcaaataatcaaacagtagcagacccaggaacttttacataatgtagacggcataatgtgttaatttttgcttcttttttctaatatcatccaataacacaattctgcttctattagtttgtagtttcagatgatgatacccaaacaataagaccaagcaacaaattgataagattttgcttctctttcttccacttggtgtaactgtaacagctttgaagtttaacttcagtaatcagttgcatatttggcatatgatcaaaacaatcaaattattatgtatggaaaagcaaaaaacttccaggtttccatctgaacaaggaggccaagagggtggaagcaagcaaggatatatgatcataaaatcctatgaatatgatgtacaaaccttttctactgcaattaggtaacctaaatgataccacctaggaacagcaacaacttatttacagcactaaacctaaatcaggttaaagttaatcagaccaccatgtatctgggtggtctctcgagggaaagcgtctccatctgtatccgggtaacagaggtttcttcttctcgatcctccttggcttctgccctcttaagttcttcgaaggctctcttggcatatacagtaaacgcaacaatggtaattattgccactatgaatgaaataacattgtacacaatctccacccatgttagatgatgattcccatacttgacatctgcgaacgtccttatcagtctcccactgcaaatgaatgctatcagcgtcaatattcgagataccaactcatttaactattgaattgccaaaaacagatatctttgaccatatatttgttactaaaaataacgattgataatgtgaaactatcactgatagatttaaaagaacttttataaaagtatagtttctctaatgtataactgcagaaaatagaatggggtagacaaatgaagtaattgttttgaagaatgcaaaaggtcaattcagtaatacttttatacgtgattgggggaagcattaaaaatcccttctaagataaagatgacctcattggcaatggaatcgacatccacagacccttgcattagaacagagtggaagtttctgtgaacttacgtgtagatgtaaagaaaagcttctggcaccatccctgcaattgatccccatagataaggccaaaacgtcatacttgtcaccacaactgcgtagttgaagatagtatagggaaatggtgaaaccctaaagagtgccaccacgcggaactgatgaaaccagctaccttcggcagcaagcctaagcatagcagccttatccggccatctttgcaaccattgctaacaaggtacaaaaacataaacattgtggacttaattagacaagaaagttaaattaaaatcaacattagataatcaataaatcaaatgtaagcagggaacatatttcttacatggattctatcccggaagagcaatccaagtaaatagggaagaatcattccaatagtagttccaaccatgattatcacaaaaccaagaccataaccaaagatcatgcctgcaagccacatggatgggccagaaggaatcagaaatacagggaagattgctagggaagtaacaaggaccacagcaagaaccggacggccaaaggcagtggcttcccattgcatcattggaacaagaacctgcagagaaagtaccaaaaactttgaggcaaaaatttcctgcttgtatattgcaaaaagtagtacagcgaaggcattccgtgcagaatggcttatagattggaaatacggagaacaatgcaactataagcacaggcccatctcttgacttttgggacaataacatggacccccagattgatttataagttctcacaccatagctagattttgttggaactttcataaatcatagtgacataagtatagcataatattcatgccttcgacagaagttttcgcatatggtaaggctactattgaaaaaattcccttgtgtttgaagtacgcataaaaatatctagtggcagtcaaccaaataaaacattctaggagtccctcaaaaaattaaagagtcatcagttcagaagactttaatatcaatactttctattatccgggtttggcatgcagtaaatttcatgagaaaaggaaaaatcagctatttgattatataaggaactaattcggatgtatcactaagctttccatcgactggaacatcgggagctagtctccaatactcgtcaaggatctaacataaacatcttctccgcaatcaaaaagccaaggtcacatacatctaggcctctgtctcattctgatggcatggtatgatgcaagttagacaacactattatttggcagatgacacttaggggtctaatatttaagctcattcaagataatcaagtaatcaagttcaatctcaaggtttcagttgcgctaaaaaatgtaatacttggctcattcagaattagtttgttgaagctggttggtatttgcttcatttgttaatggaaccaggctcataaacaagctttcattaggctaaacttatttaacaaaatcaaaagcttaatactataatttttgataggatttcttttgggcagttatacatgagtaatgaacaagctctacacaatcttttttaatgaacaagctttaatcgagctagggtacgttctattcaacttattggacctgaacttattggaacttatctgaactgaacttattgaacctgaactgaacttattggaacttattaaacctgattggacctgattcaacttattggacctgattgaacctgattggaacttattggacctgattgaacctgattgaccttattggaccttattggaacttattgaccctgattgaaacttattagaccttattggacctgattgaaacttattagaccttattgaacctgattgaaacttatttgaccttattagacaaaaacattattattattattgttattattattattattattattattattattattattattattattattattattattattgttaacctgattgataacatttatatctttcatagttattagtaacgaaaacatgttatctctagttattcaaagacgaattgcaaaatattgtaataataataataataatatattattattattattattgttaaccttaattatttgaccatgattataatattattcaatagcaatatgaataatcaaataatagacaataatacaagtataatactatacattgtggtactttaataaaaaaattctaataataacataatcagctaatagtaatatgaataataaaataatagacataatacaaataaataataaaataatagacataatacagataaataataaaataatttacactaatacaagtataatactatataatcattgtggtactttaattaaaattctaataataacataatccgctaatagtgatatgaaattatgaataacaaaatagtggacaataatacaaatgtttattaaacattgactatttggaccttattggaccttattagacctgattggaacttattggaccttattagacctgattggaacttattgcacctgattggaacttattacacctgattggaacttattgcacctgattggaacttattgcacctgattggaacttattgcacttattagaccttattgcaacttatctgaacttatctgaacttattggacctgaaacttaattttttaagttgaacagaacgcacccctagtatccacgaacatagttagttgttcatcgacaagggtgttaattccttgactataaaaaaaatatctgctaatatgtcctccataccatgtcttgatctgattcccaaaatcacgtgttttcgtgtctggtgaccacgttgctagacatggaagacaggtctaattgttcagtttcaagtcaggttgattaaacatatgttagcaatatacaatcattattagtcaaactaattcaactcgggtttggtttgattcaggttatgtcgaggatcaggtccaaatcgggttaatccttccaggtcaaatatatctaagtctgttttgccaaagtctactttttgtatccgtgtccatgctaaatgacaaacaaaaagcagcttttaccaagctcgaatcagatttgttcgcttaaagagtcacttcgctcatttacagcaacaattaaaggacaaaacattgtccattcaactacttacggatattaacttattggcaactgctagcgtaataaggcaatcaacagcactcggcctcaataatgaacctacaaggagtccaatgaccaatacaaattatcactggcatcatctagcacgacaatctcttaactctaagagtctaagtgccttgacatacaaaagtattccttttaaaagtacccccgtgtggatattctgccaagcaaatgcaatcgatacacccaattagggcttttccattatgagtcctcagagcctcagattgtaaaacaggtcagtaaaagaggaaaatagtatttgattcttttgctaaacccttggatataagaatggtgacttgtattgtcacgccaagcttctttcataaaagctgatcatattattatatgagagttctgagtttcaaggtccgcattcgatctaactagacatcacttccaattaaagttgagaaacgaaactaggtgtcctctttgtttcccaaaggtgaactttagatacttattataagcatattttgttatgaatcgggctaaggagagggctactcttggtattgcataattagttaattacttagtagtagcttgaggaataaggaagcaagtaagttagaggaaagagtatgaaaatctgctataaagtgaggagaggagggatagaaggataatcacaaaattattgagttaactttggttttagttgcttaggttgggagtgtccagccactcgaatgtcttgggactgtaaacaccattgttcatgatctaattgcatcaatattacaattaactcatttctcttcttatccatattcatcttcttacaatcacaactatttccagatcatccatccaaatcttcatccacttgccttagtttctactccagatttcagtctattacaaattgatttctacaatatgtcaattcatcacaaattatcatgttttctgaacaaaagttcactgtttcaggacaaatacagaaagaactactttgatgcttagaacagatatattgtaaaattgtattcggaatttgggatacaactggagaagatatgaataaataggcattcagggagctcagaaaaacagaccgtgccatatggtgctctgctgcataacaggaaataatggataaagtatgaataacgttataacttcttaaaaacctagatgacaagtattttggttgctttttattattggtaggcaaggagaatactcaacaacagtttagccttaaactgcttcttatttctcctcttcccctttttcctgatgatttggggttgtcactcagttcttttacctctcatttccaggtactttagagttatattacacaaaggattgcaagagaagaacaggtcgccctggcatgcactcagaaagtatacgacccttcacaggaaatgtggtgctccaagacttatatctcaggctctcatgagtcatgtcaaggaccatctttaatcatttgtattctaggtttctcaggcgatgcggtgtgctggtgtgtctctccctcccacttgagtgtgtgtattgtttgtgcccctaagtttttatcttaacaatcactactagtcaattagtcattaccaaccctacccacctctcttgttactgttgttcttggagatatttcatatatgtcagcttagaacttatattacgtttcttattacatattctcttaagctcgcgcacatactctgtgatcgaagggatccatattagttatcttttagtggagttgttgtgaaaaaagactgcatagaaaaattaagatagctcatagttgtaaatgtaattgaacttttagattgatagccttgaggctgcttgcattgaaccaaccaaattcagccaggctagtctatgcctctttggtgtcacctggtaggttgaatttgtgtagctgtagttctacaagagactgatttaaaaatgttttcgcactgaaacagcttaaaccacaaaacaggaaagtgcagaacaaactccagaaaatggtgcagaacataccttctcaaaaaggaaaggaactccccattttaacagtacgaggacaactgctacagcactaatggaggagatcaagattttgatccaccagatgaaggattctgatcttgtttcagcctgagaatgtaaggttgaagcttcaggcctctttgtaatagcagatgtcaccagactaacaaattcactgtcgtcttgcatagcaggcccaacatctatgtcatgcttagttagctccattgaatttggcatctccaagagatctcaagagctgcccaaaaagacggtacaatattatgagcatacatgacatgatgacaacccataaagaatatcataacctgtcacattttttattcaaagttcaacagccctcttacaacatgattgagaatggaggggaagagagagagagttggtctcagacattgatcacataatcatttcaattagttttaaaggtgctcatgaaatagaactagtgtcttaagctggagacttctgtatttttcatggttttagattatcaatcatattcttagaatctttgatctctagaactctttcctttcctcccaatattttttccactttgtcttttgttaattacggcttcgctgcaggcctgcaataaatcttttaaatttttacagatactatgtagagttgtatacataagctctaatctgaagacgattggtttcgatgctagttaatacaaataaatatattatggatataatatgcagtaaattgggccatgggcaccagggacaacttagacaagtatagtgcaactaccaggaaatttaagctgggtacctctgattcatcatgctggttgataatattattgcttccacaagtgttcgctacggctcaaccaaactaagtcacaactcacaagctgcacaacccaactgacaattatcgcctattgtctaagctatacattacattaccccaatgccacaacgtggctcacgcctaggcatggtaaggaagttcagatgtacgcagccttacccttttaataacaaagaggctgtttccaggtgacccttaaatcttaattgcaaacaccatctgctgcttcacataaataagcgacttcaaaattgtaaattaaagaatttgaatgcaaattgtgtgaaaaacaactccatcaagaatccattaagcacgctttactattagtatcaataataggaaacccttatatcccttttgacgaaggcacacatgcaacactaatgtgtccttataaacttcatgaaagtatatctctacgaaacccttttagtcttatgtgattctttaagtgtccaactgatgattggttacaaggtatttagcccaaagtagcatttcagagagatggtgtagaatgagtagcttataaaccgaggttgaggtgtaatcctaataaattaggaactaataccacaagagagatggacatgtagagatacaatatagtacagaataagattatttgaaatctttttaccagggaaactccagaggtgttccataaaacacaataccatataactgggagatcaatattttagattaaaaaatataaaaatctatttgggttgagtatatagttggttagtccaataatatataaatttataaggtggaggtcttcggtatatgacattccaaatttgagtatcaaatgatatatatggttttccatacttgaatcccttttcatgtactacctctgtttcaaattaatagttacacttacacttttcacgcatgccaatgcagaactttgaggacatatatctttagttttgtatttgtaaaaattataaaaagtacatattaataaaatacatattaatacgaatctaacaagatcccacatgactatgattttattcacgtataaatcacaaacgagggtcaaaatgcaattgtgaatagtgtaaaatgtcaaagtgtaactattaatttgaaacggaggtagtatgtgtttatgcaacacttttcctttttccctttttgctatttagtaatttatgtaaaatacttccattgacccaaaagttgggtgattatagtttacatctatcattattatttatcattactatagattattcaccattgtaatcaactttataaaagtatacacaggtaactcaggagtcaggggtgctgggccaaacacttttatagtttaaggtgaaaaatctcgagaatcttctcctgccacgcaaaatgagtgttcttccactttaaagatgttataacacttatcttaacctactattcgtaaataacacttatcttaacctactattcgtcaagacatacttgcttcatctcactaagaacgtcttagttttcatttgaaattcgtaccagaaagattcacttcaaatctatttatttttagataaattgttattaaaaacgacgaagaaacgtcagaggacaacaaatcctctaaactccaaattataagtgagtccaactatgttgacgtaaggtaattagagtatccataaaagccctggccgctttggcccacaaagcagcttagaatactacccaaccccaaatataatcaatcaggtgaggaagctcgcaacagatgcgagagttccactccaatcaaaggcaccagaacatagccatcgacatcttctcttctttaccccccttgaaaccaacagatcttaaggaagtccactagtgaacaaggacataaccactactcatgtggaatgccaatcagcctctgtcaaagggaagtccattagtgaacaaggacatacccactgctcaaggtagtcatgtggaaattggaatcccaatcagcctttgtcaaaaggaataagccacatcgcaatgaagaaaaaggtgcaaaccagatttattgcatctccaacacgacataaatatcgagaatgaggcctttactgacaaaggaactctggatttccaatttccactgagcattggactcagttgagaagtaattggtcttgctagattctgtttacgcacatactcttaatgataaataaatgtaacaggccaattggtctggaaaaaaacagttgataaaaggctagtttgggccttggggataaatataatctggtatgagttaataaatttctgtttaaggtaaagagaatgtgttatgtgggataatttaatcaagaaaatcttagtaagatggaggtagtctaacttccattcctcaaaatgtgtaattccttataaaatcagtcagcctctagatacatagttagcaaaaatggaaggtatagaagtgggggtgagggaagaggaaggaaagagaaccgcgatcaatcatattgttcgtgctcaagtttgagttgtgcctatagctagttagagtttgtctatttcattgtttttggtcagtgttcatattctgagtgtcatcgtgtttgggttctagaatgctccttttcctaatgtcgacatttctccactttactctagaaaaatgatctcattgtagccattccagcttcaattttaatggatactaagatccctttcaggaacaatgttaaggtagatgttagtgttttaacagccatgtggatgttagtgtctagaacgagtggtcaaaacactactagcctcaaaatattgtgatcagtctgaaaactctatgttagatggttgctttttttggtaggttcgcttgttttggggggttagctttgtttattttcttcacaatttgcccttaaacttttcacaaaatctacaattgaagattcttaaatagataacagacgtgtcagctacttcaacagctaattgtacgaaaaagttcagctaccttgaaaccaaaccactaacagctagtacagtttgtttctactattacatttatctaatataacagctagtatttagtccaacgatgtataatatcaatgaaatggaactaatctgtaaattggaccttaggcataagagtcgagttgagcaggtacactccaatcaccaagttatttaagcttaaaatgtctaacttccaatgctgtttgacgatactcattgccaagtgtttgttacagatcaaccaagcaaataaagcaacaagtgaacagctgcactagtacccaactgcgaattttcgtcgattgccaagtgcatgtctgggacacaataccatcatgtccatacccattaccttgcttagccagctatcgtaatccataacacataaaaaccaacaaagtcttgatagtttcacaaatcaaaatgttcacttttcattccaaccaaaacaagcaataaatctcttcatccatactcacaagaagaacaatctctcacactacccacttgattagtaaaaaccccaatcaaaaacaaaatccaacccacataaacaaatcaaatttagtaactacccataaactcaaaaacctcaaatcacaataccaataaaagagatatacaatcaatcaaaaaaaatacaacaacagctaaacaaataacatcataaactaaagttattcattttatttcctaactagagatcaattaagcagcataaaacaacatcactaattcaagttaataatcatcaaattctatactataaaacatacataccttaccaaaactacccagctgaaaattagggtagagctccagaaatcccggcgaaaaatccggtgagaaattcagctaaatttgaaaacttctttaggttaagtagtgtacacgatgaattgaagatttttacaagcatatgaaaatggtggttgaaattgaaatgggggtttttgaaaattgttgcgacgcgtaaaagtggaaaaaaaaaaggagagaatcaaagaaatgagcaagtttttgtaggtgggtttactgttgttgcttttgtttgtgcacattactgactattcttaattcttccatgcgtgtgggggtgaaggaattgttttcctaagttgtttagccacttcatagagtcattggatttgaataatctagggaataatgatcatgtgtttagtgtatctataaattataatttatgtatgtatattgtatatgtggtgaggcatagaggacaaggtctaagaggaatagaggattgtgagggagtgtttcatgcttttaagaatgatgagtcattgagtgtattaagttataagtagtatttgatcgagtagtaaagtttgtatcacgtaaatcagagtgataattaggaattgggatttgctcaagtggtgagttttcccatctttccgagcaaggtttctagggttcaattcctacctcaagcatttccttgggatttaaggggacggctcagaggaattcttcttaccaatattttaaaaaaaaaaaaattaagagtggtaatttagttcagatcctacctttatccggttcgaaacgacttcaagaaaaaaaaatccgacatcgtttaaaattttttacttccgactcatttaatccgcctccaactttgaaacaagtagtcttatttcttttatgttaagaaaatttgccaaaaaaaccctttttaaagtccagttttgcgaaaaaaaaaaaccttataaagcattctttgtgaaaacaaaccaaaaagtaaattatttttgcaaaatgaaacctaatctcatttttcggttttgaccatggacttttcgacattgaccacttctatttatcttcttcctccataatcacagcctagccaccactaccaacacctgccgctagcccccacaacctgcacccccacaacctccatccaccccctcaagcggcaaccccccttattcccatacgcggcaaccctacaccttatcctccacccccctccgcccttaccttttctcctctcccttcttccctccatcacccctccccactctcttctccctttgccccccatcgttgcaccacccataatccctctctgtaaccccctctcctcgcagctccccctccctcccagccaaggttgaaaaattacagaggcagtcgcatatggggatgggggactatcgtctaaggggtggagagagggtttgggggctgctggtgggggtggggtaggctgaatgtggtgggggctgagggtggggggtgaaggtggggctgcaggtcgggctggcggtatggagaaagaagggaaatagaagtggttaacaccggaaagtccatgatcaacaccgaaaaatgaaattaggtttcatcttgcaaaaataatttattactttttgatttgttttcgcaaagaatgctttataaggttttttcgcataacatttagacttttatcatccctcttagatttgacacatattatacgaattatactaaaaagactccttatagtaattcgactaatgttttattaaaatgaacctttagaataactcgggtaatat

SEQ ID NO: 76 - DNA, 2000 bp, Beta vulgaris
tacgtaacaaattctgcaaaaatagagatagcaactaataacacgcatgaaaatgacaagttatattatacctttttttctcaatatatgaatatacgtaacaaattaactccagtagtttttagtaaaactattagattattgtgtaacatatactctggaaatagtactaagatccattacaatctttattgagaaatttcctcatgtaccccctgaggtttggcgtaatttccaaatacccctcatatttgaggaatttctcaaataccctgatgtttttgtttagactcaaaatacctttactatggacagtaccctaatgtcattaagttttccccttctctctccccaattttctctctcctcccattcccccacccactacccactgcccactgccaagtaggggtgtaagtggattggactggattggactttgccaaattcaaatccagtccaaagttttttggactcgagaaattgagtccaagtccgatccaaatattttttgagtccagtccaatctagtccgataattttttcttgagtccgaatccagtccagtccagtccgattattatatcttttttcccgatttaggttcaatgattcacaacattttttgagatgcttgagcatttgacatctgattcaattatcaatatccacaaataagattgaaagcttaaattaaagtaaaatactatgaataaaaagttgaattagatgcttaccttgatctaagttgagaggaagcatagagactgagaattaatctgagggacaaatagagaatgcgagagtcgagacagtgaggtagaaagaaaatgaagagtaagaggaagtgagtattaaggactgaggagtaaagtaagatagaattagttggctactagcctactaatgcagtattgctagtataatttacttatttaacaaatggagctaagtgcaatagtttagcgccaattgacatatttagagagagaaggctgaaaaatccaatatttttaaaatagtatcattatttttaatatatacattatatataaaaatatttttggactggactggacatattggactccaaagggatgagtccaaatccagacaaaaaatatttggacttgaaaatttaagtccgagtccagtccgaaaaattttcagtccaatccagtccgacaaatttggactggactggattggactctgaacttttcgtagtccgcttacacccctactgccaagtgccaaactgccaacccccttttggttgagttgatatttgacgcaaagacttggcgtgttggaaggttcattacacattttatccaagtcaactttgaagtcttcttagctagagactagagtgaacgtgttggaaggttcattacacattttatccaatcaaactttgaagtcttcttagctagagactagagtgaacgtgttggaaggttcatgttcatgacattataaaagtaataatagtgaaatttcacaaagtatttataaacccaggacagactcaagagctctacttattattagtgaaaaacaaacatacacacgacaataacacaacataaacaataatgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatccca

SEQ ID NO: 77 - DNA, 2000 bp, Beta vulgaris; identical to SEQ ID NO: 8

SEQ ID NO: 78 - DNA, 5488 bp, Artificial Sequence
  Codon-optimized LBcpf1
aagcttatcgatgtcgacaggccttaagggccagatcccccgggctgcaggaattcgatctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcgaattcggcgcgccatggctcctaagaagaagaggaaggttagcaagctcgagaagtttaccaactgctacagcctctctaagaccctcaggttcaaggctatccctgtgggaaagacccaagagaatatcgacaacaagaggctcctcgtcgaggatgagaagagagctgaagattacaagggcgtgaagaagctcctcgacaggtactacctcagcttcatcaacgatgtgctccacagcatcaagctcaagaacctcaacaactacatcagcctcttccgtaagaaaaccaggaccgagaaagagaacaaagagcttgagaacctcgagatcaacctccgtaaagagatcgccaaggctttcaagggaaacgagggatacaagagcctcttcaagaaggatattatcgagacaatcctgcctgagttcctggacgataaggatgagatcgctctcgtgaacagcttcaacggattcactactgccttcaccggattcttcgacaacagggaaaacatgttcagcgaagaggccaagagcacctctatcgctttcagatgcatcaacgagaacctcacgcgttacatcagcaacatggacatcttcgagaaggtggacgccatcttcgataagcacgaggtgcaagaaatcaaagagaagatcctcaacagcgactacgacgtcgaggacttttttgaaggggagttcttcaacttcgttctcacccaagagggcatcgacgtgtacaacgctattatcggaggattcgtgaccgagtctggggagaagattaagggactcaacgagtacatcaacctgtacaaccagaaaacgaagcagaagctcccgaagttcaagccgctctacaagcaggttctctctgatcgtgagagcctctcattttacggtgagggttacacctctgacgaggaagtgcttgaggttttccgtaacaccctcaacaagaacagcgagatcttctcgtccatcaagaagttggagaaacttttcaagaacttcgacgagtacagcagcgctgggatcttcgttaagaacggacctgctatcagcaccatcagcaaggatattttcggcgagtggaacgtgatcagggacaagtggaatgctgagtacgatgacatccacctcaagaagaaggctgtcgtcactgagaagtacgaggatgacaggcgtaagtcgttcaagaagatcggctctttcagcctcgagcagcttcaagaatacgctgatgctgatctcagcgtggtcgagaagctcaaagagatcatcatccagaaggtcgacgagatctacaaggtgtacgggtcctctgagaagttgttcgatgctgatttcgtcctcgagaagagtctgaagaagaacgacgctgtcgtcgcgatcatgaaggatttgctcgacagcgtgaagtccttcgagaactatatcaaggccttcttcggagagggcaaagagactaatagggacgagtctttctacggggatttcgtgctcgcttacgatatcctcctcaaggtggaccatatctacgacgccatcagaaactacgtgacccagaagccttacagcaaggacaagttcaagttgtactttcagaacccgcagttcatgggcggatgggacaaagacaaagagacagattacagggccaccatcctcaggtacgggtctaagtactacctggccatcatggacaagaaatacgccaagtgcctccaaaagatcgacaaggatgacgtgaacgggaactatgagaagatcaactacaagctccttccgggaccgaacaagatgcttcctaaggtgttcttcagcaagaaatggatggcctactacaacccgtctgaggacatccagaaaatctacaagaacgggaccttcaagaaaggcgacatgttcaacctcaacgactgccacaagctcatcgatttcttcaaggacagcatctcgcgttacccgaagtggtctaacgcttacgactttaacttcagcgagacagaaaagtacaaggatatcgccgggttctaccgtgaggttgaggaacagggttacaaggttagcttcgagagcgcctccaagaaagaggttgacaagttggtcgaagagggcaagctctacatgttccagatctataacaaggacttctccgacaagagccacggaactcctaacctccatacgatgtacttcaagctgcttttcgacgagaacaaccacgggcagatcagactttctggtggtgctgaactcttcatgcgtagggcctcactcaagaaagaagagttggttgttcacccggccaactctccaatcgctaacaagaatcctgacaacccgaaaaagaccaccacgctgtcttacgacgtctacaaggacaaaaggttcagcgaggaccagtacgagcttcatatcccgatcgctatcaacaagtgcccgaagaacatcttcaagatcaataccgaggtgagggtgctgctcaagcacgatgataacccttacgtgatcggaatcgatcgtggtgagagaaacctcctctacatcgttgtggtggacggaaagggaaacatcgtcgagcagtacagcctgaacgagattatcaacaatttcaacggcatcaggatcaagaccgactaccactcactcctcgataagaaagaaaaagagcgtttcgaggccaggcagaactggacttctatcgaaaacatcaaagagttgaaggccggctacatctctcaggtggtgcataagatctgcgagctggtggaaaagtacgatgctgtgatcgctcttgaggacctcaactctgggttcaagaacagtagagtgaaggttgagaagcaggtctaccaaaagttcgagaagatgctcatcgacaagctcaactacatggtggacaaaaagagcaacccttgcgctaccggtggtgctcttaagggataccagatcacgaacaagttcgagtccttcaagagcatgagcacccagaacggcttcatcttctatatccctgcttggctcaccagcaagatcgatccttctactggtttcgtgaacctgctcaagaccaagtacacctcgatcgccgacagcaagaagttcatctcgtctttcgacaggatcatgtacgtgccggaagaggatcttttcgagttcgctctcgactataagaacttcagcaggaccgacgccgactacattaagaagtggaagctctactcctacgggaaccgtatcaggatcttccgaaatccgaagaaaaacaacgtgttcgactgggaagaagtgtgcctcacctctgcctacaaagaactgttcaacaagtacggcatcaactaccagcagggtgatatcagggctcttttgtgcgagcagagcgacaaggcattctacagctcattcatggccctcatgtctctcatgctccagatgaggaactctatcaccggaaggaccgatgtggacttccttatctctccggtcaagaactctgacgggatcttctacgacagccgtaactatgaggctcaagagaacgctatcctgccgaagaatgctgatgcaaacggggcttacaacattgcgagaaaggttctctgggctatcgggcagtttaagaaagcggaagatgagaagctcgacaaggtgaagatcgccatctccaacaaagagtggcttgagtacgctcagacctccgttaagcacaagaggcctgctgctactaagaaagctggccaggccaaaaagaagaagtgaggcgcgccgagctccaggcctcccagctttcgtccgtatcatcggtttcgacaacgttcgtcaagttcaatgcatcagtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggcttgagctccctaggatcaagctt

SEQ ID NO: 79 - DNA, 989 bp, Petroselinum crispum
aattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaacc

SEQ ID NO: 80 - DNA, 8726 bp, Artificial Sequence
  Resistance gene expression cassette
tttatttaaacatgatacgtatcatattgagtactcatacgcgtaccagctgtgacttagaaaaattaaccacgctatataggttccaagccctcatgattaccttttcatagtgtaaatttcatgtagttgaatggtgggaatccaatcacaaaaacactgcaggtaatggaaatgttccaactttttccaagcattttaaaataagacatgtgattactaattagggcgtgttcggcaacagtaactgtggtgatagtttttagctgtgagaatagttgttagctgtgctgttagcttttagtggttggtgtgtaactgttagctgttagatgtccaagtagcggtgtaaaatattgatgttcgataaaagaagctgtcaaagtagctgtttaagaataactagttataaattcaaataaatctttaatatataatttatacaccactaaaagctacccaaaagctacaatctacccaaaagctacaatctacccaaaagctacaaattgtagcttttgacaaacactactaaaacactacttgtaccactaaaagctacttacaccactatcttgccaaacgctcttattttttctaattagtgttttgacctaatcaagacactaaaagctacttaaaaagcttgtgccgaacacgccaattctgaaccaaggaacaaactataacaaaaaagtgctatgtggaacttttgtaggcaacagaagtaaggcatttttggaatgtactaacaaatccgtattaagacttgtacatgaaaattaccgtggtaacatttgcccacacttcctcattcacgtactccgattcattctgataaggcacatcaagatccatgtatctaatagtttaatttgcctctgtgtttctgtattaacaatgagcatagtgagtgcaaaagccatggaagctagattaaaaaggccatcattctaagttagacaattggaaacaacatcgagatacacgtacacataagggctgctcttctctattactccctctgttcctaatcatttgcttttttagcgggttccaaaggcctatgtttgaccactaatatatttaaattaaaactggtgatatatattaaaagaaaattatgatgaatttaacaaaaaccatatatgttatgtccttttttttcctatattaatgaatttttacagtcaaagttggtgaactttgacccaaaaaaagaaatggagcaaaaaaaaaaaaaaaaaaaaaaaactagggacaatgagtaacatttttatctatgtctttttaatatgaatatacgtaacaaattctgcaaaaatagagatagcaactaataacacgcatgaaaatgacaagttatattatacctttttttctcaatatatgaatatacgtaacaaattaactccagtagtttttagtaaaactattagattattgtgtaacatatactctggaaatagtactaagatccattacaatctttattgagaaatttcctcatgtaccccctgaggtttggcgtaatttccaaatacccctcatatttgaggaatttctcaaataccctgatgtttttgtttagactcaaaatacctttactatggacagtaccctaatgtcattaagttttccccttctctctccccaattttctctctcctcccattcccccacccactacccactgcccactgccaagtaggggtgtaagtggattggactggattggactttgccaaattcaaatccagtccaaagttttttggactcgagaaattgagtccaagtccgatccaaatattttttgagtccagtccaatctagtccgataattttttcttgagtccgaatccagtccagtccagtccgattattatatcttttttcccgatttaggttcaatgattcacaacattttttgagatgcttgagcatttgacatctgattcaattatcaatatccacaaataagattgaaagcttaaattaaagtaaaatactatgaataaaaagttgaattagatgcttaccttgatctaagttgagaggaagcatagagactgagaattaatctgagggacaaatagagaatgcgagagtcgagacagtgaggtagaaagaaaatgaagagtaagaggaagtgagtattaaggactgaggagtaaagtaagatagaattagttggctactagcctactaatgcagtattgctagtataatttacttatttaacaaatggagctaagtgcaatagtttagcgccaattgacatatttagagagagaaggctgaaaaatccaatatttttaaaatagtatcattatttttaatatatacattatatataaaaatatttttggactggactggacatattggactccaaagggatgagtccaaatccagacaaaaaatatttggacttgaaaatttaagtccgagtccagtccgaaaaattttcagtccaatccagtccgacaaatttggactggactggattggactctgaacttttcgtagtccgcttacacccctactgccaagtgccaaactgccaacccccttttggttgagttgatatttgacgcaaagacttggcgtgttggaaggttcattacacattttatccaagtcaactttgaagtcttcttagctagagactagagtgaacgtgttggaaggttcattacacattttatccaatcaaactttgaagtcttcttagctagagactagagtgaacgtgttggaaggttcatgttcatgacattataaaagtaataatagtgaaatttcacaaagtatttataaacccaggacagactcaagagctctacttattattagtgaaaaacaaacatacacacgacaataacacaacataaacaataatgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcggtcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggtaagaaagtatattaaacttgttacttttgaaaatattcgctctagtttttgtttcagttggtccattctcactttgtattattgaaatatatcccaaaaaagtaaatataattatataaaagaatcttgctaaaaataatatgaattatttttgtatgtgcaaaataatgtacaaatctaactaatttgttgtggataataatattaattgtgtgaaatagtaaatgtgtggagatatataactttatttatcatattcactcaggtttttaggtatttattatgagttttgcattggagatatccaacttgacaatagtatttttgtaatataccaatatataaagattactgtacataaccaaaatgtatacttttcttatttttataaacttatatattcctcttctttgtatttatcacaacattttttatacccttttgcctcatattaatagcaacacttataatttatttatttactttttatttcttggtctataacctcatctacccacatatgacacaccctataaaggacccacatgattaaccaaaatatacaaatatcttcaatgaaattaactttaacactaatatgataaaaatcatgtcccgctttttatcctctaactaagactctgcataaaggtatattgcaattaatatgagatggaagaggtataataattatatgatcaaattcctggattgaaaaataaatatgagattaaaagtggtatgtttttggttaaaagaaactatccataaagtatgtttttggttaaaagaaactatgcaacataccaatcaaatgtttatacgcttacaatttatgtaccacttttttgtcattgtttttctattgtttgccatacgtacgttactaaatcatgttgtcttttcacattttaactaacaataaattactattgatacaccaaaaaaatctatgagcattggagtacgttgtttgatagaagcttcgtgctattatttcttgtcaaagaatttcatatctcaatatcttctaatttaacaatctaacgaaatttttttgacccaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctgaccaacaatttgttagccgatgaagagcatcaaaaccaaaaaaaacaaaaaaaattgattaatatgcatgagtgtgaccttgttttccaaagtttagcattactattagtgtctcaattcataataataaaaaaattagcttgttcaagatttgtatttttattcaaagattttttttgtctcttgtgcttcttttatcttatatatattttttgtatggtttgtttttgtttaatattagtccctccgctcaaaatgatctttcacgcttgagattggcattaaggtcaagagatgttgctaagctttagaataaaaaaattccaaatgcatagagggaaagaaagcgagacaaaatgttggagaaggcagagtaaatgatgtgatggaggataaatagtagaagtgtgataccgaaagtttgaaaataataaggaattttatttcttgctggcactttgttctagtacaggtttttagcccttcaaaatgtttataatgtagagtcaaaattaatatccttaactagtttttaagtccgggttatatcctagatattaataatattcatttattagtaacattttattttataaatataatactaagcattatttggtttgctggttaagactttagtgtatatctatttctttttttttttattgtatgcgtgtttacataaactaaagactataagggatagtaccacgtggcgcagttccttgcttaggaacgtcttttaatatattaactagtatttgggcccgggcgttgctccgggttggtattgtgtttccgaacatgatgtgcagtttttcccattcccactaaaatatataaaggaaaactcaacatttaaaagatacaaatataataatatggacacttaaaacatgattaaaagttgattgagatggtaattgtgtcatgttataatagtaagaggttgcctaattgaggttgaggtggtggagtagtggtatcgcttcccatctgttatccctgagatataaggatcaaacctcataggactcatttgagtaatttcccatatcctcctctcaaatgagtccttttcatctgacaaaaaaaaatgtctaattttaaattaaaattagacgatcttttataaaatcggcactttctgcacataggtcacaatttttttgtttctatctctctgctttctttaattctacagtctccaactctccatcaacatcttacttattttagaatagatgatgtatggtagtattaaatggtaaagtactaaagctcctataatacacagaagcttacatagtatagattcgtacatgagacaaggttacaatatactttctccgttctttttatattacaataattactattttaagtagtttcacatctattgtaacaattccaattttgttatagaaagcaactttaataattgacaatattgcccttactttatcttattaaaaccatcattaattactcactttctcttataaaattgcttttattttctaaggataatttctctcctattctagttaattaaagagttacttttgtgctaaactgctcatttgttccaaatccttaaaaattgtgtccaaacgcattgttgtaatataaaaagaacagaggtactattagtttgaataaattttgatcggattaggtcacctttagggggcgtttggttaggggtattctggaaacggtaagggaatcaacttacttaattcccttacttgttgtttgtttgctcaatttaatgattccctttacccaccccttactcccaaagtcctttactctcattccccccaccccccaaggtttcacttaccctttcttgattcatcattgaccatatctttgaccacccaactaccaccaccacttgaccacctaatcacctaaccacctaacccaaccactattaccacccaacccctccacctgcccaccaatcggcaccagaactgcccaaccgtcgcccaatcaagccacccaaccggcaccataaccgcccaaccaagccacccaaccggcaccagaaattgtaccaagctacccacacacgtgaaaaccacccacccacaaa

SEQ ID NO: 81 - DNA, 20 bp, Artificial Sequence
  Primer
atgttatctttaccacagtt

SEQ ID NO: 82 - DNA, 24 bp, Artificial Sequence
  Primer
gtccctaaatgaaatacgtaaaac

SEQ ID NO: 83 - DNA, 3706 bp, Artificial Sequence
  PCR Product
atgttatctttaccacagtttgttgctctgacacaaccggtaaatgcattggcctttgtttttgatggcatcaactttggagcatctgattttgcatattcagccttttccatggtaattcttttacaagaattttcattctttcttaagtataaacacttagcttgggacaaacttctgatcctatttcttaatttttgcaggtgatggtggctgttatgagcattttgtgtttgatgtttctttcttctcattacggttttattgggatctgggtggctctaactatttacatgagcctccgcgcgtttgctgaaggcgggaaacgacaatctgatccccatcaagcttgagctcaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactcaaccaatccttctagaagatctaagcttatcgataagcttgatgtaattggaggaagatcaaaattttcaatccccattcttcgattgcttcaattgaagtttctccgatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaacccgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgatgagctcaagaattcgagctcggtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccgccactcgagtggtggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaattggtaattactctttctttttctccatattgaccatcatactcattgctgatccatgtagatttcccggacatgaagccatttacaattgaatatatcctaagtaaaacctcataggttttacgtatttcatttagggac

SEQ ID NO: 84 - DNA, 26 bp, Artificial Sequence
  Primer
cgctgcggacatctacatttttgaat

SEQ ID NO: 85 - DNA, 28 bp, Artificial Sequence
  agttaacttt ccacttatcg gggcactg
agttaactttccacttatcggggcactg

SEQ ID NO: 86 - DNA, 751 bp, Artificial Sequence
  PCR Product
cgctgcggacatctacatttttgaattgaaaaaaaattggtaattactctttctttttctccatattgaccatcatactcattgctgatccatgtagatttcccggacatgaagccatttacaattgaatatatcctaagtaaaacctcataggttttacgtatttcatttagggactaaaatggtttaggataattactttagctaacataagataataaataaataaataaataaaaataaaatggttgtagataaataaggaaatcaataatgaatatgagtgtgagtgataggacgggaatgggaaacttttacactactttaacgctattgaacgagtatgagtatgttataaacgtaaaatgttttatgtgttagacaatggcctcaagtgaaagtgaccctattaatggaggaaatgcaaaccacgagtctgaggtcacgctcgaagaaatgagggcaaggatcgacgcattgcgtagcgaccctgtttttggagatgccacgggagatgctagtgataaccgaatggatttaatgaggttgatgatgatggagcttttacaaggaaatcgacaaaggcctagaactgaacaagaagagtgctcaaacatgttcaagaggttttcggctcataagcccccaacttatgatggaaagccagaccccactgagtttgaagaatggctcaacggcatggaaaaattgttcgatgccacccagtgccccgataagtggaaagttaact

SEQ ID NO: 87 - DNA, 30 bp, Artificial Sequence
  Primer
gctctgacacaaccggtaaatgcattggcc

SEQ ID NO: 88 - DNA, 29 bp, Artificial Sequence
  Primer
gcagattctgctaacttgcgccatcggag

SEQ ID NO: 89 - DNA, 1042 bp, Artificial Sequence
  PCR Product
gctctgacacaaccggtaaatgcattggcctttgtttttgatggcatcaactttggagcatctgattttgcatattcagccttttccatggtaattcttttacaagaattttcattctttcttaagtataaacacttagcttgggacaaacttctgatcctatttcttaatttttgcaggcgatggtggctgttatgagcattttgtgtttgatgtttctctcttctcattacggttttattgggatctgggtggctctaactatttacatgagcctccgcgcgtttgctgaaggcgggaaacgacaatctgatccccatcaagcttgagctcaggatttagcagcattccagattgggttcaatcaacaaggtacgagccatatcactttattcaaattggtatcgccaaaaccaagaaggaactcccatcctcaaaggtttgtaaggaagaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattccctctatataagaaggcattcattcccatttgaaggatcatcagatactgaaccaatccttctagaagatctaagcttatcgataagcttgatgtaattggaggaagatcaaaattttcaatccccattcttcgattgcttcaattgaagtttctccgatggcgcaagttagcagaatctgc

SEQ ID NO: 90 - protein, 665 aa, Beta vulgaris
MAATFTNPTFSPSSTPLTKTLKSQSSISSTLPFSTPPKTPTPLFHRPLQISSSQSHKSSAIKTQTQAPSSPAIEDSSFVSRFGPDEPRKGSDVLVEALEREGVTNVFAYPGGASMEIHQALTRSKTIRNVLPRHEQGGVFAAEGYARATGKVGVCIATSGPGATNLVSGLADALLDSVPLVAITGQVPRRMIGTDAFQETPIVEVTRSITKHNYLVLDVEDIPRIVKEAFFLANSGRPGPVLIDLPKDIQQQLVVPDWDRPFKLGGYMSRLPKSKFSTNEVGLLEQIVRLMSESKKPVLYVGGGCLNSSEELRRFVELTGIPVASTLMGLGSYPCNDELSLHMLGMHGTVYANYAVDKADLLLAFGVRFDDRVTGKLEAFASRAKIVHIDIDSAEIGKNKQPHVSICADVKLALRGMNKILESRIGKLNLDFSKWREELGEQKKEFPLSFKTFGDAIPPQYAIQVLDELTNGNAIISTGVGQHQMWAAQHYKYRNPRQWLTSGGLGAMGFGLPAAIGAAVARPDAVVVDIDGDGSFIMNVQELATIRVENLPVKIMLLNNQHLGMVVQWEDRFYKANRAHTYLGNPSKSADIFPDMLKFAEACDIPSARVSNVADLRAAIQTMLDTPGPYLLDVIVPHQEHVLPMIPSGAGFKDTITEGDGRTSY

SEQ ID NO: 91 - DNA, 1998 bp, Artificial Sequence
  Acetolactate synthase with mutation
atggcggctaccttcacaaacccaacattttccccttcctcaactccattaaccaaaaccctaaaatcccaatcttccatctcttcaaccctccccttttccacccctcccaaaaccccaactccactctttcaccgtcccctccaaatctcatcctcccaatcccacaaatcatccgccattaaaacacaaactcaagcaccttcttctccagctattgaagattcatctttcgtttctcgatttggccctgatgaacccagaaaagggtccgatgtcctcgttgaagctcttgagcgtgaaggtgttaccaatgtgtttgcttaccctggtggtgcatctatggaaatccaccaagctctcacacgctctaaaaccatccgcaatgtcctccctcgccatgaacaaggcggggttttcgccgccgagggatatgctagagctactggaaaggttggtgtctgcattgcgacttctggtcctggtgctaccaacctcgtatcaggtcttgctgacgctctccttgattctgtccctcttgttgccatcactggccaagttccacgccgtatgattggcactgatgcttttcaggagactccaattgttgaggtgacaaggtctattactaagcataattatttagttttggatgtagaggatattcctagaattgttaaggaagccttttttttagctaattctggtaggcctggacctgttttgattgatcttcctaaagatattcagcagcaattggttgttcctgattgggataggccttttaagttgggtgggtatatgtctaggctgccaaagtccaagttttcgacgaatgaggttggacttcttgagcagattgtgaggttgatgagtgagtcgaagaagcctgtcttgtatgtgggaggtgggtgtttgaattctagtgaggagttgaggagatttgttgagttgacagggattccggtggctagtactttgatggggttggggtcttacccttgtaatgatgaactgtctcttcatatgttggggatgcacgggactgtttatgccaattatgcggtggataaggcggatttgttgcttgctttcggggttaggtttgatgatcgtgtgaccgggaagctcgaggcgtttgctagccgtgctaagattgtgcatattgatattgactctgctgagattgggaagaacaagcagccccatgtgtccatttgtgctgatgttaaattggcattgcggggtatgaataagattctggagtctagaatagggaagctgaatttggatttctccaagtggagagaagaattaggtgagcagaagaaggaattcccactgagttttaagacatttggggatgcaattcctccacaatatgccattcaggtgcttgatgagttgaccaatggtaatgctattataagtactggtgttgggcagcaccaaatgtgggctgcgcagcattacaagtacagaaaccctcgccaatggctgacctctggtgggttgggggctatggggtttgggctaccagccgccattggagctgcagttgctcgaccagatgcagtggttgtcgatattgatggggatggcagttttattatgaatgttcaagagttggctacaattagggtggaaaatctcccagttaagataatgctgctaaacaatcaacatttaggtatggttgtccaattggaagataggttctataaagctaaccgggcacatacataccttggaaacccttccaaatctgctgatatcttccctgatatgctcaaattcgctgaggcatgtgatattccttctgcccgtgttagcaacgtggctgatttgagggccgccattcaaacaatgttggatactccagggccgtacctgctcgatgtgattgtaccgcatcaagagcatgtgttgcctatgattccaagtggtgccggtttcaaggataccattacagagggtgatggaagaacctcttattga

SEQ ID NO: 92 - DNA, 21 bp, Beta vulgaris
cctgagagtttgggaagttgc

SEQ ID NO: 93 - DNA, 23 bp, Beta vulgaris
atgtcccgaagattaacgcaatc

SEQ ID NO: 94 - DNA, 3720 bp, Artificial Sequence
  modified genomic sequence of the gene mediating resistance towards cercospora
atgaacatgaaaatcttacttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcggtcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggtaagaaagtatattaaacttgttacttttgaaaatattcgctctagtttttgtttcagttggtccattctcactttgtattattgaaatatatcccaaaaaagtaaatataattatataaaagaatcttgctaaaaataatatgaattatttttgtatgtgcaaaataatgtacaaatctaactaatttgttgtggataataatattaattgtgtgaaatagtaaatgtgtggagatatataactttatttatcatattcactcaggtttttaggtatttattatgagttttgcattggagatatccaacttgacaatagtatttttgtaatataccaatatataaagattactgtacataaccaaaatgtatacttttcttatttttataaacttatatattcctcttctttgtatttatcacaacattttttatacccttttgcctcatattaatagcaacacttataatttatttatttactttttatttcttggtctataacctcatctacccacatatgacacaccctataaaggacccacatgattaaccaaaatatacaaatatcttcaatgaaattaactttaacactaatatgataaaaatcatgtcccgctttttatcctctaactaagactctgcataaaggtatattgcaattaatatgagatggaagaggtataataattatatgatcaaattcctggattgaaaaataaatatgagattaaaagtggtatgtttttggttaaaagaaactatccataaagtatgtttttggttaaaagaaactatgcaacataccaatcaaatgtttatacgcttacaatttatgtaccacttttttgtcattgtttttctattgtttgccatacgtacgttactaaatcatgttgtcttttcacattttaactaacaataaattactattgatacaccaaaaaaatctatgagcattggagtacgttgtttgatagaagcttcgtgctattatttcttgtcaaagaatttcatatctcaatatcttctaatttaacaatctaacgaaatttttttgacccaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctga

SEQ ID NO: 95 - DNA, 2652 bp, Artificial Sequence
  modified cDNA sequence of the gene mediating resistance towards cercospora
  variation (55)..(57)
atgaacatgaaaatcctccttttgtttgtcttccttcatcacctccactacttcatacatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcggtcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctga

SEQ ID NO: 96 - protein, 883 aa, Artificial Sequence
  modified protein sequence of the gene mediating resistance towards cercospora
  VARIANT (209)..(209)
MNMKILLLFVFLHHLHYFIHGRTLTERQALLSIKSAITYDYYNSLSSWKNTTHHCSWPYITCSSSSSSSSVISLNFTMLFLEGILSPDIGFLTNLQNLSIRSNLFSGPLPHSLSLLTQLRYLDVSQNSFTGPIPSSLSLLTQLRYLHVSGNSFTGPIPSFLSLLTQLRYLDVSDNSFTGPIPSSLSLLTQLRYLDVSYNNLNGTLPLSVLEKMSELSYLNLRYNSFYGEIPPEFGKLKKLETLNLGNNTLSGSLPSELGSLKSLKHMDFSSNMLFGEIPQSYSLLRNLIDIDLNRNKLYGSIPDYIGDFPELESLLLDSNNFTGSIPQKLGTNGKLQYLDISNNNFSGSLPLSLCKGDKLQDLDASYNLLVGSIPESLGSCKSLEGVYMGNNFLNGSIPKGLFGSDVSLNDKLLSGGLDEKFGDCVNLRDIDLSNNKLSGKLPATIGNCIHLRSLTLYNNTCTGRIPQEISKCKQLQTLDLSQNQFSGVIPNDITGNKSICNLEKIQTLKLSNNALTGEIPHCVGNIELIALFLQSNKLNGTIPANFSKLCDSLIYLDLSDNQLEGVLPKSLSKCQSLELLNVGNNRLRDKFPSWLDNLPRLQVFSVRFNAFYGPITSSPKVSHPFPMLQIIDLSNNKFCGKLPRRYIKNFATMRNMNESGVGNPQYLGDSSIYSITYSMVLTFNGLQQKYEKLIVTMSTFDISSNNFTGQIPYVIGGLRSLRNLNLSHNVLTGNIPPSIAKLSLLQDLDLSSNRLTGRIPQELVSLTFLGSFNVSNNLLEGSIPHGFNFDTYTANSYQGNLELCGKPLPECGERRAKGTTNNQDDPKNDNERMLSMSEIVVMGFGSGVLVGLAWGYYMFSVGKPFWFIKMASKMESILIGFF

SEQ ID NO: 97 - DNA, 2652 bp, Artificial Sequence
  modified cDNA of the gene mediating resistance towards cercospora
  variation (625)..(627)
atgaacatgaaaatcctccttttgtttgtcttccttcatcacctccactacttcatccatggcagaacacttacagaacgccaagctttactaagtatcaaatctgccattacttatgattattataactctctctcctcatggaaaaacacaacacaccactgcagttggccatacatcacttgctcctcctcttcttcttcttcttctgttatttctctcaacttcaccatgttatttctcgaaggaattctctcccctgatataggcttcctcaccaacctgcaaaacctctctattcgatctaaccttttttctggcccactcccccattctctctctctcctcacccaactccgctatctcgacgtttcccaaaacagtttcacaggtccaatcccatcttctctctctctcctcacccaactccgctatctccacgtttccggcaacagtttcacaggtccaatcccatcttttctctctctcctcacccaactccgctatctcgacgtttccgacaacagtttcacaggtccaatcccatcttctctctctctcctcacccaactccgctatctcgacgtttcctacaacaatctaaatggcactcttcccttatcgctcgttgagaagatgtcggagctcagctaccttaaccttaggtataactctttctacggtgagattccaccggagtttgggaaacttaagaagcttgaaacattgaatcttggtaacaacactctttctgggagtcttccatctgagttgggttcattaaagagtttgaaacatatggacttttctagtaatatgctatttggtgagatcccacaatcttattctcttcttcgaaacttaatcgatattgatcttaatagaaacaagttatatgggagtatacctgattatattggagattttccggagttggaatcacttttattagactcgaataacttcacagggagtatcccacaaaagttaggtacaaacgggaagttgcaatatctagatataagtaacaacaattttagtggtagtttgccactaagtctttgcaaaggagacaaactccaagatctggacgcatcctataatttgttggttgggtcaattcctgagagtttgggaagttgcaagtcacttgaaggagtgtacatgggaaataatttcttaaacgggtcgattcctaagggcttgtttgggagtgatgtttcacttaatgacaaacttcttagtggaggtctcgatgagaaattcggtgattgcgttaatcttcgggacattgatctctctaataataagctatcagggaagttacctgcgaccatcggaaactgtattcatcttcggtccttgacgctttataataacacctgtaccggacgtatccctcaagagattagcaagtgtaagcagctacagaccctcgatctcagccaaaatcagttctctggtgtgatacccaatgatattacaggaaacaaatccatttgcaatctggaaaagatacaaacacttaaattatcaaacaatgctttgactggtgaaatccctcattgtgttggaaatatcgagctcatagcattatttctccaatcaaacaaactgaacggtaccatacccgcaaacttctcaaagttatgtgattcattgatatatctagatcttagtgacaatcaactcgaaggagttctacctaagtccttgtccaaatgtcaaagtctagaactcctaaatgtcgggaacaataggctaagagataaatttccttcatggttagacaacctcccacgtctccaagttttcagtgtgcgttttaacgccttctacggtcctataactagctcaccaaaagttagtcacccatttcctatgctacaaattatcgacctatctaacaataagttttgtggcaagttgccaagaagatatatcaaaaactttgcaaccatgcgcaatatgaatgagtctggtgttgggaatccacagtacctgggggactcatcaatatatagtattacgtactctatggtattgacattcaatgggttacaacaaaaatatgaaaagcttattgtgacgatgtcgacctttgatatatccagcaacaactttactggacagattccatatgttatagggggattacgctcacttcgtaaccttaatctctctcataatgtcttaaccgggaacattcctccatcaattgcaaaattgtctttgcttcaagatttggacctttcatcaaacagacttactggtcgtatccctcaagaattagttagtttaacatttcttgggagtttcaatgtttcgaacaatctattggaggggtctatacctcatggtttcaacttcgacacgtacacagctaattcataccaggggaatctcgaattatgtggaaaaccattacctgagtgtggagaaagaagggcaaaaggcaccactaataatcaagatgatcctaaaaatgataatgaacgaatgttgtcgatgtccgaaatcgtagttatggggtttggcagtggtgtactagttgggttggcttggggatactatatgttttcagtgggaaagcccttttggtttatcaagatggctagcaaaatggaatcaatattgattggttttttctga

SEQ ID NO: 98 - DNA, 21 bp, Beta vulgaris
cgtttccggcaacagtttcac

SEQ ID NO: 99 - DNA, 21 bp, Beta vulgaris
agagagagagaggagtgggtt

SEQ ID NO: 100 - DNA, 201 bp, Beta vulgaris
tgagtagtactgttgaatcttcacatgygattaaaatyaaycatcatcatmatcatcatmggaaagagcacgtgactagtcacgtgccgatggattgtagaagtgattgtgattcttcatcgtcggtgattgatgacgtggacggttgcgatgatgataatattgggtgttsttcttctgttttagccgttggattaacga

SEQ ID NO: 101 - DNA, 201 bp, Beta vulgaris
tgagtagtactgttgaatcttcacatgygattaaaatyaaycatcatcatmatcatcatmggaaagagcacgtgactagtcacgtgccgatggattgtaggagtgattgtgattcttcatcgtcggtgattgatgacgtggacggttgcgatgatgataatattgggtgttsttcttctgttttagccgttggattaacga

SEQ ID NO: 102 - DNA, 201 bp, Beta vulgaris
ttgggtgcattcctgtcattatmgcagatgatatcgttcttccatttgcagatgccattccatgggaagaaattgggttgtttgtggatgaaaaggatgtrccraatttggatacaatacttacttcaatccctcctgaagtcatcttgaggaaacaaaggttacttgctaacccttcagttaaacaagctatgctgttcc

SEQ ID NO: 103 - DNA, 201 bp, Beta vulgaris
aaccgatggttggatctaatctgaggtgccaaaatcacaaaaattgatgattagttcattaaatagtgatggtttttttkagcagccagagtactaaggayaactcaacaccaaaaatgagtcgaagccaagttctatccaacattgatccatacaatgtarycccagctttgtttttctccaaatatgtcacaagagcgt

SEQ ID NO: 104 - DNA, 201 bp, Beta vulgaris
aaccgatggttggatctaatctgaggtgccaaaatcacaaaaattgatgattagttcattaaatagtgatggtttttttkagcagccagagtactaaggataactcaacaccaaaaatgagtcgaagccaagttctatccaacattgatccatacaatgtarycccagctttgtttttctccaaatatgtcacaagagcgt

SEQ ID NO: 105 - DNA, 201 bp, Beta vulgaris; identical to SEQ ID NO: 103

SEQ ID NO: 106 - DNA, 201 bp, Beta vulgaris
ctgctctttcatctaactctctcatctcttcatcwtctcttcaacaatctttkatcaatgtcaataatccttcccttacactctcaccattttcactbtcctcttcttgtttttcctcatccaactcctcttttatctctcccttacattcgtcgaatcgctctgcwttccctctggttttcagaaacagcagcaatsctg

SEQ ID NO: 107 - DNA, 201 bp, Beta vulgaris
ctgctctttcatctaactctctcatctcttcatcwtctcttcaacaatctttkatcaatgtcaataatccttcccttacactctcaccattttcactbtcttcttcttgtttttcctcatccaactcctcttttatctctcccttacattcgtcgaatcgctctgcwttccctctggttttcagaaacagcagcaatsctg

SEQ ID NO: 108 - DNA, 201 bp, Beta vulgaris
ctgctctttcatctaactctctcatctcttcatcwtctcttcaacaatctttkatcaatgtcaataatccttcccttacactctcaccattttcactbtcytcttcttgtttttcctcatccaactcctcttttatctctcccttacattcgtcgaatcgctctgcwttccctctggttttcagaaacagcagcaatsctg

SEQ ID NO: 109 - DNA, 201 bp, Beta vulgaris
ttgcttgtatcttgtgcagttgtgctaatgatttgtaccttccatavargtttgatgtcgtggttgatgtgttgcctgaagtgagatggactcctgagaacggatacaaraacttaaaaattgttgttgaaatagataaygaaataactgctgaggtagcctgcgagcgagagctaaaacgtcgtcataagacattaggtc

SEQ ID NO: 110 - DNA, 201 bp, Beta vulgaris
ttgcttgtatcttgtgcagttgtgctaatgatttgtaccttccatavargtttgatgtcgtggttgatgtgttgcctgaagtgagatggactcctgagaatggatacaaraacttaaaaattgttgttgaaatagataaygaaataactgctgaggtagcctgcgagcgagagctaaaacgtcgtcataagacattaggtc

SEQ ID NO: 111 - DNA, 201 bp, Beta vulgaris
ttgcttgtatcttgtgcagttgtgctaatgatttgtaccttccatavargtttgatgtcgtggttgatgtgttgcctgaagtgagatggactcctgagaayggatacaaraacttaaaaattgttgttgaaatagataaygaaataactgctgaggtagcctgcgagcgagagctaaaacgtcgtcataagacattaggtc

SEQ ID NO: 112 - DNA, 201 bp, Beta vulgaris
accaagagttgtgctgattgtcataatttggtgggatgtagcgaatatgggaygaagttggtgaaagygatgaagacagggacaagatgcttcttcagatagagcaggaatgtttagatgtctacaaaaggaaggttgacctggctgtcaattcaagagcccaacttctccagaccttggcggatgctaatgttgaacttt

SEQ ID NO: 113 - DNA, 201 bp, Beta vulgaris
accaagagttgtgctgattgtcataatttggtgggatgtagcgaatatgggaygaagttggtgaaagygatgaagacagggacaagatgcttcttcagattgagcaggaatgtttagatgtctacaaaaggaaggttgacctggctgtcaattcaagagcccaacttctccagaccttggcggatgctaatgttgaacttt

SEQ ID NO: 114 - DNA, 201 bp, Beta vulgaris
accaagagttgtgctgattgtcataatttggtgggatgtagcgaatatgggaygaagttggtgaaagygatgaagacagggacaagatgcttcttcagatwgagcaggaatgtttagatgtctacaaaaggaaggttgacctggctgtcaattcaagagcccaacttctccagaccttggcggatgctaatgttgaacttt

SEQ ID NO: 115 - DNA, 201 bp, Beta vulgaris
aatgtcaatgaatcrcattawtttttctccaaaaccakcttgagcagaagcagaaatatgcagtatggaaagcagctgatataaggaaggctctaaaagaaggaaggaagcctgttccrggtcctcctggtggygaacaagattcatccactccrtcaagtgctccaagcaatgaatatgtaagatcccaacatggcatgg

SEQ ID NO: 116 - DNA, 201 bp, Beta vulgaris
aatgtcaatgaatcrcattawtttttctccaaaaccakcttgagcagaagcagaaatatgcagtatggaaagcagctgatataaggaaggctctaaaagagggaaggaagcctgttccrggtcctcctggtggygaacaagattcatccactccrtcaagtgctccaagcaatgaatatgtaagatcccaacatggcatgg

SEQ ID NO: 117 - DNA, 201 bp, Beta vulgaris
aatgtcaatgaatcrcattawtttttctccaaaaccakcttgagcagaagcagaaatatgcagtatggaaagcagctgatataaggaaggctctaaaagadggaaggaagcctgttccrggtcctcctggtggygaacaagattcatccactccrtcaagtgctccaagcaatgaatatgtaagatcccaacatggcatgg

SEQ ID NO: 118 - DNA, 201 bp, Beta vulgaris
agcagtgaaataatcttcgyttggaaagagcaagtaatgctgccataatctaatcatatatccaaccacctgacgaaccatgaggaaggaaaggattggcgggacctggggcaaagtccatkggaatgccaggataaccctgtctarggggttgaggatgatggtagaaactagcaccaacggacctagggcccaggtctg

SEQ ID NO: 119 - DNA, 201 bp, Beta vulgaris
agcagtgaaataatcttcgyttggaaagagcaagtaatgctgccataatctaatcatatatccaaccacctgacgaaccatgaggaaggaaaggattggctggacctggggcaaagtccatkggaatgccaggataaccctgtctarggggttgaggatgatggtagaaactagcaccaacggacctagggcccaggtctg

SEQ ID NO: 120 - DNA, 201 bp, Beta vulgaris
agcagtgaaataatcttcgyttggaaagagcaagtaatgctgccataatctaatcatatatccaaccacctgacgaaccatgaggaaggaaaggattggckggacctggggcaaagtccatkggaatgccaggataaccctgtctarggggttgaggatgatggtagaaactagcaccaacggacctagggcccaggtctg

SEQ ID NO: 121 - DNA, 201 bp, Beta vulgaris
agatttaacatttttgtgcwggatttccgccgctagtaatagcacataatggattctcaggattgttccctccttctagtagttatgcctacaatttggcgatgatgactagtgtgccggatgtkgttctatggtgtgacgtccagttaacgaaggatgcggctggcatatgctttccagatataaagctggataattcct

SEQ ID NO: 122 - DNA, 201 bp, Beta vulgaris
agatttaacatttttgtgcwggatttccgccgctagtaatagcacataatggattctcaggattgttccctccttctagtagttatgcctacaatttggcaatgatgactagtgtgccggatgtkgttctatggtgtgacgtccagttaacgaaggatgcggctggcatatgctttccagatataaagctggataattcct

SEQ ID NO: 123 - DNA, 201 bp, Beta vulgaris
agatttaacatttttgtgcwggatttccgccgctagtaatagcacataatggattctcaggattgttccctccttctagtagttatgcctacaatttggcratgatgactagtgtgccggatgtkgttctatggtgtgacgtccagttaacgaaggatgcggctggcatatgctttccagatataaagctggataattcct

SEQ ID NO: 124 - DNA, 201 bp, Beta vulgaris
gatgtaaccctatcaaggagttattygagggagaggagaagacttggaccctctagaagtatcggctctgactggagccgygagaatgagaaggaaggatcattgactgaagaagcaattccttcgagggccatggaaattgataatgatactttaaacaaggatacwtctaaagatactgatactagttttcagaattct

SEQ ID NO: 125 - DNA, 201 bp, Beta vulgaris
gatgtaaccctatcaaggagttattygagggagaggagaagacttggaccctctagaagtatcggctctgactggagccgygagaatgagaaggaaggattattgactgaagaagcaattccttcgagggccatggaaattgataatgatactttaaacaaggatacwtctaaagatactgatactagttttcagaattct

SEQ ID NO: 126 - DNA, 201 bp, Beta vulgaris
gatgtaaccctatcaaggagttattygagggagaggagaagacttggaccctctagaagtatcggctctgactggagccgygagaatgagaaggaaggatyattgactgaagaagcaattccttcgagggccatggaaattgataatgatactttaaacaaggatacwtctaaagatactgatactagttttcagaattct

SEQ ID NO: 127 - DNA, 201 bp, Beta vulgaris
ttcgagggccatggaaattgataatgatactttaaacaaggatacwtctaaagatactgatactagttttcagaattctcatccagctgagccagctaatgaggtgatgaaaccttctgatgatttagacttaaagatgacttckttggtgcaaaacaaggatgataatgagatagtggaascttcagatgtggtgaatgc

SEQ ID NO: 128 - DNA, 201 bp, Beta vulgaris
ttcgagggccatggaaattgataatgatactttaaacaaggatacwtctaaagatactgatactagttttcagaattctcatccagctgagccagctaataaggtgatgaaaccttctgatgatttagacttaaagatgacttckttggtgcaaaacaaggatgataatgagatagtggaascttcagatgtggtgaatgc

SEQ ID NO: 129 - DNA, 403 bp, Beta vulgaris
ttcgagggccatggaaattgataatgatactttaaacaaggatacwtctaaagatactgatactagttttcagaattctcatccagctgagccagctaatdaggtgatgaaaccttctgatgatttagacttaaagatgacttckttggtgcaaaacaaggatgataatgagatagtggaascttcagatgtggtgaatgcttcgagggccatggaaattgataatgatactttaaacaaggatacwtctaaagatactgatactagttttcagaattctcatccagctgagccagctaatagaggtgatgaaaccttctgatgatttagacttaaagatgacttckttggtgcaaaacaaggatgataatgagatagtggaascttcagatgtggtgaatgc

SEQ ID NO: 130 - DNA, 201 bp, Beta vulgaris
actgttgatctrcaaaaatcyatggaatctctgccatttaccgataaaacgttttcttatgagcaagaatctggtggattgcctgaactggggatgtgcagttctattgttaaagatagaggtcaaaagcgrcctgttgaggaggatagtatgatggagggagccaagaaagctaagcaacaatggctttctgttgctcca

SEQ ID NO: 131 - DNA, 201 bp, Beta vulgaris
actgttgatctrcaaaaatcyatggaatctctgccatttaccgataaaacgttttcttatgagcaagaatctggtggattgcctgaactggggatgtgcaattctattgttaaagatagaggtcaaaagcgrcctgttgaggaggatagtatgatggagggagccaagaaagctaagcaacaatggctttctgttgctcca

SEQ ID NO: 132 - DNA, 201 bp, Beta vulgaris
actgttgatctrcaaaaatcyatggaatctctgccatttaccgataaaacgttttcttatgagcaagaatctggtggattgcctgaactggggatgtgcarttctattgttaaagatagaggtcaaaagcgrcctgttgaggaggatagtatgatggagggagccaagaaagctaagcaacaatggctttctgttgctcca

SEQ ID NO: 133 - DNA, 201 bp, Beta vulgaris
aaagatgtctttgtaacattgatctgtaaggaagatttgatgaygattattcgtcaaatgaaagctgataatcttcatggaagaaagtttggattaggagcactagcaagaacaagcttggaaggatgttctctatcctactctgtagaatcaatgtataaaagctgatagtctcttgtgttgtaaacgggkggataatct

SEQ ID NO: 134 - DNA, 201 bp, Beta vulgaris
aaagatgtctttgtaacattgatctgtaaggaagatttgatgaygattattcgtcaaatgaaagctgataatcttcatggaagaaagtttggattaggaggactagcaagaacaagcttggaaggatgttctctatcctactctgtagaatcaatgtataaaagctgatagtctcttgtgttgtaaacgggkggataatct

SEQ ID NO: 135 - DNA, 201 bp, Beta vulgaris
aaagatgtctttgtaacattgatctgtaaggaagatttgatgaygattattcgtcaaatgaaagctgataatcttcatggaagaaagtttggattaggagsactagcaagaacaagcttggaaggatgttctctatcctactctgtagaatcaatgtataaaagctgatagtctcttgtgttgtaaacgggkggataatct

SEQ ID NO: 136 - DNA, 201 bp, Beta vulgaris
ttgaatcaccgtaccctgayccaccacgatatagctccccataagatgtgtcaaaaccacctgttgatgaggcaaatgtagatgccggagctgcatcagtagtatgwgttcttccataacctccacctcccaacccataactatcaccaaaaccatacccaagattcccactgctatatccagaaccrttacctccrcctt

SEQ ID NO: 137 - DNA, 201 bp, Beta vulgaris
ttgaatcaccgtaccctgayccaccacgatatagctccccataagatgtgtcaaaaccacctgttgatgaggcaaatgtagatgccggagctgcatcagtggtatgwgttcttccataacctccacctcccaacccataactatcaccaaaaccatacccaagattcccactgctatatccagaaccrttacctccrcctt

SEQ ID NO: 138 - DNA, 201 bp, Beta vulgaris
ttgaatcaccgtaccctgayccaccacgatatagctccccataagatgtgtcaaaaccacctgttgatgaggcaaatgtagatgccggagctgcatcagtrgtatgwgttcttccataacctccacctcccaacccataactatcaccaaaaccatacccaagattcccactgctatatccagaaccrttacctccrcctt

SEQ ID NO: 139 - DNA, 201 bp, Beta vulgaris
caccgtaccctgayccaccacgatatagctccccataagatgtgtcaaaaccacctgttgatgaggcaaatgtagatgccggagctgcatcagtrgtatgagttcttccataacctccacctcccaacccataactatcaccaaaaccatacccaagattcccactgctatatccagaaccrttacctccrccttgggatg

SEQ ID NO: 140 - DNA, 201 bp, Beta vulgaris
caccgtaccctgayccaccacgatatagctccccataagatgtgtcaaaaccacctgttgatgaggcaaatgtagatgccggagctgcatcagtrgtatgtgttcttccataacctccacctcccaacccataactatcaccaaaaccatacccaagattcccactgctatatccagaaccrttacctccrccttgggatg

SEQ ID NO: 141 - DNA, 201 bp, Beta vulgaris
caccgtaccctgayccaccacgatatagctccccataagatgtgtcaaaaccacctgttgatgaggcaaatgtagatgccggagctgcatcagtrgtatgwgttcttccataacctccacctcccaacccataactatcaccaaaaccatacccaagattcccactgctatatccagaaccrttacctccrccttgggatg

SEQ ID NO: 142 - DNA, 201 bp, Beta vulgaris
tgaaggagggagcaattaccattcctccatatgctcctccacagaaaatgcaagctcatttaatccctccagatacacctcgctccatctttgtttacttccgaggtctcttytatgatgtcggaaatgaccccgaaggaggttactatgcaagrtttgctctctctctctctctctctctttctctctctcatagaagat

SEQ ID NO: 143 - DNA, 201 bp, Beta vulgaris
tgaaggagggagcaattaccattcctccatatgctcctccacagaaaatgcaagctcatttaatccctccagatacacctcgctccatctttgtttactttcgaggtctcttytatgatgtcggaaatgaccccgaaggaggttactatgcaagrtttgctctctctctctctctctctctttctctctctcatagaagat

SEQ ID NO: 144 - DNA, 201 bp, Beta vulgaris
tgaaggagggagcaattaccattcctccatatgctcctccacagaaaatgcaagctcatttaatccctccagatacacctcgctccatctttgtttacttycgaggtctcttytatgatgtcggaaatgaccccgaaggaggttactatgcaagrtttgctctctctctctctctctctctttctctctctcatagaagat

SEQ ID NO: 145 - DNA, 201 bp, Beta vulgaris
ttgggtgcattcctgtcattatmgcagatgatatcgttcttccatttgcagatgccattccatgggaagaaattgggttgtttgtggatgaaaaggatgtgccraatttggatacaatacttacttcaatccctcctgaagtcatcttgaggaaacaaaggttacttgctaacccttcagttaaacaagctatgctgttcc

SEQ ID NO: 146 - DNA, 201 bp, Beta vulgaris
ttgggtgcattcctgtcattatmgcagatgatatcgttcttccatttgcagatgccattccatgggaagaaattgggttgtttgtggatgaaaaggatgttccraatttggatacaatacttacttcaatccctcctgaagtcatcttgaggaaacaaaggttacttgctaacccttcagttaaacaagctatgctgttcc

SEQ ID NO: 147 - DNA, 201 bp, Beta vulgaris
ttgggtgcattcctgtcattatmgcagatgatatcgttcttccatttgcagatgccattccatgggaagaaattgggttgtttgtggatgaaaaggatgtkccraatttggatacaatacttacttcaatccctcctgaagtcatcttgaggaaacaaaggttacttgctaacccttcagttaaacaagctatgctgttcc

SEQ ID NO: 148 - DNA, 201 bp, Beta vulgaris
tgaccacttttgggatgaaagagtctcattttgttcagatgtaccgaagacacgcaaatgcttttcatatcaatgtatattctgcacggraaaggctggattacttgttgaaggttggcgtcaaacacaacgacatcaagaggatactattacggcaacctcagrttcttgggtatactgtggacagcaatatgaagtccc

SEQ ID NO: 149 - DNA, 201 bp, Beta vulgaris
tgaccacttttgggatgaaagagtctcattttgttcagatgtaccgaagacacgcaaatgcttttcatatcaatgtatattctgcacggraaaggctggagtacttgttgaaggttggcgtcaaacacaacgacatcaagaggatactattacggcaacctcagrttcttgggtatactgtggacagcaatatgaagtccc

SEQ ID NO: 150 - DNA, 201 bp, Beta vulgaris
tgaccacttttgggatgaaagagtctcattttgttcagatgtaccgaagacacgcaaatgcttttcatatcaatgtatattctgcacggraaaggctggaktacttgttgaaggttggcgtcaaacacaacgacatcaagaggatactattacggcaacctcagrttcttgggtatactgtggacagcaatatgaagtccc

SEQ ID NO: 151 - DNA, 201 bp, Beta vulgaris
tgaaattwtgcatcttctctgtagcttcagaccgtgatgaagagctcaaacagtggcacgaccagtacwccgatctcgcaaattctgagctttacttggagcaacatccttcagtttgtgaccgtggaagttggaactagaggctgatggaatgcaattcaggttactctggtgtatatgctgtcagattttcaacagagt

SEQ ID NO: 152 - DNA, 201 bp, Beta vulgaris
tgaaattwtgcatcttctctgtagcttcagaccgtgatgaagagctcaaacagtggcacgaccagtacwccgatctcgcaaattctgagctttacttggaacaacatccttcagtttgtgaccgtggaagttggaactagaggctgatggaatgcaattcaggttactctggtgtatatgctgtcagattttcaacagagt

SEQ ID NO: 153 - DNA, 201 bp, Beta vulgaris
tgaaattwtgcatcttctctgtagcttcagaccgtgatgaagagctcaaacagtggcacgaccagtacwccgatctcgcaaattctgagctttacttggarcaacatccttcagtttgtgaccgtggaagttggaactagaggctgatggaatgcaattcaggttactctggtgtatatgctgtcagattttcaacagagt

SEQ ID NO: 154 - DNA, 201 bp, Beta vulgaris
tatatcagttgtgatggaagaatttgaagacttggatgttgatcaatcaggaaccttatctgtttctgatttagcactagctcagtcatcttcacaagcaagasttccgrcttctggycaggagagacctatttgacattatttctgaatatattctagactttgatttgtctatatctctgtgaatattcaagatcatat

SEQ ID NO: 155 - DNA, 201 bp, Beta vulgaris
tatatcagttgtgatggaagaatttgaagacttggatgttgatcaatcaggaaccttatctgtttctgatttagcactagctcagtcatcttcacaagcaggasttccgrcttctggycaggagagacctatttgacattatttctgaatatattctagactttgatttgtctatatctctgtgaatattcaagatcatat

SEQ ID NO: 156 - DNA, 201 bp, Beta vulgaris
tatatcagttgtgatggaagaatttgaagacttggatgttgatcaatcaggaaccttatctgtttctgatttagcactagctcagtcatcttcacaagcargasttccgrcttctggycaggagagacctatttgacattatttctgaatatattctagactttgatttgtctatatctctgtgaatattcaagatcatat

SEQ ID NO: 157 - DNA, 201 bp, Beta vulgaris
tggccgtttgctcacccagggcttgaacgaggaactcccttcacttttctcttctcctggtgtggtcaaaatatcaacaaagaaatctgtctgcatttctcataatttttgctgccatggcaacatcttatcttctctcttcaccttcatctcttaattttattcccaaaaccctaaattcatcttctcttcttcctctca

SEQ ID NO: 158 - DNA, 201 bp, Beta vulgaris
tggccgtttgctcacccagggcttgaacgaggaactcccttcacttttctcttctcctggtgtggtcaaaatatcaacaaagaaatctgtctgcatttctgataatttttgctgccatggcaacatcttatcttctctcttcaccttcatctcttaattttattcccaaaaccctaaattcatcttctcttcttcctctca

SEQ ID NO: 159 - DNA, 201 bp, Beta vulgaris
tggccgtttgctcacccagggcttgaacgaggaactcccttcacttttctcttctcctggtgtggtcaaaatatcaacaaagaaatctgtctgcatttctsataatttttgctgccatggcaacatcttatcttctctcttcaccttcatctcttaattttattcccaaaaccctaaattcatcttctcttcttcctctca

SEQ ID NO: 160 - DNA, 201 bp, Beta vulgaris
ttttttgtcaatccaagaaaaatgatgaaattgttgatgccccagatagattaatctcagcaatttgctatttttaccccttttttgatggtgttcaatatggaaaatatgtcattacccaatttgcycctcttcaactcattatycaacctttagttccagctataagggtgtttaaaagcttcccttttaatgggattt

SEQ ID NO: 161 - DNA, 201 bp, Beta vulgaris
ttttttgtcaatccaagaaaaatgatgaaattgttgatgccccagatagattaatctcagcaatttgctatttttaccccttttttgatggtgttcaatacggaaaatatgtcattacccaatttgcycctcttcaactcattatycaacctttagttccagctataagggtgtttaaaagcttcccttttaatgggattt

SEQ ID NO: 162 - DNA, 201 bp, Beta vulgaris
ttttttgtcaatccaagaaaaatgatgaaattgttgatgccccagatagattaatctcagcaatttgctatttttaccccttttttgatggtgttcaatayggaaaatatgtcattacccaatttgcycctcttcaactcattatycaacctttagttccagctataagggtgtttaaaagcttcccttttaatgggattt

SEQ ID NO: 163 - DNA, 201 bp, Beta vulgaris
cagtgacagcacaactcatackcrtcaagaataaggatgaaagatacctcatgcttccattgagacattgaagctctaagtgctccaaggaagacttyggaatccataggatcctccatattcacagtgacacctccatggttgtcgttgactgcaggaagcarctggmctctatcttctgcacccatattatctgtcaat

SEQ ID NO: 164 - DNA, 201 bp, Beta vulgaris
cagtgacagcacaactcatackcrtcaagaataaggatgaaagatacctcatgcttccattgagacattgaagctctaagtgctccaaggaagacttygggatccataggatcctccatattcacagtgacacctccatggttgtcgttgactgcaggaagcarctggmctctatcttctgcacccatattatctgtcaat

SEQ ID NO: 165 - DNA, 201 bp, Beta vulgaris
cagtgacagcacaactcatackcrtcaagaataaggatgaaagatacctcatgcttccattgagacattgaagctctaagtgctccaaggaagacttyggratccataggatcctccatattcacagtgacacctccatggttgtcgttgactgcaggaagcarctggmctctatcttctgcacccatattatctgtcaat

SEQ ID NO: 166 - DNA, 201 bp, Beta vulgaris
acaggctatcttatcttcacycacagattctaaactaccattctcaactgcatgatctagagccatacagctcaacttctgcacattgatatcaaaaggtggttccctgccggcactatttytcatactcagatctgtcattttcacacaattgtcaacagcatcatctaaatcttcctcgctgctttcagratcctccag

SEQ ID NO: 167 - DNA, 201 bp, Beta vulgaris
acaggctatcttatcttcacycacagattctaaactaccattctcaactgcatgatctagagccatacagctcaacttctgcacattgatatcaaaaggttgttccctgccggcactatttytcatactcagatctgtcattttcacacaattgtcaacagcatcatctaaatcttcctcgctgctttcagratcctccag

SEQ ID NO: 168 - DNA, 201 bp, Beta vulgaris
acaggctatcttatcttcacycacagattctaaactaccattctcaactgcatgatctagagccatacagctcaacttctgcacattgatatcaaaaggtkgttccctgccggcactatttytcatactcagatctgtcattttcacacaattgtcaacagcatcatctaaatcttcctcgctgctttcagratcctccag

SEQ ID NO: 169 - DNA, 201 bp, Beta vulgaris
aaatcaaaactatgttaggatgcaaaaygttaagaaacctccttttagctgaactgataacgggaatttcattcatatcaccagtgcttagattgatcacgtctccactgttccaactaagatagagcagattggcatacttrtgaatattctcactggctattaaattctcagaagaaaaatcaacaccaagattatgac

SEQ ID NO: 170 - DNA, 201 bp, Beta vulgaris
aaatcaaaactatgttaggatgcaaaaygttaagaaacctccttttagctgaactgataacgggaatttcattcatatcaccagtgcttagattgatcacatctccactgttccaactaagatagagcagattggcatacttrtgaatattctcactggctattaaattctcagaagaaaaatcaacaccaagattatgac

SEQ ID NO: 171 - DNA, 201 bp, Beta vulgaris
aaatcaaaactatgttaggatgcaaaaygttaagaaacctccttttagctgaactgataacgggaatttcattcatatcaccagtgcttagattgatcacrtctccactgttccaactaagatagagcagattggcatacttrtgaatattctcactggctattaaattctcagaagaaaaatcaacaccaagattatgac

SEQ ID NO: 172 - DNA, 201 bp, Beta vulgaris
ttttagctgaactgataacgggaatttcattcatatcaccagtgcttagattgatcacrtctccactgttccaactaagatagagcagattggcatacttatgaatattctcactggctattaaattctcagaagaaaaatcaacaccaagattatgacatgcttgtgcaaagacacacccrgtcatgaatgcatcatagc

SEQ ID NO: 173 - DNA, 201 bp, Beta vulgaris
ttttagctgaactgataacgggaatttcattcatatcaccagtgcttagattgatcacrtctccactgttccaactaagatagagcagattggcatacttgtgaatattctcactggctattaaattctcagaagaaaaatcaacaccaagattatgacatgcttgtgcaaagacacacccrgtcatgaatgcatcatagc

SEQ ID NO: 174 - DNA, 201 bp, Beta vulgaris
ttttagctgaactgataacgggaatttcattcatatcaccagtgcttagattgatcacrtctccactgttccaactaagatagagcagattggcatacttrtgaatattctcactggctattaaattctcagaagaaaaatcaacaccaagattatgacatgcttgtgcaaagacacacccrgtcatgaatgcatcatagc

SEQ ID NO: 175 - DNA, 162 bp, Beta vulgaris
tcaaaccaattcgacttttgctcraagttgcaaactaaagaaaaaggctgaatgcaaagcaagttcaycaatgaacaatagaccggtgttggcctgaggccacatcaagtgaagttgcctaattgcggccctctcatctgttcacaggaatcattttccata

SEQ ID NO: 176 - DNA, 162 bp, Beta vulgaris
tcaaaccaattcgacttttgctcraagttgcaaactaaagaaaaaggctgaatgcaaagcaagttcaycaatgaacaatagatcggtgttggcctgaggccacatcaagtgaagttgcctaattgcggccctctcatctgttcacaggaatcattttccata

SEQ ID NO: 177 - DNA, 162 bp, Beta vulgaris
tcaaaccaattcgacttttgctcraagttgcaaactaaagaaaaaggctgaatgcaaagcaagttcaycaatgaacaatagaycggtgttggcctgaggccacatcaagtgaagttgcctaattgcggccctctcatctgttcacaggaatcattttccata

SEQ ID NO: 178 - DNA, 128 bp, Beta vulgaris
attaaagctttaattgccatttgacatccccttgctcaactttagcatgtttttaggctacaacaaatacgcactgtctacatggacatacaaattacaagcgtatggaaaagcaataagcgcaagga

SEQ ID NO: 179 - DNA, 127 bp, Beta vulgaris
attaaagctttaattgccatttgacatccccttgctcaactttagcatgtttttaggctacaacaatacgcactgtctacatggacatacaaattacaagcgtatggaaaagcaataagcgcaagga

SEQ ID NO: 180 - DNA, 128 bp, Beta vulgaris; identical to SEQ ID NO: 178
  misc_recomb (65)..(65): deletion

SEQ ID NO: 181 - DNA, 201 bp, Beta vulgaris
gggagtattatccaatatccatgcaggttgttggtacatgttaattgttacatgaagccrtccaaaaccttcaaaagcagtaagtcttgctgctatgycaaattcaaatattcaactccaaaaaaaaaaaaaaaaaaattcaaaaatccgcttttcagcgaawawataggaaataatccaagaatcgaaatcgaaataaag

SEQ ID NO: 182 - DNA, 201 bp, Beta vulgaris
gggagtattatccaatatccatgcaggttgttggtacatgttaattgttacatgaagccrtccaaaaccttcaaaagcagtaagtcttgctgctatgycatattcaaatattcaactccaaaaaaaaaaaaaaaaaaattcaaaaatccgcttttcagcgaawawataggaaataatccaagaatcgaaatcgaaataaag

SEQ ID NO: 183 - DNA, 201 bp, Beta vulgaris
gggagtattatccaatatccatgcaggttgttggtacatgttaattgttacatgaagccrtccaaaaccttcaaaagcagtaagtcttgctgctatgycawattcaaatattcaactccaaaaaaaaaaaaaaaaaaattcaaaaatccgcttttcagcgaawawataggaaataatccaagaatcgaaatcgaaataaag

SEQ ID NO: 184 - DNA, 134 bp, Beta vulgaris
gctagattttgttggaactttcataaatcatastgacataagtatagcataatattcatgscktccgacagaagttttcgcatatggtaaggctactattgaaaaaattcccttgtgtttgaagtacgcataaa

SEQ ID NO: 185 - DNA, 133 bp, Beta vulgaris
gctagattttgttggaactttcataaatcatastgacataagtatagcataatattcatgscktcgacagaagttttcgcatatggtaaggctactattgaaaaaattcccttgtgtttgaagtacgcataaa

SEQ ID NO: 186 - DNA, 134 bp, Beta vulgaris; identical to SEQ ID NO: 184
  misc_recomb (65)..(65): deletion

SEQ ID NO: 187 - DNA, 144 bp, Beta vulgaris
aatataaaaatctatttgggttgagtatatagttggttagtccaataatatataaatttataaggttgaggtcttcggtatatgacattccaaatttgagtatcaamtgatatatatggktttccatacttgaattacttgaat

SEQ ID NO: 188 - DNA, 135 bp, Beta vulgaris
aatataaaaatctatttgggttgagtatatagttggttagtccaataatatataaatttataaggtggaggtcttcggtatatgacattccaaatttgagtatcaamtgatatatatggktttccatacttgaat

SEQ ID NO: 189 - DNA, 135 bp, Beta vulgaris
aatataaaaatctatttgggttgagtatatagttggttagtccaataatatataaatttataaggtkgaggtcttcggtatatgacattccaaatttgagtatcaamtgatatatatggktttccatacttgaat

SEQ ID NO: 190 - DNA, 201 bp, Beta vulgaris
cttatattattaaaaagttcaggaaaatgacaaatttaataacaaaaagaacatgcagggatttgtctaaaattaccacacggccaacaaatttgaagtacgaaagatgttcagtytggtagacrgaattagggttaggctggaaggtcgcattgttccctactgtggtgaatagtagagctcccttatcaaataagaccc

SEQ ID NO: 191 - DNA, 201 bp, Beta vulgaris
cttatattattaaaaagttcaggaaaatgacaaatttaataacaaaaagaacatgcagggatttgtctaaaattaccacacggccaacaaatttgaagtatgaaagatgttcagtytggtagacrgaattagggttaggctggaaggtcgcattgttccctactgtggtgaatagtagagctcccttatcaaataagaccc

SEQ ID NO: 192 - DNA, 201 bp, Beta vulgaris
cttatattattaaaaagttcaggaaaatgacaaatttaataacaaaaagaacatgcagggatttgtctaaaattaccacacggccaacaaatttgaagtaygaaagatgttcagtytggtagacrgaattagggttaggctggaaggtcgcattgttccctactgtggtgaatagtagagctcccttatcaaataagaccc

SEQ ID NO: 193 - DNA, 201 bp, Beta vulgaris
agkggagagcatggaagcaaaaggtaytgcagttagttgtaccatgtacacgatgatgatggatggattttgcaaaaaggggatgctcactgaagctgtagatttgctagctgcaatatcaaaggttkgcgcttktgctgacgtgatcacatattctgtgctaatcaatggattctgcaggttggggaagatgcataatgc

SEQ ID NO: 194 - DNA, 201 bp, Beta vulgaris
agkggagagcatggaagcaaaaggtaytgcagttagttgtaccatgtacacgatgatgatggatggattttgcaaaaaggggatgctcactgaagctgtaaatttgctagctgcaatatcaaaggttkgcgcttktgctgacgtgatcacatattctgtgctaatcaatggattctgcaggttggggaagatgcataatgc

SEQ ID NO: 195 - DNA, 201 bp, Beta vulgaris
agkggagagcatggaagcaaaaggtaytgcagttagttgtaccatgtacacgatgatgatggatggattttgcaaaaaggggatgctcactgaagctgtaratttgctagctgcaatatcaaaggttkgcgcttktgctgacgtgatcacatattctgtgctaatcaatggattctgcaggttggggaagatgcataatgc

SEQ ID NO: 196 - DNA, 201 bp, Beta vulgaris
agtaaatagaatttgttcaactatcttaaggtctctggtcttgtactcaaaatgtatcatatcctcaagtgtagagggttgagatagtgtggactgtggactgttcactccggatgaagggagaaaaacaaaaggagaggygagacgtgagagtatttctagagttgacaagaactgaaagagtgacatctggtcatctac

SEQ ID NO: 197 - DNA, 201 bp, Beta vulgaris
agtaaatagaatttgttcaactatcttaaggtctctggtcttgtactcaaaatgtatcatatcctcaagtgtagagggttgagatagtgtggactgtggattgttcactccggatgaagggagaaaaacaaaaggagaggygagacgtgagagtatttctagagttgacaagaactgaaagagtgacatctggtcatctac

SEQ ID NO: 198 - DNA, 201 bp, Beta vulgaris
agtaaatagaatttgttcaactatcttaaggtctctggtcttgtactcaaaatgtatcatatcctcaagtgtagagggttgagatagtgtggactgtggaytgttcactccggatgaagggagaaaaacaaaaggagaggygagacgtgagagtatttctagagttgacaagaactgaaagagtgacatctggtcatctac

SEQ ID NO: 199 - DNA, 201 bp, Beta vulgaris
aagargargagaagaaagatgaggarggtaaggtagaggatgttgatgaggaaaaagagaaagaagagaagaagaaaaagaagattaargaggtctccaacgaatggtctttggttaacaagcaaaagcccatctggatgaggaagccagaagagatcaccaaagaggagtacgctgcattttacaagagtcttaccaatg

SEQ ID NO: 200 - DNA, 201 bp, Beta vulgaris
aagargargagaagaaagatgaggarggtaaggtagaggatgttgatgaggaaaaagagaaagaagagaagaagaaaaagaagattaargaggtctccaatgaatggtctttggttaacaagcaaaagcccatctggatgaggaagccagaagagatcaccaaagaggagtacgctgcattttacaagagtcttaccaatg

SEQ ID NO: 201 - DNA, 201 bp, Beta vulgaris
aagargargagaagaaagatgaggarggtaaggtagaggatgttgatgaggaaaaagagaaagaagagaagaagaaaaagaagattaargaggtctccaaygaatggtctttggttaacaagcaaaagcccatctggatgaggaagccagaagagatcaccaaagaggagtacgctgcattttacaagagtcttaccaatg

SEQ ID NO: 202 - DNA, 201 bp, Beta vulgaris
aacgaaaacaagagaaaactaacaagcatttgcagatagccagagacatamagtcgattttttgttcaataggagcttgtggatggaaaacagagagtccacgagcgaactcttcaaaacctaaaactccattatgctttgtgtcaaacaagtcaaagaccygcaasaagtaaacaayattagcattkctgagaaaaatac

SEQ ID NO: 203 - DNA, 201 bp, Beta vulgaris
aacgaaaacaagagaaaactaacaagcatttgcagatagccagagacatamagtcgattttttgttcaataggagcttgtggatggaaaacagagagtccccgagcgaactcttcaaaacctaaaactccattatgctttgtgtcaaacaagtcaaagaccygcaasaagtaaacaayattagcattkctgagaaaaatac

SEQ ID NO: 204 - DNA, 201 bp, Beta vulgaris
aacgaaaacaagagaaaactaacaagcatttgcagatagccagagacatamagtcgattttttgttcaataggagcttgtggatggaaaacagagagtccmcgagcgaactcttcaaaacctaaaactccattatgctttgtgtcaaacaagtcaaagaccygcaasaagtaaacaayattagcattkctgagaaaaatac

SEQ ID NO: 205 - DNA, 201 bp, Beta vulgaris
tatcccaccactctcgatccttaaacttcatcctcaaayyaataagcttctccgcataaggagaaggtgacataagctcagtagaacgctcagactcattcgtaggcttaatcacaaaatgcttaatcccagtaacaaactcagtcatattatactgcattgccaacccatcaatctgatcagacacygtctcataaaact

SEQ ID NO: 206 - DNA, 201 bp, Beta vulgaris
tatcccaccactctcgatccttaaacttcatcctcaaayyaataagcttctccgcataaggagaaggtgacataagctcagtagaacgctcagactcatttgtaggcttaatcacaaaatgcttaatcccagtaacaaactcagtcatattatactgcattgccaacccatcaatctgatcagacacygtctcataaaact

SEQ ID NO: 207 - DNA, 201 bp, Beta vulgaris
tatcccaccactctcgatccttaaacttcatcctcaaayyaataagcttctccgcataaggagaaggtgacataagctcagtagaacgctcagactcattygtaggcttaatcacaaaatgcttaatcccagtaacaaactcagtcatattatactgcattgccaacccatcaatctgatcagacacygtctcataaaact

SEQ ID NO: 208 - DNA, 201 bp, Beta vulgaris
cagaggaggtggktgtttcaaagttaaagctccatttttctagagaaattgtcaaaaaaaaaagaagcaaaaaaaggggcaarctttyacctctatctctctcttccactactattgttcttgtttctacacctctccttggtttttgcttcaaatctcttcaaggattgaaaagggttcttaaagatcccaawtttaagg

SEQ ID NO: 209 - DNA, 201 bp, Beta vulgaris
cagaggaggtggktgtttcaaagttaaagctccatttttctagagaaattgtcaaaaaaaaaagaagcaaaaaaaggggcaarctttyacctctatctctatcttccactactattgttcttgtttctacacctctccttggtttttgcttcaaatctcttcaaggattgaaaagggttcttaaagatcccaawtttaagg

SEQ ID NO: 210 - DNA, 201 bp, Beta vulgaris
cagaggaggtggktgtttcaaagttaaagctccatttttctagagaaattgtcaaaaaaaaaagaagcaaaaaaaggggcaarctttyacctctatctctmtcttccactactattgttcttgtttctacacctctccttggtttttgcttcaaatctcttcaaggattgaaaagggttcttaaagatcccaawtttaagg

SEQ ID NO: 211 - DNA, 201 bp, Beta vulgaris
tatgcacaacgcatttagctgcataaaggaccgattggrtcaatactaatattacaacatgaaggctgaagctgttaccgttcaaaaggtggcaacgctcgayttatagttgcttgtagcaactatatgcgacaccaactgagacagcagcacaracaattgcaagggttgcrtatgtatcttcacgttcccagctctcga

SEQ ID NO: 212 - DNA, 201 bp, Beta vulgaris
tatgcacaacgcatttagctgcataaaggaccgattggrtcaatactaatattacaacatgaaggctgaagctgttaccgttcaaaaggtggcaacgctcaayttatagttgcttgtagcaactatatgcgacaccaactgagacagcagcacaracaattgcaagggttgcrtatgtatcttcacgttcccagctctcga

SEQ ID NO: 213 - DNA, 201 bp, Beta vulgaris
tatgcacaacgcatttagctgcataaaggaccgattggrtcaatactaatattacaacatgaaggctgaagctgttaccgttcaaaaggtggcaacgctcrayttatagttgcttgtagcaactatatgcgacaccaactgagacagcagcacaracaattgcaagggttgcrtatgtatcttcacgttcccagctctcga

SEQ ID NO: 214 - DNA, 201 bp, Beta vulgaris
gttcgagagctgggaacgtgaagatacataygcaacccttgcaattgtytgtgctgctgtctcagttggtgtcgcatatagttgctacaagcaactataaatygagcgttgccaccttttgaacggtaacagcttcagccttcatgttgtaatattagtattgayccaatcggtcctttatgcagctaaatgcgttgtgca

SEQ ID NO: 215 - DNA, 201 bp, Beta vulgaris
gttcgagagctgggaacgtgaagatacataygcaacccttgcaattgtytgtgctgctgtctcagttggtgtcgcatatagttgctacaagcaactataagtygagcgttgccaccttttgaacggtaacagcttcagccttcatgttgtaatattagtattgayccaatcggtcctttatgcagctaaatgcgttgtgca

SEQ ID NO: 216 - DNA, 201 bp, Beta vulgaris
gttcgagagctgggaacgtgaagatacataygcaacccttgcaattgtytgtgctgctgtctcagttggtgtcgcatatagttgctacaagcaactataartygagcgttgccaccttttgaacggtaacagcttcagccttcatgttgtaatattagtattgayccaatcggtcctttatgcagctaaatgcgttgtgca

SEQ ID NO: 217 - DNA, 201 bp, Beta vulgaris
acatgcagattcccagcaatggattctgtttctgcttctttytcttttaattttgcatgctcttttctcaaattctctagttcctctttcagagaatccaccatgttttggagggcacttccctcttccaacactttttgsagagattctttcgcaccatcsagctccaatgtgacmgttctcacagagtctacatccaat

SEQ ID NO: 218 - DNA, 201 bp, Beta vulgaris
acatgcagattcccagcaatggattctgtttctgcttctttytcttttaattttgcatgctcttttctcaaattctctagttcctctttcagagaatccaacatgttttggagggcacttccctcttccaacactttttgsagagattctttcgcaccatcsagctccaatgtgacmgttctcacagagtctacatccaat

SEQ ID NO: 219 - DNA, 201 bp, Beta vulgaris
acatgcagattcccagcaatggattctgtttctgcttctttytcttttaattttgcatgctcttttctcaaattctctagttcctctttcagagaatccamcatgttttggagggcacttccctcttccaacactttttgsagagattctttcgcaccatcsagctccaatgtgacmgttctcacagagtctacatccaat

SEQ ID NO: 220 - DNA, 201 bp, Beta vulgaris
tccctgaggatgcatgaagttgcttgtaaaattttgaaatacttgaccaacggtgcagttgtactcggagagtactgttacgaccttcaaaagctataaactwgagtttcatctttattctattgacatgcttcacaaaacaacgtagttatacaggatttttatattatgacatgctrcgctctttgtcacaagtagagc

SEQ ID NO: 221 - DNA, 201 bp, Beta vulgaris
tccctgaggatgcatgaagttgcttgtaaaattttgaaatacttgaccaacggtgcagttgtactcggagagtactgttacgaccttcaaaagctataaattwgagtttcatctttattctattgacatgcttcacaaaacaacgtagttatacaggatttttatattatgacatgctrcgctctttgtcacaagtagagc

SEQ ID NO: 222 - DNA, 201 bp, Beta vulgaris
tccctgaggatgcatgaagttgcttgtaaaattttgaaatacttgaccaacggtgcagttgtactcggagagtactgttacgaccttcaaaagctataaaytwgagtttcatctttattctattgacatgcttcacaaaacaacgtagttatacaggatttttatattatgacatgctrcgctctttgtcacaagtagagc

SEQ ID NO: 223 - protein, 517 aa, Artificial Sequence
  mutated EPSP
MAQASTINNGVKSTQLCPNLPKTHLSKSSKSVKFGSNLRFSPKLKSFNNERVGGNSSVVFRVRASVAAAAEKSSTVPEIVLQPIKEISGTIQLPGSKSLSNRILLLAALSQGTTVVDNLLYSDDIRYMLDALRTLGLNVEDDNIAKRAIVEGCGGLFPVGKDGKEIELFLGNAGTAMRSLTAAVAVAGGNSSYVLDGVPRMRERPIGDLVAGLKQLGADVDCYLGTNCPPVRVNAKGGLPGGKVKLSGSVSSQYLTALLMATPLGLGDVEVEIIDKLISVPYVEMTIKLMERFGVSVEHSADWGRFLIRGGQKYKSPGNAYVEGDASSASYFIGGAAVTGGTVTVEGCGTSSLQGDVKFAEVLEKMGCKVSWTENSVTVTGPPRDASGRKHLRAVDVNMNKMPDVAMTLAVVALYADGPTTIRDVASWRVKETERMIAICTELRKLGATVEEGSDYCVITPPEKLNVTAIDTYDDHRMAMAFSLAACADVPVTIKDPGCTRKTFPDYFDVLERFAKH

SEQ ID NO: 224 - DNA, 7001 bp, Artificial Sequence
  genomic DNA of mutated EPSPS gene
aggaagtatttgaatttgatatagatattgtgtctttgtgtgtgttgaatttcaattcccagttccctaaaaaaaatttacaattgcaatttcgagattatgatgtaaattaaatttgagagactagaaagtatttggtcaacccaaaaaaaaaatatcaatacttatataaatcaaaaacataatagagaatccaattttactaaaaatattagtaattttgattaaaataatctattaaaatgaactctaaccttcacataatttccacatattattaatcaacaaaataagcatcacaaattattagaataggcgatctaattttaacataaaattagacgaattcaaattgaatttttctaacaagctcattccatttcacgcaacccaaaattatcctagtcagtagtcatccattcttttctcattcctttattcttgattatcgaactacaacagataatttcaaaaaaaaactaaattggtagtcttaactgattaaactacttactaaatggattaaagaatgtcattactgaatagattaaactgattacgaaatagattaacttggtccctaaatagattaaattagttactatattaaaattaggcgatctcttacaaaaccaactgaataagcatagctctgtatattacctagatttcaactaaatcaaaaccccttacagttcaatctagagctgatcattttggctcggcccgtcccatttttgggccgggttttagtcagatttttttggcccgcggtcgggcccggcccgatttttttggctttgggcaagccaaaaacgacttttcagtttattttttggcccgacccgtttttacccgcaaaagcccgctaatttaggtccgcactttgggcacaaaaatttagcccgaacttaaacctggcccgacccatgatcacctctagtttaatccaaactaaaaaactacacaagttagccaaaaattatgtctactttgtacaactttataaaatacacacagtagttgatatcttgatgattaactccttttgaagtttgactacacaccaaccccaaacacacccactttttcccccctcttgtcaccaaccccccctcctctttagccaccaaagtttggttggtgagtcctccataactgctaaattctctcttttttctctctcctaaaaaactaaaacccaccaaaatttcagacatcaaaaaaattacaagtgaaggaaacaataatggctcaagctagcaccataaacaatggtgtcaaaagcacccaattatgccccaatttacccaaaacccacttatccaaatcttcaaaatctgttaaatttggatcaaatttgagattttctccaaagttgaaatcttttaacaatgaaagagttggtgggaattcatcagttgttttcagggttagggcttcagttgcagcagcagctgaaaaatcatcaactgtaccagaaattgtgttacaacccatcaaagagatctcgggtaccattcaattgcccggatccaagtcattatctaatcggattctacttcttgctgccctttctcaggtacttttcaattgtttgatttctttttttcttagaacttgtgaatttgtatactttatccgtttctaaatacgtgcaacatttgaatagtaacgagtatttatctaccaacttatttaatattctctcacgaatgtatatgaaaaaatatagtcatgcgtggttttatttgattgatctgcggacttttataatatcaactttttataatttagaggacaaagtagtgtattgggtagcgtgtaaggaggttgggaaactggaggaattttttaacaattcaagtttgatatttttcatagtgaaatgtttattagcatagaatcatgcttttagtttttagtggagtgtgcatttattctttaacttgttggatggctatgattaagaattggattctggttatttgcttgagtatttagaaattaattgtgggtgttggtgataatgtaacaaaattgttttgaaggtgtgagaatgtgattttaattatatgaggaaatgatgggttatttattgtaatgtgggaatttatgataatatgttggaggatgaaacaattgatgattttgaagtggggtatgaaggctttcccccatttttttacctttcatgtgttgtatgtagcagactgccaaagcaattcttctgagcagcttggcatatttccattgcaactcataatctcaatccaagcacaatcaattacctgccacgctagcactttaagtagcaaaatcgcgcttgaagaaaagataataccacaggcttctatagttctattccttgtgatataggacacaagtatcatcctagatgacttccctagccctgcaaataattcctggaaggaagtccgttgagtatccttgatcaagtaaaattagcgctttcttttgggtaacacaaactatactcgaaccactttatgctaagggatcacattgcgtctcttgcagaacttttaacatttctaacatggcataagcacggaaacaccctgtgaagttgctagtgaccatcttttttgataaatcaagaatccctgttcttatttgcatacaaagggttaagtcttgtctgagagggattgacatatctcctgctggatagactttgtgttcatctctaatgttattctgagtttgcattgtatgtgttgtgttttcgtttttcttcttgtttgtgtgtgtgtgtgtgtgtgtgtgtgtgtgttacccaaacttgtacggaaatttccatcctaacgagaattatgctgcagggaacaacagtggttgacaacttgctatatagtgatgatatccgttacatgctggatgctttgagaactcttgggttgaatgtggaggatgataatatagccaaaagggcaatcgtggagggttgcggtggtttgtttcctgttggaaaagatgggaaagaaattgaactttttcttggaaatgcaggaacagcaatgcgctcattgacagctgcagttgctgttgctggaggaaattctaggtttgtctactaaggccttccatgtcatcgtgaggctgttaatattcttttattttgtgtggagacttgtttcctcttgctacaattgcaaagggggctgggattgattaacaatgcagaactcaaacactatctttggtatcaccgtctggcaggtggcatctgatcttgcaaaacagatatggagggttttgccggaaaggggttgggggtgggacttgggagtatagcctcaatgttatagaagattgtgctatactactgtaaaattaacttttggttggaaccagatatagttaataaagtatttttttatctggtgaggggtgcttctaggtcctgaaaagttccaccatagaaaaaaattgaatttcattgtgaaaagctaaggtgaattcattaaattcaatacatgttgttgattgcttgaactagaatacttcagatatcagttaattgaatttatttcttttggagaacatgcatgaagagcttttaactcgcctctactatcctgctcttaattatttttataaatagatttagcaattgggaatatcctgtaatttgctggttgctttttcatatggtatatattgaattggtgtggaggcgttttacaaagagtgaaggtaattgttgatgggtggatattgaatggaaatactttgatggcggatagcatattcatctgattggatgtctgaaatttgatgagctctagtaaatgttcctccatgtatgcttgtgtattgggaatttgcaatccagtagtagagaacacccctgataattgcactattattcctgagttgtgagagctctcaagaagatagatggggttcttcaagttttttaattgtctttaggtagtgtaaaggttgcaatatttcaaaggatctgtctgatccaacaagtctgctaaatccttcaccaatctgaaaggggagaagatactgctctctctgacctgcttgcatgcatataaagcttctaaaacaaataggttaaatgtaaaacaagcgcagaagttttcacttctcttgggaaatgtgtgccaagatctattgcatgacatgaattcatatttattgattatatgctcaacatgaatgggattaagaatgaaataaagtagcatgagtaggagacaatccgcctgctcttttaataggcatatacaatcagtcatcaacatcattgctgctttagaatttgatttatgttgatcttggtttttgcagctatgtacttgatggagtgccgagaatgagggagcgacccattggggatttggtagcgggtctaaagcaacttggcgccgatgttgactgttatcttggcacaaattgtcctcctgttcgagtgaatgctaaaggaggccttcccgggggcaaggtgaattctcttgttttgattagagaaaaattataagttagtgttctatcatgcttgacacgaacaagtttttttatctgaaagagaaagaagtgtatgcctaatgattaacagtcatattcaaaatgatataaaagagggtggaaggttacttgctctatgatgatttatggtagtaccgtgttaccaatagcagaattcaagcaatattctttgaactggtaataacaagttcaagcaatcaagcatagtttagattagattctatcaatctcttagcatagtttaaattcgcttccatcaatctcttttgtgacgtaagaagttctgttccattatacttatgctttttgtgaaattttatttgcaggtcaagctctctggatcagttagtagccaatatttgactgcactgcttatggctactcctttgggtcttggagacgtggaggttgaaatcattgataaattgatttctgtaccatatgtggagatgacaataaagctaatggaaaggtttggagtgtctgtagagcatagtgctgactggggtaggttcttgatccgaggtggtcagaagtacaagtaagtctctcttttttactacgtgtcctgaaaaatctatgttttagtagactaagatactataaaatacatcagatctcctggaaatgcgtatgttgagggtgatgcttcaagtgctagttacttcataggaggggccgcagtcactggtgggactgtgactgttgagggttgtggaacaagtagtttacaggtatgatttaaagccttatttcacatccttttacttctctccttactgcatctcagcttaattctgaagaactcttgttgctcaatctctaggcataagcttgttctttcgaccttctaaatttgcaaaatcattctgttaatgtaacaaacagaagatgagatggtgtttttgatcctgataaaaaaaattaggacattcatgattggtttatgttctatgacaatttcgatgatctcttcaattgtaacttgaggacccttttctgctagtaaagattgtgtataagctattctagactgttgtatacacctcatattccggtattgtttatacctaataaacttcttatgattagtatcttacttgtctcatgtgttttgcatagggtgatgtaaaatttgctgaagttcttgagaagatgggttgcaaggtatcctggacagagaacagtgtcactgtcactggaccacccagggatgcatctggaagaaaacacttgcgcgccgttgatgtcaacatgaacaaaatgccagatgttgcaatgactcttgctgttgttgctctttatgcagatggacccaccaccattagagacggtatgcttaattcctttctgtgaacatgacactcttcttgtgccataggtagattccgtagttattggtcacaatccaaatttgcattggtatttaaagcaagtcttaggtggtgagaccgcttgaaacccataatataaaagaaaggtcaccttggtctcaaagttaattcttgtgagaccatctcatgcaagattttgccgggtatctaatccttgtttgcactgaggaaaaggaaaaaaattaacatgacctattagtaagccaaaaaaattctaacaagagaagtagccgaataggtaataatttttgtgatataatgatcagtggctagctggagagtgaaggaaacagaacggatgattgcgatttgcacagagctcagaaaggttagccattttggatatacacattcttgaaggttttcaaccttgtagaagattgtactattgataaaaaaaattgatgtttatccggtgtacttcgaaatttcttttcagctgggggcaacagttgaggaaggatcagattactgtgtgatcactccacctgagaaactaaatgtgacggccattgatacatacgatgatcaccgaatggccatggcattctctcttgctgcctgcgccgatgttcctgttaccatcaaggacccgggttgcactcgcaagactttcccagactactttgatgtgttggaaaggtttgcaaagcattaagtggtctcctacatattctataaagcataagctgagattttttgagagaattaggagatgaaaaatgctttctgcttgagttatcatcacattctttgtattatgattgtaagattattatagtatagagtttacaaagtactactaataattgttatgtatccgattgatcagaaataagttaattggaaggctggactttgaaaatgtgaccaagacactagtgtgaccaagtcattttgttaatgtgagttcaatgttattgattcaacatgtagagccaaatctcaattctatcgtcacttcatatgaccaaaaatctaaagatgaaaaagtaaaaaagagcatgttggatcaaactctagctgtatctctgaaattcaatcacgcagttagatcaaatgaggattaaagggagt

SEQ ID NO: 225 - DNA, 210 bp, Cauliflower mosaic virus
tccactgacgtaagggatgacgcacaatcccactatccttcgcaagacccttcctctatataaggaagttcatttcatttggagaggacacgctgaaatcaccagtctctctctacaaatctatctctctctattttctccataataatgtgtgagtagttcccagataagggaattagggttcttatagggtttcgctcacgtgttgag

SEQ ID NO: 226 - DNA, 5009 bp, Beta vulgaris
caaatcttctggcatcaatggcggtgttgccgttcatcaatttaacatcaatggaggtaagagtcatgttttttcaacaatataaaacttatacttcctctgttctgttttaaatgaaacgtttgttttctcacgcaacccaacccacttttttaataataaatatttttagttgtgtgcacgtaaaaaatataaaaaagttataatttgatagtatcttgtttgagattgtgattattaagagagtcaagtctcacaatattcgaaagtctacgtaatccacctcaaattgacgaagaaaacaagcaggaaaggattaagtaagttcgtggaaccactagaattgattttcaaatatagctctacctaatatatggcctacttttaattttaaataaggagaaggtaatgtgattagaaacaaattggtcttaaattattcattaagcttaataatgtataaacataatcaagtgctatcttcttttcagggccgtcttgaagattttggggcccggttctattatgaaaattgggcccctaaatttatagaaaataaagatggaaggttagagttctaaagatagaaagttgaaaatctaaatataaatcattgacaaatttattaagggtgagaaacaagggtgttttcttcaaatatgaagcaaaattttcaaaataatacttcctccgtttctaaataagtgcaacatttgcataatgtttactattcacagtttaaactttaattagctttggtgatttacattttaggaaaaaacatagtcatgtgggatcttgttagattcgtctgaatgtgaatttttttaatatcaactttttataatttttacttattgataattgaagatattaatggttaaaataatgcattggcaaacgtgaaaacaagaagtgttgcacttatttagaaacggaggaagtatattttgggcctttataattttggagaccctgcgctgttgggctccttgcacaccttcatctacccctctgcttcttttgatacaatttttcagcgacatgattgtcgattgatgcatatattattgtatactcgatccatattgtttaagatgaattgtttgtctttgatggtctccaatgcatattttgtatacttaggaattctaattatgtactattagtagacattgagatgaatacataattgccataatgaagtatgattattttagttatatactttctccattccaaatatataaatgtaacacttgtgtactttatgcgtactaatgcataacaacgtgcactctcatgtgtttaattatatactttttgagagaagtgttacattggggaccatgggactgtgtataatttgaccgcaaaatcgaagtgtcgcatttgattgaaaatggagagagtagtatatagatggaacacagcagagactgctggtcatctttggccaacaaacccaaaattgatattaatcccttattcaggtcatttcatctttttgacacaaaatggatgttgtaggcactgcgctatctgctgcccaatctctgtttgcagccctgcaaagttctgagctcaaagagatcctctcgatctttggctacaaatcccgacttgatgacctccaacgcactgtctctaccatcaacgctgtattccgtgatgctgagaccaaacaggagctcactcatgaagcacagcattggctcgaggaactcaaggatgctgtctttgaagcagatgatctgttcgacgagtttgtcactcttgccgagcagaagcaacttgtagaggctggtggcagtctttccaaaaagatgcgccaattcttttctgattccaacccccttggcattgcttataggatgtcacgaggggttaagaagatcaagaagaagttggatgctatcgcttacaatcatcaatttagctttaagattgatcttgagcctatgaaagagagaaggctagagactggttctgtcgtgaacgcaggtgatatcattggaagagaggacgacttggagaagattgtaggtttgttgcttgattctaacatccaacgtgatgtgtctttccttactattgtgggaatgggagggttgggtaaaactgctcttgcccaactcgtgtacaatgatccaagggtcagaactgcttttccattgagatgttggaattgtgtgtctgatcaagatcaaaagcaactagatgtgaaagaaattttgggtaagattctggctacagctactggtaagaatcatgagggttcaaccatggatcaggtgcaaacccaactacgagaacaactatgtggcaagagatacttgcttgttttggatgatgtatggaatgagaatcctaatcaattgcgtgatctggtagaattcttcatgggaggtcgaagcagaaattggattgtggtaactacgcgttcgcacgagacagcgagaattataagagatggtccattgcacaagctccaaggtttgtctgaggaaaactcttggcgtttatttgtcaggtggaccttcggatcagtgcaagcaaaattccctaatgactttatcatgattgcacgagatatagttgacaaatgtgctcgaaaccctctggctataagagtggtaggaagtcttttgtgtggtcaagacaagagtaagtggctttcatttcatgagatcgatttaggcaacattagaaagagccataatgatatcatgccaatactgaacctaagttaccatcatcttgaacctccaattaagagatgctttagttattgtgcagtgtttccaaaggatttccttatagggaagcagacgctgataaacctctggatggcacaaggttatattgttccgttagacaaagatcaaagcatagatgatgctagtgaggaatacatatcaattttgttgcggagatgttttttcgaaaatgtcggagcagaaaaagatggtgttattaagatccatgatctcatgcatgatattgctcaaaatgtcatggggaaggagctttgtacgactaaaaacattagtggcagcttggataaaagtgttcgccatctatctcttgccagaactagttttgcaagatactctttcaatgcaactcatattcgctcctatttctgtgctggctactggtgtcaggatgctgagataaaccagttttcagttgaggcattagtaccaaactgtttgtacctaagggcaatggacctcgcttggtcgaagataaaaagtttaccagactcgattggtggattgttgcatttgaggtacttagatctttcgtataacgaagatctggaagtacttccaaactcaattgctaaactatataatctacaaaccttacaattgaagggttgcaagagattggaagggttaccaaaacatttgagcaggctggttaagcttcaaactttggatatacatggttgcaacaatgtaacttatatgcccaaaggcatgggtaagttgacttgccttcacactctcagtaagtttatagtgggtggagaagggagttgttcaagttggaagcaatgttttgatgggttggaagatctaaaggctctcaataacctaaagggtcatctggaaatccaaatcaggtggcccaaaaatactacagatgctgtcaaagaagatgttacgagggaaggattatacctgaatcataaggaacatctcaatcacattgtggttgattttagatgtgaggagggtggtggaagaatggatgatgaggaagcaagaagattgatggaagagttgcggccacatccttatcttgaaaatttggctgtgaaagcatattatggtgtgaaaatgcctggttgggcaacccttctcccaaatcttacagagctttttctttctgattgtggggaactggagaaccttccatgcctgggaaacttggatcatctaaaagtcctccgactttcgcatttggcaaaattggagtacattgaagaagatagctcatcagctaatttcaggtgtaggcctggaccagaaagtgcaggactatcattatacttcccctcccttgaacgccttgagttgaagcgtttgtgtaagttaaaaggatggaggagaggggaagggttaggagatgatcaccagccttttaatgaaagcagcagcaatacacaagtccaattacaattatgtcttcctcaattgaagtcattgagaatagaaagatgcccattgctgacatttatgccgctgtgtcccaagacagaaaaactgcatttagttgtatttaatgaacgactccggatagtgcatgctaagagagatgagaatttctatgctccattacattcatcatcatctgatcctgaaaacccgaggaacactattcccattcccatgtttagagaggtatacataaacaatgtggcgtggctaaattcgctgcctatggaggcttttaggtgtctcactcatatgacaataaaaaacgacgaggtagagagtttgggagaagttggagaggtgtttcggagctgctcatcttctttgcgatccttgaatatcacaggttgctccaacttaagaagtgtttctggagggctggagcatctcactgctttggagatgttagaaatatacgacacccataagctgagtctatcagaagacccagaaggtgttgtgccatggaaatcccttcatcactccctcagctacttgcaactgatgaatctcccacagctggtcaacctgcctgattcgatgcagttcttggctgccctccgaactctttcaatagtgcattgcactaaactgcaatcagtgccagattggatgcccagactcacttctctcaggaagcttatggtttcattctgttccgcacatctggagagaagatgccaaaatccaactggggtggactggcctaacattcaacacatcccctccattgatgtcacctctagccttcctaagtttttagtgttgccgtatgaatag

SEQ ID NO: 227 - protein, 1163 aa, Beta vulgaris
MDVVGTALSAAQSLFAALQSSELKEILSIFGYKSRLDDLQRTVSTINAVFRDAETKQELTHEAQHWLEELKDAVFEADDLFDEFVTLAEQKQLVEAGGSLSKKMRQFFSDSNPLGIAYRMSRGVKKIKKKLDAIAYNHQFSFKIDLEPMKERRLETGSVVNAGDIIGREDDLEKIVGLLLDSNIQRDVSFLTIVGMGGLGKTALAQLVYNDPRVRTAFPLRCWNCVSDQDQKQLDVKEILGKILATATGKNHEGSTMDQVQTQLREQLCGKRYLLVLDDVWNENPNQLRDLVEFFMGGRSRNWIVVTTRSHETARIIRDGPLHKLQGLSEENSWRLFVRWTFGSVQAKFPNDFIMIARDIVDKCARNPLAIRVVGSLLCGQDKSKWLSFHEIDLGNIRKSHNDIMPILNLSYHHLEPPIKRCFSYCAVFPKDFLIGKQTLINLWMAQGYIVPLDKDQSIDDASEEYISILLRRCFFENVGAEKDGVIKIHDLMHDIAQNVMGKELCTTKNISGSLDKSVRHLSLARTSFARYSFNATHIRSYFCAGYWCQDAEINQFSVEALVPNCLYLRAMDLAWSKIKSLPDSIGGLLHLRYLDLSYNEDLEVLPNSIAKLYNLQTLQLKGCKRLEGLPKHLSRLVKLQTLDIHGCNNVTYMPKGMGKLTCLHTLSKFIVGGEGSCSSWKQCFDGLEDLKALNNLKGHLEIQIRWPKNTTDAVKEDVTREGLYLNHKEHLNHIVVDFRCEEGGGRMDDEEARRLMEELRPHPYLENLAVKAYYGVKMPGWATLLPNLTELFLSDCGELENLPCLGNLDHLKVLRLSHLAKLEYIEEDSSSANFRCRPGPESAGLSLYFPSLERLELKRLCKLKGWRRGEGLGDDHQPFNESSSNTQVQLQLCLPQLKSLRIERCPLLTFMPLCPKTEKLHLVVFNERLRIVHAKRDENFYAPLHSSSSDPENPRNTIPIPMFREVYINNVAWLNSLPMEAFRCLTHMTIKNDEVESLGEVGEVFRSCSSSLRSLNITGCSNLRSVSGGLEHLTALEMLEIYDTHKLSLSEDPEGVVPWKSLHHSLSYLQLMNLPQLVNLPDSMQFLAALRTLSIVHCTKLQSVPDWMPRLTSLRKLMVSFCSAHLERRCQNPTGVDWPNIQHIPSIDVTSSLPKFLVLPYE

SEQ ID NO: 228 - protein, 1177 aa, Beta vulgaris
MERVVYRWNTAETAGHLWPTNPKLILIPYSALQSSELKEILSIFGYKSRLDDLQRTVSTINAVFRDAETKQELTHEAQHWLEELKDAVFEADDLFDEFVTLAEQKQLVEAGGSLSKKMRQFFSDSNPLGIAYRMSRGVKKIKKKLDAIAYNHQFSFKIDLEPMKERRLETGSVVNAGDIIGREDDLEKIVGLLLDSNIQRDVSFLTIVGMGGLGKTALAQLVYNDPRVRTAFPLRCWNCVSDQDQKQLDVKEILGKILATATGKNHEGSTMDQVQTQLREQLCGKRYLLVLDDVWNENPNQLRDLVEFFMGGRSRNWIVVTTRSHETARIIRDGPLHKLQGLSEENSWRLFVRWTFGSVQAKFPNDFIMIARDIVDKCARNPLAIRVVGSLLCGQDKSKWLSFHEIDLGNIRKSHNDIMPILNLSYHHLEPPIKRCFSYCAVFPKDFLIGKQTLINLWMAQGYIVPLDKDQSIDDASEEYISILLRRCFFENVGAEKDGVIKIHDLMHDIAQNVMGKELCTTKNISGSLDKSVRHLSLARTSFARYSFNATHIRSYFCAGYWCQDAEINQFSVEALVPNCLYLRAMDLAWSKIKSLPDSIGGLLHLRYLDLSYNEDLEVLPNSIAKLYNLQTLQLKGCKRLEGLPKHLSRLVKLQTLDIHGCNNVTYMPKGMGKLTCLHTLSKFIVGGEGSCSSWKQCFDGLEDLKALNNLKGHLEIQIRWPKNTTDAVKEDVTREGLYLNHKEHLNHIVVDFRCEEGGGRMDDEEARRLMEELRPHPYLENLAVKAYYGVKMPGWATLLPNLTELFLSDCGELENLPCLGNLDHLKVLRLSHLAKLEYIEEDSSSANFRCRPGPESAGLSLYFPSLERLELKRLCKLKGWRRGEGLGDDHQPFNESSSNTQVQLQLCLPQLKSLRIERCPLLTFMPLCPKTEKLHLVVFNERLRIVHAKRDENFYAPLHSSSSDPENPRNTIPIPMFREVYINNVAWLNSLPMEAFRCLTHMTIKNDEVESLGEVGEVFRSCSSSLRSLNITGCSNLRSVSGGLEHLTALEMLEIYDTHKLSLSEDPEGVVPWKSLHHSLSYLQLMNLPQLVNLPDSMQFLAALRTLSIVHCTKLQSVPDWMPRLTSLRKLMVSFCSAHLERRCQNPTGVDWPNIQHIPSIDVTSSLPKFLVLPYE